(19) United States
(12) Patent Application Publication
(10) Pub. No.: US 2025/0082607 A2
(43) Pub. Date: Mar. 13, 2025

(54) METHODS OF TREATING DISORDERS WITH A PSILOCYBIN ANALOG

(71) Applicant: Cybin IRL Limited, Dublin (IE)

(72) Inventors: Alex Nivorozhkin, West Roxbury, MA (US); Michael Palfreyman, St. Petersburg, FL (US); Pradip M. Pathare, Lexington, MA (US); Kenneth L. Avery, Merrimack, NH (US); Mohammed I. Shukoor, Lexington, MA (US); Michael E. Morgan, San Diego, CA (US); Joan M. Krakowsky, North Palm Beach, FL (US); Amir Inamdar, Tiptree, Essex (UK); Pradeep J. Nathan, Cambridge (UK); Sebastian Krempien, Darmstadt (DE)

(73) Assignee: Cybin IRL Limited, Dublin (IE)

(21) Appl. No.: 18/588,132

(22) Filed: Feb. 27, 2024

Related U.S. Application Data
(60) Provisional application No. 63/553,321, filed on Feb. 14, 2024.
(60) Provisional application No. 63/603,886, filed on Nov. 29, 2023.
(60) Provisional application No. 63/603,262, filed on Nov. 28, 2023.
(60) Provisional application No. 63/602,888, filed on Nov. 27, 2023.
(60) Provisional application No. 63/519,992, filed on Aug. 16, 2023.
(60) Provisional application No. 63/512,466, filed on Jul. 7, 2023.
(60) Provisional application No. 63/487,078, filed on Feb. 27, 2023.

(51) Int. Cl. A61K 31/4045; A61K 9/00; A61P 25/24

(52) U.S. Cl. CPC A61K 31/4045; A61K 9/0053; A61P 25/24

(57) ABSTRACT

The present disclosure relates generally to methods of treating various diseases, disorders, and conditions, such as depressive disorders (e.g., Major Depressive Disorder (MDD)), substance use disorders, anxiety disorders, eating disorders, pain, and headache disorders via administration of deuterated psilocin and pharmaceutically acceptable salts, polymorphs, or solvates thereof.

### CROSS-REFERENCE

This application claims priority to U.S. Provisional Application No. 63/487,078, filed on Feb. 27, 2023, U.S. Provisional Application No. 63/512,466, filed on Jul. 7, 2023, U.S. Provisional Application No. 63/519,992, filed on Aug. 16, 2023, U.S. Provisional Application No. 63/602,888, filed on Nov. 27, 2023, U.S. Provisional Application No. 63/603,262, filed on Nov. 28, 2023, U.S. Provisional Application No. 63/603,886, filed on Nov. 29, 2023, and U.S. Provisional Application No. 63/553,321, filed on Feb. 14, 2024, each incorporated herein by reference in their entireties.

### FIELD

The present disclosure relates generally to methods of using deuterated psilocin and pharmaceutically acceptable salts, polymorphs, or solvates thereof, in the treatment of various diseases, disorders, and conditions, such as depressive disorders (e.g., Major Depressive Disorder (MDD)), substance use disorders, anxiety disorders, eating disorders, pain, and headache disorders.

### BACKGROUND

Major Depressive Disorder (MDD) is the leading cause of disability worldwide (Cosgrove 2020), and results in a significant economic and societal cost (Vigo D, Thornicroft G, Atun R (2016). Estimating the true global burden of mental illness. The Lancet Psychiatry, 3(2), 171-178). In the United States, MDD is a significant public health issue, with an estimated 21.0 million adults having at least one major depressive episode in 2020, which represents 8.4% of all US adults (National Institute of Mental Health, 2020 Statistics: Major Depression. Updated January 2022. https://www.nimh.nih.gov/health/statistics/major-depression; accessed: 22 Apr. 2022). The prevalence of major depressive episode was higher among adult females (10.5%) compared to males (6.2%) and was highest among individuals aged 18-25 years (17.0%).

While multiple medications and psychological interventions are available to treat MDD, up to 30% of patients do not respond to first line treatment with up to 30% not benefitting despite multiple treatments (treatment resistant) (Rizvi S J, Grima E, Tan M, et al. (2014). Treatment-resistant depression in primary care across Canada. Can J Psychiatry, 59, 349-357; Rush A J, Trivedi M H, Wisniewski S R, et al. (2006). Acute and longer-term outcomes in depressed outpatients requiring one or several treatment steps: A STAR*D Report. Am J Psychiatry, 163, 1905-1917). Further, available antidepressant medications require daily dosing, have a slow onset of action, take several weeks to show any beneficial effect (Cipriani A, Furukawa T A, Salanti G, et al. (2018). Comparative efficacy and acceptability of 21 antidepressant drugs for the acute treatment of adults with major depressive disorder: a systematic review and network meta-analysis. Lancet, 391, 1357-1366), require long term treatment, and are associated with dose-limiting side effects such as gastrointestinal (GI) disturbances, sedation, and sexual dysfunction (FDA 2019). There is therefore a significant unmet need for treatments that start working swiftly, are well tolerated, and are not required to be taken long term.

Treatments for MDD have traditionally focused on the monoamine transmitters, preventing the reuptake of serotonin and/or norepinephrine and thereby increasing their availability at the synaptic cleft. While this has been met with some success, these treatments are symptomatic and do not address the underlying biopsychosocial causes. The resurrection of research into serotonergic psychedelic medicines offers the opportunity to explore an alternative approach where the use of psychedelics opens a therapeutic window to facilitate insight and, with psychotherapeutic support, carry out the necessary emotional work to improve depressive symptomatology (Nutt D, Erritzoe D, Carhart-Harris R (2020). Psychedelic Psychiatry's Brave New World. Cell, 181, 24-28).

Psilocybin (PY) and psilocin (PI) are tryptamine alkaloids and structural analogs of the neurotransmitter serotonin. Psilocybin is a prodrug of psilocin. That is, when consumed, psilocybin is rapidly metabolized into the active form, psilocin (4-hydroxy-N,N-dimethyltryptamine). Specifically, a chemical process called dephosphorylation removes the phosphate group on psilocybin, creating psilocin.

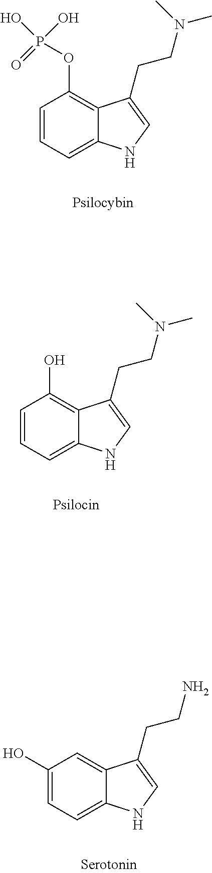 [SMILES: CN(C)CCc1c[nH]c2cccc(OP(=O)(O)O)c12 ; CN(C)CCc1c[nH]c2cccc(O)c12 ; NCCc1c[nH]c2ccc(O)cc12]

Outside the body, psilocin is reported to be a short-lived and unstable molecule. For this reason, psilocin has been rarely studied and not generally recognized as a viable therapeutic option. Vaupel et al. studied the effects of psilocin ascorbate on food intake on dogs (D. B. Vaupel, M. Nozaki, W. R. Martin, L. D. Bright, E. C. Morton, The inhibition of food intake in the dog by LSD, mescaline, psilocin, d-amphetamine and phenylisopropylamine derivatives, Life Sciences, Volume 24, Issue 26, 1979, 2427-2431).

Migliaccio et al. studied the solution confirmation of psilocin monooxalate in water (Gerald P. Migliaccio, Tiee-Leou N. Shieh, Stephen R. Byrn, Bruce A. Hathaway, and David E. Nichols, Comparison of solution conformational preferences for the hallucinogens bufotenin and psilocin using 360-MHz proton NMR spectroscopy, Journal of Medicinal Chemistry, 1981 24, 2, 206-209).

Aghajanian et al. studied the effects of psilocin tartrate on serotonergic neurons in rats using microiontophoretic techniques (Aghajanian G K, Hailgler H J. Hallucinogenic indoleamines: Preferential action upon presynaptic serotonin receptors. Psychopharmacol Commun. 1975, 1, 6, 619-29).

Kuhnert-Brandstatter et al. describe the preparation of three polymorphs of psilocin (Kuhnert, M. et al., Polymorphe Modifikationen und Solvate von Psilocin und Psilocybin [Polymorphic Modifications and Solvates of Psilocin and Psilocybin], 1976, Archiv der Pharmazie, 309:625-631).

U.S. Pat. No. 11,312,684 B1 describes psilocin salts with improved physical properties and handling characteristics.

Therefore, therapeutic applications involving the use of psilocin are generally accomplished by administration of the precursor, psilocybin, or other prodrug approaches. However, psilocybin has slow onset and a long duration of drug action, often requiring 7-8 hours of supervised clinical observation of a patient before discharge. Psilocybin is also associated with high levels of variability in delivery as it requires metabolism to release the active. There is thus a need for new treatment options that overcome the limitations of psilocybin and related prodrugs.

### SUMMARY

The present disclosure is based at least in part on methods of treating various diseases, disorders, and conditions with stabilized forms of psilocin, including psilocin-d₁₀ and novel crystalline forms/polymorphs thereof, novel salt forms of psilocin-d₁₀ and novel crystalline forms/polymorphs thereof, as well as compositions thereof. More specifically, the present disclosure provides methods of treating depressive disorders (e.g., Major Depressive Disorder (MDD)), substance use disorders, anxiety disorders, eating disorders, and headache disorders with stabilized forms of psilocin-d₁₀, through various dosing regimens including one or two dose regimens (e.g., two doses separated by three weeks in a treatment course, etc.).

The disclosed stabilized forms of psilocin such as psilocin-d₁₀ do not rely on prodrug metabolism for release of active agent, as is the case with psilocybin administration or related prodrug approaches, and thus can provide a faster/quicker therapeutic onset, a shorter duration of drug action (e.g., short duration of effect), and less inter-subject variability. Accordingly, human clinical trials (ClinicalTrials.gov Identifier: NCT05385783) were initiated based upon these characteristics and potential advantages over psilocybin or related prodrug approaches. During these clinical trials, it was discovered that treatment involving psilocin-d₁₀ or a pharmaceutically acceptable salt, polymorph, or solvate thereof, provides unexpected levels of efficacy in treating the MDD patient population, e.g., in terms of effect size. Additionally, the unexpected efficacy is achieved at surprisingly low dosage levels—far lower dosages than the inventors could have predicted based on pre-clinical and clinical simulation studies. These discoveries led the therapy to be granted breakthrough therapy designation (BTD) by the U.S. Food and Drug Administration (FDA) for the treatment of MDD.

Thus, the present disclosure provides:

(1) A method of treating a depressive disorder in a subject in need thereof, comprising administering orally to the subject about 8 mg to about 16 mg (free base equivalence) of a compound of Formula (I-3)

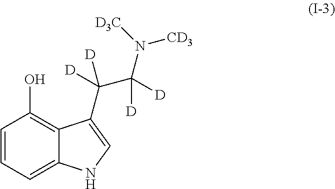 [SMILES: [2H]C([2H])([2H])N(C([2H])([2H])[2H])C([2H])([2H])C([2H])([2H])c1c[nH]c2cccc(O)c12]

or a pharmaceutically acceptable salt, polymorph, or solvate thereof.

(2) The method of (1), comprising administering about 12 mg to about 16 mg (free base equivalence) of the compound of Formula (I-3), or a pharmaceutically acceptable salt, polymorph, or solvate thereof, to the subject.

(3) The method of (1), comprising administering about 12 mg (free base equivalence) of the compound of Formula (I-3), or a pharmaceutically acceptable salt, polymorph, or solvate thereof, to the subject.

(4) The method of (1), comprising administering about 14 mg (free base equivalence) of the compound of Formula (I-3), or a pharmaceutically acceptable salt, polymorph, or solvate thereof, to the subject.

(5) The method of (1), comprising administering about 16 mg (free base equivalence) of the compound of Formula (I-3), or a pharmaceutically acceptable salt, polymorph, or solvate thereof, to the subject.

(6) The method of any one of (1) to (5), wherein a pharmaceutically acceptable salt of the compound of Formula (I-3) is administered.

(7) The method of (6), wherein the pharmaceutically acceptable salt of the compound of Formula (I-3) is selected from the group consisting of a benzenesulfonate, a tartrate, a hemi-fumarate, an acetate, a citrate, a malonate, a fumarate, a succinate, an oxalate, a benzoate, a salicylate, an ascorbate, a hydrochloride, a maleate, a malate, a methanesulfonate, a toluenesulfonate, a glucuronate, and a glutarate salt, of the compound of Formula (I-3).

(8) The method of (6) or (7), wherein the pharmaceutically acceptable salt of the compound of Formula (I-3) is a benzenesulfonate salt of the compound of Formula (I-3).

(9) The method of (8), wherein the benzenesulfonate salt of the compound of Formula (I-3) is a crystalline benzenesulfonate salt (I-3a), characterized by an X-ray powder diffraction pattern containing at least three characteristic peaks selected from 7.023, 7.767, 11.822, 12.550, 12.860, 13.994, 15.521, 18.436, 19.503, 20.760, 21.070, 22.007, 22.745, 23.340, 24.187, 25.532, 26.880, 27.856, 28.163, 31.267, 33.024, 35.030, 36.835, 39.312, 40.545, and 40.988 °2θ (±0.2° 2θ).

(10) The method of (6) or (7), wherein the pharmaceutically acceptable salt of the compound of Formula (I-3) is a tartrate salt of the compound of Formula (I-3).

(11) The method of (10), wherein the tartrate salt of the compound of Formula (I-3) is a crystalline tartrate salt (I-3b), characterized by an X-ray powder diffraction pattern containing at least three characteristic peaks selected from 6.732, 12.708, 13.470, 14.774, 15.921, 16.268, 17.295, 18.869, 20.079, 20.208, 20.877, 21.894, 22.657, 23.491, 23.702, 24.636, 24.882, 25.569, 26.685, 27.060, 27.502, 28.179, 28.597, 29.035, 29.257, 29.527, 31.017, 31.527, 32.059, 32.307, 33.012, 34.024, 34.388, 34.905, 35.361, 36.183, 37.372, 37.764, 38.657, and 41.049 °2θ (±0.2° 2θ).

(12) The method of (6) or (7), wherein the pharmaceutically acceptable salt of the compound of Formula (I-3) is a hemi-fumarate salt of the compound of Formula (I-3).

(13) The method of (12), wherein the hemi-fumarate salt of the compound of Formula (I-3) is a crystalline hemi-fumarate salt (I-3c), characterized by an X-ray powder diffraction pattern containing at least three characteristic peaks selected from 9.713, 11.209, 11.605, 12.338, 12.852, 13.718, 15.117, 16.066, 16.627, 19.026, 19.427, 20.108, 21.068, 21.335, 21.837, 22.429, 23.262, 23.478, 23.900, 24.720, 25.318, 27.912, 28.532, 29.565, 30.457°, 32.698, 34.155, 37.910, 39.566, and 40.999 °2θ (±0.2° 2θ).

(14) The method of (6) or (7), wherein the pharmaceutically acceptable salt of the compound of Formula (I-3) is a citrate salt of the compound of Formula (I-3).

(15) The method of (14), wherein the citrate salt (I-3e) is amorphous by X-ray powder diffraction.

(16) The method of (6) or (7), wherein the pharmaceutically acceptable salt of the compound of Formula (I-3) is a benzoate salt of the compound of Formula (I-3).

(17) The method of (16), wherein the benzoate salt of the compound of Formula (I-3) is a crystalline benzoate salt (I-3j), characterized by an X-ray powder diffraction pattern containing at least three characteristic peaks selected from 9.486, 11.006, 12.379, 13.428, 14.608, 15.446, 16.389, 18.247, 18.977, 19.346, 19.831, 20.868, 21.447, 22.860, 23.878, 24.944, 25.737, 26.144, 26.341, 26.990°, 27.708, 28.595, 30.048, 30.763, 31.127, 31.839, 32.800, 34.460, 35.444, 37.725, and 38.597°2θ (±0.2° 2θ).

(18) The method of any one of (1) to (17), wherein the depressive disorder is major depressive disorder (MDD).

(19) The method of (18), wherein, prior to treatment, the subject has been diagnosed with moderate to severe major depressive disorder as defined by the Diagnostic and Statistical Manual of Mental Disorders, 5th edition (DSM-5).

(20) The method of any one of (1) to (19), wherein, prior to treatment, the subject has scored greater than or equal to 21 on the Montgomery-Åsberg Depression Scale (MADRS).

(21) The method of any one of (1) to (20), wherein a first dose and a second dose, each being about 8 mg to about 16 mg (free base equivalence) of the compound of Formula (I-3), or a pharmaceutically acceptable salt, polymorph, or solvate thereof, are administered to the subject one to four weeks apart (±3 days).

(22) The method of any one of (1) to (Error! Reference source not found.), wherein a first dose and a second dose, each being about 8 mg to about 16 mg (free base equivalence) of the compound of Formula (I-3), or a pharmaceutically acceptable salt, polymorph, or solvate thereof, are administered to the subject three weeks apart (±3 days).

(23) The method of any one of (1) to (22), wherein the subject is taking antidepressant medication as part of ongoing treatment and the method is used as adjunctive therapy.

(24) A pharmaceutical composition, comprising:
about 8 mg to about 16 mg (free base equivalence) of a compound of Formula (I-3)

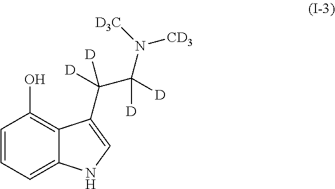 [SMILES: [2H]C([2H])([2H])N(C([2H])([2H])[2H])C([2H])([2H])C([2H])([2H])c1c[nH]c2cccc(O)c12]

or a pharmaceutically acceptable salt, polymorph, or solvate thereof; and
a pharmaceutically acceptable vehicle.

(25) The pharmaceutical composition of (24), wherein the pharmaceutical composition comprises about 12 mg to about 16 mg (free base equivalence) of the compound of Formula (I-3), or a pharmaceutically acceptable salt, polymorph, or solvate thereof.

(26) The pharmaceutical composition of (24), wherein the pharmaceutical composition comprises about 12 mg (free base equivalence) of the compound of Formula (I-3), or a pharmaceutically acceptable salt, polymorph, or solvate thereof.

(27) The pharmaceutical composition of (24), wherein the pharmaceutical composition comprises about 14 mg (free base equivalence) of the compound of Formula (I-3), or a pharmaceutically acceptable salt, polymorph, or solvate thereof.

(28) The pharmaceutical composition of (24), wherein the pharmaceutical composition comprises about 16 mg (free base equivalence) of the compound of Formula (I-3), or a pharmaceutically acceptable salt, polymorph, or solvate thereof.

(29) The pharmaceutical composition of any one of (24) to (28), wherein the pharmaceutical composition comprises a pharmaceutically acceptable salt of the compound of Formula (I-3).

(30) The pharmaceutical composition of (29), wherein the pharmaceutically acceptable salt of the compound of Formula (I-3) is selected from the group consisting of a benzenesulfonate, a tartrate, a hemi-fumarate, an acetate, a citrate, a malonate, a fumarate, a succinate, an oxalate, a benzoate, a salicylate, an ascorbate, a hydrochloride, a maleate, a malate, a methanesulfonate, a toluenesulfonate, a glucuronate, and a glutarate salt, of the compound of Formula (I-3).

(31) The pharmaceutical composition of (29) or (30), wherein the pharmaceutically acceptable salt of the compound of Formula (I-3) is a benzenesulfonate salt of the compound of Formula (I-3).

(32) The pharmaceutical composition of (31), wherein the benzenesulfonate salt of the compound of Formula (I-3) is a crystalline benzenesulfonate salt (I-3a), characterized by an X-ray powder diffraction pattern containing at least three characteristic peaks selected from 7.023, 7.767, 11.822, 12.550, 12.860, 13.994, 15.521, 18.436, 19.503, 20.760, 21.070, 22.007, 22.745, 23.340, 24.187, 25.532, 26.880, 27.856, 28.163, 31.267, 33.024, 35.030, 36.835, 39.312, 40.545, and 40.988 °2θ (±0.2° 2θ).

(33) The pharmaceutical composition of (29) or (30), wherein the pharmaceutically acceptable salt of the compound of Formula (I-3) is a tartrate salt of the compound of Formula (I-3).

(34) The pharmaceutical composition of (33), wherein the tartrate salt of the compound of Formula (I-3) is a crystalline tartrate salt (I-3b), characterized by an X-ray powder diffraction pattern containing at least three characteristic peaks selected from 6.732, 12.708, 13.470, 14.774, 15.921, 16.268, 17.295, 18.869, 20.079, 20.208, 20.877, 21.894, 22.657, 23.491, 23.702, 24.636, 24.882, 25.569, 26.685, 27.060, 27.502, 28.179, 28.597, 29.035, 29.257, 29.527, 31.017, 31.527, 32.059, 32.307, 33.012, 34.024, 34.388, 34.905, 35.361, 36.183, 37.372, 37.764, 38.657, and 41.049 °2θ (10.2 °2θ).

(35) The pharmaceutical composition of (29) or (30), wherein the pharmaceutically acceptable salt of the compound of Formula (I-3) is a hemi-fumarate salt of the compound of Formula (I-3).

(36) The pharmaceutical composition of (35), wherein the hemi-fumarate salt of the compound of Formula (I-3) is a crystalline hemi-fumarate salt (I-3c), characterized by an X-ray powder diffraction pattern containing at least three characteristic peaks selected from 9.713, 11.209, 11.605, 12.338, 12.852, 13.718, 15.117, 16.066, 16.627, 19.026, 19.427, 20.108, 21.068, 21.335, 21.837, 22.429, 23.262, 23.478, 23.900, 24.720, 25.318, 27.912, 28.532, 29.565, 30.457°, 32.698, 34.155, 37.910, 39.566, and 40.999°2θ (±0.2° 2θ).

(37) The pharmaceutical composition of (29) or (30), wherein the pharmaceutically acceptable salt of the compound of Formula (I-3) is a citrate salt of the compound of Formula (I-3).

(38) The pharmaceutical composition of (37), wherein the citrate salt (I-3e) is amorphous by X-ray powder diffraction.

(39) The pharmaceutical composition of (29) or (30), wherein the pharmaceutically acceptable salt of the compound of Formula (I-3) is a benzoate salt of the compound of Formula (I-3).

(40) The pharmaceutical composition of (39), wherein the benzoate salt of the compound of Formula (I-3) is a crystalline benzoate salt (I-3j), characterized by an X-ray powder diffraction pattern containing at least three characteristic peaks selected from 9.486, 11.006, 12.379, 13.428, 14.608, 15.446, 16.389, 18.247, 18.977, 19.346, 19.831, 20.868, 21.447, 22.860, 23.878, 24.944, 25.737, 26.144, 26.341, 26.990°, 27.708, 28.595, 30.048, 30.763, 31.127, 31.839, 32.800, 34.460, 35.444, 37.725, and 38.597°2θ (±0.2° 2θ).

(41) The pharmaceutical composition of any one of (24) to (40), wherein the pharmaceutically acceptable vehicle comprises an organic acid agent.

(42) The pharmaceutical composition of (41), wherein the organic acid agent is citric acid.

(43) The pharmaceutical composition of (41) or (42), wherein the organic acid agent is present in the pharmaceutical composition in an amount of at least 2% by weight and up to 10% by weight, based on a total weight of the pharmaceutical composition (on a dry basis).

(44) The pharmaceutical composition of any one of (24) to (43), wherein the pharmaceutical composition is in a solid dosage form.

(45) The pharmaceutical composition of (44), wherein the solid dosage form is a solid dosage form adapted for oral administration.

(46) The pharmaceutical composition of (44) or (45), wherein the solid dosage form is a powder in capsule dosage form.

(47) The pharmaceutical composition of any one of (24) to (43), wherein the pharmaceutical composition is an oral liquid dosage form.

(48) A method of treating a depressive disorder in a subject in need thereof, comprising administering to the subject the pharmaceutical composition of any one of (24) to (47).

(49) The method of (48), wherein the pharmaceutical composition is administered orally to the subject.

(50) The method of (48) or (49), wherein the pharmaceutical composition is administered by reconstituting the pharmaceutical composition in solid dosage form in a pharmaceutically acceptable aqueous medium to form an oral liquid dosage form, followed by administering orally to the subject the oral liquid dosage form.

(51) The method of any one of (48) to (50), wherein the depressive disorder is major depressive disorder (MDD).

(52) The method of (51), wherein, prior to treatment, the subject has been diagnosed with moderate to severe major depressive disorder as defined by the Diagnostic and Statistical Manual of Mental Disorders, 5th edition (DSM-5).

(53) The method of any one of (48) to (52), wherein, prior to treatment, the subject has scored greater than or equal to 21 on the Montgomery-Åsberg Depression Scale (MADRS).

(54) The method of any one of (48) to (53), wherein a first dose and a second dose of the pharmaceutical composition, each comprising about 8 mg to about 16 mg (free base equivalence) of the compound of Formula (I-3), or a pharmaceutically acceptable salt, polymorph, or solvate thereof, are administered to the subject one to four weeks apart (±3 days).

(55) The method of any one of (48) to (54), wherein a first dose and a second dose of the pharmaceutical composition, each comprising about 8 mg to about 16 mg (free base equivalence) of the compound of Formula (I-3), or a pharmaceutically acceptable salt, polymorph, or solvate thereof, are administered to the subject three weeks apart (±3 days).

(56) The method of any one of (48) to (55), wherein the subject is taking antidepressant medication as part of ongoing treatment and the method is used as adjunctive therapy.

(57) An adjunctive therapy method for treating a depressive disorder in a subject taking an antidepressant medication as part of ongoing treatment, the adjunctive therapy method comprising administering to the subject about 8 mg to about 16 mg (free base equivalence) of a compound of Formula (I-3)

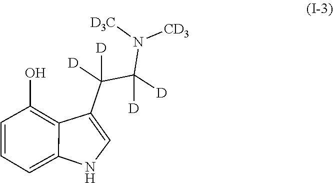 [SMILES: [2H]C([2H])([2H])N(C([2H])([2H])[2H])C([2H])([2H])C([2H])([2H])c1c[nH]c2cccc(O)c12]

or a pharmaceutically acceptable salt, polymorph, or solvate thereof.

(58) The adjunctive therapy method of (57), wherein the depressive disorder is major depressive disorder (MDD).

(59) The adjunctive therapy method of (58), wherein, prior to the adjunctive therapy, the subject has been diagnosed with moderate to severe major depressive disorder as defined by the Diagnostic and Statistical Manual of Mental Disorders, 5th edition (DSM-5).

(60) The adjunctive therapy method of any one of (57) to (59), wherein, prior to the adjunctive therapy, the subject has scored greater than or equal to 21 on the Montgomery-Åsberg Depression Scale (MADRS).

(61) The adjunctive therapy method of any one of (57) to (60), wherein a first dose and a second dose, each being about 8 mg to about 16 mg (free base equivalence) of the compound of Formula (I-3), or a pharmaceutically acceptable salt, polymorph, or solvate thereof, are administered to the subject one to four weeks apart (=3 days).

(62) The adjunctive therapy method of any one of (57) to (61), wherein a first dose and a second dose, each being about 8 mg to about 16 mg (free base equivalence) of the compound of Formula (I-3), or a pharmaceutically acceptable salt, polymorph, or solvate thereof, are administered to the subject three weeks apart (±3 days).

(63) The adjunctive therapy method of any one of (57) to (62), wherein the antidepressant medication is a selective serotonin reuptake inhibitor (SSRI), a serotonin and noradrenaline reuptake inhibitor (SNRI), or a combination thereof.

(64) The adjunctive therapy method of any one of (57) to (63), wherein the compound of Formula (I-3), or a pharmaceutically acceptable salt, polymorph, or solvate thereof, is administered orally to the subject.

(65) A method of treating major depressive disorder (MDD) in a subject in need thereof, comprising administering orally to the subject a capsule comprising about 8 mg to about 16 mg (free base equivalence) of a benzenesulfonate salt of a compound of Formula (I-3)

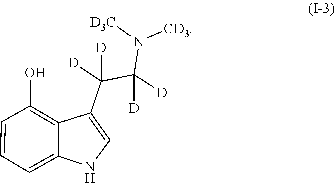 [SMILES: [2H]C([2H])([2H])N(C([2H])([2H])[2H])C([2H])([2H])C([2H])([2H])c1c[nH]c2cccc(O)c12]

(66) A method of treating major depressive disorder (MDD) in a subject in need thereof, comprising administering orally to the subject an oral liquid dosage form comprising:
(i) about 8 mg to about 16 mg (free base equivalence) of a compound of Formula (I-3)

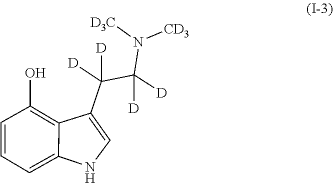 [SMILES: [2H]C([2H])([2H])N(C([2H])([2H])[2H])C([2H])([2H])C([2H])([2H])c1c[nH]c2cccc(O)c12]

or a pharmaceutically acceptable salt, polymorph, or solvate thereof; and
(ii) a pharmaceutically acceptable aqueous medium.

(67) A medicament comprising about 8 mg to about 16 mg (free base equivalence) of a compound of Formula (I-3)

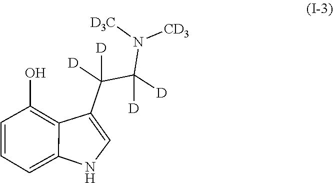 [SMILES: [2H]C([2H])([2H])N(C([2H])([2H])[2H])C([2H])([2H])C([2H])([2H])c1c[nH]c2cccc(O)c12]

or a pharmaceutically acceptable salt, polymorph, or solvate thereof, for use in therapy, such as for treating a subject with a depressive disorder, preferably major depressive disorder (MDD).

(68) Use of a medicament comprising about 8 mg to about 16 mg (free base equivalence) of a compound of Formula (I-3)

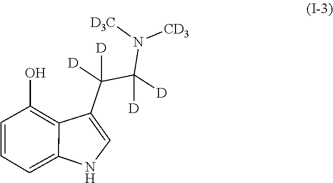 [SMILES: [2H]C([2H])([2H])N(C([2H])([2H])[2H])C([2H])([2H])C([2H])([2H])c1c[nH]c2cccc(O)c12]

or a pharmaceutically acceptable salt, polymorph, or solvate thereof, for therapy, such as for treating a subject with a depressive disorder, preferably major depressive disorder (MDD).

(69) The pharmaceutical composition of any one of (24) to (47), for use in therapy, such as for treating a subject with a depressive disorder, preferably major depressive disorder (MDD).

(70) Use of the pharmaceutical composition of any one of (24) to (47) for treating a subject with a depressive disorder, preferably major depressive disorder (MDD).

### BRIEF DESCRIPTION OF THE DRAWINGS

The forgoing paragraphs have been provided by way of general introduction and are not intended to limit the scope of the following claims. The described embodiments, together with further advantages, will be best understood by reference to the following detailed description when considered in conjunction with the accompanying drawings.

FIGS. 1A-1C show the Brown et al. 2017 (N=12) simulations for concentration (ng/mL) versus time after dose (h) for 25 mg (FIG. 1A), 37.5 mg (FIG. 1B), and 50 mg (FIG. 1C) oral psilocybin; solid line=mean simulation, dashed lines=standard deviation, black dots=extracted mean (Usona institute—FIGS. 5.3-1); N=1000 per treatment.

FIG. 2 shows the Hasler et al. 1997 (N=6) simulations for concentration (ng/mL) versus time after dose (h) for 10-20 mg oral psilocybin; solid line=mean simulation, dashed lines=standard deviation, black dots=extracted mean (Hasler FIG. 5), error bars=extracted SD (Hasler FIG. 5); N=200 per individual dose, N=1200 in total.

FIGS. 3A-3B show the Holze et al. 2022 (N=28) simulations for concentration (ng/mL) versus time after dose (h) for 13.83 mg (FIG. 3A) and 27.66 mg (FIG. 3B) oral psilocybin; solid line=mean simulation, dashed lines=standard deviation, black dots=extracted mean (Holze Figure S6), error bars=extracted SD (Holze Figure S6), N=1000 per treatment.

FIG. 4 shows a modeled linear effect relationship between VAS—Any drug effect score and mean psilocin concentration (ng/ml) after psilocybin administration (15 mg and 30 mg psilocybin) based on mean PK and PD data extracted from Holze et al. 2022.

FIG. 5 is reproduced from Madsen et al. 2019 (FIG. 3 in Madsen et al. 2019), showing the relationship between within-scan plasma psilocin after psilocybin levels and neocortical 5-HT_{2A}R occupancy.

FIGS. 6A-6B is reproduced from Madsen et al. 2019 (FIG. 4 in Madsen et al. 2019), showing subjective intensity of the psychedelic experience at the time of the PET scan, neocortical 5-HT_{2A}R occupancy and plasma psilocin after psilocybin concentration, with FIG. 6A showing the relationship between intensity ratings and neocortical 5-HT_{2A}R occupancy %, the fitted line being obtained using a quadratic function, and FIG. 6B showing the relationship between intensity and psilocin concentration, fitted to a single site receptor binding model.

FIGS. 7A-7D show simulated concentration (ng/mL) time after dose (h) profiles for psilocin-d₁₀ using Scenario 1 (FIG. 7A), Scenario 2 (FIG. 7B), Scenario 3 (FIG. 7C), and Scenario 4 (FIG. 7D) according to Table 5, benchmarked with various PD thresholds from available data; simulated profiles present the median of the simulated population (N=1000 per treatment).

FIG. 8 shows mean (±SD) plasma psilocin-d₁₀ concentration-time profiles for cohorts 1, 2, and 3 subjects by dose (linear scale); excludes 2 psilocin-d₁₀ 8 mg subjects (1 Cohort 3, Day 1 subject who vomited postdose and 1 Cohort 3, Day 1 subject with only 0-2 h PK samples).

FIG. 9 shows mean (±SD) plasma psilocin-d₁₀ concentration-time profiles for cohorts 1, 2, and 3 subjects by dose (semi-log scale); excludes 2 psilocin-d₁₀ 8 mg subjects (1 Cohort 3, Day 1 subject who vomited postdose and 1 Cohort 3, Day 1 subject with only 0-2 h PK samples).

FIG. 10 shows a study schematic for MDD Participants disclosed in the clinical trial protocol (Example III. Clinical Trial Protocol).

FIG. 11 shows a study schematic for normal healthy volunteer (NHV) Participants (Cohorts 2-6), disclosed in the clinical trial protocol (Example III. Clinical Trial Protocol).

FIG. 12 shows a study schematic for NHV Participants (Relative BA Cohort) disclosed in the clinical trial protocol (Example III. Clinical Trial Protocol).

FIG. 13 shows a plot of the mean (±SD) MADRS change from baseline score through Day 21 (D21) following a single 12 mg dose of psilocin-d₁₀ versus placebo, and the between-group Least Square (LS) Means difference.

FIG. 14 shows a plot of the mean (±SD) MADRS change from baseline score through Day 21 (D21) following a single 16 mg dose of psilocin-d₁₀ versus placebo, and the between-group Least Square (LS) Means difference.

FIG. 15 shows a bar graph of the mean (±SD) MADRS change from baseline score at Day 21 (following a single dose of psilocin-d₁₀), and further improvements at Day 42 (following a second dose of psilocin-d₁₀), for both the 12 mg and 16 mg dosage levels.

FIG. 16 shows a bar graph of the response rates (%; defined as ≥50% reduction from baseline MADRS) at Day 21 (D21) following a single dose of psilocin-d₁₀, further improvements at Day 42 (D42) following a second dose of psilocin-d₁₀, and the durability of these effects at Day 126 (D126) following these two doses, for both the 12 mg and 16 mg dosage levels; Day 126, 12 mg (N=15); Day 126, 16 mg (N=8).

FIG. 17 shows a bar graph of the remission rates (%; defined as a MADRS score of ≤10) at Day 21 (D21) following a single dose of psilocin-d₁₀, further improvements at Day 42 (D42) following a second dose of psilocin-d₁₀, and the durability of these effects at Day 126 (D126) following these two doses, for both the 12 mg and 16 mg dosage levels; Day 126, 12 mg (N=15); Day 126, 16 mg (N=8).

FIG. 18 shows a plot of the mean (±SD) MADRS change from baseline score through Day 126 (D126) following two 12 mg or two 16 mg doses of psilocin-d₁₀ (dosing on Day 1 and Day 22); 12 mg (N=15); 16 mg (N=8).

FIG. 19 shows the placebo subtracted change from baseline in MADRS following 12 mg psilocin-d₁₀ treatment after Day 21 compared to pivotal studies of approved antidepressant medications.

### DETAILED DESCRIPTION

In the following detailed description of the embodiments of the instant disclosure, numerous specific details are set forth in order to provide a thorough understanding of the disclosed embodiments. However, it will be obvious to one skilled in the art that the embodiments of this disclosure may be practiced without these specific details. In other instances, well known methods, procedures, components, and circuits have not been described in detail so as not to unnecessarily obscure aspects of the embodiments of the instant disclosure.

### Definitions

Unless defined otherwise, all technical and scientific terms used herein have the same meaning as is commonly understood by one of skill in the art to which this disclosure belongs.

As used herein, the term “major depressive disorder” refers to a condition characterized by a time period of low mood that is present across most situations. Major depressive disorder is often accompanied by low self-esteem, loss of interest in normally enjoyable activities, low energy, and pain without a clear cause. In some instances, major depressive order is characterized by symptoms of depression lasting at least two weeks. In some instances, an individual experiences periods of depression separated by years. In some instances, an individual experiences symptoms of depression that are nearly always present. Major depressive disorder can negatively affect a person's personal, work, or school life, as well as sleeping, eating habits, and general health. Approximately 2-7% of adults with major depressive disorder commit suicide, and up to 60% of people who commit suicide had major depressive disorder or another related mood disorder. Dysthymia is a subtype of major depressive disorder consisting of the same cognitive and physical problems as major depressive disorder with less severe but longer-lasting symptoms. Exemplary symptoms of a major depressive disorder include, but are not limited to, feelings of sadness, tearfulness, emptiness or hopelessness, angry outbursts, irritability or frustration, even over small matters, loss of interest or pleasure in most or all normal activities, sleep disturbances, including insomnia or sleeping too much, tiredness and lack of energy, reduced appetite, weight loss or gain, anxiety, agitation or restlessness, slowed thinking, speaking, or body movements, feelings of worthlessness or guilt, fixating on past failures or self-blame, trouble thinking, concentrating, making decisions, and remembering things, frequent thoughts of death, suicidal thoughts, suicide attempts, or suicide, and unexplained physical problems, such as back pain or headaches.

As used herein, the term “fatty” describes a compound with a long-chain (linear) hydrophobic portion made up of hydrogen and anywhere from 4 to 26 carbon atoms, which may be fully saturated or partially unsaturated.

The phrases “pharmaceutically acceptable,” “physiologically acceptable,” and the like, are employed herein to refer to those compounds, materials, compositions, and/or dosage forms which are, within the scope of sound medical judgment, suitable for use in contact with the tissues of human beings without excessive toxicity, irritation, allergic response, or other problem or complication, commensurate with a reasonable benefit/risk ratio. When referencing salts, the phrases “pharmaceutically acceptable salt,” “physiologically acceptable salt,” and the like, means a salt which is acceptable for administration to a patient, such as a mammal (salts with counterions having acceptable mammalian safety for a given dosage regime). As is well known in the art, such salts can be derived from pharmaceutically acceptable inorganic or organic bases, by way of example, sodium, potassium, calcium, magnesium, ammonium, and tetraalkylammonium salts, and the like, and when the molecule contains a basic functionality, addition salts with inorganic acids, such as hydrochloride, hydrobromide, sulfate, sulfamate, phosphate, nitrate, perchlorate salts, and the like, and addition salts with organic acids, such as formate, tartrate, besylate, mesylate, acetate, maleate, malonate, oxalate, fumarate, benzoate, salicylate, succinate, oxalate, glycolate, hemi-oxalate, hemi-fumarate, propionate, stearate, tartrate, lactate, citrate, ascorbate, pamoate, hydroxymaleate, phenylacetate, glutamate, 2-acetoxybenzoate, tosylate, ethanedisulfonate, isethionate salts, and the like. The term “salt thereof” means a compound formed when a proton of an acid is replaced by a cation, such as a metal cation or an organic cation and the like. Where applicable, the salt is a pharmaceutically acceptable salt, although this is not required for salts of intermediate compounds that are not intended for administration to a patient. By way of example, salts of the present compounds include those wherein the compound is protonated by an inorganic or organic acid to form a cation, with the conjugate base of the inorganic or organic acid as the anionic component of the salt.

“Solvate” refers to a physical association of a compound or salt of the present disclosure with one or more solvent molecules, whether organic, inorganic, or a mixture of both. This physical association includes hydrogen bonding. In certain instances, the solvate will be capable of isolation, for example when one or more solvent molecules are incorporated in the crystal lattice of a crystalline solid. The solvent molecules in the solvate may be present in a regular arrangement and/or a non-ordered arrangement. The solvate may comprise either a stoichiometric or nonstoichiometric amount of the solvent molecules. “Solvate” encompasses both solution-phase and isolable solvates. Some examples of solvents include, but are not limited to, methanol, ethanol, isopropanol, N,N-dimethylformamide, tetrahydrofuran, dimethylsulfoxide, and water. When the solvent is water, the solvate formed is a hydrate (e.g., monohydrate, dihydrate, etc.). Exemplary solvates thus include, but are not limited to, hydrates, methanolates, ethanolates, isopropanolates, etc. Methods of solvation are generally known in the art.

“Stereoisomer” and “stereoisomers” refer to compounds that have same atomic connectivity but different atomic arrangement in space. Stereoisomers include cis-trans isomers, E and Z isomers, enantiomers, and diastereomers. All forms such as racemates and optically pure stereoisomers of the compounds are contemplated herein. Chemical formulas and compounds which possess at least one stereogenic center, but are drawn without reference to stereochemistry, are intended to encompass both the racemic compound, as well as the separate stereoisomers, e.g., R- and/or S-stereoisomers, each permutation of diastereomers so long as those diastereomers are geometrically feasible, etc.

“Tautomer” refers to alternate forms of a molecule that differ only in electronic bonding of atoms and/or in the position of a proton, such as enol-keto, imine-enamine, and neutral/zwitterionic tautomers, or the tautomeric forms of heteroaryl groups containing a —N═C(H)—NH— ring atom arrangement, such as pyrazoles, imidazoles, benzimidazoles, triazoles, and tetrazoles. Other tautomeric ring atom arrangements are also possible.

A “crystalline” solid is a type of solid whose fundamental three-dimensional structure contains a highly regular pattern of atoms or molecules—with long range order—forming a crystal lattice, and thus displays sharp characteristic crystalline peak(s) in its X-ray power diffraction (XRPD) pattern. In some instances, crystalline solids can exist in different crystalline forms known as “polymorphs,” which have the same chemical composition, but differ in packing, geometric arrangement, and other descriptive properties of the crystalline solid state. As such, polymorphs may have different solid-state physical properties to affect, for example, the solubility, dissolution rate, bioavailability, chemical and physical stability, flowability, and compressibility, etc. of the compound as well as the safety and efficacy of drug products based on the compound. In the process of preparing a polymorph, further purification, in terms of gross physical purity or optical purity, may be accomplished as well. A material's crystalline form, including polymorphic forms, may be designated by “pattern” number throughout the present disclosure (e.g., pattern 1, pattern 2, etc.) based on its characterized X-ray power diffraction (XRPD) pattern. As used herein, the term “amorphous” refers to a solid material having substantially no long-range order in the position of its molecules—the molecules are arranged in a random manner so that there is effectively no well-defined arrangement, e.g., molecular packing, and no long-range order. Amorphous solids are generally isotropic, i.e., exhibit similar properties in all directions and do not have definite melting points. For example, an amorphous material is a solid material having substantially no sharp characteristic crystalline peak(s) in its X-ray power diffraction (XRPD) pattern (i.e., is not crystalline as determined by XRPD). Instead, one or several broad peaks (e.g., halos) appear in its XRPD pattern. Broad peaks are characteristic of an amorphous solid. Thus, an “amorphous” subject compound/material is one characterized as having substantially no crystallinity—less than 10% crystallinity, less than 8% crystallinity, less than 6% crystallinity, less than 4% crystallinity, less than 2% crystallinity, less than 1% crystallinity, or 0% crystallinity—i.e., is at least 90%, at least 92%, at least 94%, at least 96%, at least 98%, or 100% amorphous, as determined for example by XRPD. For example, the % crystallinity can in some embodiments be determined by measuring the intensity of one or more peaks in the XRPD diffractogram compared to a reference peak, which may be that of a known standard or an internal standard. Other characterization techniques, such as modulated differential scanning calorimetry (mDSC) analysis, Fourier transform infrared spectroscopy (FTIR), and other quantitative methods, may also be employed to determine the percent a subject compound/material is amorphous or crystalline, including quantitative methods which provide the above percentages in terms of weight percent.

References to X-ray powder diffraction (XRPD) patterns of materials, compounds, salts, etc. of the present disclosure being characterized by an X-ray powder diffraction pattern containing “at least three characteristic peaks” should be understood to include those materials/compounds/salts characterized as having 3, 4, 5, 6, 7, 8, 9, 10, 11, 12, 13, 14, 15 or more (including all) of the recited characteristic XRPD diffraction peaks. Further, materials/compounds/salts containing “at least three characteristic peaks at diffraction angles (2θ±0.2°) selected from . . . ” are open to inclusion of other XRPD diffraction peaks not recited.

It will be appreciated that the compounds herein can exist in different salt, solvate, crystalline/amorphous (or polymorphic) forms, and the present disclosure is intended to include all permutations thereof, such as a solvate of a pharmaceutically acceptable salt. Thus, references to a compound, or a pharmaceutically acceptable salt, polymorph, or salt thereof, is intended to include all permutations thereof, for example, a pharmaceutically acceptable salt of the compound in a crystalline form, a pharmaceutically acceptable salt of the compound in solvated form, a crystalline free base compound, a crystalline free base compound as a solvate, etc.

As used herein, the term “steady” describes the stable or steady-state level of a molecule concentration, e.g., concentration of any compound described herein.

The term “stable,” “stability,” and the like, as used herein includes chemical stability and solid state (physical) stability. The term “chemical stability” means that the compound can be stored in an isolated form, or in the form of a formulation in which it is provided in admixture with for example, pharmaceutically acceptable carriers, diluents or adjuvants as described herein, under normal storage conditions, with little or no chemical degradation or decomposition. “Solid-state stability” means the compound can be stored in an isolated solid form, or the form of a solid formulation in which it is provided in admixture with, for example, pharmaceutically acceptable carriers, diluents or adjuvants as described herein, under normal storage conditions, with little or no solid-state transformation (e.g., hydration, dehydration, solvatization, desolvatization, crystallization, recrystallization or solid-state phase transition).

A “psilocybin-based” drug is any prodrug of a psilocin-type compound, such as an alkyl/aryl ester, an α-amino ester (e.g., an amino acid ester), a hemi-ester, a bis-ester, a phosphate ester, a sulfate ester, etc., that when administered releases psilocin or a deuterated analog thereof (e.g., a compound of Formula (I-3)) as the active component. A psilocybin-based drug includes psilocybin itself (dihydrogen phosphate ester of psilocin, in either neutral or zwitterionic form).

As used herein, the term “composition” is equivalent to the term “formulation.”

As used herein, the term “active ingredient” is equivalent to the term “active pharmaceutical ingredient” (API).

The language “tamper resistant” is art-recognized to describe aspects of a drug formulation that make it more difficult to use the formulation to abuse the drug moiety of the formulation through extraction for intravenous use, intradermal use, etc. use, or crushing for freebase use; and therefore reduce the risk for abuse of the drug.

The term “treating” or “treatment” as used herein means the treating or treatment of a disease or medical condition in a patient, such as a mammal (particularly a human) that includes: ameliorating the disease or medical condition, such as, eliminating or causing regression of the disease or medical condition in a patient; suppressing the disease or medical condition, for example by, slowing or arresting the development of the disease or medical condition in a patient; or alleviating one or more symptoms of the disease or medical condition in a patient. In an embodiment, prophylactic treatment can result in preventing the disease or medical condition from occurring, in a subject.

A “patient” or “subject,” used interchangeably herein, can be any mammal including, for example, a human and non-human subjects. A patient or subject can have a condition to be treated or can be susceptible to a condition to be treated.

As used herein, and unless otherwise specified, the terms “prevent,” “preventing” and “prevention” refer to the prevention of the onset, recurrence or spread of a disease, disorder, or condition, or of one or more symptoms thereof. The terms encompass the inhibition or reduction of a symptom of the particular disease, disorder, or condition. Subjects with familial history of a disease, disorder, or condition, in particular, are candidates for preventive regimens in certain embodiments. In addition, subjects who have a history of recurring symptoms are also potential candidates for the prevention. In this regard, the term “prevention” may be interchangeably used with the term “prophylactic treatment.”

As used herein, and unless otherwise specified, the terms “manage,” “managing” and “management” refer to preventing or slowing the progression, spread or worsening of a disease, disorder, or condition, or of one or more symptoms thereof. Often, the beneficial effects that a subject derives from a prophylactic and/or therapeutic agent do not result in a cure of the disease, disorder, or condition. In this regard, the term “managing” encompasses treating a subject who had suffered from the particular disease, disorder, or condition in an attempt to prevent or minimize the recurrence of the disease, disorder, or condition, or of one or more symptoms thereof.

“Therapeutically effective amount” refers to an amount of a compound(s), or a pharmaceutically acceptable salt, polymorph, or solvate thereof, sufficient to treat a specified disorder or disease or one or more of its symptoms and/or to prevent the occurrence of the disease or disorder (prophylactically effective amount).

As used herein, and unless otherwise specified, a “prophylactically effective amount” of an active ingredient, is an amount sufficient to prevent a disease, disorder, or condition, or prevent its recurrence. The term “prophylactically effective amount” can encompass an amount that improves overall prophylaxis or enhances the prophylactic efficacy of another prophylactic agent.

The term “administration schedule” is a plan in which the type, amount, period, procedure, etc. of the drug in the drug treatment are shown in time series, and the dosage, administration method, administration order, administration date, and the like of each drug are indicated. The date specified to be administered is determined before the start of the drug administration. The administration is continued by repeating the course with the set of administration schedules as “courses”. A “continuous” administration schedule means administration every day without interruption during the treatment course. If the administration schedule follows an “intermittent” administration schedule (where dosing occurs less than daily), then days of administration may be followed by “rest days” or days of non-administration of drug within the course. A “drug holiday” indicates that the drug is not administered in a predetermined administration schedule. For example, after undergoing one or several courses of treatment, a subject may be prescribed a regulated drug holiday as part of the administration schedule, e.g., prior to re-recommencing active treatment.

The language “toxic spikes” is used herein to describe neurological spikes in concentration of any compound described herein that would produce side-effects of sedation or psychotomimetic effects (e.g., hallucination, dizziness, and nausea), or any unwanted and/or unintended secondary effects caused by the administration of a medicament to an individual resulting in subjective experiences being qualitatively different from those of ordinary consciousness. These experiences can include derealization, depersonalization, hallucinations and/or sensory distortions in the visual, auditory, olfactory, tactile, proprioceptive and/or interoceptive spheres and/or any other perceptual modifications, and/or any other substantial subjective changes in cognition, memory, emotion and consciousness. Such side effects, when unwanted and/or unintended, can not only have immediate repercussions, but also effect treatment compliance. In particular, side effects may become more pronounced at blood concentration levels of about 250, 300, 400, 500 ng/L or more.

All diseases and disorders listed herein may be defined as described in the Diagnostic and Statistical Manual of Mental Disorders, 5th edition (DSM-5), published by the American Psychiatric Association, or in International Classification of Diseases (ICD), published by the World Health Organization.

As used herein “adjunctive therapy,” “adjuvant therapy,” and the like, refers to a therapy that is given in addition to a primary or initial therapy to improve or maximize effectiveness. For example, a subject diagnosed with a depressive disorder that is taking one or more antidepressant medications (e.g., an SSRI) as a primary or initial therapy, but has an inadequate response to or has otherwise failed to achieve a desirable outcome with the antidepressant medication may be administered, as “adjunctive therapy,” a compound of Formula (I-3), or a pharmaceutically acceptable salt, polymorph, or solvate thereof, to improve or maximize treatment effectiveness. In this example with a depressive disorder, the adjunctive therapy may improve or maximize treatment effectiveness by reducing depressive symptom(s) compared to the primary or initial therapy alone. The primary or initial therapy and the adjunctive therapy involving a compound of Formula (I-3), or a pharmaceutically acceptable salt, polymorph, or solvate thereof, can be, but need not be, prescribed and/or administered by the same person (e.g., clinician). For example, the primary or initial therapy (e.g., SSRI therapy) may be prescribed by a first clinician and self-administered by the patient, while the adjunctive therapy involving a compound of Formula (I-3), or a pharmaceutically acceptable salt, polymorph, or solvate thereof, may be prescribed and/or administered by a second clinician. Alternatively, the primary or initial therapy (e.g., SSRI therapy) may be prescribed by a first clinician and self-administered by the patient, while the adjunctive therapy involving a compound of Formula (I-3), or a pharmaceutically acceptable salt, polymorph, or solvate thereof, may be prescribed and/or administered by the same (first) clinician.

The term “inadequate response” as used herein refers to a lack of clinically meaningful improvement in symptoms, for example as measured by one or more of the rating scales described herein. The inadequate response to an adequate course of treatment with an antidepressant medication(s) may be determined retrospectively or prospectively. Prospective determination of inadequate response refers to a determination made by the prescribing clinician or therapist following administration of part of a course of treatment. Retrospective determination refers to a determination made by the prescribing clinician or therapist following administration of a full adequate course of treatment.

Unless specified otherwise, references to dosing of the compound of Formula (I-3), or a pharmaceutically acceptable salt, polymorph, or solvate thereof (psilocin-d₁₀) are with respect to free base equivalence. Thus, the recited amounts preceding the phrase “ . . . mg of a compound of Formula (I-3), or a pharmaceutically acceptable salt, polymorph, or solvate thereof”, and the like, refer to the amount of psilocin-d₁₀ (free base equivalence). That is, if the psilocin-d₁₀ is administered as a pharmaceutically acceptable salt, the recited dose refers to the amount of psilocin-d₁₀ (free base) that is present without weight contribution from the salt counterion. For example, if administering the benzoate salt of psilocin-d₁₀ to a subject, administration of “8 mg of a compound of Formula (I-3), or a pharmaceutically acceptable salt, polymorph, or solvate thereof” would be accomplished upon administration of 12.6 mg of the benzoate salt of psilocin-d₁₀. Likewise, if administering the benzenesulfonate salt of psilocin-d₁₀ to a subject, administration of “8 mg of a compound of Formula (I-3), or a pharmaceutically acceptable salt, polymorph, or solvate thereof” would be accomplished upon administration of 13.9 mg of the benzenesulfonate salt of psilocin-d₁₀. In another example, if administering the benzenesulfonate salt of psilocin-d₁₀ to a subject, administration of “16 mg of a compound of Formula (I-3), or a pharmaceutically acceptable salt, polymorph, or solvate thereof” would be accomplished upon administration of 27.8 mg of the benzenesulfonate salt of psilocin-d₁₀.

The term “compound” when referring to a compound of Formula (I-3) (IUPAC name of 3-(2-(bis(methyl-d₃)amino)ethyl-1,1,2,2-d₄)-1H-indol-4-ol; or psilocin-d₁₀), refers to a collection or population of molecules having an identical chemical structure, except that there may be isotopic variation among the constituent atoms of the molecules. This is because, in practice, it is generally not possible to achieve deuterium enrichment with 100% isotopic purity. Thus, it will be clear to those of skill in the art that a compound represented by a particular chemical structure (the subject compound) containing indicated deuterium atoms, will also contain lesser amounts of isotopologues having hydrogen atoms at one or more of the designated deuterium positions in that structure. The relative amount of such isotopologues in the compound of Formula (I-3) will depend on a number of factors including the isotopic purity of deuterated reagents used to make the compound of Formula (I-3) and the efficiency of incorporation of deuterium in the various synthetic steps used to prepare the compound of (1-3). However, as set forth herein, the relative amount of such isotopologues in total will preferably be less than 50% of the compound. With respect to the dosage amounts recited herein (e.g., 10 mg, 12 mg, 14 mg, 16 mg, etc.), it should be understood that the recited dosage amounts of the compound of Formula (I-3) (or a pharmaceutically acceptable salt, polymorph, or solvate thereof) refer to the amount of the subject compound plus the total amount of any isotopologues(s) thereof that is dosed. For example, administration of 10 mg of a compound of Formula (I-3) (or a pharmaceutically acceptable salt, polymorph, or solvate thereof) which has an isotopic purity of 90% is considered herein to be a dose of 10 mg (i.e., 9 mg of psilocin-d₁₀ as the subject compound+1 mg of total isotopologues of the subject compound).

The phrase “effect size” refers to a statistical calculation that can be used to compare the efficacy of different agents by quantifying the size of the difference between treatments (“between-group”). It is a dimensionless measure of the difference in outcomes under two different treatment interventions. Effect sizes thus inform clinicians about the magnitude of treatment effects. Unless stated otherwise, as used herein, the effect size is calculated from the difference between the mean within-group efficacy endpoint change resulting from a treatment group (e.g., administration of a compound of Formula (I-3), or a pharmaceutically acceptable salt, polymorph, or solvate thereof) and the mean within-group efficacy endpoint change resulting from placebo, using Cohen's d method with the following formula:

$$Cohen ’ s d = \frac{\begin{matrix}Mean endpoint change from treatment - \\ Mean endpoint change from placebo\end{matrix}}{Pooled standard deviation for entire sample}$$

Cohen's d is also known as the standardized mean difference. An effect size (Cohen's d score) of zero means that the treatment and placebo have no differences in effect. An effect size (Cohen's d score) greater than zero indicates the degree to which treatment is more efficacious than placebo. Conventionally, it is considered that an effect size (Cohen's d score) of 0.2 is small, 0.5 is medium, and 0.8 or higher is large.

As used herein, the term “and/or” includes any and all combinations of one or more of the associated listed items. As used in the description herein and throughout the claims that follow, the meaning of “a”, “an”, and “the” includes plural reference as well as the singular reference unless the context clearly dictates otherwise. The term “about” in association with a numerical value means that the value varies up or down by 5%. For example, for a value of about 100, means 95 to 105 (or any value between 95 and 105).

### Therapeutic Applications and Methods

Applicants recently discovered a series of stabilized forms of psilocin and deuterated psilocin, including novel polymorphs of psilocin/deuterated psilocin, novel salt forms of psilocin/deuterated psilocin and their polymorphs, as well as compositions thereof (see WO2022195011 and WO2023078604, herein incorporated by reference in their entirety).

Compounds previously identified, include the compound of Formula (I-3)

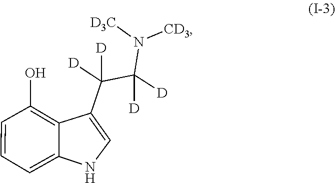 [SMILES: [2H]C([2H])([2H])N(C([2H])([2H])[2H])C([2H])([2H])C([2H])([2H])c1c[nH]c2cccc(O)c12]

and pharmaceutically acceptable salts, polymorphs, and solvates thereof. The compound of Formula (I-3) has an IUPAC name of 3-(2-(bis(methyl-d₃)amino)ethyl-1,1,2,2-d₄)-1H-indol-4-ol, and is also referred to as psilocin-d₁₀. It will be understood that reference to any of the above identifiers, is meant to convey the compound of Formula (I-3). The compound of Formula (I-3) may be aptly considered a psychedelic agent in the present disclosure.

The present disclosure relates to the unexpected discovery that the compound of Formula (I-3)

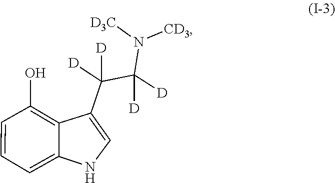 [SMILES: [2H]C([2H])([2H])N(C([2H])([2H])[2H])C([2H])([2H])C([2H])([2H])c1c[nH]c2cccc(O)c12]

or a pharmaceutically acceptable salt, a polymorph, or solvate thereof, is more efficacious and has a better therapeutic window than predicted, based on animal models of the compound of Formula (I-3), literature references describing data of the non-deuterated counterpart psilocin and psilocin-d₁₀ clinical simulations. This discovery allows for methods of treatment that comprise administering lower doses of the compounds of the disclosure, which is beneficial to the subject undergoing treatment, while achieving therapeutic benefits (e.g., an effect size) far greater than could have been expected. As side effects and adverse drug reactions can be substantially dose related, a lower dose of a compound is predicted to be beneficial in decreasing risks to the subject being treated.

Disclosed herein is a method of treating various diseases, disorders, and conditions, such as depressive disorders (e.g., Major Depressive Disorder (MDD)), substance use disorders, anxiety disorders, eating disorders, and headache disorders in a subject in need thereof, comprising administering to the subject a therapeutically effective amount of a compound of Formula (I-3)

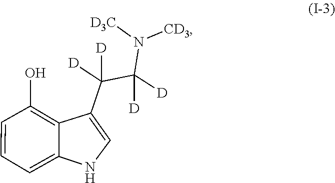 [SMILES: [2H]C([2H])([2H])N(C([2H])([2H])[2H])C([2H])([2H])C([2H])([2H])c1c[nH]c2cccc(O)c12]

or a pharmaceutically acceptable salt, polymorph, or solvate thereof. In some embodiments, a therapeutically effective amount of a compound of Formula (I-3), or a pharmaceutically acceptable salt, polymorph, or solvate thereof, administered to the subject in need thereof is about 8 mg, about 9 mg, about 10 mg, about 11 mg, about 12 mg, about 13 mg, about 14 mg, about 15 mg, about 16 mg, or any range therebetween, such as from about 8 to about 16 mg, about 8 to about 14 mg, about 8 to about 12 mg, about 10 to about 16 mg, about 11 to about 15 mg, about 12 to about 14 mg, about 11 to about 13 mg, about 14 to about 16 mg, or about 12 to about 16 mg (free base equivalence). In some embodiments, the method comprises administering 8 mg to 16 mg of a compound of Formula (I-3), or a pharmaceutically acceptable salt, polymorph, or solvate thereof. In some embodiments, the method comprises administering 8 mg to 14 mg of a compound of Formula (I-3), or a pharmaceutically acceptable salt, polymorph, or solvate thereof. In some embodiments, the method comprises administering 8 mg to 12 mg of a compound of Formula (I-3), or a pharmaceutically acceptable salt, polymorph, or solvate thereof. In some embodiments, the method comprises administering 8 mg to 10 mg of a compound of Formula (I-3), or a pharmaceutically acceptable salt, polymorph, or solvate thereof. In some embodiments, the method comprises administering 10 mg to 16 mg of a compound of Formula (I-3), or a pharmaceutically acceptable salt, polymorph, or solvate thereof. In some embodiments, the method comprises administering 12 mg to 16 mg of a compound of Formula (I-3), or a pharmaceutically acceptable salt, polymorph, or solvate thereof. In some embodiments, the method comprises administering 10 mg to 14 mg of a compound of Formula (I-3), or a pharmaceutically acceptable salt, polymorph, or solvate thereof. In some embodiments, the method comprises administering 12 mg to 14 mg of a compound of Formula (I-3), or a pharmaceutically acceptable salt, polymorph, or solvate thereof. In some embodiments, the method comprises administering 11 mg to 16 mg of a compound of Formula (I-3), or a pharmaceutically acceptable salt, polymorph, or solvate thereof. In some embodiments, the method comprises administering 11 mg to 15 mg of a compound of Formula (I-3), or a pharmaceutically acceptable salt, polymorph, or solvate thereof. In some embodiments, the method comprises administering 11 mg to 13 mg of a compound of Formula (I-3), or a pharmaceutically acceptable salt, polymorph, or solvate thereof. In some embodiments, the method comprises administering about 8 mg of a compound of Formula (I-3), or a pharmaceutically acceptable salt, polymorph, or solvate thereof. In some embodiments, the method comprises administering 8 mg of a compound of Formula (I-3), or a pharmaceutically acceptable salt, polymorph, or solvate thereof. In some embodiments, the method comprises administering about 9 mg of a compound of Formula (I-3), or a pharmaceutically acceptable salt, polymorph, or solvate thereof. In some embodiments, the method comprises administering 9 mg of a compound of Formula (I-3), or a pharmaceutically acceptable salt, polymorph, or solvate thereof. In some embodiments, the method comprises administering about 10 mg of a compound of Formula (I-3), or a pharmaceutically acceptable salt, polymorph, or solvate thereof. In some embodiments, the method comprises administering 10 mg of a compound of Formula (I-3), or a pharmaceutically acceptable salt, polymorph, or solvate thereof. In some embodiments, the method comprises administering about 11 mg of a compound of Formula (I-3), or a pharmaceutically acceptable salt, polymorph, or solvate thereof. In some embodiments, the method comprises administering 11 mg of a compound of Formula (I-3), or a pharmaceutically acceptable salt, polymorph, or solvate thereof. In some embodiments, the method comprises administering about 12 mg of a compound of Formula (I-3), or a pharmaceutically acceptable salt, polymorph, or solvate thereof. In some embodiments, the method comprises administering 12 mg of a compound of Formula (I-3), or a pharmaceutically acceptable salt, polymorph, or solvate thereof. In some embodiments, the method comprises administering about 13 mg of a compound of Formula (I-3), or a pharmaceutically acceptable salt, polymorph, or solvate thereof. In some embodiments, the method comprises administering 13 mg of a compound of Formula (I-3), or a pharmaceutically acceptable salt, polymorph, or solvate thereof. In some embodiments, the method comprises administering about 14 mg of a compound of Formula (I-3), or a pharmaceutically acceptable salt, polymorph, or solvate thereof. In some embodiments, the method comprises administering 14 mg of a compound of Formula (I-3), or a pharmaceutically acceptable salt, polymorph, or solvate thereof. In some embodiments, the method comprises administering about 15 mg of a compound of Formula (I-3), or a pharmaceutically acceptable salt, polymorph, or solvate thereof. In some embodiments, the method comprises administering 15 mg of a compound of Formula (I-3), or a pharmaceutically acceptable salt, polymorph, or solvate thereof. In some embodiments, the method comprises administering about 16 mg of a compound of Formula (I-3), or a pharmaceutically acceptable salt, polymorph, or solvate thereof. In some embodiments, the method comprises administering 16 mg of a compound of Formula (I-3), or a pharmaceutically acceptable salt, polymorph, or solvate thereof. In some embodiments, the compound of Formula (I-3), or a pharmaceutically acceptable salt, polymorph, or solvate thereof is administered orally, and the doses recited above are oral doses.

### Compounds of Formula (I-3) for Use in the Pharmaceutical Compositions and the Methods of the Invention

In some embodiments, the compound of Formula (I-3) is a compound of Formula (I-3), or a pharmaceutically acceptable salt, polymorph, or solvate thereof as described in WO2022195011 and/or WO2023078604.

In some embodiments, the compound of Formula (I-3) according to methods described herein, is a free base in crystalline form, e.g., as determined by XRPD and/or mDSC. Accordingly, the compound of Formula (I-3) as a free base, in one or more crystalline (e.g., polymorphic) forms, may be used for treatment as set forth herein. In some embodiments, a crystalline form of a compound of Formula (I-3) as a free base is provided. For example, the compound may comprise a free base of a compound of Formula (I-3), wherein at least 50%, at least 60%, at least 70%, at least 80%, at least 90%, at least 95%, at least 99%, or at least 99.5% by weight of the free base of the compound of Formula (I-3) present is in crystalline form, e.g., as determined by X-ray powder diffraction and/or mDSC. In some embodiments, a highly pure crystalline form of a compound of Formula (I-3) as a free base is provided. For example, the compound may comprise a free base of a compound of Formula (I-3), wherein at least 90%, at least 95%, at least 99%, or at least 99.5% by weight of the free base of the compound of Formula (I-3) is in crystalline form, e.g., as determined by X-ray powder diffraction and/or mDSC.

In some embodiments, the compound of Formula (I-3) according to methods described herein, is a crystalline form of 3-(2-(bis(methyl-d₃)amino)ethyl-1,1,2,2-d₄)-1H-indol-4-ol (1-3), as determined by X-ray powder diffraction. In some embodiments, I-3 is a crystalline solid form (pattern 1) characterized by an X-ray powder diffraction pattern containing at least three characteristic peaks at diffraction angles (2θ±0.2°) selected from 7.582°, 8.395°, 9.647°, 10.444°, 11.319°, 12.614°, 13.372°, 14.222°, 15.157°, 16.524°, 16.787°, 17.693°, 19.468°, 19.699°, 20.901°, 21.132°, 21.859°, 22.547°, 23.699°, 24.630°, 25.034°, 25.264°, 26.867°, 27.399°, 27.929°, 28.219°, 28.871°, 29.430°, 30.120°, 30.675°, 31.373°, 32.365°, 33.880°, 34.418°, 34.792°, 35.884°, 36.254°, 37.156°, 38.200°, and 38.417°, as determined by XRPD using a CuKα radiation source, for example, as shown in WO2022195011 and/or WO2023078604. In some embodiments, I-3 is a crystalline solid form (pattern 2) characterized by an X-ray powder diffraction pattern containing at least three characteristic peaks at diffraction angles (2θ±0.2°) selected from 8.124°, 8.357°, 10.059°, 12.630°, 13.420°, 13.743°, 14.053°, 15.220°, 16.272°, 16.763°, 16.954°, 17.328°, 17.662°, 18.062°, 18.742°, 19.413°, 19.658°, 20.172°, 20.836°, 21.267°, 21.833°, 22.213°, 22.504°, 23.334°, 23.701°, 24.385°, 25.431°, 25.721°, 26.049°, 27.291°, 28.368°, 30.349°, 30.656°, 31.337°, 31.538°, 32.091°, 35.870°, 38.514°, and 41.361°, as determined by XRPD using a CuKα radiation source, for example, as shown in WO2022195011 and/or WO2023078604.

In some embodiments, the compounds according to methods described herein, are provided as a free base in amorphous form, e.g., as determined by XRPD and/or mDSC. Accordingly, compounds of Formula (I-3) as a free base, in one or more amorphic forms, and may be used for treatment as set forth herein. In some embodiments, a highly pure amorphous form of a compound of Formula (I-3) as a free base is provided. For example, the free base of a compound of Formula (I-3), may be at least 92%, at least 94%, at least 96%, at least 98%, at least 99%, or at least 99.5% amorphous form by weight of the free base of the compound of Formula (I-3) e.g., as determined by X-ray powder diffraction and/or mDSC.

In some embodiments, the compound according to methods described herein, is an amorphous form of 3-(2-(bis(methyl-d₃)amino)ethyl-1,1,2,2-d₄)-1H-indol-4-ol (1-3), as determined by X-ray powder diffraction.

Such amorphous forms of the compounds of Formula (I-3) (free base) may be advantageous in terms of dissolution rates in water, compared to crystalline forms, thereby enabling rapid systemic absorption for quick therapeutic onset and a short duration of drug action. Further, in some embodiments, pharmaceutical compositions may be prepared which comprise the amorphous form of the free base of the compound of Formula (I-3) (vide infra). The pharmaceutical compositions of the present disclosure, such as those set forth herein, may act to stabilize the amorphous form of the compound of Formula (I-3), which tend to be unstable and have a tendency to crystallize. Accordingly, the pharmaceutical compositions can be used to stabilize and deliver these amorphous forms to subjects in need of treatment, i.e., for the treatment of a condition or disease associate with major depressive disorder.

### Salt Forms

In some embodiments, the compound according to methods described herein, is a pharmaceutically acceptable salt of the compound of Formula (I-3), or a pharmaceutically acceptable polymorph, or solvate thereof. The acid used to form the pharmaceutically acceptable salt of the compound of Formula (I-3) may be a monoacid, a diacid, a triacid, a tetraacid, or may contain a higher number of acid groups. The acid groups may be, e.g., a carboxylic acid, a sulfonic acid, a phosphonic acid, or other acidic moieties containing at least one replaceable hydrogen atom. Examples of acids, which may be organic or inorganic acids, for use in the preparation of the pharmaceutically acceptable (acid addition) salts disclosed herein include, but are not limited to, acetic acid, 2,2-dichloroacetic acid, phenylacetic acid, acylated amino acids, alginic acid, ascorbic acid, L-aspartic acid, sulfonic acids (e.g., benzenesulfonic acid, camphorsulfonic acid, (+)-(1S)-camphor-10-sulfonic acid, ethane-1,2-disulfonic acid, ethanesulfonic acid, 2-hydroxy-ethanesulfonic acid, methanesulfonic acid, naphthalene-2-sulfonic acid, naphthalene-1,5-disulfonic acid, p-toluenesulfonic acid, ethanedisulfonic acid, etc.), benzoic acids (e.g., benzoic acid, 4-acetamidobenzoic acid, 2-acetoxybenzoic acid, salicylic acid, 4-amino-salicylic acid, gentisic acid, etc.), boric acid, (±)-camphoric acid, cinnamic acid, citric acid, cyclamic acid, cyclohexanesulfamic acid, dodecylsulfuric acid, formic acid, fumaric acid, galactaric acid, glucoheptonic acid, D-gluconic acid, D-glucuronic acid, L-glutamic acid, α-oxo-glutaric acid, glycolic acid, hippuric acid, hydrobromic acid, hydrochloric acid, hydroiodic acid, (+)-L-lactic acid, (−)-D-lactic acid, (±)-DL-lactic acid, lactobionic acid, maleic acid, malic acid, (−)-L-malic acid, (+)-D-malic acid, hydroxymaleic acid, malonic acid, (±)-DL-mandelic acid, isethionic acid, 1-hydroxy-2-naphthoic acid, nicotinic acid, nitric acid, orotic acid, oxalic acid, pamoic acid, perchloric acid, phosphoric acid, L-pyroglutamic acid, saccharic acid, succinic acid, sulfuric acid, sulfamic acid, tannic acid, tartaric acids (e.g., DL-tartaric acid, (+)-L-tartaric acid, (−)-D-tartaric acid), thiocyanic acid, propionic acid, valeric acid, and fatty acids (including fatty mono- and di-acids, e.g., adipic (hexandioic) acid, lauric (dodecanoic) acid, linoleic acid, myristic (tetradecanoic) acid, capric (decanoic) acid, stearic (octadecanoic) acid, oleic acid, caprylic (octanoic) acid, palmitic (hexadecenoic) acid, sebacic acid, undecylenic acid, caproic acid, etc.).

Certain salts are preferred among the list above because they possess physical and pharmaceutical characteristics/properties which make them more suitable for pharmaceutical preparation and administration. For example, preferred salt forms of the compounds disclosed herein (e.g., compounds of Formula (I-3)) are those that possess one or more of the following characteristics: are easy to prepare in high yield with a propensity towards salt formation; are stable and have well-defined physical properties such as crystallinity, defined and reproducible polymorphism insofar as polymorphism exists, and high melting/enthalpy of fusion; have slight or no hygroscopicity; are free flowing, do not cohere/adhere to surfaces, and possess a regular morphology; have acceptable aqueous solubility and rate of dissolution for the intended dosage form; and/or are physiologically acceptable, e.g., do not cause excessive irritation.

### Crystallinity

In some embodiments, the pharmaceutically acceptable salt of the compound of Formula (I-3) according to methods described herein, may be crystalline or amorphous, as determined e.g., by X-ray powder diffraction (XRPD) and/or mDSC. In some embodiments, the salt of the compound of Formula (I-3) is amorphous. Amorphous forms typically possess higher aqueous solubility and rates of dissolution compared to their crystalline counterparts, and thus may be well suited for quick acting dosage forms adapted to rapidly release the active ingredient, such as orodispersible dosage forms (ODxs), immediate release (IR) dosage forms, and the like. The salts of the compound of Formula (I-3) can be in a stable amorphous form. In some embodiments, the pharmaceutically acceptable salt of the compound of Formula (I-3) is provided in amorphous form, e.g., as determined by XRPD and/or mDSC. Accordingly, pharmaceutically acceptable salt forms of compounds of Formula (I-3), in one or more amorphic forms, and may be used for treatment as set forth herein. In some embodiments, a highly pure amorphous form of a pharmaceutically acceptable salt of a compound of Formula (I-3) is provided. For example, the pharmaceutically acceptable salt of a compound of Formula (I-3), may be at least 92%, at least 94%, at least 96%, at least 98%, at least 99%, or at least 99.5% amorphous form by weight of pharmaceutically acceptable salt of a compound of Formula (I-3), e.g., as determined by X-ray powder diffraction and/or mDSC.

In some embodiments, the pharmaceutically acceptable salt of the compound of Formula (I-3) according to methods described herein, is crystalline. Crystalline forms are advantageous in terms of stability and providing well-defined physical properties, which is desirable for pharmaceutical preparation and administration. The salts of the compound of Formula (I-3) can be in a stable crystalline form. In some embodiments, the pharmaceutically acceptable salt of the compound of Formula (I-3) has a percent crystallinity of at least 50%, at least 60%, at least 70%, at least 80%, at least 90%, at least 95%, at least 99%, or at least 99.5%, and up to 100%, as determined by XRPD and/or mDSC analysis. For example, the pharmaceutically acceptable salt of a compound of Formula (I-3) may be at least 50%, at least 60%, at least 70%, at least 80%, at least 90%, at least 95%, at least 99%, or at least 99.5% in crystalline form by weight of the pharmaceutically acceptable salt of the compound of Formula (I-3), e.g., as determined by X-ray powder diffraction and/or mDSC. In some embodiments, a highly pure crystalline form of a pharmaceutically acceptable salt of a compound of Formula (I-3) is provided. For example, the pharmaceutically acceptable salt of a compound of Formula (I-3), may be at least 90%, at least 95%, at least 99%, or at least 99.5% in crystalline form by weight of the pharmaceutically acceptable salt of the compound of Formula (I-3) present in, e.g., as determined by X-ray powder diffraction and/or mDSC. Preference is given to salt forms with high crystallinity, as determined e.g., by discrete and sharp Bragg diffractions in the X-ray diffractograms.

XRPD analyses can be carried out, e.g., on a Bruker AXS D2 diffractometer using CuKα radiation (wavelength=1.54060 Å). The instrument may be equipped with a fine focus X-ray tube. The tube voltage and amperage can be set to 30 kV and 10 mA, respectively, and a θ-θ geometry can be used, using a LynxEye detector from 5-42 °2θ, with a step size of 0.024 °2θ and a collection time of 0.1 seconds per step.

In terms of pharmaceutical production processes, advantageous salt forms of the compounds of Formula (I-3) according to methods described herein, are those that readily afford a solid material, either a crystalline solid or an amorphous solid, in acceptable yield without proceeding via an oil, and with favorable volume factors, making them suitable for mass production.

Salts forms of the compound of Formula (I-3) according to methods described herein, can in some cases exist in different polymorphs (i.e., forms having a different crystal structure), however, preferred salt forms of the present disclosure are those which can be generated as a single crystalline form or single polymorph or single amorphous form, as determined by XRPD and/or mDSC and/or differential scanning calorimetry (DSC), for example. It is also generally desirable for the salts to be free flowing, not cohere/adhere to surfaces, and possess a regular morphology.

### Chemical/Solid-State Stability

In some embodiments, the pharmaceutically acceptable salt of the compound of Formula (I-3), according to methods described herein, has a melt onset of from about 90° C., from about 100° C., from about 110° C., from about 120° C., from about 130° C., from about 140° C., from about 150° C., from about 160° C., from about 170° C., from about 180° C., from about 190° C., and up to about 250° C., up to about 240° C., up to about 230° C., up to about 225° C., up to about 210° C., up to about 200° C., as determined by DSC.

Pharmaceutically acceptable salts of the compound of Formula (I-3) according to methods described herein, may also be characterized as non-hygroscopic or slightly hygroscopic, preferably non-hygroscopic. The hygroscopicity may be measured herein by performing a moisture adsorption-desorption isotherm using a dynamic vapor sorption (DVS) analyzer with a starting exposure of 40% relative humidity (RH), increasing humidity up to 90% RH, decreasing humidity to 0% RH, increasing humidity to 90% RH, decreasing humidity to 0% RH, and finally increasing the humidity back to the starting 40% RH, and classified according to the following:
non-hygroscopic: <0.2%; slightly hygroscopic: ≥0.2% and <2%; hygroscopic: ≥2% and <15%; very hygroscopic: ≥15%; deliquescent: sufficient water is absorbed to form a liquid; all values measured as weight increase (w/w due to acquisition of water) at >90% RH and 25° C.

In some embodiments, the pharmaceutically acceptable salt of the compound of Formula (I-3) according to methods described herein, has a weight increase at >90% RH of less than 1% w/w, less than 0.8% w/w, less than 0.6% w/w, less than 0.5% w/w, less than 0.4% w/w, less than 0.3% w/w, less than 0.2% w/w, less than 0.1% w/w, less than 0.08% w/w, less than 0.06% w/w, less than 0.05% w/w, less than 0.02% w/w, as determined by DVS.

Dry powder samples of free base and salts can be maintained/stored in open or closed environments, such as in open or closed flasks/vials, under ambient or stress conditions e.g., 25° C./90+% RH, 40° C./75% RH, etc. without appreciable degradation or physical changes (e.g., changed forms, deliquesced, etc.). For example, dry powder samples of free base and salts forms disclosed herein may have a purity or form change of less than 10%, less than 5%, less than 1%, when stored under ambient conditions or stress conditions (e.g., increased temperature, e.g., 40° C., and/or humidity).

Solution-phase compositions of the free base and salts can be maintained/stored in open or closed environments, such as in open or closed flasks/vials, under ambient or stress conditions e.g., 25° C./90+% RH, 40° C./75% RH, etc. without appreciable degradation. Thus, in some embodiments, the present disclosure provides stable solution-phase compositions of free base and salt forms of the compounds of Formula (I-3) (e.g., stable solvates of free base or salt forms of compounds of Formula (I-3) which are in solvated form, preferably fully solvated form), which can be stored as a solution, such as in the form of an aqueous solution, an organic solvent solution, or a mixed aqueous-organic solvent solution, for prolonged periods of time without appreciable degradation or physical changes, such as oiling out of solution. Solvents which can be used to form the solution-phase compositions can be any one or more solvents set forth herein, e.g., water, ethanol, fruit juice, etc. In some embodiments, the solution-phase composition is an aqueous solution-phase composition comprising the free base or a pharmaceutically acceptable salt of the compound of Formula (I-3) solvated with water (and optionally comprising other components such as those found in fruit juice). The identification of stable solution-phase compositions of compounds of Formula (I-3) and their salts is advantageous at least because such compositions do not require use immediately after being prepared, such as within 5 minutes, within 4 minutes, within 3 minutes, within 2 minutes, within 1 minute, within 45 seconds, within 30 seconds, within 15 seconds, within 10 seconds of being prepared. Instead, the stable solution-phase compositions of the compounds of Formula (I-3) and salts thereof described herein can be prepared in advance, when desired, optionally stored, and can be administered hours, days, or even weeks after being prepared, without materially effecting efficacy, e.g., without appreciable degradation of the psilocin or psilocin-type active.

In some embodiments, aqueous solutions formed from the pharmaceutically acceptable salt of the compound of Formula (I-3) are characterized by increased stability compared to aqueous solutions that are prepared from the compound of Formula (I-3) (free base) but are otherwise substantially the same. For example, the pharmaceutically acceptable salt of the compound of Formula (I-3) may be at least 5%, at least 10%, at least 15%, at least 20%, at least 25%, at least 30%, at least 35%, at least 40%, at least 45%, at least 50%, at least 55%, at least 60%, at least 65%, at least 70% more stable in aqueous solution subjected to 40° C. for 24 hours, with or without the presence of metal ions, in terms of % (active) remaining, compared to aqueous solutions prepared with the compound of Formula (I-3) (free base) but are otherwise substantially the same. Such improved stability behavior can also be found in pharmaceutical compositions of the present disclosure.

Samples can be pulled at pre-determined time-points and analyzed for stability, changes in form, etc. for example, by 1H NMR, XRPD, HPLC with UV-visible multiple wavelength detector, UPLC, etc.

### Physiologically Acceptability

Suitable salt forms of the compounds of Formula (I-3) are physiologically acceptable. Accordingly, preferred addition salts of the compound of Formula (I-3) are those formed with an organic acid, preferably an organic acid with a medium or mild acidity, for example an organic acid with a pKₐ in water of no less than-3.0, no less than-2.0, no less than-1.0, no less than 0, no less than 1.0, no less than 1.5, no less than 2.0, no less than 2.5, no less than 3.0, no less than 3.5, no less than 4.0, no less than 4.5, for example, from 3.0 to 6.5. Further, it may also be desirable to use acid addition salts that impart a pleasant taste profile (e.g., sweet, citrus flavored, etc.), although poor tasting salt forms (e.g., bitter, harsh, etc.) may still be acceptable depending on, for example, the route of administration and the optional use of taste masking agents such as sweetening agents, flavoring agents, etc.

### Solubility

The aqueous solubility of the salt forms of the compounds of Formula (I-3) can be determined by equilibrating excess solid with 1 mL of water for 24 hours at 22° C. A 200 μL aliquot can be centrifuged at 15,000 rpm for 15 minutes. The supernatant can be analyzed by HPLC and the solubility can be expressed as its free base equivalent (mg FB/mL). For example, pharmaceutically acceptable salts of compound of Formula (I-3) can be prepared and the solubility and solution pH can be measured.

In some embodiments, the pharmaceutically acceptable salt of the compound of Formula (I-3) has a water solubility at 22° C. of from about 1 mg/mL to about 400 mg/mL. In some embodiments, the pharmaceutically acceptable salt of the compound of Formula (I-3) has a water solubility of from about 1 mg/mL, from about 2 mg/mL, from about 3 mg/mL, from about 5 mg/mL, from about 10 mg/mL, from about 20 mg/mL, from about 30 mg/mL, from about 40 mg/mL, from about 50 mg/mL, from about 60 mg/mL, from about 70 mg/mL, from about 80 mg/mL, from about 90 mg/mL, from about 100 mg/mL, from about 110 mg/mL, from about 120 mg/mL, from about 130 mg/mL, from about 140 mg/mL, from about 150 mg/mL, and up to about 400 mg/mL, up to about 380 mg/mL, up to about 360 mg/mL, up to about 340 mg/mL, up to about 320 mg/mL, up to about 300 mg/mL, up to about 280 mg/mL, up to about 260 mg/mL, up to about 250 mg/mL. Several salt forms of the compounds described herein can exhibit the above solubilities, yielding a final water pH approximately between pH 3 to 6 without gelling.

In some embodiments, the salt of the compound of Formula (I-3) has a water solubility from about 200 mg/mL to about 400 mg/mL. In some embodiments, the salt of the compound of Formula (I-3) has a water solubility from about 150 mg/mL to about 250 mg/mL. In some embodiments, the salt of the compound of Formula (I-3) has a water solubility of greater than about 1 mg/mL, 10 mg/mL, 20 mg/mL, 30 mg/mL, 40 mg/mL, 50 mg/mL, 60 mg/mL, 70 mg/mL, 80 mg/mL, 90 mg/mL, 100 mg/mL, 110 mg/mL, 120 mg/mL, 130 mg/mL, 140 mg/mL, or 150 mg/mL.

In some embodiments, salt forms of the compounds of Formula (I-3) possess dissolution rates which enable rapid systemic absorption for quick therapeutic onset and a short duration of drug action. In some embodiments, the salt of the compound of Formula (I-3) is capable of dissolution in an aqueous medium below about pH 7.5, such as from pH 1-7, from pH 3-7, or from pH 4-7.

In some embodiments, the pharmaceutically acceptable salt of the compound of Formula (I-3) is a benzenesulfonate salt, a tartrate salt, a hemi-fumarate salt, an acetate salt, a citrate salt, a hemi-malonate salt, a malonate salt, a fumarate salt, a succinate salt, a hemi-succinate salt, an oxalate salt, a benzoate salt, a salicylate salt, an ascorbate salt, a hydrochloride salt, a maleate salt, a malate salt, a methanesulfonate salt, a toluenesulfonate salt, a glucuronate salt, or a glutarate salt of the compound of Formula (I-3). In some embodiments, the pharmaceutically acceptable salt of the compound of Formula (I-3) is a salt formed from a sulfonic acid (e.g., benzenesulfonic acid, camphorsulfonic acid, (+)-(1S)-camphor-10-sulfonic acid, ethane-1,2-disulfonic acid, ethanesulfonic acid, 2-hydroxy-ethanesulfonic acid, methanesulfonic acid, naphthalene-2-sulfonic acid, naphthalene-1,5-disulfonic acid, p-toluenesulfonic acid, ethanedisulfonic acid, etc.). In some embodiments, the pharmaceutically acceptable salt of the compound of Formula (I-3) is a salt formed from a benzoic acid (e.g., benzoic acid, 4-acetamidobenzoic acid, 2-acetoxybenzoic acid, salicylic acid, 4-amino-salicylic acid, etc.). The pharmaceutically acceptable salt of the compound of Formula (I-3) may be a hemi-acid salt of any of the salts listed above when the acid used to form the salt contains more than one acidic group (e.g., more than one carboxylic acid moiety).

In some embodiments, the pharmaceutically acceptable salt of the compound of Formula (I-3) is a benzenesulfonate salt. In some embodiments, the pharmaceutically acceptable salt of the compound of Formula (I-3) is a tartrate salt. In some embodiments, the pharmaceutically acceptable salt of the compound of Formula (I-3) is a hemi-fumarate salt. In some embodiments, the pharmaceutically acceptable salt of the compound of Formula (I-3) is an acetate salt. In some embodiments, the pharmaceutically acceptable salt of the compound of Formula (I-3) is a citrate salt. In some embodiments, the pharmaceutically acceptable salt of the compound of Formula (I-3) is a hemi-malonate salt. In some embodiments, the pharmaceutically acceptable salt of the compound of Formula (I-3) is a fumarate salt. In some embodiments, the pharmaceutically acceptable salt of the compound of Formula (I-3) is a hemi-succinate salt. In some embodiments, the pharmaceutically acceptable salt of the compound of Formula (I-3) is an oxalate salt. In some embodiments, the pharmaceutically acceptable salt of the compound of Formula (I-3) is a benzoate salt. In some embodiments, the pharmaceutically acceptable salt of the compound of Formula (I-3) is a salicylate salt. In some embodiments, the pharmaceutically acceptable salt of the compound of Formula (I-3) is an ascorbate salt. In some embodiments, the pharmaceutically acceptable salt of the compound of Formula (I-3) is a hydrochloride salt. In some embodiments, the pharmaceutically acceptable salt of the compound of Formula (I-3) is a maleate salt. In some embodiments, the pharmaceutically acceptable salt of the compound of Formula (I-3) is a malate salt. In some embodiments, the pharmaceutically acceptable salt of the compound of Formula (I-3) is a methanesulfonate salt. In some embodiments, the pharmaceutically acceptable salt of the compound of Formula (I-3) is a toluenesulfonate salt. In some embodiments, the pharmaceutically acceptable salt of the compound of Formula (I-3) is a glucuronate salt. In some embodiments, the pharmaceutically acceptable salt of the compound of Formula (I-3) is a glutarate salt.

In some embodiments, the pharmaceutically acceptable salt of the compound of Formula (I-3) is a benzenesulfonate salt, a tartrate salt, a hemi-fumarate salt, an acetate salt, a citrate salt, a hemi-malonate salt, a fumarate salt, a hemi-succinate salt, an oxalate salt, a benzoate salt, or a salicylate salt of the compound of Formula (I-3), with a benzenesulfonate salt, a hemi-succinate salt, or a benzoate salt of the compound of Formula (I-3) being preferred, and with a benzenesulfonate salt or a benzoate salt of the compound of Formula (I-3) being particularly preferred.

Exemplary pharmaceutically acceptable salt forms (i.e., addition salt forms) of the above-identified compounds are provided in Table 1.

TABLE 1
Exemplary pharmaceutically
acceptable salts of compounds
of Formula (I-3)

| | |
|---|---|
| I-3a | Benzenesulfonate of I-3 |
| I-3b | Tartrate of I-3 |
| I-3c | Hemi-fumarate of I-3 |
| I-3d | Acetate of I-3 |
| I-3e | Citrate of I-3 |
| I-3f | Hemi-malonate of I-3 |
| I-3g | Fumarate of I-3 |
| I-3h | Hemi-succinate of I-3 |
| I-3i | Oxalate of I-3 |
| I-3j | Benzoate of I-3 |
| I-3k | Salicylate of I-3 |

In some embodiments, the pharmaceutically acceptable salt is a benzenesulfonate salt of 3-(2-(bis(methyl-d₃)amino)ethyl-1,1,2,2-d₄)-1H-indol-4-ol (I-3a). In some embodiments, salt I-3a is in a crystalline solid form (pattern 1) characterized by an X-ray powder diffraction pattern containing at least three characteristic peaks at diffraction angles (2θ±0.2°) selected from 7.023°, 7.767°, 11.822°, 12.550°, 12.860°, 13.994°, 15.521°, 18.436°, 19.503°, 20.760°, 21.070°, 22.007°, 22.745°, 23.340°, 24.187°, 25.532°, 26.880°, 27.856°, 28.163°, 31.267°, 33.024°, 35.030°, 36.835°, 39.312°, 40.545°, and 40.988°, as determined by XRPD using a CuKα radiation source, for example, as shown in WO2022195011 and/or WO2023078604.

In some embodiments, the pharmaceutically acceptable salt is a tartrate salt of 3-(2-(bis(methyl-d₃)amino)ethyl-1,1,2,2-d₄)-1H-indol-4-ol (I-3b). In some embodiments, salt I-3b is in a crystalline solid form of pattern 1 characterized by, e.g., an X-ray powder diffraction pattern shown in WO2022195011 and/or WO2023078604. In some embodiments, salt I-3b is in a crystalline solid form (pattern 2) characterized by an X-ray powder diffraction pattern containing at least three characteristic peaks at diffraction angles (2θ±0.2°) selected from 6.732°, 12.708°, 13.470°, 14.774°, 15.921°, 16.268°, 17.295°, 18.869°, 20.079°, 20.208°, 20.877°, 21.894°, 22.657°, 23.491°, 23.702°, 24.636°, 24.882°, 25.569°, 26.685°, 27.060°, 27.502°, 28.179°, 28.597°, 29.035°, 29.257°, 29.527°, 31.017°, 31.527°, 32.059°, 32.307°, 33.012°, 34.024°, 34.388°, 34.905°, 35.361°, 36.183°, 37.372°, 37.764°, 38.657°, and 41.049°, as determined by XRPD using a CuKα radiation source, for example, as shown in WO2022195011 and/or WO2023078604.

In some embodiments, the pharmaceutically acceptable salt is a hemi-fumarate salt of 3-(2-(bis(methyl-d₃)amino)ethyl-1,1,2,2-d₄)-1H-indol-4-ol (1-3c). In some embodiments, salt I-3c is in a crystalline solid form of pattern 1 characterized by, e.g., an X-ray powder diffraction pattern as shown in WO2022195011 and/or WO2023078604. In some embodiments, salt I-3c is in a crystalline solid form (pattern 2) characterized by an X-ray powder diffraction pattern containing at least three characteristic peaks at diffraction angles (2θ±0.2°) selected from 9.713°, 11.209°, 11.605°, 12.338°, 12.852°, 13.718°, 15.117°, 16.066°, 16.627°, 19.026°, 19.427°, 20.108°, 21.068°, 21.335°, 21.837°, 22.429°, 23.262°, 23.478°, 23.900°, 24.720°, 25.318°, 27.912°, 28.532°, 29.565°, 30.457°, 32.698°, 34.155°, 37.910°, 39.566°, and 40.999°, as determined by XRPD using a CuKα radiation source, for example, as shown in WO2022195011 and/or WO2023078604.

In some embodiments, the pharmaceutically acceptable salt is a citrate salt of 3-(2-(bis(methyl-d₃)amino)ethyl-1,1,2,2-d₄)-1H-indol-4-ol (I-3e). In some embodiments, salt I-3e is in the form of an amorphous solid as characterized by an X-ray powder diffraction (XRPD).

In some embodiments, the pharmaceutically acceptable salt is a benzoate salt of 3-(2-(bis(methyl-d₃)amino)ethyl-1,1,2,2-d₄)-1H-indol-4-ol (1-3j). In some embodiments, salt I-3j is in a crystalline solid form (pattern 1) characterized by an X-ray powder diffraction pattern containing at least three characteristic peaks at diffraction angles (2θ±0.2°) selected from 9.486°, 11.006°, 12.379°, 13.428°, 14.608°, 15.446°, 16.389°, 18.247°, 18.977°, 19.346°, 19.831°, 20.868°, 21.447°, 22.860°, 23.878°, 24.944°, 25.737°, 26.144°, 26.341°, 26.990°, 27.708°, 28.595°, 30.048°, 30.763°, 31.127°, 31.839°, 32.800°, 34.460°, 35.444°, 37.725°, and 38.597°, as determined by XRPD using a CuKα radiation source, for example, as shown in WO2022195011 and/or WO2023078604.

Preferred pharmaceutically acceptable salts of the compounds of Formula (I-3) enhance the stability, aqueous solubility, and rate of dissolution compared to the free base compound, while also possessing advantageous pharmaceutical characteristics (e.g., well-defined physical properties such as crystallinity, reproducible polymorphism insofar as polymorphism exists, high melt onset such as greater than 160° C., slight or no hygroscopicity, free flowing, etc.), which enables direct oral administration to patients without the need for prodrug approaches. As a result, pharmaceutically acceptable salts of the compounds of Formula (I-3) may have a faster/quicker therapeutic onset, a shorter duration of drug action (e.g., short duration of effect), and less variability in exposures than psilocybin-based drugs (e.g., psilocybin). Above all, the pharmaceutically acceptable salts of the compounds of Formula (I-3) have demonstrated surprising efficacy in the treatment of MDD.

In some embodiments, the pharmaceutically acceptable salt of the compound of Formula (I-3) is a fatty acid salt. The fatty acid used to make the fatty acid salt of the compound of Formula (1-3) may be a fatty monoacid or a fatty diacid, and may contain a fatty hydrocarbon portion made up of hydrogen and anywhere from 4, from 6, from 8, from 10, from 12, from 14, from 16, and up to 26, up to 24, up to 22, up to 20, up to 18 carbon atoms, which may be fully saturated or partially unsaturated. In some embodiments, the pharmaceutically acceptable salt of the compound of Formula (I-3) is an adipate salt, a laurate salt, a linoleate salt, a myristate salt, a caprate salt, a stearate salt, an oleate salt, a caprylate salt, a palmitate salt, a sebacate salt, an undecylenate salt, or a caproate salt of the compound of Formula (I-3). In some embodiments, the pharmaceutically acceptable salt of the compound of Formula (I-3) is an adipate salt, a laurate salt, a linoleate salt, a myristate salt, a caprate salt, a stearate salt, an oleate salt, or a caprylate salt of the compound of Formula (I-3), with a laurate salt, a linoleate salt, a caprate salt, or a caprylate salt of the compound of Formula (I-3) being preferred.

Exemplary pharmaceutically acceptable fatty acid salt forms (i.e., addition salt forms) of the above-identified compounds are provided in Table 2.

TABLE 2
Exemplary pharmaceutically
acceptable fatty acid salts of
compounds of Formula (1-3)

| Salt form | Salt type of |
|---|---|
| identifier | compound |
| I-3l | Adipate of I-3 |
| I-3m | Laurate of I-3 |
| I-3n | Linoleate of I-3 |
| I-30 | Myristate of I-3 |
| I-3p | Caprate of I-3 |
| I-3q | Stearate of I-3 |
| I-3r | Oleate of I-3 |
| I-3s | Caprylate of I-3 |

In some embodiments, the pharmaceutically acceptable salt is a laurate salt of 3-(2-(bis(methyl-d₃)amino)ethyl-1,1,2,2-d₄)-1H-indol-4-ol (I-3m). In some embodiments, salt I-3m is in a crystalline solid form of pattern 1 characterized by, e.g., an X-ray powder diffraction pattern as shown in WO2022195011 and/or WO2023078604.

In some embodiments, the pharmaceutically acceptable salt is a linoleate salt of 3-(2-(bis(methyl-d₃)amino)ethyl-1,1,2,2-d₄)-1H-indol-4-ol (I-3n). In some embodiments, salt I-3n is in a crystalline solid form of pattern 1 characterized by, e.g., an X-ray powder diffraction pattern as shown in WO2022195011 and/or WO2023078604.

In some embodiments, the pharmaceutically acceptable salt is a myristate salt of 3-(2-(bis(methyl-d₃)amino)ethyl-1,1,2,2-d₄)-1H-indol-4-ol (I-3o). In some embodiments, salt I-3o is in a crystalline solid form of pattern 1 characterized by, e.g., an X-ray powder diffraction pattern as shown in WO2022195011 and/or WO2023078604.

In some embodiments, the pharmaceutically acceptable salt is a caprate salt of 3-(2-(bis(methyl-d₃)amino)ethyl-1,1,2,2-d₄)-1H-indol-4-ol (I-3p). In some embodiments, salt I-3p is in a crystalline solid form of pattern 1 characterized by, e.g., an X-ray powder diffraction pattern as shown in WO2022195011 and/or WO2023078604.

In some embodiments, the pharmaceutically acceptable salt is a stearate salt of 3-(2-(bis(methyl-d₃)amino)ethyl-1,1,2,2-d₄)-1H-indol-4-ol (1-3q). In some embodiments, salt I-3q is in a crystalline solid form of pattern 1 or 2 characterized by, e.g., an X-ray powder diffraction pattern as shown in WO2022195011 and/or WO2023078604.

In some embodiments, the pharmaceutically acceptable salt is a oleate salt of 3-(2-(bis(methyl-d₃)amino)ethyl-1,1,2,2-d₄)-1H-indol-4-ol (I-3r). In some embodiments, salt I-3r is in a crystalline solid form of pattern 1 or 2 characterized by, e.g., an X-ray powder diffraction pattern as shown in WO2022195011 and/or WO2023078604.

In some embodiments, the pharmaceutically acceptable salt is a caprylate salt of 3-(2-(bis(methyl-d₃)amino)ethyl-1,1,2,2-d₄)-1H-indol-4-ol (1-3s). In some embodiments, salt I-3s is in a crystalline solid form of pattern 1 characterized by, e.g., an X-ray powder diffraction pattern as shown in WO2022195011 and/or WO2023078604.

In some embodiments, the pharmaceutically acceptable salt of the compound of Formula (I-3) has a solubility in corn oil at 22° C. of from about 0.4 mg/mL, from about 0.5 mg/mL, from about 0.6 mg/mL, from about 0.7 mg/mL, from about 0.8 mg/mL, from about 0.9 mg/mL, from about 1 mg/mL, and up to about 2 mg/mL, up to about 1.9 mg/mL, up to about 1.8 mg/mL, up to about 1.7 mg/mL, up to about 1.6 mg/mL, up to about 1.5 mg/mL, up to about 1.4 mg/mL, up to about 1.3 mg/mL, up to about 1.2 mg/mL.

In some embodiments, the pharmaceutically acceptable salt of the compound of Formula (I-3) has a solubility in Crodamol® GTCC (medium chain glyceride, from Croda) at 22° C. of from about 0.4 mg/mL, from about 0.6 mg/mL, from about 0.8 mg/mL, from about 1 mg/mL, from about 1.2 mg/mL, from about 1.4 mg/mL, from about 1.6 mg/mL, and up to about 4 mg/mL, up to about 3.8 mg/mL, up to about 3.6 mg/mL, up to about 3.4 mg/mL, up to about 3.2 mg/mL, up to about 3 mg/mL, up to about 2.8 mg/mL, up to about 2.6 mg/mL, up to about 2.4 mg/mL, up to about 2.2 mg/mL.

In some embodiments, the pharmaceutically acceptable salt of the compound of Formula (I-3) has a solubility in Maisine® CC (mixture of unsaturated mono-, di-, and triglycerides, from Gattefosse) at 22° C. of from about 0.8 mg/mL, from about 1 mg/mL, from about 1.2 mg/mL, from about 1.4 mg/mL, from about 1.6 mg/mL, from about 1.8 mg/mL, from about 2 mg/mL, and up to about 5 mg/mL, up to about 4.8 mg/mL, up to about 4.6 mg/mL, up to about 4.4 mg/mL, up to about 4.2 mg/mL, up to about 4 mg/mL, up to about 3.8 mg/mL, up to about 3.6 mg/mL, up to about 3.4 mg/mL, up to about 3.2 mg/mL, up to about 3 mg/mL, up to about 2.8 mg/mL, up to about 2.6 mg/mL, up to about 2.4 mg/mL, up to about 2.2 mg/mL.

Owing to their relatively hydrophobic nature, fatty acid salts of the compounds of Formula (I-3) may be advantageous when used in medications adapted for a modified, controlled, slow, or extended release profile. As a result, the fatty acid salts of the compounds of Formula (I-3) may be well suited for routes of administration and/or dosage forms adapted for providing low doses of active pharmaceutical ingredient (API) over extended periods of time, as may be the case for sub-psychedelic dosing regimens. Non-limiting examples of such dosage forms include, but are not limited to, liposomes, micelles, microspheres, nanosystems, or other controlled release devices, such as those set forth herein.

Also disclosed herein is a method for stabilizing a compound of Formula (I-3). The method includes preparing a pharmaceutically acceptable salt of the compound of Formula (I-3).

Also disclosed herein is a method for preparing a pharmaceutically acceptable salt of the compound of Formula (I-3). In some embodiments, the method includes:
(a) suspending the free base of the compound of Formula (I-3) in a solvent or mixture of solvents;
(b) contacting an acid with the compound of Formula (I-3) to provide a mixture;
(c) optionally heating the mixture;
(d) optionally cooling the mixture; and
(e) isolating the salt.

Various solvents may be used in the disclosed methods, including one or more protic solvents, one or more aprotic solvents, or mixtures thereof. In some embodiments, the solvent(s) used in the method of preparing the salt is/are a protic solvent(s). In some embodiments, the solvent used in the method of preparing the salt is selected from the group consisting of methanol, ethanol, propanol, isopropanol, butanol, 2-butanol, acetone, butanone, dioxanes (1,4-dioxane), water, tetrahydrofuran (THF), acetonitrile (MeCN), ether solvents (e.g., t-butylmethyl ether (TBME)), hexane, heptane, and octane, and combinations thereof. In some embodiments, the solvent is ethanol. In some embodiments, the solvent is 1,4-dioxane. In some embodiments, the solvent is acetonitrile. In some embodiments, the solvent is tetrahydrofuran.

Suitable acids for use in the preparation of pharmaceutically acceptable acid addition salts may include those described heretofore. The acid may be an inorganic acid such as hydrochloric acid, or an organic acid, with organic acids being preferred. In some embodiments, the acid is an organic acid selected from the group consisting of ascorbic acid, citric acid, fumaric acid, maleic acid, malonic acid, (−)-L-malic acid, (+)-L-tartaric acid, methanesulfonic acid, benzenesulfonic acid, toluenesulfonic acid, benzoic acid, salicylic acid, succinic acid, oxalic acid, D-glucuronic acid, glutaric acid salt, and acetic acid. In some embodiments, the acid is an organic acid selected from the group consisting of benzenesulfonic acid, (+)-L-tartaric acid, fumaric acid, acetic acid, citric acid, malonic acid, succinic acid, oxalic acid, benzoic acid, and salicylic acid, with benzenesulfonic acid, succinic acid, and benzoic acid being preferred. In some embodiments, the acid is a fatty acid, such as adipic (hexandioic) acid, lauric (dodecanoic) acid, linoleic acid, myristic (tetradecanoic) acid, capric (decanoic) acid, stearic (octadecanoic) acid, oleic acid, caprylic (octanoic) acid, palmitic (hexadecenoic) acid, sebacic acid, undecylenic acid, caproic acid, etc., with particular mention being made to adipic (hexandioic) acid, lauric (dodecanoic) acid, linoleic acid, myristic (tetradecanoic) acid, capric (decanoic) acid, stearic (octadecanoic) acid, oleic acid, and caprylic (octanoic) acid.

In some embodiments, a stoichiometric (or superstoichiometric) quantity of the acid is contacted with the compound of Formula (I-3). In some embodiments, a sub-stoichiometric (e.g., 0.5 molar equivalents) quantity of the acid is contacted with the compound of Formula (I-3). The use of sub-stoichiometric quantities of the acid may be desirable when, for example, the acid contains at least two acidic protons (e.g., two or more carboxylic acid groups) and the target salt is a hemi-acid salt.

In some embodiments, the mixture is heated, e.g., refluxed, prior to cooling.

In some embodiments, the mixture is cooled, and the salt is precipitated out of the solution. In some embodiments, the salt is precipitated out of solution in crystalline form. In some embodiments, the salt is precipitated out of solution in amorphous form.

Isolation of the salt may be performed by various well-known isolation techniques, such as filtration, decantation, and the like. In some embodiments, the isolating step includes filtering the mixture.

After isolation, additional crystallization and/or recrystallization steps may also optionally be performed, if desired, for example to increase purity, crystallinity, etc.

In some embodiments, compounds of the present disclosure, e.g., a compound of Formula (I-3), or a pharmaceutically acceptable salt, or a polymorph, thereof, is in the form of a solvate. Examples of solvate forms include, but are not limited to, hydrates, methanolates, ethanolates, isopropanolates, etc., with hydrates and ethanolates being preferred. The solvate may be formed from stoichiometric or nonstoichiometric quantities of solvent molecules. Solvates of the compounds herein may be in the form of isolable solvates. In one non-limiting example, as a hydrate, the compound may be a monohydrate, a dihydrate, etc. Solvates of the compounds herein also include solution-phase forms. Thus, in some embodiments, the present disclosure provides solution-phase compositions of the compounds of the present disclosure, or any pharmaceutically acceptable salts thereof, which are in solvated form, preferably fully solvated form.

In some embodiments, any position in the compound of Formula (I-3) indicated as having deuterium has a minimum deuterium incorporation that is greater than that found naturally occurring in hydrogen (about 0.016 atom %). In some embodiments, any position in the compound of Formula (I-3) indicated as having deuterium has a minimum deuterium incorporation of at least 10 atom %, at least 20 atom %, at least 25 atom %, at least 30 atom %, at least 40 atom %, at least 45 atom %, at least 50 atom %, at least 60 atom %, at least 70 atom %, at least 80 atom %, at least 90 atom %, at least 95 atom %, at least 99 atom % at the site of deuteration. Typically, any position in the compound of Formula (I-3) indicated as having deuterium has a minimum deuterium incorporation of at least 40 atom %, at least 45 atom %, at least 50 atom %, at least 60 atom %, at least 70 atom %, at least 80 atom %, at least 90 atom %, at least 95 atom %, at least 99 atom % at the site of deuteration.

A compound of Formula (I-3), or a pharmaceutically acceptable salt, polymorph, or solvate thereof, with less than 100% isotopic purity may be used. In some embodiments, the compound of Formula (I-3) (or a pharmaceutically acceptable salt, polymorph, or solvate thereof) has an isotopic purity of at least 50% by weight, at least 60% by weight, at least 70% by weight, at least 80% by weight, at least 90% by weight, at least 95% by weight, at least 99% by weight, based on a total weight of isotopologues of the compound of Formula (I-3) present. For example, the compound of Formula (I-3) may exist as an isotopologue mixture comprising psilocin-d₁₀ (compound I-3; 3-(2-(bis(methyl-d₃)amino)ethyl-1,1,2,2-d₄)-1H-indol-4-ol), in either free base or salt form, solvates, or mixtures thereof as the subject compound, and which may additionally contain lesser amounts of isotopologues of the subject compound, e.g., psilocin-d₉ (one or more of 3-(2-(bis(methyl-d₃)amino)ethyl-1,1,2-d₃)-1H-indol-4-ol and 3-(2-(bis(methyl-d₃)amino)ethyl-1,2,2-d₃)-1H-indol-4-ol), psilocin-d₈ (one or more of 3-(2-(bis(methyl-d₃)amino)ethyl-2,2-d₂)-1H-indol-4-ol, 3-(2-(bis(methyl-d₃)amino)ethyl-1,1-d₂)-1H-indol-4-ol, and 3-(2-(bis(methyl-d₃)amino)ethyl-1,2-d₂)-1H-indol-4-ol) etc., as free-base or salt forms, polymorphs, stereoisomers, solvates, or mixtures thereof. In some embodiments, the compound of Formula (I-3) is substantially free of other isotopologues of the compound, in either free base or salt form, e.g., has less than 20, 15, 10, 9, 8, 7, 6, 5, 4, 3, 2 or 1 or 0.5 mole percent of other isotopologues of the compound. In some embodiments, the compound of Formula (I-3), or a pharmaceutically acceptable salt, polymorph, or solvate thereof, has an isotopic purity by weight of at least 60%, at least 65%, at least 70%, at least 75%, at least 77%, at least 79%, at least 80%, at least 82%, at least 84%, at least 85%, at least 87%, at least 89%, at least 90%, with an isotopic purity by weight of up to 99%, up to 98%, up to 97%, up to 96%, up to 95%, up to 94%, up to 93%, up to 92%, up to 91%, up to 90%, up to 89%, up to 88%, up to 87%, up to 86%, up to 85%, up to 84%, up to 83%, up to 82%, up to 81%, up to 80%. In some embodiments, the compound of Formula (I-3), or a pharmaceutically acceptable salt, polymorph, or solvate thereof, is an isotopologue mixture (or active mixture) comprising: (i) from 60% to 99% by weight, from 60% to 98% by weight, from 65% to 97% by weight, from 70% to 96% by weight, from 75% to 95% by weight, from 80% to 94% by weight, from 85% to 93% by weight, from 90% to 92% by weight, from 75% to 90% by weight, from 76% to 89% by weight, from 77% to 88% by weight, from 78% to 87% by weight, from 79% to 86% by weight of psilocin-d₁₀, or a pharmaceutically acceptable salt, polymorph, or solvate thereof, based on a total weight of the isotopologue mixture, (ii) from 1% to 40% by weight, from 2% to 40% by weight, from 3% to 35% by weight, from 4% to 30% by weight, from 5% to 25% by weight, from 6% to 20% by weight, from 7% to 15% by weight, from 8% to 10% by weight, from 10% to 25% by weight, from 11% to 24% by weight, from 12% to 23% by weight, from 13% to 22% by weight, from 14% to 21% by weight, from 13% to 17% by weight of psilocin-d₉ (one or more of 3-(2-(bis(methyl-d₃)amino)ethyl-1,1,2-d₃)-1H-indol-4-ol and 3-(2-(bis(methyl-d₃)amino)ethyl-1,2,2-d₃)-1H-indol-4-ol), or a pharmaceutically acceptable salt, polymorph, or solvate thereof, based on a total weight of the isotopologue mixture, and (iii) less than 10% by weight, less than 5% by weight, less than 3% by weight, less than 2% by weight, less than 1% by weight, less than 0.5% by weight, less than 0.25% by weight, or 0% by weight, or 1% to 3% by weight of psilocin-d₈ (one or more of 3-(2-(bis(methyl-d₃)amino)ethyl-2,2-d₂)-1H-indol-4-ol, 3-(2-(bis(methyl-d₃)amino)ethyl-1,1-d₂)-1H-indol-4-ol, and 3-(2-(bis(methyl-d₃)amino)ethyl-1,2-d₂)-1H-indol-4-ol), or a pharmaceutically acceptable salt, polymorph, or solvate thereof, based on a total weight of the isotopologue mixture.

In some embodiments, the compound of Formula (I-3), or a pharmaceutically acceptable salt, polymorph, or solvate thereof is chemically pure, for example has a chemical purity of greater than 90%, 92%, 94%, 96%, 97%, 98%, or 99% by HPLC. In some embodiments, the compound of Formula (I-3), or a pharmaceutically acceptable salt, polymorph, or solvate thereof has no single impurity of greater than 1%, greater than 0.5%, greater than 0.4%, greater than 0.3%, or greater than 0.2%, measured by HPLC. In some embodiments, the compound of Formula (I-3), or a pharmaceutically acceptable salt, polymorph, or solvate thereof has a chemical purity of greater than 97 area %, greater than 98 area %, or greater than 99 area % by HPLC. In some embodiments, the compound of Formula (I-3), or a pharmaceutically acceptable salt, polymorph, or solvate thereof has no single impurity greater than 1 area %, greater than 0.5 area %, greater than 0.4 area %, greater than 0.3 area %, or greater than 0.2 area % as measured by HPLC.

### Pharmaceutical Compositions Useful in the Methods of the Invention

In any embodiment of the present invention, the compound of Formula (I-3) or a pharmaceutically acceptable salt, polymorph, or solvate thereof can be administered as a pharmaceutical composition comprising the compound of Formula (I-3) or a pharmaceutically acceptable salt, polymorph, or solvate thereof according to any embodiment described herein. Thus, the present invention describes a pharmaceutical composition and methods useful for treating various diseases, disorders, and conditions, such as depressive disorders (e.g., Major Depressive Disorder (MDD)), substance use disorders, anxiety disorders, eating disorders, and headache disorders, in a subject in need thereof, the pharmaceutical composition comprising about 8-16 mg, or about 8-14 mg, or about 8-12 mg, or about 12-16 mg, or about 12 mg, or about 16 mg of a compound of Formula (I-3)

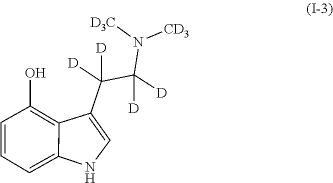 [SMILES: [2H]C([2H])([2H])N(C([2H])([2H])[2H])C([2H])([2H])C([2H])([2H])c1c[nH]c2cccc(O)c12]

or a pharmaceutically acceptable salt, polymorph, or solvate thereof; and
a pharmaceutically acceptable vehicle. In some embodiments, the compound of Formula (I-3) is a compound of Formula (I-3), or a salt or polymorph thereof as described in any embodiment described herein.

The pharmaceutical compositions according to any embodiment described herein, may contain one, or more than one, compound, salt form, polymorph, and/or solvate of the present disclosure.

The pharmaceutical compositions of the present disclosure may be formulated with a therapeutically effective amount of a compound of Formula (I-3)

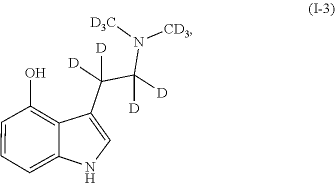 [SMILES: [2H]C([2H])([2H])N(C([2H])([2H])[2H])C([2H])([2H])C([2H])([2H])c1c[nH]c2cccc(O)c12]

or a pharmaceutically acceptable salt, polymorph, or solvate thereof. In some embodiments, a therapeutically effective amount of a compound of Formula (I-3), or a pharmaceutically acceptable salt, polymorph, or solvate thereof, is about 8 mg, about 9 mg, about 10 mg, about 11 mg, about 12 mg, about 13 mg, about 14 mg, about 15 mg, about 16 mg, or any range therebetween, such as from about 8 to about 16 mg, about 8 to about 14 mg, about 8 to about 12 mg, about 10 to about 16 mg, about 11 to about 15 mg, about 12 to about 14 mg, about 11 to about 13 mg, about 14 to about 16 mg, or about 12 to about 16 mg (free base equivalence). In some embodiments, pharmaceutical composition comprises 8 mg to 16 mg of a compound of Formula (I-3), or a pharmaceutically acceptable salt, polymorph, or solvate thereof. In some embodiments, the pharmaceutical composition comprises 8 mg to 14 mg of a compound of Formula (I-3), or a pharmaceutically acceptable salt, polymorph, or solvate thereof. In some embodiments, the pharmaceutical composition comprises 8 mg to 12 mg of a compound of Formula (I-3), or a pharmaceutically acceptable salt, polymorph, or solvate thereof. In some embodiments, the pharmaceutical composition comprises 8 mg to 10 mg of a compound of Formula (I-3), or a pharmaceutically acceptable salt, polymorph, or solvate thereof. In some embodiments, the pharmaceutical composition comprises 10 mg to 16 mg of a compound of Formula (I-3), or a pharmaceutically acceptable salt, polymorph, or solvate thereof. In some embodiments, the pharmaceutical composition comprises 12 mg to 16 mg of a compound of Formula (I-3), or a pharmaceutically acceptable salt, polymorph, or solvate thereof. In some embodiments, the pharmaceutical composition comprises 10 mg to 14 mg of a compound of Formula (I-3), or a pharmaceutically acceptable salt, polymorph, or solvate thereof. In some embodiments, the pharmaceutical composition comprises 12 mg to 14 mg of a compound of Formula (I-3), or a pharmaceutically acceptable salt, polymorph, or solvate thereof. In some embodiments, the pharmaceutical composition comprises 11 mg to 16 mg of a compound of Formula (I-3), or a pharmaceutically acceptable salt, polymorph, or solvate thereof. In some embodiments, the pharmaceutical composition comprises 11 mg to 15 mg of a compound of Formula (I-3), or a pharmaceutically acceptable salt, polymorph, or solvate thereof. In some embodiments, the pharmaceutical composition comprises 11 mg to 13 mg of a compound of Formula (I-3), or a pharmaceutically acceptable salt, polymorph, or solvate thereof. In some embodiments, the pharmaceutical composition comprises about 8 mg of a compound of Formula (I-3), or a pharmaceutically acceptable salt, polymorph, or solvate thereof. In some embodiments, the pharmaceutical composition comprises 8 mg of a compound of Formula (I-3), or a pharmaceutically acceptable salt, polymorph, or solvate thereof. In some embodiments, the pharmaceutical composition comprises about 9 mg of a compound of Formula (I-3), or a pharmaceutically acceptable salt, polymorph, or solvate thereof. In some embodiments, the pharmaceutical composition comprises 9 mg of a compound of Formula (I-3), or a pharmaceutically acceptable salt, polymorph, or solvate thereof. In some embodiments, the pharmaceutical composition comprises about 10 mg of a compound of Formula (I-3), or a pharmaceutically acceptable salt, polymorph, or solvate thereof. In some embodiments, the pharmaceutical composition comprises 10 mg of a compound of Formula (I-3), or a pharmaceutically acceptable salt, polymorph, or solvate thereof. In some embodiments, the pharmaceutical composition comprises about 11 mg of a compound of Formula (I-3), or a pharmaceutically acceptable salt, polymorph, or solvate thereof. In some embodiments, the pharmaceutical composition comprises 11 mg of a compound of Formula (I-3), or a pharmaceutically acceptable salt, polymorph, or solvate thereof. In some embodiments, the pharmaceutical composition comprises about 12 mg of a compound of Formula (I-3), or a pharmaceutically acceptable salt, polymorph, or solvate thereof. In some embodiments, the pharmaceutical composition comprises 12 mg of a compound of Formula (I-3), or a pharmaceutically acceptable salt, polymorph, or solvate thereof. In some embodiments, the pharmaceutical composition comprises about 13 mg of a compound of Formula (I-3), or a pharmaceutically acceptable salt, polymorph, or solvate thereof. In some embodiments, the pharmaceutical composition comprises 13 mg of a compound of Formula (I-3), or a pharmaceutically acceptable salt, polymorph, or solvate thereof. In some embodiments, the pharmaceutical composition comprises about 14 mg of a compound of Formula (I-3), or a pharmaceutically acceptable salt, polymorph, or solvate thereof. In some embodiments, the pharmaceutical composition comprises 14 mg of a compound of Formula (I-3), or a pharmaceutically acceptable salt, polymorph, or solvate thereof. In some embodiments, the pharmaceutical composition comprises about 15 mg of a compound of Formula (I-3), or a pharmaceutically acceptable salt, polymorph, or solvate thereof. In some embodiments, the pharmaceutical composition comprises 15 mg of a compound of Formula (I-3), or a pharmaceutically acceptable salt, polymorph, or solvate thereof. In some embodiments, the pharmaceutical composition comprises about 16 mg of a compound of Formula (I-3), or a pharmaceutically acceptable salt, polymorph, or solvate thereof. In some embodiments, the pharmaceutical composition comprises 16 mg of a compound of Formula (I-3), or a pharmaceutically acceptable salt, polymorph, or solvate thereof. In some embodiments, the pharmaceutical composition is administered orally, and the dosages of the compound of Formula (I-3), or a pharmaceutically acceptable salt, polymorph, or solvate thereof within the pharmaceutical composition recited above are oral dosages.

The pharmaceutical composition may comprise a single compound of Formula (I-3), or a pharmaceutically acceptable salt, polymorph, or solvate thereof, or a mixture of compounds of Formula (I-3), in either free base or salt form, including one or more polymorphs of such materials.

The pharmaceutical composition may be formed from a compound of Formula (I-3), or a pharmaceutically acceptable salt, polymorph, or solvate thereof, with less than 100% isotopic purity, i.e., with an isotopologue mixture. In some embodiments, the compound of Formula (I-3) (or a pharmaceutically acceptable salt, polymorph, or solvate thereof) has an isotopic purity of at least 50% by weight, at least 60% by weight, at least 70% by weight, at least 80% by weight, at least 90% by weight, at least 95% by weight, at least 99% by weight, based on a total weight of isotopologues of the compound of Formula (I-3) present, e.g., in a pharmaceutical composition. For example, a pharmaceutical composition formulated with psilocin-d₁₀ (compound I-3; 3-(2-(bis(methyl-d₃)amino)ethyl-1,1,2,2-d₄)-1H-indol-4-ol), in either free base or salt form, solvates, or mixtures thereof as the subject compound, may additionally contain lesser amounts of isotopologues of the subject compound, e.g., psilocin-d₉ (one or more of 3-(2-(bis(methyl-d₃)amino)ethyl-1,1,2-d₃)-1H-indol-4-ol and 3-(2-(bis(methyl-d₃)amino)ethyl-1,2,2-d₃)-1H-indol-4-ol), psilocin-d₈ (one or more of 3-(2-(bis(methyl-d₃)amino)ethyl-2,2-d₂)-1H-indol-4-ol, 3-(2-(bis(methyl-d₃)amino)ethyl-1,1-d₂)-1H-indol-4-ol, and 3-(2-(bis(methyl-d₃)amino)ethyl-1,2-d₂)-1H-indol-4-ol) etc., as free-base or salt forms, polymorphs, stereoisomers, solvates, or mixtures thereof. In some embodiments, the composition is substantially free of other isotopologues of the compound, in either free base or salt form, e.g., the composition has less than 20, 15, 10, 9, 8, 7, 6, 5, 4, 3, 2 or 1 or 0.5 mole percent of other isotopologues of the compound. In some embodiments, the pharmaceutical composition comprises the compound of Formula (I-3), or a pharmaceutically acceptable salt, polymorph, or solvate thereof, with an isotopic purity by weight of at least 60%, at least 65%, at least 70%, at least 75%, at least 77%, at least 79%, at least 80%, at least 82%, at least 84%, at least 85%, at least 87%, at least 89%, at least 90%, with an isotopic purity by weight of up to 99%, up to 98%, up to 97%, up to 96%, up to 95%, up to 94%, up to 93%, up to 92%, up to 91%, up to 90%, up to 89%, up to 88%, up to 87%, up to 86%, up to 85%, up to 84%, up to 83%, up to 82%, up to 81%, up to 80%. In some embodiments, the compound of Formula (I-3), or a pharmaceutically acceptable salt, polymorph, or solvate thereof, is an isotopologue mixture (or active mixture) comprising: (i) from 60% to 99% by weight, from 60% to 98% by weight, from 65% to 97% by weight, from 70% to 96% by weight, from 75% to 95% by weight, from 80% to 94% by weight, from 85% to 93% by weight, from 90% to 92% by weight, from 75% to 90% by weight, from 76% to 89% by weight, from 77% to 88% by weight, from 78% to 87% by weight, from 79% to 86% by weight of psilocin-d₁₀, or a pharmaceutically acceptable salt, polymorph, or solvate thereof, based on a total weight of the isotopologue mixture, (ii) from 1% to 40% by weight, from 2% to 40% by weight, from 3% to 35% by weight, from 4% to 30% by weight, from 5% to 25% by weight, from 6% to 20% by weight, from 7% to 15% by weight, from 8% to 10% by weight, from 10% to 25% by weight, from 11% to 24% by weight, from 12% to 23% by weight, from 13% to 22% by weight, from 14% to 21% by weight, from 13% to 17% by weight of psilocin-do (one or more of 3-(2-(bis(methyl-d₃)amino)ethyl-1,1,2-d₃)-1H-indol-4-ol and 3-(2-(bis(methyl-d₃)amino)ethyl-1,2,2-d₃)-1H-indol-4-ol), or a pharmaceutically acceptable salt, polymorph, or solvate thereof, based on a total weight of the isotopologue mixture, and (iii) less than 10% by weight, less than 5% by weight, less than 3% by weight, less than 2% by weight, less than 1% by weight, less than 0.5% by weight, less than 0.25% by weight, or 0% by weight, or 1% to 3% by weight of psilocin-d₈ (one or more of 3-(2-(bis(methyl-d₃)amino)ethyl-2,2-d₂)-1H-indol-4-ol, 3-(2-(bis(methyl-d₃)amino)ethyl-1,1-d₂)-1H-indol-4-ol, and 3-(2-(bis(methyl-d₃)amino)ethyl-1,2-d₂)-1H-indol-4-ol), or a pharmaceutically acceptable salt, polymorph, or solvate thereof, based on a total weight of the isotopologue mixture.

The pharmaceutical composition may be formulated with one or more polymorphs of the compounds of Formula (I-3) and/or their salt forms, including crystalline and/or amorphous polymorphs of the compounds or salts thereof. In some embodiments, the pharmaceutical composition includes a mixture of crystalline polymorphs. In some embodiments, the pharmaceutical composition includes a single crystalline polymorph. In some embodiments, the pharmaceutical composition includes a mixture of amorphous polymorphs. In some embodiments, the pharmaceutical composition includes a single amorphous polymorph. In some embodiments, the pharmaceutical composition includes a mixture of crystalline and amorphous polymorphs.

In some embodiments, the pharmaceutical composition comprises a compound of Formula (I-3), or a pharmaceutically acceptable salt or solvate thereof in crystalline form. In some embodiments, the pharmaceutical composition comprises a highly pure crystalline form of a compound of Formula (I-3) as a free base. For example, the pharmaceutical composition may comprise a free base of a compound of Formula (I-3), wherein at least 90%, at least 95%, at least 99%, or at least 99.5% by weight of the free base of the compound of Formula (I-3) present in the pharmaceutical composition is in crystalline form, e.g., as determined by X-ray powder diffraction and/or mDSC. In some embodiments, the pharmaceutical composition comprises a highly pure crystalline form of a pharmaceutically acceptable salt of a compound of Formula (I-3). For example, the pharmaceutical composition may comprise a pharmaceutically acceptable salt of a compound of Formula (I-3), wherein at least 90%, at least 95%, at least 99%, or at least 99.5% by weight of the pharmaceutically acceptable salt of the compound of Formula (I-3) present in the pharmaceutical composition is in crystalline form, e.g., as determined by X-ray powder diffraction and/or mDSC.

In some embodiments, the pharmaceutical composition comprises a compound of Formula (I-3), or a pharmaceutically acceptable salt or solvate thereof in amorphous form. In some embodiments, only the amorphous form of the compound of Formula (I-3), or a pharmaceutically acceptable salt, or solvate thereof is present in the pharmaceutical composition, e.g., no crystalline forms of the compound of Formula (I-3) are detectable, for example by XRPD. In some embodiments, the pharmaceutical composition comprises a highly pure amorphous form of a compound of Formula (I-3) as a free base. For example, the pharmaceutical composition may comprise a free base of a compound of Formula (I-3), wherein at least 92%, at least 94%, at least 96%, at least 98%, at least 99%, or at least 99.5% by weight of the free base of the compound of Formula (I-3) present in the pharmaceutical composition is in amorphous form, e.g., as determined by X-ray powder diffraction and/or mDSC. In some embodiments, the pharmaceutical composition comprises a highly pure amorphous form of a pharmaceutically acceptable salt of a compound of Formula (I-3). For example, the pharmaceutical composition may comprise a pharmaceutically acceptable salt of a compound of Formula (I-3), wherein at least 92%, at least 94%, at least 96%, at least 98%, at least 99%, or at least 99.5% by weight of the pharmaceutically acceptable salt of the compound of Formula (I-3) present in the pharmaceutical composition is in amorphous form, e.g., as determined by X-ray powder diffraction and/or mDSC.

In addition to a compound of Formula (I-3), or a pharmaceutically acceptable salt, polymorph, or solvate thereof, pharmaceutical compositions of the present disclosure may also comprise a pharmaceutically acceptable vehicle. “Pharmaceutically acceptable vehicles” may be vehicles approved by a regulatory agency of the Federal or a state government or listed in the U.S. Pharmacopeia or other generally recognized pharmacopeia for use in mammals, such as humans. The term “vehicle” refers to a diluent, adjuvant, excipient, or carrier with which a compound of the present disclosure is formulated for administration to a mammal. Such pharmaceutically acceptable vehicles can be solids or liquids. The pharmaceutically acceptable vehicles can include water, saline, juice including fruit juice particularly a fruit juice comprising citric acid (e.g., orange juice such as Tang, grape juice, apple juice, cranberry juice, pineapple juice, etc.), oils, including those of petroleum, animal, vegetable or synthetic origin, such as peanut oil, soybean oil, mineral oil, sesame oil and the like, gum acacia, gelatin, starch paste, talc, keratin, colloidal silica, urea, and the like. Pharmaceutically acceptable vehicles can include, but are not limited to, auxiliary agents, stabilizing agents, solubilizing agents, thickening agents, lubricants, binders, granulators, fillers, diluents, disintegrants, wetting agents, glidants, anti-caking agents, coloring agents, sweetening agents, dye-migration inhibitors, preservatives, antioxidants, lyoprotectants, complexing agents, flavoring agents, matrix-forming agents, dispersing agents, performance modifiers, controlled-release polymers, solvents, pH modifiers, sources of carbon dioxide, or other pharmaceutical additives set forth herein. In some embodiments, Tang orange drink is used as a pharmaceutically acceptable vehicle. In addition to water, Tang orange drink may contain sugar, fructose, citric acid, maltodextrin, calcium phosphate, sodium acid pyrophosphate, ascorbic acid (vitamin c), natural flavor, artificial color, guar gum, yellow 5, yellow 6, and xanthan gum.

Of these pharmaceutically acceptable vehicles, some organic acids have been identified as providing both a stabilizing function and a solubilizing function to the psilocin-d₁₀ compounds of the present disclosure (i.e., compounds of Formula (I-3), or a pharmaceutically acceptable salt, polymorph, or solvate thereof), thereby improving the delivery and therapeutic characteristics of the disclosed dosage forms. These organic acid vehicles which provide the unique stabilizing and solubilizing effect (act as a stabilizing/solubilizing agent) may be referred to herein as an “organic acid agent.” In preferred embodiments, the pharmaceutical composition comprises a compound of Formula (I-3), or a pharmaceutically acceptable salt, polymorph, or solvate thereof, and an organic acid agent. The pharmaceutical composition can optionally be formulated with other pharmaceutically acceptable vehicles as needed or desired.

In some embodiments, solid dosage forms are formulated with an organic acid agent, wherein the organic acid agent is considered separate and distinct from the compound of Formula (I-3), or a pharmaceutically acceptable salt, polymorph, or solvate thereof, i.e., when formulated in solid dosage form, the organic acid agent is not considered to form a salt with the compound of Formula (I-3). For example, in these embodiments where the pharmaceutical composition is a solid dosage form formulated with a free base of a compound of Formula (I-3), the organic acid agent is not considered to form an addition salt with the compound of Formula (I-3), and instead the compound of Formula (I-3) remains as a free base, at least until the point of dissolution/disintegration in an appropriate medium (e.g., water, juice, saline, saliva, etc.). In another example, where the pharmaceutical composition is formulated with a salt form of a compound of Formula (I-3), the organic acid agent remains separate from the salt form and provides a stabilizing/solubilizing effect above that provided by the salt form of the compound of Formula (I-3) alone.

Organic acid agents may be any organic acid described herein, and may be a monoacid, a diacid, a triacid, a tetraacid, or may contain a higher number of acid groups. One organic acid agent or mixtures of organic acid agents may be used. In addition to an acid group(s) (e.g., one or more carboxylic acid moieties), the organic acid agent may also contain one or more hydroxyl functionalities as part of its structure (i.e., the organic acid agent may be a hydroxy acid). In some embodiments, the organic acid agent is an α-hydroxy acid. In some embodiments, the organic acid agent is a β-hydroxy acid. In some embodiments, the organic acid agent is a γ-hydroxy acid. Examples of hydroxy acids include, but are not limited to, glycolic acid, lactic acid, citric acid, tartaric acid, and malic acid. In some embodiments, the organic acid agent is citric acid and/or tartaric acid. In some embodiments, the organic acid agent is citric acid. In some embodiments, the organic acid agent is tartaric acid. In some embodiments, the organic acid agent is an enedioic acid, examples of which may include, but are not limited to, fumaric acid and maleic acid. In some embodiments, the organic acid agent is fumaric acid. In some embodiments, the organic acid agent is maleic acid. Mixtures and/or hydrates of the disclosed organic acid agent may also be used in the disclosed pharmaceutical compositions. In some embodiments, the organic acid agent is not a sulfonic acid (e.g., benzenesulfonic acid, camphorsulfonic acid, (+)-(1S)-camphor-10-sulfonic acid, ethane-1,2-disulfonic acid, ethanesulfonic acid, 2-hydroxy-ethanesulfonic acid, methanesulfonic acid, naphthalene-2-sulfonic acid, naphthalene-1,5-disulfonic acid, p-toluenesulfonic acid, ethanedisulfonic acid, etc.). In some embodiments, the organic acid agent is not a benzoic acid (e.g., benzoic acid, 4-acetamidobenzoic acid, 2-acetoxybenzoic acid, salicylic acid, 4-amino-salicylic acid, gentisic acid, etc.).

In some embodiments, the pharmaceutical composition comprises at least 0.5% by weight, at least 1% by weight, at least 2% by weight, at least 3% by weight, at least 4% by weight, at least 5% by weight, at least 6% by weight, at least 7% by weight, at least 8% by weight, at least 9% by weight, at least 10% by weight, at least 11% by weight, at least 12% by weight, at least 13% by weight, at least 14% by weight, at least 15% by weight, and up to 60% by weight, up to 55% by weight, up to 50% by weight, up to 45% by weight, up to 40% by weight, up to 35% by weight, up to 30% by weight, up to 27% by weight, up to 25% by weight, up to 23% by weight, up to 20% by weight, up to 18% by weight, up to 16% by weight of the organic acid agent, based on a total weight of the pharmaceutical composition (on a dry basis), or any range therebetween. For example, the pharmaceutical composition may contain from 1% to 20% by weight of organic agent, or from 2% to 10% by weight of organic agent, or from 5% to 40% by weight of the organic acid agent, or from 10% to 30% by weight of organic agent, or from 15 to 20% of organic acid agent, or from 2% to 3% by weight of organic acid agent, or about 2.5% to 3% by weight of organic acid agent, based on a total weight of the pharmaceutical composition (on a dry basis). Dry basis may refer to pharmaceutical compositions which are in solid dosage form, or liquid dosage forms after subtracting the weight contribution from water or other pharmaceutically acceptable aqueous medium (e.g., fruit juice).

In some embodiments, a weight ratio of the organic acid agent to the compound of Formula (I-3) (active basis) is from 1:1, from 1.5:1, from 2:1, from 2.5:1, from 3:1, from 3.5:1, from 4:1, from 4.5:1, from 5:1, and up to 20:1, up to 15:1, up to 10:1, up to 9:1, up to 8:1, up to 7:1, up to 6:1, or any range therebetween.

When the pharmaceutical composition is formulated with a pharmaceutically acceptable salt of a compound of Formula (I-3), the acid used in forming the pharmaceutically acceptable salt of a compound of Formula (I-3) and the organic acid agent (vehicle) can be the same. For example, the pharmaceutical composition may comprise a tartrate salt of a compound of Formula (I-3) (e.g., I-3b), and tartaric acid as organic acid agent (vehicle). In another example, the pharmaceutical composition may comprise a citrate salt of a compound of Formula (I-3) (e.g., I-3e), and citric acid as organic acid agent (vehicle).

When the pharmaceutical composition is formulated with a pharmaceutically acceptable salt of a compound of Formula (I-3), the acid used in forming the pharmaceutically acceptable salt of a compound of Formula (I-3) and the organic acid agent (vehicle) can be different. For example, the pharmaceutical composition may comprise a benzenesulfonate salt of a compound of Formula (I-3) (1-3a), and citric acid and/or tartaric acid, etc., as organic acid agent (vehicle). In another example, the pharmaceutical composition may comprise a benzoate salt of a compound of Formula (I-3) (e.g., I-3j), and citric acid and/or tartaric acid, etc., as organic acid agent (vehicle).

Any of the pharmaceutical compositions disclosed herein formulated with an organic acid agent may contain an organic acid agent which is uncoated, or alternatively, may contain an organic acid agent which is coated (a “coated organic acid agent”) with a pharmaceutically acceptable vehicle. Examples of coated organic acid agents are set forth hereinafter.

The pharmaceutical compositions disclosed herein may be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

In the case wherein the patient's condition does not improve, upon the doctor's discretion the compounds may be administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the compounds may be given continuously or temporarily suspended for a certain length of time (i.e., a “drug holiday”).

Once improvement of the patient's conditions has occurred, a maintenance dose is administered if necessary. Subsequently, the dosage or the frequency of administration, or both, can be reduced, as a function of the symptoms, to a level at which the improved disorder is retained. Patients can, however, require intermittent treatment on a long-term basis upon any recurrence of symptoms.

Pharmaceutical compositions can take the form of capsules, tablets, pills, pellets, lozenges, powders, granules, syrups, elixirs, solutions, suspensions, emulsions, or sustained-release formulations thereof, or any other form suitable for administration to a mammal. Administration of the subject compounds may be systemic or local. In some instances, the pharmaceutical compositions are formulated for administration in accordance with routine procedures as a pharmaceutical composition adapted for oral administration, or other routes of administration as set forth herein, to humans. Examples of suitable pharmaceutically acceptable vehicles and methods for formulation thereof are described in Remington: The Science and Practice of Pharmacy, Alfonso R. Gennaro ed., Mack Publishing Co. Easton, Pa., 19th ed., 1995, Chapters 86, 87, 88, 91, and 92, incorporated herein by reference. The choice of vehicle will be determined in part by the particular compound, salt form, as well as by the particular method used to administer the composition. Accordingly, there is a wide variety of suitable formulations of the subject pharmaceutical compositions. Liquid form preparations include solutions and emulsions, for example, water, water/propylene glycol solutions, or organic solvents. When administered to a mammal, the compounds and compositions of the present disclosure and pharmaceutically acceptable vehicles may be sterile. In some instances, an aqueous medium is employed as a vehicle e.g., when the subject compound is administered orally, such as water, or fruit juices.

Any of the pharmaceutical compositions described herein can comprise (as the active component) at least one compound of Formula (I-3), or a pharmaceutically acceptable salt, polymorph, or solvate thereof. As described below, pharmaceutical compositions comprising a compound disclosed herein may be formulated in various dosage forms, and specially formulated for administration in solid, semi-solid, or liquid form, including those adapted for the following:
A. Oral administration, for example, drenches (aqueous or non-aqueous solutions or suspensions), tablets, films, or capsules, e.g., those targeted for buccal, sublingual, and systemic absorption, boluses, powders, granules, syrups, pastes for application to the tongue;
B. Modified release dosage forms, including delayed-, extended-, prolonged-, sustained-, pulsatile-, controlled-, accelerated-, fast-, targeted-, programmed-release, and gastric retention dosage forms, such modified release dosage forms can be prepared according to conventional methods and techniques known to those skilled in the art (see, Remington: The Science and Practice of Pharmacy, supra; Modified-Release Drug Delivery Technology, Rathbone et al., Eds., Drugs and the Pharmaceutical Science, Marcel Dekker, Inc.: New York, N.Y., 2002; Vol. 126).
   Tamper resistant dosage forms/packaging of any of the disclosed pharmaceutical compositions are contemplated.

### A. Oral Administration

The pharmaceutical compositions disclosed herein may be provided in solid, semisolid, or liquid dosage forms for oral administration. As used herein, oral administration includes gastric (enteral) delivery, for example whereby the medication is taken by mouth and swallowed, as well as intraoral administration such as through the mucosal linings of the oral cavity, e.g., buccal, lingual, and sublingual administration. Suitable oral dosage forms include, but are not limited to, tablets, capsules, pills, troches, lozenges, pastilles, cachets, pellets, medicated chewing gum, granules, bulk powders, effervescent or non-effervescent dosage forms (e.g., effervescent or non-effervescent tablets, films, powders or granules), solutions, emulsions, suspensions, solutions, wafers, films, sprinkles, elixirs, and syrups. In addition to the active ingredient(s), the pharmaceutical compositions may contain one or more pharmaceutically acceptable vehicles (e.g., carriers or excipients), including, but not limited to, auxiliary agents, stabilizing agents, solubilizing agents, thickening agents, lubricants, binders, granulators, fillers, diluents, disintegrants, wetting agents, glidants, anti-caking agents, coloring agents, sweetening agents, dye-migration inhibitors, preservatives, antioxidants, lyoprotectants, complexing agents, flavoring agents, matrix-forming agents, dispersing agents, performance modifiers, controlled-release polymers, solvents, pH modifiers, and sources of carbon dioxide. In some embodiments, the pharmaceutically acceptable vehicle comprises an organic acid agent, which as discussed herein, has been found to provide unique benefits as both a stabilizing agent and a solubilizing agent to aid release from the disclosed dosage forms and to provide stabilization of the compounds herein.

In some embodiments, pharmaceutical compositions of the present disclosure may be in orodispersible dosage forms (ODxs), including sublingual dosage forms, buccal dosage forms, e.g., orally disintegrating tablets (ODTs) (also sometimes referred to as fast disintegrating tablets, orodispersible tablets, or fast dispersible tablets) or orodispersible films (ODFs) (or wafers). Such dosage forms may be particularly advantageous in the present disclosure as they allow for pre-gastric absorption of the compounds/salts herein, e.g., when administered intraorally through the mucosal linings of the oral cavity, e.g., buccal, lingual, and sublingual administration, for increased bioavailability and faster onset compared to oral administration through the gastrointestinal tract. Additionally, orodispersible dosage forms may be advantageous for the treatment of pediatric/adolescent patients or patients that have general difficulty swallowing traditional dosage forms such as general tablets or capsules.

In some embodiments, the orodispersible dosage form (ODx) is a sublingual dosage form to be disintegrated/dissolved under the tongue, whereby the contents (e.g., the compounds of the present disclosure) are absorbed through the mucous membrane beneath the tongue where they enter venous circulation. In some embodiments, the sublingual dosage form is disintegrated/dissolved under the tongue, whereby the contents are converted into a liquid or semi-solid dosage form, such as a solution, syrup, or paste upon mixing with the saliva, and subsequently swallowed. In some embodiments, the orodispersible dosage form (ODx) is a buccal dosage form to be disintegrated/dissolved in the buccal cavity, whereby the contents (e.g., the compounds of the present disclosure) are absorbed through the oral mucosa lining the mouth where they enter venous circulation. In some embodiments, the buccal dosage form is disintegrated/dissolved in the buccal cavity, whereby the contents are converted into a liquid or semi-solid dosage form, such as a solution, syrup, or paste upon mixing with the saliva, and subsequently swallowed. In addition to the active ingredient(s), the pharmaceutical compositions in orodispersible dosage form (ODxs) may contain one or more pharmaceutically acceptable vehicles (e.g., one or more of a binder, a filler, a diluent, a disintegrant, a lyoprotectant, a preservative, an antioxidant, a stabilizing agent, a solubilizing agent, a flavoring agent, a source of carbon dioxide, a bioadhesive agent, etc., and/or any other pharmaceutically acceptable vehicle set forth herein, with specific mention being made to an organic acid agent).

Orodispersible dosage forms can be prepared by different techniques, such as freeze drying (lyophilization), molding, spray drying, mass extrusion or compressing. In some embodiments, the orodispersible dosage forms are prepared by lyophilization. In some embodiments, the orodispersible dosage forms disintegrate in less than about 90 seconds, in less than about 60 seconds, in less than about 30 seconds, in less than about 20, in less than about 10 seconds, in less than about 5 seconds, or in less than about 2 seconds after being received in the oral cavity. In some embodiments, the orodispersible dosage forms dissolve in less than about 90 seconds, in less than about 60 seconds, or in less than about 30 seconds after being received in the oral cavity. In some embodiments, the orodispersible dosage forms disperse in less than about 90 seconds, in less than about 60 seconds, in less than about 30 seconds, in less than about 20, in less than about 10 seconds, in less than about 5 seconds, or in less than about 2 seconds after being received in the oral cavity. In some embodiments, the pharmaceutical compositions are in the form of orodispersible dosage forms, such as oral disintegrating tablets (ODTs), having a disintegration time according to the United States Phamacopeia (USP) disintegration test <701> of not more than about 30 seconds, not more than about 20, not more than about 10 seconds, not more than about 5 seconds, not more than about 2 seconds. Orodispersible dosage forms having longer disintegration times according to the United States Phamacopeia (USP) disintegration test <701>, such as when adapted for extended release, for example 2 minutes, 3 minutes, 4 minutes, 5 minutes, 10 minutes, 15 minutes, 20 minutes, 25 minutes, 30 minutes, 45 minutes, 60 minutes, or any range therebetween, or longer, are also contemplated.

In some embodiments, the pharmaceutical compositions are in the form of sublingual tablets, prepared by direct compression, compression molding, or lyophilization. In some embodiments, the sublingual tablets are created by direct compression, whereby directly compressible pharmaceutical vehicles such as organic acid agent (optionally coated), binder, filler, lubricant, etc. are mixed with the compound of Formula (I-3) (or a pharmaceutically acceptable salt, polymorph, or solvate thereof) and compressed into tablets by direct compression. In some embodiments, the sublingual tablet contains one or more binders/fillers/diluents such as lactose, mannitol, microcrystalline cellulose including silicified microcrystalline cellulose (SMCC), polyvinylpyrrolidone (PVP). In some embodiments, the sublingual tablet contains a lubricant e.g., magnesium stearate. Other pharmaceutically acceptable vehicles such as soluble excipients, dry binders, pH modifiers/buffers, surface-active agents, sweetening agents, flavoring agents, etc. may also be used. A non-limiting example of sublingual tablet formulation is one that includes a compound of Formula (I-3) (or a pharmaceutically acceptable salt, polymorph, or solvate thereof), an organic acid agent such as citric acid (which may be optionally coated), lactose, mannitol, PVP, and magnesium stearate, and optionally one or more additional pharmaceutically acceptable vehicles set forth herein.

In some embodiments, the sublingual tablet can comprise a monolayer, bilayer, or trilayer. In some embodiments, the monolayer sublingual tablet contains an active ingredient (e.g., a compound of Formula (I-3), or a pharmaceutically acceptable salt, polymorph, or solvate thereof) and one or more pharmaceutically acceptable vehicles (e.g., an organic acid agent such as citric acid). In some embodiments, the monolayer sublingual tablet is effervescent and is formulated with an “effervescent couple,” i.e., a combination of an organic acid agent and a source of carbon dioxide. In some embodiments, the bilayer sublingual tablet contains one or more pharmaceutically acceptable vehicles (e.g., an organic acid agent such as citric acid) in a first layer, and an active ingredient (e.g., a compound of Formula (I-3), or a pharmaceutically acceptable salt, polymorph, or solvate thereof) in the second layer. The second layer may optionally contain one or more pharmaceutically acceptable vehicles. This configuration allows the active ingredient to be stored separately from all, or certain, pharmaceutically acceptable vehicles so that contact between the active ingredient and those vehicles is minimized or altogether prevented, which can in some instances increase the stability of the active ingredient and optionally increase the shelf life of the composition compared to the case where the vehicles and the active ingredient were contained in a single layer. In some embodiments, the bilayer sublingual tablet is an effervescent sublingual tablet whereby the first layer is effervescent comprising an effervescent couple and optionally other pharmaceutically acceptable vehicles, and the second layer comprises the active ingredient (e.g., a compound of Formula (I-3), or a pharmaceutically acceptable salt, polymorph, or solvate thereof, and optionally one or more pharmaceutically acceptable vehicles, the second layer being either non-effervescent or effervescent. For trilayer sublingual tablets, each of the layers may be different or two of the layers, such as the upper and lower layers, may have substantially the same composition. In some embodiments, the lower and upper layers surround a core layer containing the active ingredient (e.g., a compound of Formula (I-3), or a pharmaceutically acceptable salt, polymorph, or solvate thereof). In some embodiments, the lower and upper layers may contain one or more vehicle components such as a solubilizing agent, stabilizing agent, etc. (e.g., an organic acid agent such as citric acid). In some embodiments, the lower and upper layers have the same composition. Alternatively, the lower and upper layers may contain different vehicles or different amounts of the same vehicle. The core layer typically contains the active ingredient, optionally with one or more pharmaceutically acceptable vehicles. As described above, such a trilayer sublingual tablet configuration allows the active ingredient to be stored separately from all, or certain, pharmaceutically acceptable vehicles so that contact between the active ingredient and those vehicles is minimized or altogether prevented. In some embodiments, the trilayer sublingual tablet is an effervescent sublingual tablet whereby at least one of, at least two of, or all three of the layers are effervescent (formulated with an effervescent couple). In some embodiments, the lower and upper layers are effervescent, comprising an organic acid agent (e.g., citric acid), a source of carbon dioxide, and optionally other pharmaceutically acceptable vehicles, and the core layer comprises the active ingredient (e.g., a compound of Formula (I-3), or a pharmaceutically acceptable salt, polymorph, or solvate thereof, and optionally one or more pharmaceutically acceptable vehicles, the core layer being either non-effervescent or effervescent.

In some embodiments, the pharmaceutical compositions are in the form of lyophilized orodispersible dosage forms, such as lyopholized ODTs. In some embodiments, the lyophilized orodispersible dosage forms (e.g., lyophilized ODTs) are created by creating a porous matrix by subliming the water from pre-frozen aqueous formulation of the drug containing matrix-forming agents and other vehicles such as those set forth herein, e.g., one or more lyoprotectants, preservatives, antioxidants, stabilizing agents, solubilizing agents, flavoring agents, etc. In some embodiments, the orodispersible dosage forms comprise two component frameworks of a lyophilized matrix system that work together to ensure the development of a successful formulation. In some embodiments, the first component is a water-soluble polymer such as gelatin, dextran, alginate, and maltodextrin. This component maintains the shape and provides mechanical strength to the dosage form (binder). In some embodiments, the second constituent is a matrix-supporting/disintegration-enhancing agent such as sucrose, lactose, mannitol, xylitol, microcrystalline cellulose including silicified microcrystalline cellulose (SMCC), calcium diphosphate, and/or starch, which acts by cementing the porous framework, provided by the water-soluble polymer and accelerates the disintegration of the orodispersible dosage forms. In some embodiments, the lyophilized orodispersible dosage form (e.g., lyophilized ODT) includes gelatin and mannitol. In some embodiments, the lyophilized orodispersible dosage form (e.g., lyophilized ODT) includes gelatin, mannitol, and one or more of a lyoprotectant, a preservative, an antioxidant, a stabilizing agent, a solubilizing agent, a flavoring agent, and/or another pharmaceutically acceptable vehicle set forth herein, with particular mention being made to an organic acid agent (e.g., citric acid). A non-limiting example of an ODT formulation is Zydis® orally dispersible tablets (available from Catalent). In some embodiments, the ODT formulation (e.g., Zydis® orally dispersible tablets) includes one or more water-soluble polymers, such as gelatin, one or more matrix materials, fillers, or diluents, such as mannitol, a compound of Formula (I-3), or a pharmaceutically acceptable salt, polymorph, or solvate thereof, and optionally a lyoprotectant, a preservative, an antioxidant, a stabilizing agent, a solubilizing agent, a flavoring agent, and/or another pharmaceutically acceptable vehicle set forth herein. In some embodiments, the ODT formulation (e.g., Zydis® orally dispersible tablets) includes gelatin, mannitol, a compound of Formula (I-3), or a pharmaceutically acceptable salt, polymorph, or solvate thereof, and citric acid and/or tartaric acid.

In some embodiments, the ODT can comprise a monolayer, bilayer, or trilayer. In some embodiments, the monolayer ODT contains an active ingredient (e.g., a compound of Formula (I-3), or a pharmaceutically acceptable salt, polymorph, or solvate thereof) and one or more pharmaceutically acceptable vehicles (e.g., an organic acid agent such as citric acid). In some embodiments, the monolayer ODT is effervescent and is formulated with an “effervescent couple,” i.e., a combination of an organic acid agent and a source of carbon dioxide. In some embodiments, the bilayer ODT contains one or more pharmaceutically acceptable vehicles (e.g., an organic acid agent such as citric acid) in a first layer, and an active ingredient (e.g., a compound of Formula (I-3), or a pharmaceutically acceptable salt, polymorph, or solvate thereof) in the second layer. The second layer may optionally contain one or more pharmaceutically acceptable vehicles. This configuration allows the active ingredient to be stored separately from all, or certain, pharmaceutically acceptable vehicles so that contact between the active ingredient and those vehicles is minimized or altogether prevented, which can in some instances increase the stability of the active ingredient and optionally increase the shelf life of the composition compared to the case where the vehicles and the active ingredient were contained in a single layer. In some embodiments, the bilayer ODT is an effervescent ODT whereby the first layer is effervescent comprising an effervescent couple and optionally other pharmaceutically acceptable vehicles, and the second layer comprises the active ingredient (e.g., a compound of Formula (I-3), or a pharmaceutically acceptable salt, polymorph, or solvate thereof, and optionally one or more pharmaceutically acceptable vehicles, the second layer being either non-effervescent or effervescent. For trilayer ODTs, each of the layers may be different or two of the layers, such as the upper and lower layers, may have substantially the same composition. In some embodiments, the lower and upper layers surround a core layer containing the active ingredient (e.g., a compound of Formula (I-3), or a pharmaceutically acceptable salt, polymorph, or solvate thereof). In some embodiments, the lower and upper layers may contain one or more vehicle components such as a solubilizing agent, stabilizing agent, etc. (e.g., an organic acid agent such as citric acid). In some embodiments, the lower and upper layers have the same composition. Alternatively, the lower and upper layers may contain different vehicles or different amounts of the same vehicle. The core layer typically contains the active ingredient, optionally with one or more pharmaceutically acceptable vehicles. As described above, such a trilayer ODT configuration allows the active ingredient to be stored separately from all, or certain, pharmaceutically acceptable vehicles so that contact between the active ingredient and those vehicles is minimized or altogether prevented. In some embodiments, the trilayer ODT is an effervescent ODT whereby at least one of, at least two of, or all three of the layers are effervescent (formulated with an effervescent couple). In some embodiments, the lower and upper layers are effervescent, comprising an organic acid agent (e.g., citric acid), a source of carbon dioxide, and optionally other pharmaceutically acceptable vehicles, and the core layer comprises the active ingredient (e.g., a compound of Formula (I-3), or a pharmaceutically acceptable salt, polymorph, or solvate thereof, and optionally one or more pharmaceutically acceptable vehicles, the core layer being either non-effervescent or effervescent.

In some embodiments, the pharmaceutical compositions are in the form of lyophilized orodispersible films (ODFs) (or wafers). In some embodiments, the pharmaceutical compositions are in the form of lyophilized ODFs protected for the long-term storage by a specialty packaging excluding moisture, oxygen, and light. In some embodiments, the lyophilized ODFs are created by creating a porous matrix by subliming the water from pre-frozen aqueous formulation of the drug containing matrix-forming agents and other vehicles such as those set forth herein, e.g., one or more of a lyoprotectant, a preservative, an antioxidant, a stabilizing agent, a solubilizing agent, a flavoring agent, and/or another pharmaceutically acceptable vehicle set forth herein. In some embodiments, the lyophilized ODF includes a thin water-soluble film matrix. In some embodiments, the ODFs comprise two component frameworks of a lyophilized matrix system that work together to ensure the development of a successful formulation. In some embodiments, the first component is a water-soluble polymer such as gelatin, dextran, alginate, and maltodextrin. This component maintains the shape and provides mechanical strength to the film/wafer (binder). In some embodiments, the second constituent is a matrix-supporting/disintegration-enhancing agent such as sucrose, lactose, mannitol, xylitol, microcrystalline cellulose including silicified microcrystalline cellulose (SMCC), calcium diphosphate, and/or starch, which acts by cementing the porous framework, provided by the water-soluble polymer and accelerates the disintegration of the wafer. In some embodiments, the lyophilized ODFs include gelatin and mannitol. In some embodiments, the lyophilized ODFs include gelatin, mannitol, and one or more of a lyoprotectant, a preservative, an antioxidant, a stabilizing agent, a solubilizing agent, a flavoring agent, and/or another pharmaceutically acceptable vehicle set forth herein, with particular mention being made to an organic acid agent (e.g., citric acid).

In some embodiments, the ODF (or wafer) can comprise a monolayer, bilayer, or trilayer. In some embodiments, the monolayer ODF (or wafer) contains an active ingredient (e.g., a compound of Formula (I-3), or a pharmaceutically acceptable salt, polymorph, or solvate thereof) and one or more pharmaceutically acceptable vehicles (e.g., an organic acid agent such as citric acid). In some embodiments, the monolayer ODF (or wafer) is effervescent and is formulated with an effervescent couple. In some embodiments, the bilayer ODF (or wafer) contains one or more pharmaceutically acceptable vehicles (e.g., an organic acid agent such as citric acid) in a first layer, and an active ingredient (e.g., a compound of Formula (I-3), or a pharmaceutically acceptable salt, polymorph, or solvate thereof) in the second layer. The second layer may optionally contain one or more pharmaceutically acceptable vehicles. This configuration allows the active ingredient to be stored separately from all, or certain, pharmaceutically acceptable vehicles so that contact between the active ingredient and those vehicles is minimized or altogether prevented, which can in some instances increase the stability of the active ingredient and optionally increase the shelf life of the composition compared to the case where the vehicles and the active ingredient were contained in a single layer. In some embodiments, the bilayer ODF (or wafer) is an effervescent ODF (or wafer) whereby the first layer is effervescent comprising an effervescent couple and optionally other pharmaceutically acceptable vehicles, and the second layer comprises the active ingredient (e.g., a compound of Formula (I-3), or a pharmaceutically acceptable salt, polymorph, or solvate thereof, and optionally one or more pharmaceutically acceptable vehicles, the second layer being either non-effervescent or effervescent. For trilayer ODFs (or wafer), each of the layers may be different or two of the layers, such as the upper and lower layers, may have substantially the same composition. In some embodiments, the lower and upper layers surround a core layer containing the active ingredient (e.g., a compound of Formula (I-3), or a pharmaceutically acceptable salt, polymorph, or solvate thereof). In some embodiments, the lower and upper layers may contain one or more vehicle components such as a solubilizing agent, stabilizing agent, etc. (e.g., an organic acid agent such as citric acid). In some embodiments, the lower and upper layers have the same composition. Alternatively, the lower and upper layers may contain different vehicles or different amounts of the same vehicle. The core layer typically contains the active ingredient, optionally with one or more pharmaceutically acceptable vehicles. As described above, such a trilayer ODF (or wafer) configuration allows the active ingredient to be stored separately from all, or certain, pharmaceutically acceptable vehicles so that contact between the active ingredient and those vehicles is minimized or altogether prevented. In some embodiments, the trilayer ODF (or wafer) is an effervescent ODF (or wafer) whereby at least one of, at least two of, or all three of the layers are effervescent (formulated with an effervescent couple). In some embodiments, the lower and upper layers are effervescent, comprising an organic acid agent (e.g., citric acid), a source of carbon dioxide, and optionally other pharmaceutically acceptable vehicles, and the core layer comprises the active ingredient (e.g., a compound of Formula (I-3), or a pharmaceutically acceptable salt, polymorph, or solvate thereof, and optionally one or more pharmaceutically acceptable vehicles, the core layer being either non-effervescent or effervescent.

Examples of pharmaceutically acceptable lyoprotectants include, but are not limited to, disaccharides such as sucrose and trehalose, anionic polymers such as sulfobutylether-β-cyclodextrin (SBECD) and hyaluronic acid, and hydroxylated cyclodextrins.

Examples of pharmaceutically acceptable preservatives include, but are not limited to, glycerin, methyl and propylparaben, benzoic acid, sodium benzoate and alcohol.

Examples of pharmaceutically acceptable antioxidants, which may act to further enhance stability of the composition, include, but are not limited to: (1) water-soluble antioxidants, such as ascorbic acid, cysteine or salts thereof (cysteine hydrochloride), sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of pharmaceutically acceptable stabilizing agents include, but are not limited to, organic acid agents (e.g., citric acid), fatty acids, fatty alcohols, alcohols, long chain fatty acid esters, long chain ethers, hydrophilic derivatives of fatty acids, polyvinylpyrrolidones, polyvinyl ethers, polyvinyl alcohols, hydrocarbons, hydrophobic polymers, moisture-absorbing polymers, glycerol, methionine, monothioglycerol, ascorbic acid, polysorbate, arginine, cyclodextrins, microcrystalline cellulose including silicified microcrystalline cellulose (SMCC), modified celluloses (e.g., carboxymethyl cellulose, sodium salt), sorbitol, and cellulose gel.

Examples of pharmaceutically acceptable solubilizing agents (or dissolution aids) include, but are not limited to, organic acid agents (e.g., citric acid, fumaric acid, DL-malic acid, tartaric acid, lactic acid, maleic acid, etc.), hydroxypropylcellulose, hydroxypropylmethylcellulose, sodium stearyl fumarate, methacrylic acid copolymer LD, methylcellulose, sodium lauryl sulfate, polyoxyl 40 stearate, purified shellac, sodium dehydroacetate, L-ascorbyl stearate, L-asparagine acid, adipic acid, aminoalkyl methacrylate copolymer E, propylene glycol alginate, casein, casein sodium, a carboxyvinyl polymer, carboxymethylethylcellulose, powdered agar, guar gum, succinic acid, copolyvidone, cellulose acetate phthalate, dioctylsodium sulfosuccinate, zein, powdered skim milk, sorbitan trioleate, aluminum lactate, ascorbyl palmitate, hydroxyethylmethylcellulose, hydroxypropylmethylcelluloseacetate succinate, polyoxyethylene (105) polyoxypropylene (5) glycol, polyoxyethylene hydrogenated castor oil 60, polyoxyl 35 castor oil, poly(sodium 4-styrenesulfonate), polyvinylacetaldiethylamino acetate, polyvinyl alcohol, methacrylic acid copolymer S, lauromacrogol, sulfuric acid, aluminum sulfate, phosphoric acid, calcium dihydrogen phosphate, sodium dodecylbenzenesulfonate, a vinyl pyrrolidone-vinyl acetate copolymer, sodium lauroyl sarcosinate, acetyl tryptophan, sodium methyl sulfate, sodium ethyl sulfate, sodium butyl sulfate, sodium octyl sulfate, sodium decyl sulfate, sodium tetradecyl sulfate, sodium hexadecyl sulfate, and sodium octadecyl sulfate. Of these, in some embodiments, citric acid is preferred.

Flavoring agents include natural flavors extracted from plants, such as fruits, and synthetic blends of compounds which produce a pleasant taste sensation or taste masking effect. Examples of flavoring agents include, but are not limited to, aspartame, saccharin (as sodium, potassium or calcium saccharin), cyclamate (as a sodium, potassium or calcium salt), sucralose, acesulfame-K, thaumatin, neohisperidin, dihydrochalcone, ammoniated glycyrrhizin, dextrose, maltodextrin, fructose, levulose, sucrose, glucose, wild orange peel, citric acid, tartaric acid, oil of wintergreen, oil of peppermint, methyl salicylate, oil of spearmint, oil of sassafras, oil of clove, cinnamon, anethole, menthol, thymol, eugenol, eucalyptol, orange flavor, lemon, lime, and lemon-lime.

Cyclodextrins such as α-cyclodextrin, β-cyclodextrin, γ-cyclodextrin, methyl-β-cyclodextrin, hydroxyethyl β-cyclodextrin, hydroxypropyl-β-cyclodextrin, hydroxypropyl γ-cyclodextrin, sulfated β-cyclodextrin, sulfated α-cyclodextrin, sulfobutyl ether β-cyclodextrin, or other solubilized derivatives can also be advantageously used to enhance delivery of compositions described herein.

Pharmaceutical compositions adapted for oral administration, e.g., capsules and tablets, including compressed tablets, may be formulated with various vehicles such as those set forth herein. Examples of suitable vehicles may include, but are not limited to, binders, fillers, diluents, disintegrants, wetting agents, lubricants, glidants, anti-caking agents, coloring agents, dye-migration inhibitors, sweetening agents, preservatives, antioxidants, stabilizing agents, solubilizing agents, flavoring agents, auxiliary agents, thickening agents, lubricants, granulators, lyoprotectants, complexing agents, matrix-forming agents, dispersing agents, performance modifiers, controlled-release polymers, solvents, pH modifiers, and sources of carbon dioxide.

Binders or granulators impart cohesiveness to a capsule or tablet to ensure the dosage form remains intact and uniform even after compression. Suitable binders or granulators include, but are not limited to, starches, such as corn starch, potato starch, and pre-gelatinized starch (e.g., STARCH 1500); gelatin; sugars, such as sucrose, glucose, dextrose, dextrins, molasses, and lactose; natural and synthetic gums, such as acacia (gum arabic), alginic acid, alginates, extract of Irish moss, Panwar gum, ghatti gum, mucilage of isabgol husks, carboxymethyl cellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses including silicified microcrystalline cellulose (SMCC), such as AVICEL-PH-101, AVICEL-PH-103, AVICEL RC-581, AVICEL-PH-105 (FMC Corp., Marcus Hook, Pa.); and mixtures thereof. Suitable fillers include, but are not limited to, talc, calcium carbonate, microcrystalline cellulose including silicified microcrystalline cellulose (SMCC), powdered cellulose, dextrates, kaolin, mannitol, silicic acid, sorbitol, starch, pre-gelatinized starch, partially hydrolyzed starch (e.g., maltodextrin) and mixtures thereof. In some embodiments, the binder, granulator, or filler is present from about 1%, about 5%, about 10%, about 20%, about 30%, about 40%, about 50% to about 99%, about 90%, about 80%, about 70%, about 60% by weight, based on a total weight of the pharmaceutical compositions disclosed herein, or any range therebetween.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets.

Suitable disintegrants include, but are not limited to, agar; bentonite; celluloses, such as methylcellulose and carboxymethyl cellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose including silicified microcrystalline cellulose (SMCC), such as sodium starch glycolate; polacrilin potassium; starches, such as corn starch, potato starch, tapioca starch, pre-gelatinized starch, and partially hydrolyzed starch; clays; aligns; and mixtures thereof. The amount of disintegrant in the pharmaceutical compositions disclosed herein varies upon the type of formulation, and is readily discernible to those of ordinary skill in the art. In some embodiments, the pharmaceutical compositions disclosed herein contain e.g., from about 0.5%, about 1%, about 3%, about 5%, about 10%, about 15%, to about 50%, about 40%, about 30%, about 20% by weight of a disintegrant, based on a total weight of the pharmaceutical composition, e.g., from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG) (e.g., PEG 4,000, PEG 6,000, PEG 8,000, etc., where the number refers to the approximate average molecular weight of the PEG); stearic acid; sodium lauryl sulfate; sodium stearyl fumarate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL® 200 (W.R. Grace Co., Baltimore, Md.) and CAB-O-SIL® (Cabot Co. of Boston, Mass.); and mixtures thereof. In some embodiments, the pharmaceutical compositions disclosed herein contain e.g., from about 0.1%, about 0.5%, about 1%, about 2%, about 3%, about 4%, about 5%, to about 20%, about 15%, about 10%, about 7% by weight of a lubricant, based on a total weight of the pharmaceutical composition, e.g., from about 0.1% to about 5% by weight of a lubricant.

Suitable glidants include, but are not limited to, colloidal silicon dioxide, CAB-O-SIL® (Cabot Co. of Boston, Mass.), and asbestos-free talc.

Suitable anti-caking agents include, but are not limited to, silicon dioxide.

Coloring agents include any of the approved, certified, water-soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye.

Sweetening agents include, but are not limited to, sucrose, lactose, mannitol, syrups, glycerin, sucralose, and artificial sweeteners, such as saccharin and aspartame.

Suitable emulsifying agents include, but are not limited to, gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate.

Suspending and dispersing agents include, but are not limited to, sodium carboxymethyl cellulose, pectin, tragacanth, Veegum, acacia, sodium carbomethylcellulose, hydroxypropyl methylcellulose, and polyvinylpyrolidone.

Preservatives include, but are not limited to, glycerin, methyl and propylparaben, benzoic acid sodium benzoate and alcohol.

Wetting agents include, but are not limited to, propylene glycol monostearate, sorbitan monooleate, diethylene glycol monolaurate, and polyoxyethylene lauryl ether.

Solvents include, but are not limited to, glycerin, sorbitol, ethyl alcohol, and syrup. Examples of non-aqueous liquids utilized in emulsions include, but are not limited to, mineral oil and cottonseed oil.

Examples of pH modifiers include acids (including organic acid agents), such as citric acid, acetic acid, ascorbic acid, lactic acid, aspartic acid, succinic acid, phosphoric acid, and the like; bases including salts of organic acid agents, such as sodium acetate, potassium acetate, sodium citrate (e.g., monosodium citrate, disodium citrate, and/or trisodium citrate), potassium citrate (e.g., monopotassium citrate, dipotassium citrate, and/or tripotassium citrate), sodium tartrate (e.g., monosodium tartrate and/or disodium tartrate), potassium tartrate (e.g., monopotassium tartrate and/or dipotassium tartrate), potassium sodium tartrate, ammonium citrate (e.g., monoammonium citrate, diammonium citrate, and/or triammonium citrate), ammonium tartrate (e.g., monoammonium tartrate and/or diammonium tartrate), sodium fumarate (e.g., monosodium fumarate and/or disodium fumarate), potassium fumarate (e.g., monopotassium fumarate and/or dipotassium fumarate), sodium maleate (e.g., monosodium maleate and/or disodium maleate), potassium maleate (e.g., monopotassium maleate and/or dipotassium maleate), sodium lactate, potassium lactate, calcium oxide, magnesium oxide, trisodium phosphate, sodium hydroxide, calcium hydroxide, aluminum hydroxide, and the like, and buffers generally comprising mixtures of acids and the salts of said acids.

The source of carbon dioxide may include, but is not limited to, sodium bicarbonate, sodium carbonate, potassium carbonate, potassium bicarbonate, magnesium carbonate, calcium carbonate, and sesquicarbonate. The source of carbon dioxide can be used singly, or in combination.

As described above, preferred dosage forms are those formulated with an organic acid agent, which may act as a stabilizing agent and/or solubilizing agent in the disclosed pharmaceutical compositions. The organic acid agent may be any set forth herein, with specific mention being made to citric and/or tartaric acid.

In some embodiments, the dosage form is a tablet. In some embodiments, the tablet (e.g., general tablets including compressed tablets) can comprise a monolayer, bilayer, or trilayer. In some embodiments, the monolayer tablet contains an active ingredient (e.g., a compound of Formula (I-3), or a pharmaceutically acceptable salt, polymorph, or solvate thereof) and one or more pharmaceutically acceptable vehicles (e.g., an organic acid agent such as citric acid). In some embodiments, the monolayer tablet is effervescent and is formulated with an effervescent couple.

In some embodiments, the bilayer tablet contains one or more pharmaceutically acceptable vehicles (e.g., an organic acid agent such as citric acid) in a first layer, and an active ingredient (e.g., a compound of Formula (I-3), or a pharmaceutically acceptable salt, polymorph, or solvate thereof) in the second layer. The second layer may optionally contain one or more pharmaceutically acceptable vehicles. This configuration allows the active ingredient to be stored separately from all, or certain, pharmaceutically acceptable vehicles so that contact between the active ingredient and those vehicles is minimized or altogether prevented, which can in some instances increase the stability of the active ingredient and optionally increase the shelf life of the composition compared to the case where the vehicles and the active ingredient were contained in a single layer. In some embodiments, the bilayer tablet is an effervescent tablet whereby the first layer is effervescent comprising an effervescent couple and optionally other pharmaceutically acceptable vehicles, and the second layer comprises the active ingredient (e.g., a compound of Formula (I-3), or a pharmaceutically acceptable salt, polymorph, or solvate thereof, and optionally one or more pharmaceutically acceptable vehicles, the second layer being either non-effervescent or effervescent. For trilayer tablets, each of the layers may be different or two of the layers, such as the upper and lower layers, may have substantially the same composition. In some embodiments, the lower and upper layers surround a core layer containing the active ingredient (e.g., a compound of Formula (I-3), or a pharmaceutically acceptable salt, polymorph, or solvate thereof). In some embodiments, the lower and upper layers may contain one or more vehicle components such as a solubilizing agent, stabilizing agent, etc. (e.g., an organic acid agent such as citric acid). In some embodiments, the lower and upper layers have the same composition. Alternatively, the lower and upper layers may contain different vehicles or different amounts of the same vehicle. The core layer typically contains the active ingredient, optionally with one or more pharmaceutically acceptable vehicles. As described above, such a trilayer tablet configuration allows the active ingredient to be stored separately from all, or certain, pharmaceutically acceptable vehicles so that contact between the active ingredient and those vehicles is minimized or altogether prevented. In some embodiments, the trilayer tablet is an effervescent tablet whereby at least one of, at least two of, or all three of the layers are effervescent (formulated with an effervescent couple). In some embodiments, the lower and upper layers are effervescent, comprising an organic acid agent (e.g., citric acid), a source of carbon dioxide, and optionally other pharmaceutically acceptable vehicles, and the core layer comprises the active ingredient (e.g., a compound of Formula (I-3), or a pharmaceutically acceptable salt, polymorph, or solvate thereof, and optionally one or more pharmaceutically acceptable vehicles, the core layer being either non-effervescent or effervescent.

It should be understood that many vehicles (carriers, excipients, etc.) may serve several functions, even within the same formulation. Particular mention is made to pharmaceutical compositions herein containing an organic acid agent such as citric acid, which may play multiple roles as a stabilizing agent, e.g., to stabilize the psilocin compound of the present disclosure in free base or salt form, as a solubilizing agent to provide fast dissolution of the active for rapid onset, etc., particularly for dosage forms adapted for rapid onset and a shorter duration of drug action, such as orodispersible dosage forms (e.g., ODTs and ODFs), as a flavoring agent, a pH modifier, and/or as an antioxidant.

The tablet dosage forms may be prepared from the active ingredient in powdered, crystalline, or granular forms, alone or in combination with one or more vehicles (e.g., carriers or excipients) described herein, including binders, disintegrants, controlled-release polymers, pH modifiers, lubricants, diluents, and/or coloring agents. Flavoring and sweetening agents are especially useful in the formation of chewable tablets and lozenges.

The pharmaceutical compositions herein may be in the form of compressed tablets, tablet triturates, chewable lozenges, rapidly dissolving tablets, multiple compressed tablets, or any of the above which are coated, such as enteric-coating tablets, sugar-coated, or film-coated tablets. Coated tablets are tablets covered with one or more layers of pharmaceutically acceptable vehicle or mixtures of vehicles such as natural or synthetic resins, polymers, gums, fillers, sugars, plasticizers, polyols, waxes, organic bases, coloring matters authorized by the appropriate national or regional authority, and flavoring substances. Such coating materials generally do not contain any active ingredient, e.g., any of the compounds described herein (e.g., compound of Formula (I-3), or a pharmaceutically acceptable salt, polymorph, or solvate thereof). The tablets may be coated for a variety of reasons such as protection of the active ingredients from burst release from the matrix, air, moisture or light, masking of unpleasant tastes and odors or improvement of appearance. The substance used for coating may be applied as a solution or suspension. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenylsalicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethyl cellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

In some embodiments, the pharmaceutical composition (e.g., a tablet composition formulated for oral administration such as a monolayer tablet composition), comprises any of the compounds described herein (e.g., compound of Formula (I-3), or a pharmaceutically acceptable salt, polymorph, or solvate thereof), and a polymer.

In some embodiments, the tablet composition is a modified-release tablet adapted for sustained release and preferably maximum sustained release. In some embodiments, the release period of any of the compounds described herein (e.g., compound of Formula (I-3), or a pharmaceutically acceptable salt, polymorph, or solvate thereof), in the formulations of the disclosure is greater than 4 hours, greater than 6 hours, greater than 8 hours, greater than 10 hours, greater than 12 hours, greater than 16 hours, greater than 20 hours, greater than 24 hours, greater than 28 hours, greater than 32 hours, greater than 36 hours, greater than 48 hours.

In some embodiments, the tablet composition is adapted for tamper resistance. In some embodiments, the tablet composition comprises polyethylene oxide (PEO), e.g., MW about 2,000 to about 7,000 KDa, in combination with HPMC. In some embodiments, the tablet composition may further comprise polyethylene glycol (PEG), e.g., PEG 8,000. In some embodiments, the tablet composition may further comprise a polymer carrying one or more negatively charged groups, e.g., polyacrylic acid. In some embodiments, the tablet composition comprising PEO is further subjected to heating/annealing, e.g., extrusion conditions.

In some embodiments, the pharmaceutical composition comprises a combination of (i) a water-insoluble neutrally charged non-ionic matrix; (ii) a polymer carrying one or more negatively charged groups; and (iii) any of the compounds described herein (e.g., compound of Formula (I-3), or a pharmaceutically acceptable salt, polymorph, or solvate thereof). Other pharmaceutical vehicle(s), such as an organic acid agent, a lubricant, etc. may also be optionally included.

In some embodiments, the water-insoluble neutrally charged non-ionic matrix is selected from cellulose-based polymers such as microcrystalline cellulose polymers or HPMC, alone or enhanced by mixing with components selected from the group consisting of starches; waxes; neutral gums; polymethacrylates; PVA; PVA/PVP blends; silicon dioxide, and mixtures thereof. In some embodiments, the cellulose-based polymer is hydroxypropyl methylcellulose (HPMC). In some embodiments, the cellulose-based polymer is a microcrystalline cellulose polymer such as silicified microcrystalline cellulose (SMCC). In some embodiments, the pharmaceutical composition (e.g., tablet or capsule) comprises about 10%, about 15%, about 20%, about 25%, about 30%, about 35%, about 40%, about 45%, about 50%, about 55%, about 60%, about 65%, about 70%, about 75%, about 80%, about 85%, about 90% of the water-insoluble neutrally charged non-ionic matrix by weight, based on a total weight of the pharmaceutical composition, or any range therebetween. In some embodiments, the pharmaceutical composition comprises a microcrystalline cellulose polymer such as silicified microcrystalline cellulose (SMCC), e.g., in an amount of about 70%, about 75%, about 80%, about 85%, about 90% by weight, based on a total weight of the pharmaceutical composition, or any range therebetween. In some embodiments, the pharmaceutical comprises a combination of HPMC and starch.

In some embodiments, the polymer carrying one or more negatively charged groups is selected from the group consisting of polyacrylic acid, polylactic acid, polyglycolic acid, polymethacrylate carboxylates, cation-exchange resins, clays, zeolites, hyaluronic acid, anionic gums, salts thereof, and mixtures thereof. In some embodiments, the anionic gum is selected from the group consisting of naturally occurring materials and semi-synthetic materials. In some embodiments, the naturally occurring material is selected from the group consisting of alginic acid, pectin, xanthan gum, carrageenan, locust bean gum, gum arabic, gum karaya, guar gum, and gum tragacanth. In some embodiments, the semi-synthetic material is selected from the group consisting of carboxymethyl-chitin and cellulose gum (sodium carboxymethyl cellulose). In some embodiments, the pharmaceutical composition comprises about 0.5%, about 1%, about 1.5%, about 2%, about 2.5%, about 3%, about 3.5%, about 4%, about 4.5%, about 5%, about 5.5%, 6%, about 6.5%, about 7%, about 7.5%, about 8%, about 8.5%, about 9%, about 9.5%, about 10%, of the polymer carrying one or more negatively charged groups by weight, based on a total weight of the pharmaceutical composition, or any range therebetween. In some embodiments, the pharmaceutical composition comprises cellulose gum (sodium carboxymethyl cellulose), e.g., about 0.5%, about 1%, about 1.5%, about 2%, about 2.5%, about 3%, about 3.5%, about 4%, about 4.5%, about 5%, about 5.5% cellulose gum by weight, based on a total weight of the pharmaceutical composition, or any range therebetween. In some embodiments, the pharmaceutical comprises a combination of HPMC and starch.

Moreover, without wishing to be bound by theory, in some embodiments, the role of the polymer carrying one or more negatively charged groups, e.g., moieties of acidic nature as in those of the acidic polymers described herein, surprisingly offers significant retention of any of the compounds described herein (e.g., compound of Formula (I-3), or a pharmaceutically acceptable salt, polymorph, or solvate thereof), in the matrix. In some embodiments, this negative charge may be created in situ, for example, based on release of a proton due to pKa and under certain pH conditions or through electrostatic interaction/creation of negative charge. Further noting that acidic polymers may be the salts of the corresponding weak acids that will be the related protonated acids in the stomach; which, and without wishing to be bound by theory, will neutralize the charge and may reduce the interactions of any of the compounds described herein (e.g., a compound of compound of Formula (I-3), or a pharmaceutically acceptable salt, polymorph, or solvate thereof), with the matrix. In addition, the release matrix may be further complemented by other inactive pharmaceutical ingredients to aid in preparation of the appropriate solid dose form such as fillers, disintegrants, flow improving agents, lubricants, colorants, and taste maskers.

Disclosed herein are pharmaceutical compositions in modified release dosage forms, which comprise a compound as disclosed herein (e.g., a compound of Formula (I-3), or a pharmaceutically acceptable salt, polymorph, or solvate thereof) and one or more release controlling vehicles as described herein. Suitable modified release controlling vehicles include, but are not limited to, hydrophilic or hydrophobic matrix devices, water-soluble separating layer coatings, enteric coatings, osmotic devices, multiparticulate devices, and combinations thereof. The pharmaceutical compositions may also comprise non-release controlling vehicles.

In some embodiments, the oral pharmaceutical composition is for low dose maintenance therapy that can be constructed using the compounds described herein, capitalizing on their ability to bind with anionic polymers.

Further disclosed herein are pharmaceutical compositions in enteric coated dosage forms, which comprise a compound as disclosed herein (e.g., a compound of Formula (I-3), or a pharmaceutically acceptable salt, polymorph, or solvate thereof) and one or more release controlling vehicles for use in an enteric coated dosage form. The pharmaceutical compositions may also comprise non-release controlling vehicles.

Further disclosed herein are pharmaceutical compositions in effervescent dosage form, which comprise a compound as disclosed herein (e.g., a compound of Formula (I-3), or a pharmaceutically acceptable salt, polymorph, or solvate thereof) and one or more pharmaceutically acceptable vehicles, which may be release controlling vehicles and/or non-release controlling vehicles. Effervescent means that the dosage form, when mixed with liquid, including water, juice, saliva, etc., evolves a gas. In general, the effervescent dosage forms of the present disclosure comprise an organic acid agent and a source of carbon dioxide, referred to herein as an “effervescent couple.” Such effervescent dosage forms effervesce (evolve gas) through chemical reaction between the organic acid agent and the source of carbon dioxide, which takes place upon exposure to an aqueous environment, such as upon placement in water, juice, or other drinkable fluid, or from the aqueous environment in the oral cavity, such as saliva in the mouth. Specifically, the reaction between the organic acid agent and the source of carbon dioxide produces carbon dioxide gas upon contact with an aqueous medium such as water, juice, or saliva. While use of disintegrants are optional, effervescent dosage forms do not require a disintegrant as the evolution of the gas in situ facilitates the disintegration process.

For clarity, an “effervescent couple” refers to at least one organic acid agent and at least one source of carbon dioxide being contained in a dosage form, regardless of assembly—for example, the organic acid agent and the source of carbon dioxide can be admixed (as powders), layered on top of one another, agglomerated or otherwise “glued” together in granular form, or held separately from one another such as in separate layers within the dosage form. Further, the term “couple” in this context is not meant to be limited to only an organic acid agent and a source of carbon dioxide and is open to the inclusion of other materials unless specified otherwise; for example, effervescent agglomerates/granules made from bringing together (or “gluing”) an organic acid agent and a source of carbon dioxide may include other vehicles including binders (the “glue”) and the effervescent agglomerates/granules may nonetheless be referred to as an effervescent couple.

In some embodiments, the source of carbon dioxide is sodium bicarbonate. In some embodiments, the source of carbon dioxide is sodium carbonate. In some embodiments, the source of carbon dioxide is potassium carbonate. In some embodiments, the source of carbon dioxide is potassium bicarbonate. However, reactants which evolve oxygen or other gases besides carbon dioxide, and which are safe for human consumption, are also contemplated for use in the disclosed effervescent dosage forms, in addition to or in lieu of the source of carbon dioxide. While not wishing to be bound by theory, it is believed that the effervescence can help quickly break up the dosage form, and in some routes of administration such as intraoral routes, can help reduce the perception of grittiness by providing a distracting sensory experience of effervescence.

In some embodiments, the effervescent dosage form is to be reconstituted in a drinkable fluid such as water or juice, thereby forming an oral liquid dosage form (e.g., solution), prior to consumption. In some embodiments, the effervescent dosage form is to be placed in the oral cavity, where contact with the aqueous environment (saliva) causes disintegration/dissolution of the dosage form along with effervescence. Here, the contents of the effervescent dosage form may be converted into a liquid or semi-solid dosage form, such as a solution, syrup, or paste upon mixing with the saliva, and subsequently swallowed. Alternatively, the effervescent dosage form may be an intraoral dosage form, e.g., a buccal, lingual, or sublingual dosage form, whereby placement in the aqueous environment (saliva) of the oral cavity causes disintegration/dissolution of the dosage form along with effervescence, and pre-gastric absorption of the contents through the oral mucosa. Such pre-gastric absorption may provide for increased bioavailability and faster onset compared to oral administration through the gastrointestinal tract. In some embodiments, the effervescent dosage form is a sublingual dosage form to be disintegrated/dissolved under the tongue, whereby the contents (e.g., the compounds of the present disclosure) are absorbed through the mucous membrane beneath the tongue where they enter venous circulation. In some embodiments, the effervescent dosage form is a buccal dosage form to be disintegrated/dissolved in the buccal cavity, whereby the contents (e.g., the compounds of the present disclosure) are absorbed through the oral mucosa lining the mouth where they enter venous circulation. Effervescent dosage forms may be advantageous for the treatment of pediatric/adolescent patients or patients that have general difficulty swallowing traditional dosage forms such as general tablets or capsules, since effervescent dosage forms can be reconstituted into easy to swallow liquid or semi-solid dosage forms or taken intraorally.

When adapted for intraoral administration, it may be beneficial to formulate the effervescent dosage form with a bioadhesive agent, in addition to the effervescent couple. “Bioadhesive agents” are substances which promote adhesion or adherence to a biological surface, such as mucous membranes. For example, bioadhesive agents are themselves capable of adhering to a biological surface when placed in contact with that surface (e.g., mucous membrane) in order to enable compositions of the disclosure to adhere to that surface, which promotes more efficient transfer of the contents from the dosage form to the biological surface. A variety of polymers known in the art can be used as bioadhesive agents, for example polymeric substances, preferably with an average (weight average) molecular weight above 5,000 g/mol. It is preferred that such polymeric materials are capable of rapid swelling when placed in contact with an aqueous medium such a water or saliva, and/or are substantially insoluble in water at room temperature and atmospheric pressure. Examples of suitable bioadhesive agents include, but are not limited to, cyclodextrin, cellulose derivatives such as hydroxypropylmethyl cellulose (HPMC), hydroxyethyl cellulose (HEC), hydroxypropyl cellulose (HPC), methyl cellulose, ethyl hydroxyethyl cellulose, carboxymethyl cellulose, modified cellulose gum and sodium carboxymethyl cellulose (NaCMC); starch derivatives such as moderately cross-linked starch, modified starch and sodium starch glycolate; acrylic polymers such as carbomer and its derivatives (polycarbophyl, Carbopol®, etc.); polyvinylpyrrolidone (PVP); polyethylene oxide (PEO); chitosan (poly-(D-glucosamine)); natural polymers such as gelatin, sodium alginate, pectin; scleroglucan; xanthan gum; guar gum; poly co-(methylvinyl ether/maleic anhydride); and crosscarmellose (e.g. crosscarmellose sodium). Such polymers may be crosslinked. Combinations of two or more bioadhesive agents can also be used.

An effervescent couple can be coated with a pharmaceutically acceptable vehicle, e.g., with a binder, a protective coating such as a solvent protective coating, an enteric coating, an anti-caking agent, and/or a pH modifier to prevent premature reaction, e.g., with air, moisture, and/or other ingredients contained in the pharmaceutical composition. Each component of the effervescent couple, e.g., the organic acid agent and/or the source of carbon dioxide, can also individually be coated with a pharmaceutically acceptable vehicle, e.g., with a binder, a protective coating such as a solvent protective coating, an enteric coating, an anti-caking agent, and/or a pH modifier to prevent premature reaction, e.g., with air, moisture, and/or other ingredients contained in the pharmaceutical composition. The effervescent couple can also be mixed with previously lyophilized particles, such as one or more pharmaceutically active ingredients coated with a solvent protective or enteric coating.

The effervescent dosage form may be prepared by methods known to those skilled in the art, including, but not limited to, slugging, direct compression, roller compaction, dry or wet granulation, fusion granulation, melt-granulation, vacuum granulation, and fluid bed spray granulation, any of which may be optionally followed by compression/tableting.

The pharmaceutical compositions disclosed herein may be formulated as non-effervescent or effervescent granules and powders. The non-effervescent or effervescent granules and powders may be reconstituted into a liquid dosage form, or alternatively, compressed to form tablet dosage forms which are either non-effervescent or effervescent, respectively. Pharmaceutically acceptable vehicles used in the non-effervescent or effervescent granules or powders may include, but are not limited to, binders, granulators, fillers, diluents, sweetening agent, wetting agents, stabilizing agents, solubilizing agents, anti-caking agents, pH modifiers, or any other pharmaceutical vehicle described herein. In some embodiments, the pharmaceutically acceptable vehicle comprises an organic acid agent, such as glycolic acid, lactic acid, citric acid, tartaric acid, malic acid, fumaric acid, and/or maleic acid.

Pharmaceutically acceptable vehicles used in the effervescent granules or powders include an effervescent couple, i.e., an organic acid agent and a source of carbon dioxide. Effervescent powders may be produced by blending or admixing the organic acid agent and the source of carbon dioxide (the effervescent couple) and optionally any other desired pharmaceutically acceptable vehicle. Effervescent granules may be produced by physically adhering or “gluing” the effervescent couple (the organic acid agent and the source of carbon dioxide) together using an edible or pharmaceutically acceptable binder such as polyvinylpyrrolidone, polyvinyl alcohol, L-leucine, polyethylene glycol, gum arabic, or the like, including combinations thereof. These types of granules are made by processes generically known as “wet granulation.” Granulating solvents such as ethanol and/or isopropyl alcohol are often used to aid this type of granulation process. Since the effervescent couple is physically bound together in the granule, the gas generating reaction is usually quite vigorous, leading to rapid dissolution times. Another type of “wet granulation” product that is specific to effervescent products is known as “fusion” type granules. These granules are formed by reacting the organic acid agent and source of carbon dioxide with a small amount of water (or sometimes a hydrous alcohol granulating solvent, such as various commercial grades of ethanol or isopropyl alcohol) in a highly controlled way. Since the effervescent reaction generates carbon dioxide, fusion granules tend to be quite porous, which decreases their density and also their dissolution time. Accordingly, effervescent granules prepared by wet granulation or fusion type processes may be desirable for making orodispersible dosage forms (ODxs) or other dosage forms where quick dissolving/disintegrating properties are sought. Effervescent tablet dosage forms prepared through tableting, e.g., compression, of effervescent granules or powders are also included in the present disclosure.

Additionally disclosed are pharmaceutical compositions in a dosage form that has an instant releasing component and at least one delayed releasing component, and is capable of giving a discontinuous release of the compound in the form of at least two consecutive pulses separated in time from about 0.1 up to about 24 hours (e.g., about 0.1, 0.5, 1, 2, 4, 6, 8, 10, 12, 14, 16, 18, 10, 22, or 24 hours). The pharmaceutical compositions comprise a compound as disclosed herein (e.g., a compound of Formula (I-3), or a pharmaceutically acceptable salt, polymorph, or solvate thereof) and one or more release controlling and/or non-release controlling vehicles, such as those excipients or carriers suitable for a disruptable semipermeable membrane and as swellable substances.

Disclosed herein also are pharmaceutical compositions in a dosage form for oral administration to a subject, which comprise a compound disclosed herein (e.g., compound of Formula (I-3), or a pharmaceutically acceptable salt, polymorph, or solvate thereof) and one or more pharmaceutically acceptable vehicles (e.g., excipients or carriers), enclosed in an intermediate reactive layer comprising a gastric juice-resistant polymeric layered material partially neutralized with alkali and having cation exchange capacity and a gastric juice-resistant outer layer.

The dosage form may be an immediate release (IR) dosage form, examples of which include, but are not limited to, immediate release (IR) tablets or immediate release (IR) capsules. In addition to the API, dosage forms adapted for immediate release may include one or more pharmaceutically acceptable vehicles which readily disperse, dissolve, or otherwise breakdown in the gastric environment so as not to delay or prolong dissolution/absorption of the API. Examples of pharmaceutically acceptable vehicles for immediate release dosage forms include, but are not limited to, one or more auxiliary agents, stabilizing agents, solubilizing agents, thickening agents, lubricants, binders, granulators, fillers, diluents, disintegrants, wetting agents, glidants, anti-caking agents, coloring agents, sweetening agents, dye-migration inhibitors, preservatives, antioxidants, lyoprotectants, complexing agents, flavoring agents, matrix-forming agents, dispersing agents, and performance modifiers. In some embodiments, the immediate release (IR) dosage form is an immediate release (IR) tablet or capsule comprising one or more of microcrystalline cellulose including silicified microcrystalline cellulose (SMCC), sodium carboxymethyl cellulose, magnesium stearate, mannitol, crospovidone, citric acid, and sodium stearyl fumarate. In some embodiments, the immediate release (IR) dosage form comprises microcrystalline cellulose including silicified microcrystalline cellulose (SMCC), sodium carboxymethyl cellulose, and magnesium stearate. In some embodiments, the immediate release (IR) dosage form comprises silicified microcrystalline cellulose (SMCC), sodium carboxymethyl cellulose, citric acid anhydrous, and sodium stearyl fumarate. In some embodiments, the immediate release (IR) dosage form, such as powder-filled capsules, comprises psilocin-d₁₀ benzenesulfonate salt, silicified microcrystalline cellulose (SMCC), sodium carboxymethyl cellulose, citric acid anhydrous, and sodium stearyl fumarate. In some embodiments, the immediate release (IR) dosage form comprises mannitol, crospovidone, and sodium stearyl fumarate. In some embodiments, the immediate release (IR) dosage form comprises an organic acid agent, for example, citric acid.

The pharmaceutical compositions disclosed herein may be disclosed as soft or hard capsules, which can be made from gelatin, methylcellulose, hydroxypropylmethyl cellulose (HPMC), starch, or calcium alginate. The hard (e.g., gelatin, HPMC, etc.) capsule, also known as dry-filled capsule (DFC) or powder in capsule (PIC), consists of two sections, one slipping over the other, thus completely enclosing the active ingredient and any pharmaceutically acceptable vehicle(s). The soft elastic capsule (SEC) is a soft, globular shell, such as a gelatin or HPMC shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms disclosed herein may be encapsulated in a capsule. Suitable liquid and semisolid dosage forms include solutions and suspensions in propylene carbonate, vegetable oils, or triglycerides. The capsules may also be coated as known by those of skill in the art in order to modify or sustain dissolution of the active ingredient.

In some embodiments, the pharmaceutical compositions are in the form of immediate-release capsules for oral administration, and may further comprise cellulose, iron oxides, lactose, magnesium stearate, and sodium starch glycolate.

In some embodiments, the pharmaceutical compositions are in the form of delayed-release capsules for oral administration, and may further comprise cellulose, ethylcellulose, gelatin, hypromellose, iron oxide, and titanium dioxide.

In some embodiments, the pharmaceutical compositions are in the form of enteric coated delayed-release tablets for oral administration, and may further comprise carnauba wax, crospovidone, diacetylated monoglycerides, ethylcellulose, hydroxypropyl cellulose, hypromellose phthalate, magnesium stearate, mannitol, sodium hydroxide, sodium stearyl fumarate, talc, titanium dioxide, and yellow ferric oxide.

In some embodiments, the pharmaceutical compositions are in the form of enteric coated delayed-release tablets for oral administration, and may further comprise calcium stearate, crospovidone, hydroxypropyl methylcellulose, iron oxide, mannitol, methacrylic acid copolymer, polysorbate 80, povidone, propylene glycol, sodium carbonate, sodium lauryl sulfate, titanium dioxide, and triethyl citrate.

Any of the pharmaceutical compositions disclosed herein formulated with an organic acid agent may contain an organic acid agent which is uncoated, or alternatively, may contain an organic acid agent which is coated (a “coated organic acid agent”) with a pharmaceutically acceptable vehicle. Various pharmaceutical acceptable vehicles can be used as coating materials to modify the properties of the organic acid agent and/or to prevent undesired or premature reactions, e.g., with air, moisture, and/or other ingredients contained in the pharmaceutical composition, without losing the desired function of the organic acid agent. The coated organic acid agent may comprise a core of organic acid agent, and a thin film coating such as a thin film powder coating or a thin film polymeric coating. The coated organic acid agent may be in the form of a core-shell material, comprising a core of organic acid agent, and a protective coating surrounding the core, i.e., a shell. Any of the organic acid agents disclosed herein may be coated, including, but not limited to, glycolic acid, lactic acid, citric acid, tartaric acid, malic acid, fumaric acid, and maleic acid.

In some embodiments, the coated organic acid agent contains at least 0.01% by weight, at least 0.05% by weight, at least 0.1% by weight, at least 0.5% by weight, at least 1% by weight, at least 1.5% by weight, at least 2% by weight, at least 2.5% by weight, at least 3% by weight, at least 3.5% by weight, and up to 15% by weight, up to 10% by weight, up to 9% by weight, up to 8% by weight, up to 7% by weight, up to 6% by weight, up to 5% by weight, up to 4% by weight, by weight of the coating, based on a total weight of the coated organic acid agent, or any range therebetween; the balance being the organic acid agent when the coated organic acid agent is formulated substantially with only the organic acid agent and the coating.

In some embodiments, the organic acid agent is coated with a water-soluble polymer, binder, granulator, filler, and the like. A non-limiting example of this type of coated organic acid agent is Citric acid DC (available from Jungbunzlauer), which is a direct compressible granular powder type of citric acid coated with a thin layer of maltodextrin.

In some embodiments, the organic acid agent is coated with an anti-caking agent. Such coated organic acid agents display a high ability to absorb spurs of humidity. A non-limiting example of this type of coated organic acid agent is Citric acid S40 (available from Jungbunzlauer), which is very fine (pulverized) granular powder of citric acid coated with silicon dioxide.

In some embodiments, the organic acid agent is coated with a pH modifier. In some embodiments, the organic acid agent is coated with a salt of an organic acid agent (i.e., a conjugate base salt of an organic acid agent). The salt of an organic acid agent may be an alkali metal salt of an organic acid agent, an alkaline earth salt of an organic acid agent, an ammonium salt of an organic acid agent, or mixtures thereof including mixed salts (e.g., sodium and potassium mixed salt) of an organic acid agent. The salt of an organic acid agent may be monobasic, dibasic, tribasic, etc. Where the salt of the organic acid agent is polybasic (dibasic, tribasic, etc.), the salt may be formed from one type of cation (e.g., sodium cation), or two or more different cations (e.g., a mixed salt with both sodium and potassium cations). Examples of salts of an organic acid agent which may be used as coating materials, include, but are not limited to, sodium citrate (e.g., monosodium citrate, disodium citrate, and/or trisodium citrate), potassium citrate (e.g., monopotassium citrate, dipotassium citrate, and/or tripotassium citrate), sodium tartrate (e.g., monosodium tartrate and/or disodium tartrate), potassium tartrate (e.g., monopotassium tartrate and/or dipotassium tartrate), potassium sodium tartrate, ammonium citrate (e.g., monoammonium citrate, diammonium citrate, and/or triammonium citrate), ammonium tartrate (e.g., monoammonium tartrate and/or diammonium tartrate), sodium fumarate (e.g., monosodium fumarate and/or disodium fumarate), potassium fumarate (e.g., monopotassium fumarate and/or dipotassium fumarate), sodium maleate (e.g., monosodium maleate and/or disodium maleate), potassium maleate (e.g., monopotassium maleate and/or dipotassium maleate), sodium lactate, and potassium lactate, including mixtures and/or hydrates thereof. Organic acid agents coated with a salt of an organic acid agent may be in the form of core-shell materials. The organic acid agent (core) and the salt of an organic acid agent (shell) may belong to the same conjugate acid-base pair. For example, the organic acid agent (core) may be citric acid and the salt of the organic acid agent (shell) may be an alkali metal salt, an alkaline earth salt, and/or an ammonium salt of citric acid. In another example, the organic acid agent (core) may be tartaric acid and the salt of the organic acid agent (shell) may be an alkali metal salt, an alkaline earth salt, and/or an ammonium salt of tartaric acid. In yet another example, the organic acid agent (core) may be fumaric acid and the salt of the organic acid agent (shell) may be an alkali metal salt, an alkaline earth salt, and/or an ammonium salt of fumaric acid. Alternatively, the organic acid agent (core) and the salt of an organic acid agent (shell) may belong to the different conjugate acid-base pairs. For example, the organic acid agent (core) may be citric acid and the salt of the organic acid agent (shell) may be an alkali metal salt, an alkaline earth salt, and/or an ammonium salt of tartaric acid. In another example, the organic acid agent (core) may be citric acid and the salt of the organic acid agent (shell) may be an alkali metal salt, an alkaline earth salt, and/or an ammonium salt of fumaric acid. In yet another example, the organic acid agent (core) may be tartaric acid and the salt of the organic acid agent (shell) may be an alkali metal salt, an alkaline earth salt, and/or an ammonium salt of citric acid. A non-limiting example of an organic acid agent coated with a salt of an organic acid agent is Citrocoat® N (available from Jungbunzlauer), which is a granular powder made from citric acid as core material with a layer of monosodium citrate (1.5-3.5%) as a shell.

Coated organic acid agents may also be utilized in the disclosed effervescent dosage forms. Here, effervescent couples may be formed from any of the coated organic acid agents disclosed herein and a source of carbon dioxide. The use of a coated organic acid agent in the effervescent couple, as opposed to an uncoated organic acid agent, may advantageously provide improved storage stability to the effervescent dosage form without significantly sacrificing reactivity when placed into an aqueous environment, such as upon placement in water, juice, or other drinkable fluid, or from the aqueous environment in the oral cavity, such as saliva in the mouth. A non-limiting example of an effervescent couple formulated with a coated organic acid agent is Citrocoat® EP (available from Jungbunzlauer), which is an agglomerated granule made by bringing together Citrocoat® N (citric acid core coated with a layer of monosodium citrate, 1.5-3.5%, as a shell) and sodium bicarbonate using gum arabic as binder).

In some embodiments, the pharmaceutical composition comprises a compound of Formula (I-3) as a free base, in crystalline form, and a coated organic acid agent such as coated citric acid, coated tartaric acid, coated fumaric acid, etc. For effervescent dosage forms, a source of carbon dioxide (e.g., sodium bicarbonate) is also included with the coated organic acid agent. In some embodiments, the compound is a crystalline form of 3-(2-(bis(methyl-d₃)amino)ethyl-1,1,2,2-d₄)-1H-indol-4-ol (I-3), as determined by X-ray powder diffraction. In some embodiments, I-3 is a crystalline solid form (pattern 1) characterized by an X-ray powder diffraction pattern containing at least three characteristic peaks at diffraction angles (2θ±0.2°) selected from 7.582°, 8.395°, 9.647°, 10.444°, 11.319°, 12.614°, 13.372°, 14.222°, 15.157°, 16.524°, 16.787°, 17.693°, 19.468°, 19.699°, 20.901°, 21.132°, 21.859°, 22.547°, 23.699°, 24.630°, 25.034°, 25.264°, 26.867°, 27.399°, 27.929°, 28.219°, 28.871°, 29.430°, 30.120°, 30.675°, 31.373°, 32.365°, 33.880°, 34.418°, 34.792°, 35.884°, 36.254°, 37.156°, 38.200°, and 38.417°, as determined by XRPD using a CuKα radiation source, for example, as shown in WO2022195011 and/or WO2023078604. In some embodiments, I-3 is a crystalline solid form (pattern 2) characterized by an X-ray powder diffraction pattern containing at least three characteristic peaks at diffraction angles (2θ±0.2°) selected from 8.124°, 8.357°, 10.059°, 12.630°, 13.420°, 13.743°, 14.053°, 15.220°, 16.272°, 16.763°, 16.954°, 17.328°, 17.662°, 18.062°, 18.742°, 19.413°, 19.658°, 20.172°, 20.836°, 21.267°, 21.833°, 22.213°, 22.504°, 23.334°, 23.701°, 24.385°, 25.431°, 25.721°, 26.049°, 27.291°, 28.368°, 30.349°, 30.656°, 31.337°, 31.538°, 32.091°, 35.870°, 38.514°, and 41.361°, as determined by XRPD using a CuKα radiation source, WO2022195011 and/or WO2023078604.

In some embodiments, the pharmaceutical composition comprises a compound of Formula (I-3) as a free base, in amorphous form, and a coated organic acid agent such as coated citric acid, coated tartaric acid, coated fumaric acid, etc. For effervescent dosage forms, a source of carbon dioxide (e.g., sodium bicarbonate) is also included with the coated organic acid agent. In some embodiments, the is an amorphous form of 3-(2-(bis(methyl-d₃)amino)ethyl-1,1,2,2-d₄)-1H-indol-4-ol (1-3), as determined by X-ray powder diffraction.

In some embodiments, the pharmaceutical composition comprises a pharmaceutically acceptable salt of a compound of Formula (I-3), in crystalline form, and a coated organic acid agent such as coated citric acid, coated tartaric acid, coated fumaric acid, etc. For effervescent dosage forms, a source of carbon dioxide (e.g., sodium bicarbonate) is also included with the coated organic acid agent. In some embodiments, the pharmaceutically acceptable salt is a benzenesulfonate salt of 3-(2-(bis(methyl-d₃)amino)ethyl-1,1,2,2-d₄)-1H-indol-4-ol (I-3a). In some embodiments, salt I-3a is in a crystalline solid form characterized by an X-ray powder diffraction pattern containing at least three characteristic peaks at diffraction angles (2θ±0.2°) selected from 7.023°, 7.767°, 11.822°, 12.550°, 12.860°, 13.994°, 15.521°, 18.436°, 19.503°, 20.760°, 21.070°, 22.007°, 22.745°, 23.340°, 24.187°, 25.532°, 26.880°, 27.856°, 28.163°, 31.267°, 33.024°, 35.030°, 36.835°, 39.312°, 40.545°, and 40.988°, as determined by XRPD using a CuKα radiation source, for example, as shown in WO2022195011 and/or WO2023078604 (pattern 1). In some embodiments, the pharmaceutically acceptable salt is a benzoate salt of 3-(2-(bis(methyl-d₃)amino)ethyl-1,1,2,2-d₄)-1H-indol-4-ol (I-3j). In some embodiments, salt I-3j is in a crystalline solid form characterized by an X-ray powder diffraction pattern containing at least three characteristic peaks at diffraction angles (2θ±0.2°) selected from 9.486°, 11.006°, 12.379°, 13.428°, 14.608°, 15.446°, 16.389°, 18.247°, 18.977°, 19.346°, 19.831°, 20.868°, 21.447°, 22.860°, 23.878°, 24.944°, 25.737°, 26.144°, 26.341°, 26.990°, 27.708°, 28.595°, 30.048°, 30.763°, 31.127°, 31.839°, 32.800°, 34.460°, 35.444°, 37.725°, and 38.597°, as determined by XRPD using a CuKα radiation source, as shown in WO2022195011 and/or WO2023078604 (pattern 1). In some embodiments, the pharmaceutically acceptable salt is a tartrate salt of 3-(2-(bis(methyl-d₃)amino)ethyl-1,1,2,2-d₄)-1H-indol-4-ol (I-3b). In some embodiments, salt I-3b is in a crystalline solid form of pattern 1 characterized by, e.g., an X-ray powder diffraction pattern shown in WO2022195011 and/or WO2023078604. In some embodiments, salt I-3b is in a crystalline solid form characterized by an X-ray powder diffraction pattern containing at least three characteristic peaks at diffraction angles (2θ±0.2°) selected from 6.732°, 12.708°, 13.470°, 14.774°, 15.921°, 16.268°, 17.295°, 18.869°, 20.079°, 20.208°, 20.877°, 21.894°, 22.657°, 23.491°, 23.702°, 24.636°, 24.882°, 25.569°, 26.685°, 27.060°, 27.502°, 28.179°, 28.597°, 29.035°, 29.257°, 29.527°, 31.017°, 31.527°, 32.059°, 32.307°, 33.012°, 34.024°, 34.388°, 34.905°, 35.361°, 36.183°, 37.372°, 37.764°, 38.657°, and 41.049°, as determined by XRPD using a CuKα radiation source, for example, as shown in WO2022195011 and/or WO2023078604 (pattern 2). In some embodiments, the pharmaceutically acceptable salt is a hemi-fumarate salt of 3-(2-(bis(methyl-d₃)amino)ethyl-1,1,2,2-d₄)-1H-indol-4-ol (I-3c). In some embodiments, salt I-3c is in a crystalline solid form of pattern 1 characterized by, e.g., an X-ray powder diffraction pattern as shown in WO2022195011 and/or WO2023078604. In some embodiments, salt I-3c is in a crystalline solid form characterized by an X-ray powder diffraction pattern containing at least three characteristic peaks at diffraction angles (2θ±0.2°) selected from 9.713°, 11.209°, 11.605°, 12.338°, 12.852°, 13.718°, 15.117°, 16.066°, 16.627°, 19.026°, 19.427°, 20.108°, 21.068°, 21.335°, 21.837°, 22.429°, 23.262°, 23.478°, 23.900°, 24.720°, 25.318°, 27.912°, 28.532°, 29.565°, 30.457°, 32.698°, 34.155°, 37.910°, 39.566°, and 40.999°, as determined by XRPD using a CuKα radiation source, as shown in WO2022195011 and/or WO2023078604 (pattern 2).

In some embodiments, the pharmaceutical composition comprises a pharmaceutically acceptable salt of a compound of Formula (I-3), in amorphous form, and a coated organic acid agent such as coated citric acid, coated tartaric acid, coated fumaric acid, etc. For effervescent dosage forms, a source of carbon dioxide (e.g., sodium bicarbonate) is also included with the coated organic acid agent. In some embodiments, the pharmaceutically acceptable salt is a citrate salt of 3-(2-(bis(methyl-d₃)amino)ethyl-1,1,2,2-d₄)-1H-indol-4-ol (1-3e). In some embodiments, salt I-3e is in the form of an amorphous solid as characterized by an X-ray powder diffraction (XRPD).

When the pharmaceutical composition is formulated with a pharmaceutically acceptable salt of a compound of Formula (I-3), the acid used in forming the pharmaceutically acceptable salt of a compound of Formula (I-3) and the organic acid agent (vehicle) can be the same. For example, the pharmaceutical composition may comprise a tartrate salt of a compound of Formula (I-3) (e.g., I-3b), and tartaric acid as organic acid agent (vehicle). In another example, the pharmaceutical composition may comprise a citrate salt of a compound of Formula (I-3) (e.g., I-3e), and citric acid as organic acid agent (vehicle).

When the pharmaceutical composition is formulated with a pharmaceutically acceptable salt of a compound of Formula (I-3), the acid used in forming the pharmaceutically acceptable salt of a compound of Formula (I-3) and the organic acid agent (vehicle) can be different. For example, the pharmaceutical composition may comprise a benzenesulfonate salt of a compound of Formula (1-3) (e.g., I-3a), and citric acid and/or tartaric acid, etc., as organic acid agent (vehicle). In another example, the pharmaceutical composition may comprise a benzoate salt of a compound of Formula (I-3) (e.g., I-3j), and citric acid and/or tartaric acid, etc., as organic acid agent (vehicle).

The pharmaceutical compositions disclosed herein may be disclosed in liquid and semisolid dosage forms, including emulsions, solutions, suspensions, elixirs, and syrups.

In some embodiments, oral liquid dosage forms are prepared by reconstituting a solid dosage form disclosed herein (e.g., an effervescent dosage form) into a pharmaceutically acceptable aqueous medium such as water, juice, or other drinkable fluid prior to use.

In some embodiments, the oral liquid dosage form is prepared by reconstituting into a pharmaceutically acceptable aqueous medium a solid dosage form comprising a compound of Formula (I-3) as a free base (e.g., I-3), in crystalline form. The solid dosage form may additionally be formulated with an organic acid agent, including a coated organic acid agent. Effervescent solid dosage forms may additionally be formulated with an organic acid agent, including a coated organic acid agent, and a source of carbon dioxide.

In some embodiments, the oral liquid dosage form is prepared by reconstituting into a pharmaceutically acceptable aqueous medium a solid dosage form comprising a compound of Formula (I-3) as a free base, in amorphous form. The solid dosage form may additionally be formulated with an organic acid agent, including a coated organic acid agent. Effervescent solid dosage forms may additionally be formulated with an organic acid agent, including a coated organic acid agent, and a source of carbon dioxide.

In some embodiments, the oral liquid dosage form is prepared by reconstituting into a pharmaceutically acceptable aqueous medium a solid dosage form comprising a pharmaceutically acceptable salt of a compound of Formula (I-3), in crystalline form. The solid dosage form may additionally be formulated with an organic acid agent, including a coated organic acid agent. Effervescent solid dosage forms may additionally be formulated with an organic acid agent, including a coated organic acid agent, and a source of carbon dioxide.

In some embodiments, the oral liquid dosage form is prepared by reconstituting into a pharmaceutically acceptable aqueous medium a solid dosage form comprising a pharmaceutically acceptable salt of a compound of Formula (I-3), in amorphous form. The solid dosage form may additionally be formulated with an organic acid agent, including a coated organic acid agent. Effervescent solid dosage forms may additionally be formulated with an organic acid agent, including a coated organic acid agent, and a source of carbon dioxide.

In some embodiments, the oral liquid dosage form is prepared by first dissolving a solid dosage form according to any embodiment described herein, in a pharmaceutically acceptable vehicle, such as an organic acid agent, to make a stock solution, then mixing the stock solution with a pharmaceutically acceptable aqueous medium such as water, juice, or other drinkable fluid prior to use. In some embodiments, the solid dosage form is dissolved in a solution of organic acid agent. In some embodiments, the organic acid agent is citric acid. In some embodiments, the organic acid agent is tartaric acid. In some embodiments, the stock solution is a 0.01M, 0.05M, 0.1M, 0.2M, 0.3M, 0.4M, 0.5M, 0.6M, 0.7M, 0.8M, 0.9M or 1.0M solution, or any range therebetween.

In some embodiments, the oral liquid dosage form is prepared by first dissolving a solid dosage form comprising a compound of Formula (I-3) as a free base, in citric acid to make a stock solution, then mixing the stock solution with a pharmaceutically acceptable aqueous medium such as water, juice, or other drinkable fluid prior to use.

In some embodiments, the oral liquid dosage form is prepared by first dissolving a solid dosage form comprising a compound of Formula (I-3) as a free base, in citric acid to make a 0.1M stock solution, then mixing the stock solution with a pharmaceutically acceptable aqueous medium such as water, juice, or other drinkable fluid prior to use.

An emulsion is a two-phase system, in which one liquid is dispersed in the form of small globules throughout another liquid, which can be oil-in-water or water-in-oil. Emulsions may include a pharmaceutically acceptable non-aqueous liquids or solvent, emulsifying agent, and preservative. Suspensions may include a pharmaceutically acceptable suspending agent and preservative. Aqueous alcoholic solutions may include a pharmaceutically acceptable acetal, such as a di(lower alkyl) acetal of a lower alkyl aldehyde (the term “lower” means an alkyl having between 1 and 6 carbon atoms), e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) disclosed herein, and a dialkylated mono- or poly-alkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations may further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates. In some embodiments, examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Cyclodextrins such as α-cyclodextrin, β-cyclodextrin, γ-cyclodextrin, methyl-β-cyclodextrin, hydroxyethyl β-cyclodextrin, hydroxypropyl-β-cyclodextrin, hydroxypropyl γ-cyclodextrin, sulfated β-cyclodextrin, sulfated α-cyclodextrin, sulfobutyl ether β-cyclodextrin, or other solubilized derivatives can also be advantageously used to enhance delivery of compositions described herein.

The pharmaceutical compositions disclosed herein for oral administration may be also disclosed in the forms of liposomes, micelles, microspheres, or nanosystems.

Coloring and flavoring agents can be used in all of the above dosage forms.

The pharmaceutical compositions disclosed herein may be co-formulated with other active ingredients which do not impair the desired therapeutic action, or with substances that supplement the desired action.

### B. Modified Release

The pharmaceutical compositions disclosed herein may be formulated as a modified release dosage form. As used herein, the term “modified release” refers to a dosage form in which the rate or place of release of the active ingredient(s) is different from that of an immediate dosage form when administered by the same route. The pharmaceutical compositions in modified release dosage forms can be prepared using a variety of modified release devices and methods known to those skilled in the art, including, but not limited to, matrix controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphism of the active ingredient(s).

### 1. Matrix Controlled Release Devices

The pharmaceutical compositions disclosed herein in a modified release dosage form may be fabricated using a matrix controlled release device known to those skilled in the art (see, Takada et al in “Encyclopedia of Controlled Drug Delivery,” Vol. 2, Mathiowitz ed., Wiley, 1999).

In one embodiment, the pharmaceutical compositions disclosed herein in a modified release dosage form is formulated using an erodible matrix device, which is water-swellable, erodible, or soluble polymers, including synthetic polymers, and naturally occurring polymers and derivatives, such as polysaccharides and proteins.

Materials useful in forming an erodible matrix include, but are not limited to, chitin, chitosan, dextran, and pullulan; gum agar, gum arabic, gum karaya, locust bean gum, gum tragacanth, carrageenans, gum ghatti, guar gum, xanthan gum, and scleroglucan; starches, such as dextrin and maltodextrin; hydrophilic colloids, such as pectin; phosphatides, such as lecithin; alginates; propylene glycol alginate; gelatin; collagen; and cellulosics, such as ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethylhydroxy ethylcellulose (EHEC); polyvinylpyrrolidone; polyvinyl alcohol; polyvinyl acetate; glycerol fatty acid esters; polyacrylamide; polyacrylic acid; copolymers of ethacrylic acid or methacrylic acid (EUDRAGIT®, Rohm America, Inc., Piscataway, N.J.); poly(2-hydroxyethyl-methacrylate); polylactides; copolymers of L-glutamic acid and ethyl-L-glutamate; degradable lactic acid-glycolic acid copolymers; poly-D-(−)-3-hydroxybutyric acid; and other acrylic acid derivatives, such as homopolymers and copolymers of butylmethacrylate, methylmethacrylate, ethylmethacrylate, ethylacrylate, (2-dimethylaminoethyl) methacrylate, and (trimethylaminoethyl) methacrylate chloride.

In further embodiments, the pharmaceutical compositions are formulated with a non-erodible matrix device. The active ingredient(s) is dissolved or dispersed in an inert matrix and is released primarily by diffusion through the inert matrix once administered. Materials suitable for use as a non-erodible matrix device included, but are not limited to, insoluble plastics, such as polyethylene, polypropylene, polyisoprene, polyisobutylene, polybutadiene, polymethylmethacrylate, polybutylmethacrylate, chlorinated polyethylene, polyvinylchloride, methyl acrylate-methyl methacrylate copolymers, ethylene-vinylacetate copolymers, ethylene/propylene copolymers, ethylene/ethyl acrylate copolymers, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, polyvinyl chloride, plasticized nylon, plasticized polyethyleneterephthalate, natural rubber, silicone rubbers, polydimethylsiloxanes, silicone carbonate copolymers, and; hydrophilic polymers, such as ethyl cellulose, cellulose acetate, crospovidone, and cross-linked partially hydrolyzed polyvinyl acetate, and fatty compounds, such as carnauba wax, microcrystalline wax, and triglycerides.

In a matrix controlled release system, the desired release kinetics can be controlled, for example, via the polymer type employed, the polymer viscosity, the particle sizes of the polymer and/or the active ingredient(s), the ratio of the active ingredient(s) versus the polymer, and other excipients or carriers in the compositions.

The pharmaceutical compositions disclosed herein in a modified release dosage form may be prepared by methods known to those skilled in the art, including direct compression, dry or wet granulation followed by compression, melt-granulation followed by compression.

### 2. Osmotic Controlled Release Devices

The pharmaceutical compositions disclosed herein in a modified release dosage form may be fabricated using an osmotic controlled release device, including one-chamber system, two-chamber system, asymmetric membrane technology (AMT), and extruding core system (ECS). In general, such devices have at least two components: (a) the core which contains the active ingredient(s); and (b) a semipermeable membrane with at least one delivery port, which encapsulates the core. The semipermeable membrane controls the influx of water to the core from an aqueous environment of use so as to cause drug release by extrusion through the delivery port(s).

In addition to the active ingredient(s), the core of the osmotic device optionally includes an osmotic agent, which creates a driving force for transport of water from the environment of use into the core of the device. One class of osmotic agents water-swellable hydrophilic polymers, which are also referred to as “osmopolymers” and “hydrogels,” including, but not limited to, hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP), crosslinked PVP, polyvinyl alcohol (PVA), PVA/PVP copolymers, PVA/PVP copolymers with hydrophobic monomers such as methyl methacrylate and vinyl acetate, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl, cellulose (CEC), sodium alginate, polycarbophil, gelatin, xanthan gum, and sodium starch glycolate.

The other class of osmotic agents are osmogens, which are capable of imbibing water to affect an osmotic pressure gradient across the barrier of the surrounding coating. Suitable osmogens include, but are not limited to, inorganic salts, such as magnesium sulfate, magnesium chloride, calcium chloride, sodium chloride, lithium chloride, potassium sulfate, potassium phosphates, sodium carbonate, sodium sulfite, lithium sulfate, potassium chloride, and sodium sulfate; sugars, such as dextrose, fructose, glucose, inositol, lactose, maltose, mannitol, raffinose, sorbitol, sucrose, trehalose, and xylitol, organic acids, such as ascorbic acid, benzoic acid, fumaric acid, citric acid, maleic acid, sebacic acid, sorbic acid, adipic acid, edetic acid, glutamic acid, p-toluenesulfonic acid, succinic acid, and tartaric acid; urea; and mixtures thereof.

Osmotic agents of different dissolution rates may be employed to influence how rapidly the active ingredient(s) is initially delivered from the dosage form. For example, amorphous sugars, such as Mannogeme EZ (SPI Pharma, Lewes, Del.) can be used to provide faster delivery during the first couple of hours to promptly produce the desired therapeutic effect, and gradually and continually release of the remaining amount to maintain the desired level of therapeutic or prophylactic effect over an extended period of time. In this case, the active ingredient(s) is released at such a rate to replace the amount of the active ingredient metabolized and excreted.

The core may also include a wide variety of other excipients and carriers as described herein to enhance the performance of the dosage form or to promote stability or processing.

Materials useful in forming the semipermeable membrane include various grades of acrylics, vinyls, ethers, polyamides, polyesters, and cellulosic derivatives that are water-permeable and water-insoluble at physiologically relevant pHs, or are susceptible to being rendered water-insoluble by chemical alteration, such as crosslinking. Examples of suitable polymers useful in forming the coating, include plasticized, unplasticized, and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CA p-toluene sulfonate, agar acetate, amylose triacetate, beta glucan acetate, beta glucan triacetate, acetaldehyde dimethyl acetate, triacetate of locust bean gum, hydroxylated ethylene-vinylacetate, EC, PEG, PPG, PEG/PPG copolymers, PVP, HEC, HPC, CMC, CMEC, HPMC, HPMCP, HPMCAS, HPMCAT, poly(acrylic) acids and esters and poly-(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes, and synthetic waxes.

Semipermeable membrane may also be a hydrophobic microporous membrane, wherein the pores are substantially filled with a gas and are not wetted by the aqueous medium but are permeable to water vapor, as disclosed in U.S. Pat. No. 5,798,119. Such hydrophobic but water-vapor permeable membrane are typically composed of hydrophobic polymers such as polyalkenes, polyethylene, polypropylene, polytetrafluoroethylene, polyacrylic acid derivatives, polyethers, polysulfones, polyethersulfones, polystyrenes, polyvinyl halides, polyvinylidene fluoride, polyvinyl esters and ethers, natural waxes, and synthetic waxes.

The delivery port(s) on the semipermeable membrane may be formed post-coating by mechanical or laser drilling. Delivery port(s) may also be formed in situ by erosion of a plug of water-soluble material or by rupture of a thinner portion of the membrane over an indentation in the core. In addition, delivery ports may be formed during coating process, as in the case of asymmetric membrane coatings of the type disclosed in U.S. Pat. Nos. 5,612,059 and 5,698,220.

The total amount of the active ingredient(s) released, and the release rate can substantially by modulated via the thickness and porosity of the semipermeable membrane, the composition of the core, and the number, size, and position of the delivery ports.

The pharmaceutical compositions in an osmotic controlled-release dosage form may further comprise additional conventional excipients or carriers as described herein to promote performance or processing of the composition.

The osmotic controlled-release dosage forms can be prepared according to conventional methods and techniques known to those skilled in the art (see, Remington: The Science and Practice of Pharmacy, supra; Santus and Baker, J. Controlled Release 1995, 35, 1-21; Verma et al., Drug Development and Industrial Pharmacy 2000, 26, 695-708; Verma et al., J. Controlled Release 2002, 79, 7-27).

In some embodiments, the pharmaceutical compositions disclosed herein are formulated as AMT controlled-release dosage forms, which comprises an asymmetric osmotic membrane that coats a core comprising the active ingredient(s) and other pharmaceutically acceptable vehicles (e.g., excipients or carriers). The AMT controlled-release dosage forms can be prepared according to conventional methods and techniques known to those skilled in the art, including direct compression, dry granulation, wet granulation, and a dip-coating method.

In some embodiments, the pharmaceutical compositions disclosed herein are formulated as ESC controlled-release dosage form, which comprises an osmotic membrane that coats a core comprising the active ingredient(s), a hydroxylethyl cellulose, and other pharmaceutically acceptable excipients or carriers.

### 3. Multiparticulate Controlled Release Devices

The pharmaceutical compositions disclosed herein in a modified release dosage form may be fabricated a multiparticulate controlled release device, which comprises a multiplicity of particles, granules, or pellets, ranging from about 10 μm to about 3 mm, about 50 m to about 2.5 mm, or from about 100 m to about 1 mm in diameter. Such multiparticulates may be made by the processes know to those skilled in the art, including wet- and dry-granulation, extrusion/spheronization, roller-compaction, melt-congealing, and by spray-coating seed cores. See, for example, Multiparticulate Oral Drug Delivery; Marcel Dekker: 1994; and Pharmaceutical Pelletization Technology; Marcel Dekker: 1989.

Other excipients or carriers as described herein may be blended with the pharmaceutical compositions to aid in processing and forming the multiparticulates. The resulting particles may themselves constitute the multiparticulate device or may be coated by various film-forming materials, such as enteric polymers, water-swellable, and water-soluble polymers. The multiparticulates can be further processed as a capsule or a tablet.

### Pharmacokinetics

In some embodiments, the pharmaceutical composition has an onset of therapeutic action of 60 minutes or less, 50 minutes or less, 40 minutes or less, 30 minutes or less, 20 minutes or less, 10 minutes or less, or 5 minutes or less. In some embodiments, the pharmaceutical composition has an acute effects duration of 240 minutes or less, 180 minutes or less, 120 minutes or less, 60 minutes or less, 50 minutes or less, 40 minutes or less, 30 minutes or less, 20 minutes or less, 10 minutes or less, or 5 minutes or less. In some embodiments, the pharmaceutical composition has a drug dissolution time of 120 seconds or less, 90 seconds or less, 60 seconds or less, 50 seconds or less, 40 seconds or less, 30 seconds or less, 20 seconds or less, 10 seconds or less, or 5 seconds or less.

### Stabilized Compositions

In some embodiments, pharmaceutical compositions are provided which include the compound of Formula (I-3), or a pharmaceutically acceptable salt, polymorph, or solvate thereof, in a stabilized form with a pharmaceutically acceptable vehicle. For example, an amorphous form of the compound of Formula (I-3) may be stabilized in the disclosed pharmaceutical compositions. In some embodiments, formulations of the compound of Formula (I-3) in which the compound of Formula (I-3) exists stably in amorphous form may be accomplished, for example, by immobilizing the compound within a matrix formed by a polymer, e.g., as a solid dispersion or solid molecular complex of the compound of Formula (I-3) and a polymer.

Provided are solid dispersions and solid molecular complexes that include the compound of Formula (I-3), or a pharmaceutically acceptable salt, polymorph, or solvate thereof. For example, the compound of Formula (I-3), or a pharmaceutically acceptable salt, polymorph, or solvate thereof, may be dispersed within a matrix formed by a polymer in its solid state such that it is immobilized in its amorphous form. In some embodiments, the polymer may prevent intramolecular hydrogen bonding or weak dispersion forces between two or more drug molecules of the compound of Formula (I-3). In some embodiments, the solid dispersion provides for a large surface area, thus further allowing for improved dissolution and bioavailability of the compound of Formula (I-3). In some embodiments, a solid dispersion or solid molecular complex includes about 8-16 mg, or about 8-14 mg, or about 8-12 mg, or about 12-16 mg, or about 12 mg, or about 16 mg of the compound of Formula (I-3), or a pharmaceutically acceptable salt, polymorph, or solvate thereof.

In some embodiments, the compound of Formula (I-3), or a pharmaceutically acceptable salt, polymorph, or solvate thereof, is present in the solid dispersion in an amount of from about 1%, about 5%, about 10%, about 15%, about 20%, about 25%, about 30%, about 35%, about 40%, about 45%, about 50% by weight, based on a total weight of the solid dispersion, or any range therebetween, e.g., from about 1% to about 50% by weight; or from about 10% to about 40% by weight; or from about 20% to about 35% by weight; or from about 25% to about 30% by weight. In some embodiments, a polymer is present in the solid dispersion in an amount of from about 0%, about 1%, about 5%, about 10%, about 15%, about 20%, about 25%, about 30%, about 35%, about 40%, about 45%, about 50%, about 60%, about 70%, about 80%, about 90% by weight, based on a total weight of the solid dispersion, or any range therebetween, e.g., from 0% to about 50% by weight; or from about 5% to about 60% by weight; or from 10% to about 70% by weight. In some embodiments, a polymer is present in the solid dispersion in an amount greater than about 10% by weight; or greater than about 20% by weight; or greater than about 30% by weight; or greater than about 40% by weight; or greater than about 50% by weight, based on a total weight of the solid dispersion. In some embodiments, the solid dispersion is about 30% by weight of the compound of Formula (I-3), or a pharmaceutically acceptable salt, polymorph, or solvate thereof, and about 70% by weight polymer.

The solid dispersion may comprise the compound of Formula (I-3), or a pharmaceutically acceptable salt, polymorph, or solvate thereof dispersed in a non-ionic polymer. This may be accomplished by, for example, melting the polymer and dissolving the compound in the polymer and then cooling the mixture. The resulting solid dispersion may comprise the compound dispersed in the polymer in amorphous form.

A solid dispersion may be formed by dispersing the compound of Formula (I-3), or a pharmaceutically acceptable salt, polymorph, or solvate thereof in an ionic polymer. Such solid dispersion may result in increased stability of the compound of Formula (I-3), or a pharmaceutically acceptable salt, polymorph, or solvate thereof. This may be accomplished by various means, including the methods described above for use in forming a dispersion in a non-ionic polymer. Because ionic polymers have pH dependent solubility in aqueous systems, the resulting solid dispersion of the compound of Formula (I-3) and the polymer may be stable at low pH in the stomach and release the compound of Formula (I-3) in the intestine at higher pH. In some embodiments, the compound of Formula (I-3), or a pharmaceutically acceptable salt, polymorph, or solvate thereof in such solid dispersions with an ionic polymer may thus be less capable of separating from the polymer and may be immobilized by the polymer in its amorphous form. Examples of such ionic polymers include, but are not limited to, hydroxypropylmethyl cellulose acetate succinate (HPMC-AS), hydroxypropylmethyl cellulose phthalate (HPMCP), and methacrylic acid copolymers. In some embodiments, a polymer is used that is capable of immobilizing the compound of Formula (I-3), or a pharmaceutically acceptable salt, polymorph, or solvate thereof so that it exists primarily in one particular polymorph, e.g., an amorphous form, for an extended period of time.

In some embodiments, the polymer may be linear, branched, or crosslinked. In some embodiments, the polymer may be a homopolymer or copolymer. In some embodiments, the polymer may be a synthetic polymer derived from vinyl, acrylate, methacrylate, urethane, ester and oxide monomers. In some embodiments, the polymer can be a derivative of naturally occurring polymers such as polysaccharides (e.g. chitin, chitosan, dextran and pullulan; gum agar, gum arabic, gum karaya, locust bean gum, gum tragacanth, carrageenans, gum ghatti, guar gum, xanthan gum and scleroglucan), starches (e.g. dextrin and maltodextrin), hydrophilic colloids (e.g. pectin), phosphatides (e.g. lecithin), alginates (e.g. ammonium alginate, sodium, potassium or calcium alginate, propylene glycol alginate), gelatin, collagen, and cellulose polymers. In some embodiments, the cellulose polymer is selected from the group consisting of ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethylhydroxy ethylcellulose (EHEC). In some embodiments, the polymer may be selected from the group consisting of gelatin, polyvinyl alcohol, polyvinylpyrrolidone, pullulan, and the cellulose polymers already disclosed herein. In some embodiments, the cellulose polymer comprises various grades of low viscosity, e.g., MW less than or equal to 50,000 daltons.

In some embodiments, the composition can include solid dispersions and solid molecular complexes that include the compound of Formula (I-3), or a pharmaceutically acceptable salt, polymorph, or solvate thereof dispersed within a matrix formed by gelatin. In some embodiments, the composition can include solid dispersions and solid molecular complexes that include the compound of Formula (I-3), or a pharmaceutically acceptable salt, polymorph, or solvate thereof dispersed within a matrix formed by gelatin and a non-reducing sugar, e.g., mannitol. In some embodiments, the composition can include solid dispersions and solid molecular complexes that include the compound of Formula (I-3), or a pharmaceutically acceptable salt, polymorph, or solvate thereof dispersed within a matrix formed by a cellulose polymer described herein. In some embodiments, the composition can include solid dispersions and solid molecular complexes that include the compound of Formula (I-3), or a pharmaceutically acceptable salt, polymorph, or solvate thereof dispersed within a matrix formed by a cellulose polymer described herein and polyvinylpyrrolidone.

In some embodiments, the ratio of the amount by weight of the compound of Formula (I-3), or a pharmaceutically acceptable salt, polymorph, or solvate thereof within the solid complex to the amount by weight of the polymer therein is from about 1:9 to about 1:1. In some embodiments, the ratio of the amount by weight of the compound of Formula (I-3), or a pharmaceutically acceptable salt, polymorph, or solvate thereof, within the solid complex to the amount by weight of the polymer therein is from about 2:8 to about 4:6. In some embodiments, the ratio of the amount by weight of the compound of Formula (I-3), or a pharmaceutically acceptable salt, polymorph, or solvate thereof within the solid complex to the amount by weight of the polymer therein is about 3:7.

In some embodiments, the composition can further include one or more pharmaceutically acceptable vehicles, such as solubilizing agents for the compound of Formula (I-3), or a pharmaceutically acceptable salt, polymorph, or solvate thereof. Solubilizing agents include those set forth herein, such as organic acid agents (e.g., citric acid), sodium phosphate, and natural amino acids. Other solubilizing agents include, but are not limited to, acacia, cholesterol, diethanolamine (adjunct), glyceryl monostearate, lanolin alcohols, mono- and di-glycerides, monoethanolamine (adjunct), lecithin, oleic acid (adjunct), oleyl alcohol (stabilizing agent), poloxamer, polyoxyethylene 50 stearate, polyoxyl 35 castor oil, polyoxyl 40 hydrogenated castor oil, polyoxyl 10 oleyl ether, polyoxyl 20 cetostearyl ether, polyoxyl 40 stearate, polysorbate 20, polysorbate 40, polysorbate 60, polysorbate 80, diacetate, monostearate, sodium lauryl sulfate, sodium stearate, sorbitan monolaurate, sorbitan monooleate, sorbitan monopalmitate, sorbitan monostearate, stearic acid, trolamine, and emulsifying wax.

Various additives can be mixed, ground or granulated with the solid dispersion as described herein to form a material suitable for the above dosage forms. Potentially beneficial additives may fall generally into the following classes: other matrix materials or diluents, surface active agents, drug complexing agents or solubilizing agents, fillers, disintegrants, binders, lubricants, and pH modifiers (e.g., acids, bases, or buffers). Examples of other matrix materials, fillers, or diluents include lactose, mannitol, xylitol, microcrystalline cellulose including silicified microcrystalline cellulose (SMCC), calcium diphosphate, and starch. Examples of surface active agents include sodium lauryl sulfate and polysorbate 80. Examples of drug complexing agents or solubilizing agents include the polyethylene glycols, caffeine, xanthene, gentisic acid and cylodextrins. Examples of disintegrants include sodium starch gycolate, sodium alginate, carboxymethyl cellulose sodium, methyl cellulose, and croscarmellose sodium. Examples of binders include methyl cellulose, microcrystalline cellulose including silicified microcrystalline cellulose (SMCC), starch, and gums such as guar gum, and tragacanth. Examples of lubricants include magnesium stearate and calcium stearate. Examples of pH modifiers include acids (including organic acid agents), such as citric acid, acetic acid, ascorbic acid, lactic acid, aspartic acid, succinic acid, phosphoric acid, and the like; bases such as sodium acetate, potassium acetate, sodium citrate, potassium citrate, sodium tartrate, potassium tartrate, calcium oxide, magnesium oxide, trisodium phosphate, sodium hydroxide, calcium hydroxide, aluminum hydroxide, and the like, and buffers generally comprising mixtures of acids and the salts of said acids.

The composition may, in addition to the solid dispersion or solid molecular complex, also comprise therapeutically inert, inorganic or organic vehicles, such as those set forth herein.

### Dosage, Frequency and Routes of Administration

The dosage and frequency (single or multiple doses) of the compound of Formula (I-3), or a pharmaceutically acceptable salt, polymorph, or solvate thereof, that is administered can vary depending upon a variety of factors, including, but not limited to, the salt form/compound/polymorph to be administered; route of administration; size, age, sex, health, body weight, body mass index, and diet of the recipient; nature and extent of symptoms of the disease being treated; presence of other diseases or other health-related problems; kind of concurrent treatment; and complications from any disease or treatment regimen. Other therapeutic regimens or agents can be used in conjunction with the methods and compounds disclosed herein.

Therapeutically effective amounts for use in humans may be determined from animal models. For example, a dose for humans can be formulated to achieve a concentration that has been found to be effective in animals. The dosage in humans can be adjusted by monitoring response to the treatment and adjusting the dosage upwards (e.g., up-titration) or downwards (e.g., down-titration).

Dosages may be varied depending upon the requirements of the subject and the active ingredient(s) being employed. The dose administered to a subject, in the context of the pharmaceutical compositions presented herein, should be sufficient to affect a beneficial therapeutic response in the subject over time. The size of the dose also will be determined by the existence, nature, and extent of any adverse side effects. Generally, treatment is initiated with smaller dosages, which are less than the optimum dose of the compound. Thereafter, the dosage is increased by small increments until the optimum effect under circumstances is reached.

Dosage amounts and intervals can be adjusted individually to provide levels of the administered compounds effective for the particular clinical indication being treated. This will provide a therapeutic regimen that is commensurate with the severity of the individual's disease state.

Routes of administration may include oral routes (e.g., enteral/gastric delivery, intraoral administration such buccal, lingual, and sublingual routes), parenteral routes (e.g., intravenous, intradermal, intraarterial, intraperitoneal, intrathecal, intraventricular, intraurethral, intrasternal, intracranial, intramuscular, intrasynovial, and subcutaneous administration), and topical routes (e.g., (intra) dermal, conjuctival, intracorneal, intraocular, ophthalmic, auricular, transdermal, nasal, vaginal, uretheral, respiratory, and rectal administration), or others sufficient to affect a beneficial therapeutic response.

Administration may follow a continuous administration schedule (7 days of administration in a week), or an intermittent administration schedule. The administration schedule may be varied depending on the active ingredient(s) employed, the condition being treated, the administration route, the pharmacokinetics and a particular subject's clearance/accumulation of the drug, etc. For example, administration of a compound of Formula (I-3), or a pharmaceutically acceptable salt, polymorph, or solvate thereof, may be performed once a day (QD), or in divided dosages throughout the day, such as 2-times a day (BID), 3-times a day (TID), 4-times a day (QID), or more. In some embodiments administration may be performed nightly (QHS). In some embodiments, administration is performed as needed (PRN). If intermittently, the schedule may be, for example, 4 days of administration and 3 days off (rest days) in a week or any other intermittent dosing schedule deemed appropriate using sound medical judgement. Intermittent administration may also be performed on a weekly or monthly basis, e.g., once a week, twice a week, three times a week, four times a week, every other week, every two weeks, every three weeks, every four weeks, every five weeks, every six weeks, every seven weeks, every eight weeks, every nine weeks, every ten weeks, every eleven weeks, every twelve weeks, etc., or less, or any range therebetween. Such administration schedules may be provided with dosing day flexibility, e.g., ±1 day, 2 days, 3 days, etc. The (intermittent) administration schedule may also designate a defined number of treatments per treatment course, for example, the compound of Formula (I-3), or a pharmaceutically acceptable salt, polymorph, or solvate thereof, may be administered 1 time, 2 times, 3 times, 4 times, 5 times, 6 times, 7 times, or 8 times per treatment course. Other administration schedules may also be deemed appropriate using sound medical judgement.

In some embodiments, one dose of a compound of Formula (I-3), or a pharmaceutically acceptable salt, polymorph, or solvate thereof, is administered to the subject in a treatment course. In some embodiments, treatment herein may involve one dose of about 8-16 mg, about 8-14 mg, about 8-12 mg, or about 12-16 mg, or about 12 mg, or about 16 mg of the compound of Formula (I-3), or a pharmaceutically acceptable salt, polymorph, or solvate thereof, administered to the subject in a treatment course. In some embodiments, treatment herein may involve a single dose of about 12 mg, or a single dose of about 16 mg of the compound of Formula (I-3), or a pharmaceutically acceptable salt, polymorph, or solvate thereof, administered to the subject in a treatment course.

In some embodiments, multiple doses of a compound of Formula (I-3), or a pharmaceutically acceptable salt, polymorph, or solvate thereof, are administered to the subject in a treatment course. The multiple doses may involve a first dose, a second dose, a third dose, a fourth dose, etc. In some embodiments, multiple doses of a compound of Formula (I-3), or a pharmaceutically acceptable salt, polymorph, or solvate thereof, are administered to the subject one week apart, two weeks apart, three weeks apart, or four weeks apart (±3 days), in a treatment course. For example, the treatment herein may involve multiple doses of a compound of Formula (I-3), or a pharmaceutically acceptable salt, polymorph, or solvate thereof, administered three weeks apart (e.g., day 1 and day 22) (±3 days). In some embodiments, treatment herein may involve multiple doses, each of about 8-16 mg, about 8-14 mg, about 8-12 mg, about 12-16 mg, or about 12 mg, or about 16 mg of the compound of Formula (I-3), or a pharmaceutically acceptable salt, polymorph, or solvate thereof, administered to the subject three weeks apart (±3 days), in a treatment course. In some embodiments, treatment herein may involve multiple doses, each of about 12 mg or each of about 16 mg of the compound of Formula (I-3), or a pharmaceutically acceptable salt, polymorph, or solvate thereof, administered to the subject three weeks apart (±3 days).

In some embodiments, a first dose and a second dose of a compound of Formula (I-3), or a pharmaceutically acceptable salt, polymorph, or solvate thereof, are administered to the subject one week apart, two weeks apart, three weeks apart, or four weeks apart (±3 days), in a treatment course. In some embodiments, two doses of a compound of Formula (I-3), or a pharmaceutically acceptable salt, polymorph, or solvate thereof, are administered to the subject one week apart, two weeks apart, three weeks apart, or four weeks apart (±3 days), in a treatment course. For example, the treatment herein may involve two doses of a compound of Formula (I-3), or a pharmaceutically acceptable salt, polymorph, or solvate thereof, administered three weeks apart (e.g., day 1 and day 22±3 days). In some embodiments, treatment herein may involve two doses, each of about 8-16 mg, about 8-14 mg, about 8-12 mg, about 12-16 mg, or about 12 mg, or about 16 mg of the compound of Formula (I-3), or a pharmaceutically acceptable salt, polymorph, or solvate thereof, administered to the subject three weeks apart (±3 days), in a treatment course. In some embodiments, treatment herein may involve two doses, each of about 12 mg or each of about 16 mg of the compound of Formula (I-3), or a pharmaceutically acceptable salt, polymorph, or solvate thereof, administered to the subject three weeks apart (±3 days).

In some embodiments, a first dose, a second dose, and a third dose of a compound of Formula (I-3), or a pharmaceutically acceptable salt, polymorph, or solvate thereof, are administered to the subject one week apart, two weeks apart, three weeks apart, or four weeks apart (±3 days), in a treatment course. In some embodiments, three doses of a compound of Formula (I-3), or a pharmaceutically acceptable salt, polymorph, or solvate thereof, are administered to the subject one week apart, two weeks apart, three weeks apart, or four weeks apart (±3 days), in a treatment course. For example, the treatment herein may involve three doses of a compound of Formula (I-3), or a pharmaceutically acceptable salt, polymorph, or solvate thereof, administered three weeks apart (e.g., day 1, day 22, and day 43, ±3 days). In some embodiments, treatment herein may involve three doses, each of about 8-16 mg, about 8-14 mg, about 8-12 mg, about 12-16 mg, or about 12 mg, or about 16 mg of the compound of Formula (I-3), or a pharmaceutically acceptable salt, polymorph, or solvate thereof, administered to the subject three weeks apart (±3 days), in a treatment course. In some embodiments, treatment herein may involve three doses, each of about 12 mg or each of about 16 mg of the compound of Formula (I-3), or a pharmaceutically acceptable salt, polymorph, or solvate thereof, administered to the subject three weeks apart (±3 days).

In some embodiments, a first dose, a second dose, a third dose, and a fourth dose of a compound of Formula (I-3), or a pharmaceutically acceptable salt, polymorph, or solvate thereof, are administered to the subject one week apart, two weeks apart, three weeks apart, or four weeks apart (±3 days), in a treatment course. In some embodiments, four doses of a compound of Formula (I-3), or a pharmaceutically acceptable salt, polymorph, or solvate thereof, are administered to the subject one week apart, two weeks apart, three weeks apart, or four weeks apart (±3 days), in a treatment course. For example, the treatment herein may involve four doses of a compound of Formula (I-3), or a pharmaceutically acceptable salt, polymorph, or solvate thereof, administered three weeks apart (e.g., day 1, day 22, day 43, and day 64, ±3 days). In some embodiments, treatment herein may involve four doses, each of about 8-16 mg, about 8-14 mg, about 8-12 mg, about 12-16 mg, or about 12 mg, or about 16 mg of the compound of Formula (I-3), or a pharmaceutically acceptable salt, polymorph, or solvate thereof, administered to the subject three weeks apart (±3 days), in a treatment course. In some embodiments, treatment herein may involve four doses, each of about 12 mg or about 16 mg of the compound of Formula (I-3), or a pharmaceutically acceptable salt, polymorph, or solvate thereof, administered to the subject three weeks apart (±3 days).

In some embodiments, only one, two, three, or four doses of a compound of Formula (I-3), or a pharmaceutically acceptable salt, polymorph, or solvate thereof, are administered to the subject in any 2-month period. In some embodiments, only one, two, three, or four doses of a compound of Formula (I-3), or a pharmaceutically acceptable salt, polymorph, or solvate thereof, are administered to the subject in any 3-month period. In some embodiments, only one, two, three, or four doses of a compound of Formula (I-3), or a pharmaceutically acceptable salt, polymorph, or solvate thereof, are administered to the subject in any 4-month period. In some embodiments, only one, two, three, or four doses of a compound of Formula (I-3), or a pharmaceutically acceptable salt, polymorph, or solvate thereof, are administered to the subject in any 5-month period. In some embodiments, only one, two, three, or four doses of a compound of Formula (I-3), or a pharmaceutically acceptable salt, polymorph, or solvate thereof, are administered to the subject in any 6-month period. In some embodiments, only one, two, three, or four doses of a compound of Formula (I-3), or a pharmaceutically acceptable salt, polymorph, or solvate thereof, are administered to the subject in any 7-month period. In some embodiments, only one, two, three, or four doses of a compound of Formula (I-3), or a pharmaceutically acceptable salt, polymorph, or solvate thereof, are administered to the subject in any 8-month period. In some embodiments, only one, two, three, or four doses of a compound of Formula (I-3), or a pharmaceutically acceptable salt, polymorph, or solvate thereof, are administered to the subject in any 9-month period. In some embodiments, only one, two, three, or four doses of a compound of Formula (I-3), or a pharmaceutically acceptable salt, polymorph, or solvate thereof, are administered to the subject in any 10-month period. In some embodiments, only one, two, three, or four doses of a compound of Formula (I-3), or a pharmaceutically acceptable salt, polymorph, or solvate thereof, are administered to the subject in any 11-month period. In some embodiments, only one, two, three, or four doses of a compound of Formula (I-3), or a pharmaceutically acceptable salt, polymorph, or solvate thereof, are administered to the subject in any 12-month period. In some embodiments, only one, two, three, or four doses of a compound of Formula (I-3), or a pharmaceutically acceptable salt, polymorph, or solvate thereof, are administered to the subject in any 2-year period. In some embodiments, only one, two, three, or four doses of a compound of Formula (I-3), or a pharmaceutically acceptable salt, polymorph, or solvate thereof, are administered to the subject in any 3-year period. In any embodiment where multiple (e.g., two, three, or four) doses of a compound of Formula (I-3), or a pharmaceutically acceptable salt, polymorph, or solvate thereof, are administered within a certain time period, it is preferred that administration of each dose occur one week apart, two weeks apart, three weeks apart, or four weeks apart (±3 days). For example, a preferred administration schedule may involve only two doses of a compound of Formula (I-3), or a pharmaceutically acceptable salt, polymorph, or solvate thereof, in any 2-, 3-, 4-, 5-, 6-, 7-, or 8-month period, wherein the two doses, each of about 8-16 mg, about 8-14 mg, about 8-12 mg, about 12-16 mg, or about 12 mg, or about 16 mg of the compound of Formula (I-3), or a pharmaceutically acceptable salt, polymorph, or solvate thereof, are administered three weeks apart (±3 days).

Administration of each dose (e.g., a single dose in a single dosing regimen, each dose in a two-dose or multiple dose regimen, etc.) may be accompanied by psychotherapy, before, during, and/or after each dose, whereby the subject participates in one or more pre-administration psychological support session(s); the subject participates in one or more psychological support session(s) during each medicine session; and/or the subject participates in one or more post-administration psychological support session(s).

The dosing whether continuous or intermittent is continued for a particular treatment course, typically at least a 28-day cycle (1 month), which can be repeated with or without a drug holiday. Longer or shorter courses can also be used such as 14 days, 18 days, 21 days, 24 days, 28 days, 35 days, 42 days, 48 days, 52 days, 56 days, 64 days, or longer, or any range therebetween. The course may be repeated without a drug holiday or with a drug holiday depending upon the subject. In some embodiments, the method herein involves one treatment course for the lifetime of the subject, wherein administration follows an administration schedule (e.g., a single dose, two doses administered three weeks apart, etc.), with no repeat dosing during the remaining lifespan of the subject. In some embodiments, the method herein involves more than one treatment course, for example, if the subject fails to respond to initial treatment(s) or if a relapse occurs. Other schedules are possible depending upon the presence or absence of adverse events, response to the treatment, potential relapse, patient convenience, and the like.

In some embodiments, the use of compositions of the disclosure may be used as a standalone therapy. In some embodiments, the use of compositions of the disclosure may be used as an adjuvant/combination therapy.

Utilizing the teachings provided herein, an effective prophylactic or therapeutic treatment regimen can be planned that does not cause substantial toxicity or adverse side effects (e.g., caused by sedative or psychotomimetic toxic spikes in plasma concentration of any of the compounds of Formula (I-3)), and yet is entirely effective to treat the clinical symptoms demonstrated by the particular subject. This planning should involve the careful choice of active compound and salt form by considering factors such as compound potency, relative bioavailability, patient body weight, presence and severity of adverse side effects, preferred mode of administration, and the toxicity profile of the selected agent.

In some embodiments, a dose is administered once by mouth, with the possibility of repeat doses at least one week apart. In some instances, no more than 5 doses are given in any one course of treatment. Courses can be repeated as necessary, with or without a drug holiday. Such acute treatment regimens may be accompanied by psychotherapy, before, during, and/or after the psychedelic dose.

The compounds of the present disclosure (e.g., a compound of Formula (I-3), or a pharmaceutically acceptable salt, polymorph, or solvate thereof), may be used for a maintenance regimen. As used herein, a “maintenance regimen” generally refers to the administration of the compounds of the present disclosure (e.g., a compound of Formula (I-3), or a pharmaceutically acceptable salt, polymorph, or solvate thereof) following achievement of a target dose, e.g., following completion of an up-titration regimen, and/or following a positive clinical response, e.g., improvement of the patient's condition, either to the same drug or to a different drug. In some embodiments, the patient is administered a first drug for a therapeutic regimen and a second drug for a maintenance regimen, wherein the first and second drugs are different. For example, the patient may be administered a therapeutic regimen of a first drug which is not a compound of the present disclosure (e.g., the first drug is a serotonergic psychedelic such as LSD, psilocybin, MDMA, dimethyltryptamine, etc., or a non-psychedelic drug), followed by a compound of the present disclosure (as the second drug) in a maintenance regimen. In another example, a different compound of the present disclosure is used for the therapeutic regimen (first drug) than is used for the maintenance regimen (second drug). In some embodiments, the patient is administered the same compound of the present disclosure for both a therapeutic regimen and a maintenance regimen. In any case, the maintenance dose of the compounds of the present disclosure may be used to ‘maintain’ the therapeutic response and/or to prevent occurrences of relapse. When the same compound of the present disclosure is used for both the original therapeutic regimen and for the maintenance regimen, the maintenance dose of the compound may be at or below the therapeutic dose. Generally, dosing is carried out daily or intermittently for the maintenance regimen, however, maintenance regimens can also be carried out continuously, for example, over several days, weeks, months, or years. Moreover, the maintenance dose may be given to a patient over a long period of time, even chronically.

The administering physician can provide a method of treatment that is prophylactic or therapeutic by adjusting the amount and timing of any of the compounds/salt forms described herein on the basis of observations of one or more symptoms of the disorder or condition being treated. In some embodiments, the subject is a mammal. In some embodiments, the mammal is a human.

### Diseases, Disorders, and Conditions

The subjects treated herein may have a disease or disorder associated with a serotonin 5-HT₂ receptor, particularly a 5-HT_{2A} receptor. In some embodiments, the disease or disorder is a neuropsychiatric disease or disorder or an inflammatory disease or disorder. In some embodiments, the disease or disorder is a central nervous system (CNS) disorder, including, but not limited to, major depressive disorder (MDD), treatment-resistant depression (TRD), post-traumatic stress disorder (PTSD), bipolar and related disorders (including, but not limited to, bipolar I disorder, bipolar II disorder, cyclothymic disorder), obsessive-compulsive disorder (OCD), generalized anxiety disorder (GAD), social anxiety disorder, substance use disorders (including, but not limited to, alcohol use disorder, opioid use disorder, amphetamine use disorder, nicotine use disorder, smoking, and cocaine use disorder), eating disorders (including, but not limited to anorexia nervosa, bulimia nervosa, binge-eating disorder, etc.), Alzheimer's disease, cluster headache and migraine, attention deficit hyperactivity disorder (ADHD), pain and neuropathic pain, aphantasia, fluency disorders such as childhood-onset fluency disorder, major neurocognitive disorder, mild neurocognitive disorder, suicidal ideation, suicidal behavior, major depressive disorder with suicidal ideation or suicidal behavior, melancholic depression, atypical depression, dysthymia, non-suicidal self-injury disorder (NSSID), chronic fatigue syndrome, Lyme's disease, gambling disorder, paraphilic disorders (including, but not limited to, pedophilic disorder, exhibitionistic disorder, voyeuristic disorder, fetishistic disorder, sexual masochism or sadism disorder, and transvestic disorder, etc.), sexual dysfunction (e.g., low libido, hypoactive sexual desire disorder (HSDD), etc.), peripheral neuropathy, and obesity.

In some embodiments, the methods provided herein are used to treat a subject with a depressive disorder. As used herein, the terms “depressive disorder” or “depression” refers to a group of disorders characterized by low mood that can affect a person's thoughts, behavior, feelings, and sense of well-being lasting for a period of time. In some embodiments, the depressive disorder disrupts the physical and psychological functions of a person. In some embodiments, the depressive disorder causes a physical symptom such as weight loss, aches or pains, headaches, cramps, or digestive problems. In some embodiments, the depressive disorder causes a psychological symptom such as persistent sadness, anxiety, feelings of hopelessness and irritability, feelings of guilt, worthlessness, or helplessness, loss of interest or pleasure in hobbies and activities, difficulty concentrating, remembering, or making decisions. In some embodiments, the depressive disorder is major depressive disorder (MDD), atypical depression, bipolar disorder, catatonic depression, depressive disorder due to a medical condition, postpartum depression, premenstrual dysphoric disorder, seasonal affective disorder, or treatment-resistant depression (TRD). In some embodiments, the methods provided herein are used to treat a subject with a depressive disorder who is at least 18 years of age (e.g., 18 to 65 years of age). In some embodiments, the methods provided herein are used to treat a subject with a depressive disorder who is at least 21 years of age.

In some embodiments, the disease or disorder is major depressive disorder (MDD). As used herein, the term “major depressive disorder” refers to a condition characterized by a time period of low mood that is present across most situations. Major depressive disorder is often accompanied by low self-esteem, loss of interest in normally enjoyable activities, low energy, and pain without a clear cause. In some instances, major depressive order is characterized by symptoms of depression lasting at least two weeks. In some instances, an individual experiences periods of depression separated by years. In some instances, an individual experiences symptoms of depression that are nearly always present. Major depressive disorder can negatively affect a person's personal, work, or school life, as well as sleeping, eating habits, and general health. Approximately 2-7% of adults with major depressive disorder commit suicide, and up to 60% of people who commit suicide had major depressive disorder or another related mood disorder. Dysthymia is a subtype of major depressive disorder consisting of the same cognitive and physical problems as major depressive disorder with less severe but longer-lasting symptoms. Exemplary symptoms of a major depressive disorder include, but are not limited to, feelings of sadness, tearfulness, emptiness or hopelessness, angry outbursts, irritability or frustration, even over small matters, loss of interest or pleasure in most or all normal activities, sleep disturbances, including insomnia or sleeping too much, tiredness and lack of energy, reduced appetite, weight loss or gain, anxiety, agitation or restlessness, slowed thinking, speaking, or body movements, feelings of worthlessness or guilt, fixating on past failures or self-blame, trouble thinking, concentrating, making decisions, and remembering things, frequent thoughts of death, suicidal thoughts, suicide attempts, or suicide, and unexplained physical problems, such as back pain or headaches.

As used herein, the term “atypical depression” refers to a condition wherein an individual shows signs of mood reactivity (i.e., mood brightens in response to actual or potential positive events), significant weight gain, increase in appetite, hypersomnia, heavy, leaden feelings in arms or legs, and/or long-standing pattern of interpersonal rejection sensitivity that results in significant social or occupational impairment. Exemplary symptoms of atypical depression include, but are not limited to, daily sadness or depressed mood, loss of enjoyment in things that were once pleasurable, major changes in weight (gain or loss) or appetite, insomnia or excessive sleep almost every day, a state of physical restlessness or being rundown that is noticeable by others, daily fatigue or loss of energy, feelings of hopelessness, worthlessness, or excessive guilt almost every day, problems with concentration or making decisions almost every day, recurring thoughts of death or suicide, suicide plan, or suicide attempt.

As used herein, the term “bipolar disorder” refers to a condition that causes an individual to experience unusual shifts in mood, energy, activity levels, and the ability to carry out day-to day tasks. Individuals with bipolar disorder experience periods of unusually intense emotion, changes in sleep patterns and activity levels, and unusual behaviors. These distinct periods are called “mood episodes.” Mood episodes are drastically different from the moods and behaviors that are typical for the person. Exemplary symptoms of mania, excessive behavior, include, but are not limited to, abnormally upbeat, jumpy, or wired behavior; increased activity, energy, or agitation, exaggerated sense of well-being and self-confidence, decreased need for sleep, unusual talkativeness, racing thoughts, distractibility, and poor decision-making—for example, going on buying sprees, taking sexual risks, or making foolish investments. Exemplary symptoms of depressive episodes or low mood, include, but are not limited to, depressed mood, such as feelings of sadness, emptiness, hopelessness, or tearfulness; marked loss of interest or feeling no pleasure in all-or almost all-activities, significant weight loss, weight gain, or decrease or increase in appetite, insomnia or hypersomnia (excessive sleeping or excessive sleepiness), restlessness or slowed behavior, fatigue or loss of energy, feelings of worthlessness or excessive or inappropriate guilt, decreased ability to think or concentrate, or indecisiveness, and thinking about, planning or attempting suicide. Bipolar disorder includes bipolar I disorder, bipolar II disorder, and cyclothymic disorder. Bipolar I disorder is defined by manic episodes that last at least 7 days or by severe manic symptoms that require hospitalization. A subject with bipolar I disorder may also experience depressive episodes typically lasting at least 2 weeks. Episodes of depression with mixed features, i.e., depressive and manic symptoms at the same time, are also possible. Bipolar II disorder is characterized by a pattern of depressive and hypomanic episodes, but not severe manic episodes typical of bipolar I disorder. Cyclothymic disorder (also referred to as cyclothymia) is characterized by periods of hypomanic symptoms (elevated mood and euphoria) and depressive symptoms lasting over a period of at least 2 years. The mood fluctuations are not sufficient in number, severity, or duration to meet the full criteria for a hypomanic or depressive episode.

As used herein, the term “catatonic depression” refers to a condition causing an individual to remain speechless and motionless for an extended period. Exemplary symptoms of catatonic depression include, but are not limited to, feelings of sadness, which can occur daily, a loss of interest in most activities, sudden weight gain or loss, a change in appetite, trouble falling asleep, trouble getting out of bed, feelings of restlessness, irritability, feelings of worthlessness, feelings of guilt, fatigue, difficulty concentrating, difficulty thinking, difficulty making decisions, thoughts of suicide or death, and/or a suicide attempt.

As used herein, the term “depressive disorder due to a medical condition” refers to a condition wherein an individual experiences depressive symptoms caused by another illness. Examples of medical conditions known to cause a depressive disorder include, but are not limited to, HIV/AIDS, diabetes, arthritis, strokes, brain disorders such as Parkinson's disease, Huntington's disease, multiple sclerosis, and Alzheimer's disease, metabolic conditions (e.g. vitamin B12 deficiency), autoimmune conditions (e.g., lupus and rheumatoid arthritis), viral or other infections (hepatitis, mononucleosis, herpes), back pain, and cancer (e.g., pancreatic cancer).

As used herein, the term “postpartum depression” refers to a condition as the result of childbirth and hormonal changes, psychological adjustment to parenthood, and/or fatigue. Postpartum depression is often associated with women, but men can also suffer from postpartum depression as well. Exemplary symptoms of postpartum depression include, but are not limited to, feelings of sadness, hopeless, emptiness, or overwhelmed; crying more often than usual or for no apparent reason; worrying or feeling overly anxious; feeling moody, irritable, or restless; oversleeping, or being unable to sleep even when the baby is asleep; having trouble concentrating, remembering details, and making decisions; experiencing anger or rage; losing interest in activities that are usually enjoyable; suffering from physical aches and pains, including frequent headaches, stomach problems, and muscle pain; eating too little or too much; withdrawing from or avoiding friends and family; having trouble bonding or forming an emotional attachment with the baby; persistently doubting his or ability to care for the baby; and thinking about harming themselves or the baby.

As used herein, the term “premenstrual dysphoric disorder” refers to a condition wherein an individual expresses mood lability, irritability, dysphoria, and anxiety symptoms that occur repeatedly during the premenstrual phase of the cycle and remit around the onset of menses or shortly thereafter. Exemplary symptoms of premenstrual dysphoric disorder includes, but are not limited to, lability (e.g., mood swings), irritability or anger, depressed mood, anxiety and tension, decreased interest in usual activities, difficulty in concentration, lethargy and lack of energy, change in appetite (e.g., overeating or specific food cravings), hypersomnia or insomnia, feeling overwhelmed or out of control, physical symptoms (e.g., breast tenderness or swelling, joint or muscle pain, a sensation of ‘bloating’ and weight gain), self-deprecating thoughts, feelings of being keyed up or on edge, decreased interest in usual activities (e.g., work, school, friends, hobbies), subjective difficulty in concentration, and easy fatigability.

As used herein, the term “seasonal affective disorder” refers to a condition wherein an individual experiences mood changes based on the time of the year. In some instances, an individual experiences low mood, low energy, or other depressive symptoms during the fall and/or winter season. In some instances, an individual experiences low mood, low energy, or other depressive symptoms during the spring and/or summer season. Exemplary symptoms of seasonal affective disorder include, but are not limited to, feeling depressed most of the day or nearly every day, losing interest in activities once found enjoyable, having low energy, having problems with sleeping, experiencing changes in appetite or weight, feeling sluggish or agitated, having difficulty concentrating, feeling hopeless, worthless, or guilty, and having frequent thoughts of death or suicide.

In some embodiments, a depressive disorder comprises a medical diagnosis based on the criteria and classification from Diagnostic and Statistical Manual of Mental Disorders, 5th Ed (DSM-5). The subject may have received a diagnosis of a depressive disorder (e.g., MDD) as either a single or recurrent episode as defined by DSM-5. In some embodiments, a depressive disorder comprises a medical diagnosis based on an independent medical evaluation.

In some embodiments, the methods described herein are provided to a subject with depression that is resistant to treatment. In some embodiments, the subject has been diagnosed with treatment-resistant depression (TRD). The term “treatment-resistant depression” refers to a kind of depression that does not respond or is resistant to at least one or more treatment attempts of adequate dose and duration. In some embodiments, the subject with treatment-resistant depression has failed to respond to 1 treatment attempt, 2 treatment attempts, 3 treatment attempts, 4 treatment attempts, 5 treatment attempts, or more. In some embodiments, the subject with treatment-resistant depression has been diagnosed with major depressive disorder and has failed to respond to 3 or more treatment attempts. In some embodiments, the subject with treatment resistant depression has been diagnosed with bipolar disorder and has failed to respond to 1 treatment attempt.

In some embodiments, the methods provided herein reduce at least one sign or symptom of a depressive disorder. In some embodiments, the methods provided herein reduce at least one sign or symptom of a depressive disorder by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment. In some embodiments, the methods provided herein reduce at least one sign or symptom of MDD. In some embodiments, the methods provided herein reduce at least one sign or symptom of MDD by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the sign or symptom of depression is measured in a subject before, during, and/or after treatment with the methods described herein. In some embodiments, the sign or symptom of depression is measured according to a diary assessment, an assessment by clinician or caregiver, a clinical rating scale, an imaging test, or a blood of CSF test. In some embodiments, the sign or symptom of depression in a subject is measured using a neuropsychological assessment or clinical rating scale. In some embodiments, the neuropsychological assessment or clinical rating scale is the Hamilton Depression Rating Scale, the Clinical Global Impression (CGI) Scale, the Montgomery-Åsberg Depression Ratings Scale (MADRS), the Beck Depression Inventory (BDI), the Zung Self-Rating Depression Scale, the Raskin Depression Rating Scale, the Inventory of Depressive Symptomatology (IDS), the Quick Inventory of Depressive Symptomatology (QIDS), Young Mania Rating Scale (YMRS), the Columbia-Suicide Severity Rating Scale (C-SSRS), the Suicidal Ideation Attributes Scale (SIDAS), and/or Mini International Neuropsychiatric Interview (MINI).

In some embodiments, the sign or symptom of depression in a subject is measured using the Hamilton Depression Rating (HAM-D) scale. The HAM-D scale is a 17-item scale that measures depression severity before, during, or after treatment. The scoring is based on 17 items and generally takes 15-20 minutes to complete the interview and score the results. Eight items are scored on a 5-point scale, ranging from 0=not present to 4=severe. Nine items are scored on a 3-point scale, ranging from 0=not present to 2-severe. A score of 10-13 indicates mild depression, a score of 14-17 indicates mild to moderate depression, and a score over 17 indicates moderate to severe depression. In some embodiments, after treatment with the methods described herein, the subject's HAM-D score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to treatment.

In some embodiments, the sign or symptom of depression in a subject is measured using a Clinical Global Impression (CGI) scale, such as the Clinical Global Impression improvement (CGI-I) scale. The CGI scale is a 3-item scale that measures illness severity, global improvement or change, and therapeutic response. The CGI is rated on a 7-point scale, with the severity of illness measured using a range of responses from 1 (normal) through 7 (amongst the most severely ill subjects). In some embodiments, the subject to be treated has, prior to beginning treatment with the methods herein, received a CGI score of ≥4. In some embodiments, after treatment with the methods described herein, the subject's CGI score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to treatment.

In some embodiments, the sign or symptom of depression in a subject is measured using the Montgomery-Åsberg Depression Ratings Scale (MADRS). The MADRS scale is a 10-item scale that measures the core symptoms of depression. Nine of the items are based upon patient report, and 1 item is on the rater's observation during the rating interview. MADRS items are rated on a 0-6 continuum with 0=no abnormality and 6=severe abnormality. These individual item scores are added together to form a total score, which can range between 0 and 60 points. A score of 7 to 19 indicates mild depression, 20 to 34 indicates moderate depression, and over 34 indicates severe depression. In some embodiments, the subject to be treated has, prior to beginning treatment with the methods herein, received a mild diagnosis of depression according to the MADRS. In some embodiments, the subject to be treated has scored at least 7, at least 8, at least 9, at least 10, at least 11, at least 12, at least 13, at least 14, and up to 19, up to 18, up to 17, up to 16, up to 15 on the MADRS prior to treatment with the methods herein. In some embodiments, the subject to be treated has, prior to beginning treatment with the methods herein, received a moderate to severe diagnosis of depression according to the MADRS. In some embodiments, the subject to be treated has, prior to beginning treatment with the methods herein, received a moderate to severe diagnosis of MDD as defined by the DSM, 5th edition (DSM-5). In some embodiments, the subject to be treated has scored at least 20, at least 21, at least 22, at least 23, at least 24, at least 25, at least 26, at least 27, at least 28, at least 29, at least 30, and up to 60, up to 55, up to 50, up to 45, up to 40, up to 35 on the MADRS prior to treatment with the methods herein, for example a MADRS score of 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or any range therebetween. In some embodiments, the subject to be treated has scored ≥18 on the MADRS prior to treatment with the methods herein. In some embodiments, the subject to be treated has scored ≥21 on the MADRS prior to treatment with the methods herein. In some embodiments, the subject to be treated has scored ≥24 on the MADRS prior to treatment with the methods herein. The subject to be treated with the method of the present disclosure may have the above MADRS scores even while taking a stable dose of antidepressant medication.

In evaluating treatment efficacy, a clinically meaningful “within-group” change from baseline (“CFB”) on the MADRS has been reported to range between a 6- to 9-point reduction in total score (i.e., a MADRS CFB of −6 to −9). As depression is more severe at baseline, the change needed to be considered meaningful moves towards the higher end of the range. In some embodiments, after treatment with the methods of the present disclosure the patient has a lower MADRS score compared with the subject's MADRS score prior to treatment (baseline), i.e., the methods result in a negative MADRS Change From Baseline (“CFB”) score. In some embodiments, the methods described herein result in a MADRS CFB score, e.g., at three-weeks postdose, of −10 points, −11 points, −12 points, −13 points, −14 points, −15 points, −16 points, −17 points, −18 points, −19 points, −20 points, −21 points, −22 points, −23 points, −24 points, −25 points, −26 points, −27 points, −28 points, −29 points, −30 points, or more, or any range therebetween. For example, in some embodiments, the methods described herein result in a MADRS CFB score, e.g., at three-weeks postdose, of −10 to −20 points, −11 to −19 points, −12 to −18 points, or −13 to −17 points.

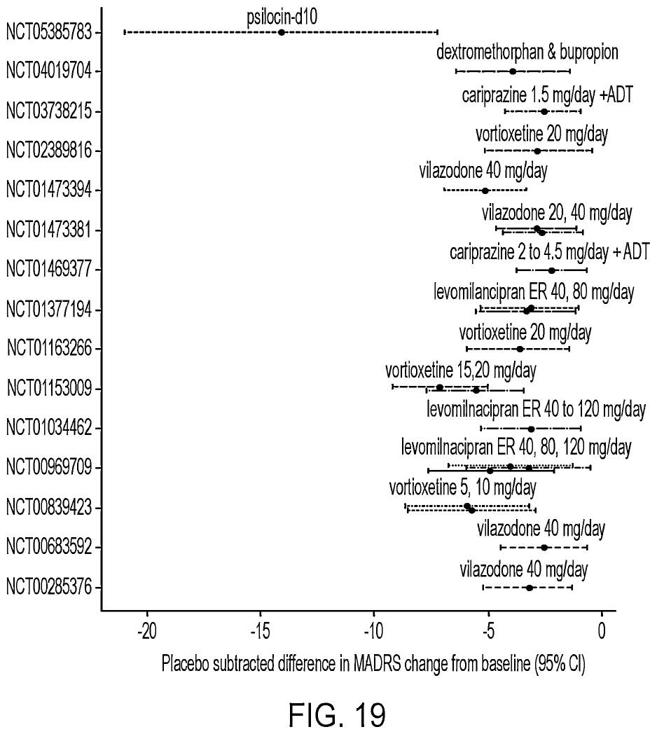

When groups are compared to each other (“between-group”), e.g., treatment versus placebo, a 2-point difference in MDRS CFB between groups has been found to be clinically meaningful. In some embodiments, the methods described herein result in a MADRS CFB score reduction over placebo (also referred to as a placebo subtracted difference in MADRS CFB), e.g., at three-weeks postdose, of −8 points, −9 points, 10 points, −11 points, −12 points, −13 points, −14 points, −15 points, −16 points, −17 points, −18 points, −19 points, −20 points, −21 points, −22 points, −23 points, −24 points, −25 points, −26 points, −27 points, −28 points, −29 points, −30 points, −32 points, or more, or any range therebetween. For example, in some embodiments, the methods described herein result in a MADRS CFB score reduction over placebo, e.g., at three-weeks postdose, of −10 to −20 points, −12 to −18 points, −13 to 16 points, or −14 to −15 points. In some embodiments, the MADRS CFB score reduction over placebo described above may be accompanied by a p-value of 0.05 or less, 0.01 or less, 0.005 or less, 0.001 or less, 0.0005 or less, or 0.0001 or less. The between group difference between treatment with the methods of the present disclosure and placebo described above is unexpectedly large and compares highly favorably to placebo subtracted differences in total MADRS CFB scores from clinical trials of 4 representative antidepressant medications approved within the last 12 years for the treatment of MDD: vortioxetine (−2.8 to −5.9), levomilnacipram (−3.1 to −4.9), vilazodone hydrochloride (−2.5 to −5.1), and combination dextromethorphan hydrobromide and bupropion hydrochloride (−3.9); as well as to the placebo subtracted differences observed in clinical trials of cariprazine (−2.2 to −2.5) for use as adjunctive therapy to antidepressant medications for the treatment of MDD (see FIG. 19 and related citations in the Examples section). By further way of comparison, ongoing clinical trials for depression with classic psychedelics report a MADRS CFB reduction for 25 mg psilocybin over 1 mg psilocybin (control) of −6.6 at three-weeks postdose (Goodwin, G M. et al. Single-Dose Psilocybin for a Treatment-Resistant Episode of Major Depression. N. Engl. J. Med. 2022; 387:1637-1648) and a MADRS CFB reduction for intravenous N,N-dimethyltryptamine (DMT) over placebo of −7.4 at two-weeks postdose (Small Pharma Inc. “Small Pharma Reports Positive Top-line Results From Phase IIa Trial of SPL026 In Major Depressive Disorder.” Small Pharma Press Release, 25 Jan. 2023).

The between-group difference in MADRS CFB score described above can be converted into an effect size (Cohen's d score) in order to inform about the magnitude of treatment effects. In some embodiments, the methods described herein provide an effect size of about 0.9, about 1, about 1.1, about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, or any range therebetween, such as from 1 to 3, 1.5 to 2.5, 1.8 to 2.4, or 2 to 2.2. In some embodiments, the methods described herein provide an effect size of 1.7 to 2.5, or 1.8 to 2.3, or 2 to 2.2. Such effect sizes are considered to be surprisingly large, especially in MDD treatment, where effect sizes typically range from 0.2 to 0.6.

In some embodiments, after treatment with the methods described herein, the subject's MADRS score decreases from prior to treatment (baseline) by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or any range therebetween, e.g., at three-weeks postdose. In some embodiments, after treatment with the methods described herein, the subject's MADRS score decreases from prior to treatment (baseline) by at least about 50%, at least about 55%, at least about 60%, at least about 65%, at least about 70%, at least about 75%, at least about 80%, at least about 85%, at least about 90%, at least about 95%, or about 100%, or any range therebetween, e.g., at three-weeks postdose. In some embodiments, at least 50%, at least 55%, at least 60%, at least 65%, at least 70%, at least 75%, at least 80%, at least 85%, at least 90% of subjects treated with the methods described herein have a MADRS score decrease from baseline by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, e.g., at three-weeks postdose.

In some embodiments, the methods described herein provide a response rate (i.e., number of “responders” versus total number of subjects treated within a group) of at least 25%, at least 30%, at least 35%, at least 40%, at least 45%, at least 50%, at least 55%, at least 60%, at least 65%, at least 70%, at least 75%, at least 80%, at least 85%, at least 90%, e.g., measured three-weeks post dose, with “responders” being defined as subjects who had a reduction of MADRS score from baseline of at least 50%. In some embodiments, the methods described herein involve administration of multiple doses (e.g., two doses) of a compound of Formula (I-3), or a pharmaceutically acceptable salt, polymorph, or solvate thereof, each dose being about 8-16 mg, about 8-14 mg, about 8-12 mg, about 12-16 mg, or about 12 mg, or about 16 mg of the compound of Formula (I-3), or a pharmaceutically acceptable salt, polymorph, or solvate thereof administered to the subject one week apart, two weeks apart, three weeks apart, or four weeks apart (±3 days), in a treatment course, wherein the method provides a response rate of at least 35%, at least 40%, at least 45%, at least 50%, at least 55%, at least 60%, at least 65%, at least 70%, at least 75%, at least 80%, at least 85%, at least 90%, e.g., as measured three-weeks post dose of the final dose (e.g., dose two of a two dose regimen).

In some embodiments, the methods described herein provide a remission rate (i.e., number of “remitters” versus total number of subjects treated within a group) of at least 10%, at least 12%, at least 14%, at least 16%, at least 18%, at least 20%, at least 22%, at least 24%, at least 26%, at least 28%, at least 30%, at least 35%, at least 40%, at least 45%, at least 50%, e.g., measured three weeks postdose, with “remitters” being defined as subjects who had a total MADRS score of 10 or less, 9 or less, 8 or less, 7 or less, 6 or less, 5 or less, 4 or less, 3 or less, 2 or less, 1 or less. In some embodiments, the methods described herein involve administration of multiple doses (e.g., two doses) of a compound of Formula (I-3), or a pharmaceutically acceptable salt, polymorph, or solvate thereof, each dose being about 8-16 mg, about 8-14 mg, about 8-12 mg, about 12-16 mg, or about 12 mg, or about 16 mg of the compound of Formula (I-3), or a pharmaceutically acceptable salt, polymorph, or solvate thereof administered to the subject one week apart, two weeks apart, three weeks apart, or four weeks apart (±3 days), in a treatment course, wherein the method provides a remission rate of at least 20%, at least 22%, at least 24%, at least 26%, at least 28%, at least 30%, at least 35%, at least 40%, at least 45%, at least 50%, at least 55%, at least 60%, at least 65%, at least 70%, at least 75%, at least 80%, at least 85%, at least 90%, e.g., as measured three-weeks post dose of the final dose (e.g., dose two of a two dose regimen).

In some embodiments, the subject has a durable and sustained response to treatment with the methods of the present disclosure, whereby the therapeutic effect following treatment (following one dose in a single dose regiment or the last dose in a multiple dose regimen) is maintained for at least four-weeks, at least five-weeks, at least six-weeks, at least seven-weeks, at least eight-weeks, at least nine-weeks, at least ten-weeks, at least eleven-weeks, at least twelve-weeks, at least thirteen-weeks, at least fourteen-weeks, at least fifteen-weeks, at least sixteen-weeks, at least seventeen-weeks, at least eighteen-weeks, at least nineteen-weeks, at least twenty-weeks, at least twenty one-weeks, at least twenty two-weeks, at least twenty three-weeks, at least twenty four-weeks, at least twenty five-weeks, at least twenty six-weeks, or longer such as for one year or longer following treatment, or any range therebetween. The therapeutic effect following treatment can be measured by a subject's sign or symptoms, such as according to a diary assessment, an assessment by clinician or caregiver, a clinical rating scale, an imaging test, or a blood of CSF test. For example, a durable and sustained response following treatment can be measured by a reduction from baseline in the MADRS, including whether the subject(s) maintains their classification for response and/or remission, for a time period set forth above.

In some embodiments, the sign or symptom of depression in a subject is measured using the Beck Depression Inventory (BDI). The BDI is a 21-item, self-report rating inventory that measures characteristic attitudes and symptoms of depression. The items are rated on 0-3 continuum with 0=no abnormality and 3-severe abnormality. A score of 17-20 indicates borderline clinical depression, a score of 21-30 indicates moderate depression, a score of 31-40 indicates severe depression, and over 40 indicates extreme depression. In some embodiments, after treatment with the methods of the present disclosure the patient has a lower BDI score compared with the subject's BDI score prior to treatment (baseline), i.e., the methods result in a negative BDI Change From Baseline (“CFB”) score. In some embodiments, the methods described herein result in a BDI CFB score, e.g., at three-weeks postdose, of −10 points, −11 points, −12 points, −13 points, −14 points, −15 points, −16 points, −17 points, −18 points, −19 points, −20 points, −21 points, −22 points, −23 points, −24 points, −25 points, −26 points, −27 points, −28 points, −29 points, −30 points, or more, or any range therebetween. For example, in some embodiments, the methods described herein result in a BDI CFB score, e.g., at three-weeks postdose, of −10 to −20 points, −11 to −19 points, −12 to −18 points, or −13 to −17 points. In some embodiments, the methods described herein result in a BDI CFB score reduction over placebo, e.g., at three-weeks postdose, of −8 points, −9 points, 10 points, −11 points, −12 points, −13 points, −14 points, −15 points, −16 points, −17 points, −18 points, −19 points, −20 points, −21 points, −22 points, −23 points, −24 points, −25 points, −26 points, −27 points, −28 points, −29 points, −30 points, or more, or any range therebetween. For example, in some embodiments, the methods described herein result in a BDI

CFB score reduction over placebo, e.g., at three-weeks postdose, of −10 to −20 points, −12 to −18 points, −13 to 16 points, or −14 to −15 points. In some embodiments, the BDI CFB score reduction over placebo described above may be accompanied by a p-value of 0.05 or less, 0.01 or less, 0.005 or less, 0.001 or less, 0.0005 or less, or 0.0001 or less. In some embodiments, after treatment with the methods described herein, the subject's BDI score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to treatment.

In some embodiments, the sign or symptom of depression in a subject is measured using the Zung Self-Rating Depression Scale. The Zung Self-Rating Depression Scale is a 20-item self-report questionnaire that measures the psychological and somatic symptoms associated with depression. The questionnaire takes about 10 minutes to complete, and items are framed in terms of positive and negative statements. Each item is scored on a Likert scale ranging from 1 to 4. A total score is derived by summing the individual item scores, and ranges from 20 to 80. Most people with depression score between 50 and 69, while a score of 70 and above indicates severe depression. In some embodiments, after treatment with the methods described herein, the subject's Zung Self-Rating Depression score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to treatment.

In some embodiments, the sign or symptom of depression in a subject is measured using the Raskin Depression Rating Scale. The Raskin Depression Rating Scale measures baseline levels of depression and change in depression severity overtime. Each item is scored on a scale ranging from 1 to 5 with 1=not at all and 5=very much. A total score is derived by summing the individual item scores, and scores of 9 or greater represents moderate depression. In some embodiments, after treatment with the methods described herein, the subject's Raskin Depression Rating Scale score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to treatment.

In some embodiments, the sign or symptom of depression in a subject is measured using the Inventory of Depressive Symptomatology (IDS). The IDS is a 30-item inventory that measures depressive signs and symptoms. Each item is scored on a scale of 0 to 3 with 0-absence of pathology and 3=severe pathology. A total score is derived by summing the individual item scores; scores between 26-38 indicates mild depression, scores between 39-48 indicate moderate depression, and scores 49 or greater indicate severe depression. In some embodiments, after treatment with the methods described herein, the subject's IDS score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to treatment.

In some embodiments, the sign or symptom of depression in a subject is measured using the Quick Inventory of Depressive Symptomatology (QIDS). The QIDS is a 16-item inventory that measures depressive signs and symptoms. Each item is scored on a scale of 0 to 3 with 0=absence of pathology and 3=severe pathology. A total score is derived by summing the individual item scores; scores between 11-15 indicate moderate depression, scores between 16-20 indicate severe depression, and scores 21 or greater indicate very severe depression. In some embodiments, after treatment with the methods described herein, the subject's QIDS score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to treatment.

In some embodiments, the sign or symptom of depression in a subject is measured using the Young Mania Rating Scale (YMRS). The YMRS is an 11-item inventory that measures manic signs and symptoms. There are 4 items that are graded on a 0 to 8 scale, ranging from 0=absent to 8=severe. The remaining 7 items are graded on a 0 to 4 scale, ranging from 0=absent to 4=severe. A total score is derived by summing the individual item scores; scores between 9-15 indicate mild mania, scores between 16-25 indicate moderate mania, and scores 26 or greater indicate severe mania. In some embodiments, after treatment with the methods described herein, the subject's YMRS score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to treatment.

In some embodiments, the sign or symptom of depression in a subject is measured using the Columbia-Suicide Severity Rating Scale (C-SSRS). The C-SSRS measures the severity of suicidal ideation and behavior. The scale contains 10 binary questions (no=0 points and yes=1 point) and each question addresses a different component of the subject's suicidal ideation severity and behavior. A subject is considered to have suicidal ideation and/or behavior if they answer “yes” to any of the 10 questions. In some embodiments, after treatment with the methods described herein, the subject's C-SSRS score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to treatment.

In some embodiments, the sign or symptom of depression in a subject is measured using the Suicidal Ideation Attributes Scale (SIDAS). The SIDAS measures the presence and severity of suicidal thoughts. The scale contains 5 questions measured on a 10-point scale with 0=never and 10=always. Total scores are calculated as the sum of the 5 items and range from 0 to 50. Scores of 21 or greater indicate high risk of suicidal behavior. In some embodiments, after treatment with the methods described herein, the subject's SIDAS score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to treatment.

The Mini International Neuropsychiatric Interview (MINI) (for example, version 7.0.2) is a diagnostic interview instrument for psychiatric disorders in the Diagnostic and Statistical Manual of Mental Disorders (DSM-5) and the International Classification of Diseases-10. In some embodiments, treating according to the methods of the disclosure results in an improvement in a psychiatric disorder (e.g., depression, anxiety, etc.) compared to pre-treatment of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, according to a Mini International Neuropsychiatric Interview assessment. In some embodiments, the subject has a lower MINI 7.0.2 score compared with the subject's MINI 7.0.2 score prior to treatment.

In some embodiments, the sign or symptom in subjects with depression is measured using a Spielberger's Trait and Anxiety Inventory, a Generalized Anxiety Disorder 7-Item Scale, a Warwick-Edinburgh Mental Wellbeing Scale, a Flourishing Scale, a Snaith Hamilton Anhedonia Pleasure Scale, a Life Orientation Test, a Meaning in Life Questionnaire, a Brief Resilience Scale, a Dysfunctional Attitudes Scale, a 44-item Big Five Inventory, a Peters 21-item Delusional Inventory, an Examination of Anomalous Self-Experience, a Ruminative Responses Scale, a White Bear Suppression Inventory, a Barrett Impulsivity Scale, a Brief Experiential Avoidance Questionnaire, a Modified Tellegen Absorption Questionnaire, a Scale to Assess Therapeutic Relationship, Credibility/Expectancy Questionnaire, a Connectedness to Nature Scale, a Political Perspective Questionnaire, a Social Connectedness Scale, a Bech-Rafaelsen Mania Rating Scale, a Revised Santa Clara brief compassion scale, a Gratitude Questionnaire, a Short Suggestibility Scale, a Rosenberg Self-Esteem Scale, a Universality Subscale of the Spiritual Transcendence Scale, an Oxford Questionnaire on the Emotional Side-effects of Antidepressants, a Lauks Emotional Intensity Scale, Sexual Dysfunction Questionnaire, a Brief Index of Sexual Functioning for Women, a Sexual Perceptions Questionnaire, a Barnes Akathisia Rating Scale, a Work Productivity and Activity Impairment Questionnaire, a Work and Social Adjustment Scale, a Connectedness Questionnaire, a Standard Assessment of Personality, a Positive and Negative Syndrome Scale, a Mastery Insight Scale, a Self-Reflection and Insight Scale, a Psychological Insight Scale, a Metaphysical Beliefs Questionnaire, a Spiritual Bypassing Scale, an Adverse Childhood Experience Questionnaire, a Therapeutic Music Experience Questionnaire, a Setting Questionnaire, an Absorption in Music Scale, a Psychedelic Predictor Scale, a Surrender Scale, a EuroQOL-5 Dimension-3 Level Scale, or any combinations thereof. In some embodiments, after treatment with the methods described herein, the sign or symptom of depression measured using any of the assessments listed above decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to treatment.

In some embodiments, the sign or symptom of depression in a subject is measured using an imaging test before, during, or after treatment with the methods described herein. In some embodiments, the imaging test is a CT scan. In some embodiments, the imaging test is a functional MRI scan. In some embodiments, the functional MRI scan measures the blood oxygen level-dependent (BOLD) response as an indicator of brain activity and/or functional connectivity. In some embodiments, the BOLD response is measured in the subject at resting state, in response to emotional faces, or in response to music. In some embodiments, after treatment with the methods described herein, the BOLD response in a region of the brain increases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment. In some embodiments, after treatment with the methods described herein, the BOLD response in the amygdala increases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment. In other embodiments, after treatment with the methods described herein, the BOLD response in a region of the brain decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to treatment. In some embodiments, after treatment with the methods described herein, the BOLD response in the amygdala decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to treatment.

In some embodiments, the sign or symptom of depression is measured using a marker for depression in the blood or cerebral spinal fluid. In some embodiments, the marker for depression is measured in the subject before, during, or after treatment with the methods or compositions described herein. In some embodiments, the marker for depression is red blood cell folate, serum folate, vitamin B12, plasma homocysteine, serum methylfolate, and/or testing for one or more of brain-derived neurotrophic factor (BDNF) Val66Met, bone morphogenetic protein rs41271330, and/or 5-HTTLPR polymorphisms. In some embodiments, the marker for depression decreases by between about 5% and about 300%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300%, or more, compared to prior to treatment. In other embodiments, the marker for depression increases by between about 5% and about 300%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300%, or more, compared to prior to treatment.

In some embodiments, the disease or disorder is an anxiety disorder. As used herein, the term “anxiety disorder” refers to a state of apprehension, uncertainty, and/or fear resulting from the anticipation of an event and/or situation. Anxiety disorders cause physiological and psychological signs or symptoms. Non-limiting examples of physiological symptoms include muscle tension, heart palpitations, sweating, dizziness, shortness of breath, tachycardia, tremor, fatigue, worry, irritability, and disturbed sleep. Non-limiting examples of psychological symptoms include fear of dying, fear of embarrassment or humiliation, fear of an event occurring, etc. Anxiety disorders also impair a subject's cognition, information processing, stress levels, and immune response. In some embodiments, the methods disclosed herein treat chronic anxiety disorders. As used herein, a “chronic” anxiety disorder is recurring. Examples of anxiety disorders include, but are not limited to, generalized anxiety disorder (GAD), social anxiety disorder, panic disorder, panic attack, a phobia-related disorder (e.g., phobias related to flying, heights, specific animals such as spiders/dogs/snakes, receiving injections, blood, etc., agoraphobia), separation anxiety disorder, selective mutism, anxiety due to a medical condition, post-traumatic stress disorder (PTSD), obsessive-compulsive disorder (OCD), substance-induced anxiety disorder, etc.

In some embodiments, the subject in need thereof develops an anxiety disorder after experiencing the effects of a disease. The effects of a disease include diagnosis of an individual with said disease, diagnosis of an individual's loved ones with said disease, social isolation due to said disease, quarantine from said disease, or social distancing as a result of said disease. In some embodiments, an individual is quarantined to prevent the spread of the disease. In some embodiments, the disease is COVID-19, SARS, or MERS. In some embodiments, a subject develops an anxiety disorder after job loss, loss of housing, or fear of not finding employment.

In some embodiments, the disease or disorder is generalized anxiety disorder (GAD). Generalized anxiety disorder is characterized by excessive anxiety and worry, fatigue, restlessness, increased muscle aches or soreness, impaired concentration, irritability, and/or difficulty sleeping. In some embodiments, a subject with generalized anxiety disorder does not have associated panic attacks. In some embodiments, after treating the symptom is reduced compared to prior to treating by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some embodiments, the disease or disorder is social anxiety disorder. As used herein, “social anxiety disorder” is a marked fear or anxiety about one or more social situations in which the individual is exposed to possible scrutiny by others. Non-limiting examples of situations which induce social anxiety include social interactions (e.g., having a conversation, meeting unfamiliar people), being observed (e.g., eating or drinking), and performing in front of others (e.g., giving a speech). In some embodiments, the social anxiety disorder is restricted to speaking or performing in public. In some embodiments, treating according to the methods of the disclosure reduces or ameliorates a symptom of social anxiety disorder. In some embodiments, after treating the symptom is reduced compared to prior to treating by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some embodiments, the disease or disorder is a compulsive disorder, such as obsessive-compulsive disorder (OCD), body-focused repetitive behavior, hoarding disorder, gambling disorder, compulsive buying, compulsive internet use, compulsive video gaming, compulsive sexual behavior, compulsive eating, compulsive exercise, body dysmorphic disorder, hoarding disorder, dermatillomania, trichotillomania, excoriation, substance-induced obsessive compulsive and related disorder, or an obsessive-compulsive disorder due to another medical condition, etc., or a combination thereof. In some embodiments, the disease or disorder is obsessive-compulsive disorder (OCD).

In some embodiments, the disease or disorder is post-traumatic stress disorder (PTSD).

In some embodiments, at least one sign or symptom of an anxiety disorder is improved following the administration of a compound as disclosed herein. In some embodiments, a sign or symptom of an anxiety disorder is measured according to a diary assessment, an assessment by a clinician or caregiver, or a clinical scale. In some embodiments, treatment causes a demonstrated improvement in one or more of the following: State-Trait Anxiety Inventory (STAI), The Dutch Temperament and Character Inventory (TCI), Dutch Personality Questionnaire-2-Revised (NPV-2-R), Beck Anxiety Inventory (BAI), Hospital Anxiety and Depression Scale (HADS), Generalized Anxiety Disorder questionnaire-IV (GADQ-IV), Hamilton Anxiety Rating Scale (HARS), Leibowitz Social Anxiety Scale (LSAS), Overall Anxiety Severity and Impairment Scale (OASIS), Hospital Anxiety and Depression Scale (HADS), Patient Health Questionnaire 4 (PHQ-4), Social Phobia Inventory (SPIN), Brief Trauma Questionnaire (BTQ), Combat Exposure Scale (CES), Mississippi Scale for Combat-Related PTSD (M-PTSD), Posttraumatic Maladaptive Beliefs Scale (PMBS), Perceived Threat Scale (DRRI-2 Section: G), PTSD Symptom Scale-Interview for DSM-5 (PSS-I-5), Structured Interview for PTSD (SI-PTSD), Davidson Trauma Scale (DTS), Impact of Event Scale-Revised (IES-R), Posttraumatic Diagnostic Scale (PDS-5), Potential Stressful Events Interview (PSEI), Stressful Life Events Screening Questionnaire (SLESQ), Spielberger's Trait and Anxiety, Generalized Anxiety Dis-order 7-Item Scale, The Psychiatric Institute Trichotillomania Scale (PITS), The MGH Hairpulling Scale (MGH-HPS), The NIMH Trichotillomania Severity Scale (NIMH-TSS), The NIMH Trichotillomania Impairment Scale (NIMH-TIS), The Clinical Global Impression (CGI), the Brief Social Phobia Scale (BSPS), The Panic Attack Questionnaire (PAQ), Panic Disorder Severity Scale, Florida Obsessive-Compulsive Inventory (FOCI), The Leyton Obsessional Inventory Survey Form, The Vancouver Obsessional Compulsive Inventory (VOCI), The Schedule of Compulsions, Obsessions, and Pathological Impulses (SCOPI), Padua Inventory-Revised (PI-R), Quality of Life (QoL), The Clinical Global Improvement (CGI) scale, The Yale-Brown Obsessive-Compulsive Scale (Y-BOCS), The Yale-Brown Obsessive-Compulsive Scale Second Edition (Y-BOCS-II), The Dimensional Yale-Brown Obsessive-Compulsive Scale (DY-BOCS), The National Institute of Mental Health-Global Obsessive-Compulsive Scale (NIMH-GOCS), The Yale-Brown Obsessive-Compulsive Scale Self-Report (Y-BOCS-SR), The Obsessive-Compulsive Inventory-Re-vised (OCI-R), and the Dimensional Obsessive-Compulsive Scale (DOCS), or a combination thereof. In some embodiments, treating according to the methods of the disclosure results in an improvement in an anxiety disorder compared to pre-treatment of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, according to any one of the diary assessments, assessments by a clinical or caregiver, or clinical scales, described herein or known in the art.

In some embodiments, the Spielberger State-Trait Anxiety Inventory (STAI) is utilized to evaluate the safety and efficacy of the methods of the present disclosure for the treatment of anxiety disorders (e.g., GAD). The STAI is a widely used instrument that contains separate self-report scales for measuring “state” and “trait” anxiety. “State” anxiety is transient anxiety due to a stressful stimulus, whereas “trait” anxiety is the predisposition of a subject to react with anxiety to stressful situations. The STAI is self-reported and contains 40 items scored by a 4 point Likert scale with semantic guides taking approximately 10 minutes. The STAI contains 20 questions related to state anxiety and 20 questions related to trait anxiety. Each section is scored between 20 and 80, with higher scores correlating with greater anxiety. Low scores indicate a mild form of anxiety and high scores indicate a severe form of anxiety. Both scales have anxiety absent and anxiety present questions. Anxiety absent questions represent the absence of anxiety in a statement like, “I feel secure.” Anxiety present questions represent the presence of anxiety in a statement like “I feel worried.” In some embodiments, after treatment with the methods of the present disclosure the patient has a lower STAI score compared with the subject's STAI score prior to treatment (baseline), i.e., the methods result in a negative STAI Change From Baseline (“CFB”) score. In some embodiments, the methods described herein result in a STAI CFB score, e.g., at three-weeks postdose, of −10 points, −11 points, −12 points, −13 points, −14 points, −15 points, −16 points, −17 points, −18 points, −19 points, −20 points, −21 points, −22 points, −23 points, −24 points, −25 points, −26 points, −27 points, −28 points, −29 points, −30 points, or more, or any range therebetween. For example, in some embodiments, the methods described herein result in a STAI CFB score, e.g., at three-weeks postdose, of −10 to −20 points, −11 to −19 points, −12 to −18 points, or −13 to −17 points. In some embodiments, the methods described herein result in a STAI CFB score reduction over placebo, e.g., at three-weeks postdose, of −8 points, −9 points, 10 points, −11 points, −12 points, −13 points, −14 points, −15 points, −16 points, −17 points, −18 points, −19 points, −20 points, −21 points, −22 points, −23 points, −24 points, −25 points, −26 points, −27 points, −28 points, −29 points, −30 points, or more, or any range therebetween. For example, in some embodiments, the methods described herein result in a STAI CFB score reduction over placebo, e.g., at three-weeks postdose, of −10 to −20 points, −12 to −18 points, −13 to 16 points, or −14 to −15 points. In some embodiments, the STAI CFB score reduction over placebo described above may be accompanied by a p-value of 0.05 or less, 0.01 or less, 0.005 or less, 0.001 or less, 0.0005 or less, or 0.0001 or less. In some embodiments, after treatment with the methods described herein, the subject's STAI score decreases from prior to treatment (baseline) by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. In some embodiments, at least 50%, at least 55%, at least 60%, at least 65%, at least 70%, at least 75%, at least 80%, at least 85%, at least 90% of subjects treated with the methods described herein have a STAI score decrease from baseline by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some embodiments, the Dutch Temperament and Character Inventory (TCI) is utilized to evaluate the safety and efficacy of the methods of the present disclosure for the treatment of anxiety disorders (e.g., GAD). The TCI involves 240 bi-directional true-false alternatives, allowing a detailed assessment of personality traits, with a good internal consistency. Temperament is characterized on four dimensions: novelty seeking, harm avoidance, reward dependence, and persistence; character on three dimensions: self-directedness, cooperativeness, and self-transcendence. The four temperament dimensions are assumed to be underlined by specific neurotransmission systems.

In some embodiments, the Dutch Personality Questionnaire-2-Revised (NPV-2-R) is utilized to evaluate the safety and efficacy of the methods of the present disclosure for the treatment of anxiety disorders (e.g., GAD). The NPV-2-R is the most frequently used (clinical) personality questionnaire in The Netherlands. The NPV-2-R has satisfactory reliability, validity and internal consistencies, and includes 140 items. All items are answered on a bi-directional three-point scale (true-?-false), equally divided into seven scales of personality aspects: neuroticism (inadequatie), social anxiety (sociale inadequatie), rigidity (rigiditeit), hostility (verongelijktheid), egoism (zelfgenoegzaamheid), dominance (dominantie), and self-esteem (zelfwaardering). The NPV-2-R also has subscales; neuroticism has the subscales: depression and anxiety (depressive en angst), social anxiety has the subscales shyness and social avoidance (verlegenheid en sociale vermijding), rigidity has subscales for neatness and inflexibility (ordelijkheid en inflexibiliteit) and dominance has subscales for leadership and independence (leidinggeven en autonomie).

In some embodiments, the disease or disorder is a headache disorder. As used herein, the term “headache disorder” refers to a disorder characterized by recurrent headaches. Headache disorders include migraine, tension-type headache, cluster headache, and chronic daily headache syndrome.

In some embodiments, a method of treating cluster headaches in a subject in need thereof is disclosed herein. In some embodiments, at least one sign or symptom of cluster headache is improved following treatment. In some embodiments, the sign or symptom of cluster headache is measured according to a diary assessment, a physical or psychological assessment by clinician, an imaging test, or a neurological examination. Cluster headache is a primary headache disorder and belongs to the trigeminal autonomic cephalalgias. The definition of cluster headaches is a unilateral headache with at least one autonomic symptom ipsilateral to the headache. Attacks are characterized by severe unilateral pain predominantly in the first division of the trigeminal nerve—the fifth cranial nerve whose primary function is to provide sensory and motor innervation to the face. Attacks are also associated with prominent unilateral cranial autonomic symptoms and subjects often experience agitation and restlessness during attacks. In some embodiments, a subject with cluster headaches also experiences nausea and/or vomiting. In some embodiments, a subject with cluster headaches experiences unilateral pain, excessive tearing, facial flushing, a droopy eyelid, a constricted pupil, eye redness, swelling under or around one or both eyes, sensitivity to light, nausea, agitation, and restlessness.

In some embodiments, a method of treating migraines in a subject in need thereof is disclosed herein. A migraine is a moderate to severe headache that affects one half or both sides of the head, is pulsating in nature, and last from 2 to 72 hours. Symptoms of migraine include headache, nausea, sensitivity to light, sensitivity to sound, sensitivity to smell, dizziness, difficulty speaking, vertigo, vomiting, seizure, distorted vision, fatigue, or loss of appetite. Some subjects also experience a prodromal phase, occurring hours or days before the headache, and/or a postdromal phase following headache resolution. Prodromal and postdromal symptoms include hyperactivity, hypoactivity, depression, cravings for particular foods, repetitive yawning, fatigue and neck stiffness and/or pain. In some embodiments, the migraine is a migraine without aura, a migraine with aura, a chronic migraine, an abdominal migraine, a basilar migraine, a menstrual migraine, an ophthalmoplegic migraine, an ocular migraine, an ophthalmic migraine, or a hemiplegic migraine. In some embodiments, the migraine is a migraine without aura. A migraine without aura involves a migraine headache that is not accompanied by a headache. In some embodiments, the migraine is a migraine with aura. A migraine with aura is primarily characterized by the transient focal neurological symptoms that usually precede or sometimes accompany the headache. Less commonly, an aura can occur without a headache, or with a non-migraine headache. In some embodiments, the migraine is a hemiplegic migraine. A hemiplegic migraine is a migraine with aura and accompanying motor weakness. In some embodiments, the hemiplegic migraine is a familial hemiplegic migraine or a sporadic hemiplegic migraine. In some embodiments, the migraine is a basilar migraine. A subject with a basilar migraine has a migraine headache and an aura accompanied by difficulty speaking, world spinning, ringing in ears, or a number of other brainstem-related symptoms, not including motor weakness. In some embodiments, the migraine is a menstrual migraine. A menstrual migraine occurs just before and during menstruation. In some embodiments, the subject has an abdominal migraine. Abdominal migraines are often experienced by children. Abdominal migraines are not headaches, but instead stomach aches. In some embodiments, a subject with abdominal migraines develops migraine headaches. In some embodiments, the subject has an ophthalmic migraine also called an “ocular migraine.” Subjects with ocular migraines experience vision or blindness in one eye for a short time with or after a migraine headache. In some embodiments, a subject has an ophthalmoplegic migraine. Ophthalmoplegic migraines are recurrent attacks of migraine headaches associated with paresis of one or more ocular cranial nerves. In some embodiments, the subject in need of treatment experiences chronic migraines. As defined herein, a subject with chronic migraines has more than fifteen headache days per month. In some embodiments, the subject in need of treatment experiences episodic migraines. As defined herein, a subject with episodic migraines has less than fifteen headache days per month.

In some embodiments, a method of treating chronic daily headache syndrome (CDHS) in a subject in need thereof is disclosed herein. A subject with CDHS has a headache for more than four hours on more than 15 days per month. Some subjects experience these headaches for a period of six months or longer. CHDS affects 4% of the general population. Chronic migraine, chronic tension-type headaches, new daily persistent headache, and medication overuse headaches account for the vast majority of chronic daily headaches.

In some embodiments, after treating according to the methods of the disclosure, the frequency of headaches and/or related symptoms decreases by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to said treating.

In some embodiments, after treating according to the methods of the disclosure, the length of a headache attack decreases by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to said treating.

In some embodiments, at least one sign or symptom of headache disorder is improved following administration of a compound disclosed herein. In some embodiments, a sign or symptom of a headache disorder is measured according to a diary assessment, an assessment by a clinician or caregiver, or a clinical scale. In some embodiments, treatment of the present disclosure causes a demonstrated improvement in one or more of the following: the Visual Analog Scale, Numeric Rating Scale, the Short Form Health Survey, Profile of Mood States, the Pittsburgh Sleep Quality Index, the Major Depression Inventory, the Perceived Stress Scale, the 5-Level EuroQOL-5D, the Headache Impact Test; the ID-migraine; the 3-item screener; the Minnesota Multiphasic Personality Inventory; the Hospital Anxiety and Depression Scale (HADS), the 50 Beck Depression Inventory (BDI; both the original BD151 and the second edition, BDI-1152), the 9-item Patient Health Questionnaire (PHQ-9), the Migraine Disability Assessment Questionnaire (MI-DAS), the Migraine-Specific Quality of Life Questionnaire version 2.1 (MSQ v2.1), the European Quality of Life-5 Dimensions (EQ-5D), the Short-form 36 (SF-36), or a combination thereof. In some embodiments, treating according to the methods of the disclosure results in an improvement in a headache disorder compared to pre-treatment of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, according to any one of the diary assessments, assessments by a clinical or caregiver, or clinical scales, described herein or known in the art. In some embodiments, the sign or symptom of the headache disorder is measured according to a diary assessment, a physical or psychological assessment by clinician, an imaging test, an electroencephalogram, a blood test, a neurological examination, or combination thereof. In some embodiments, the blood test evaluates blood chemistry and/or vitamins.

In some embodiments, the disease or disorder is a substance use disorder. Substance addictions which can be treated using the methods herein include addictions to addictive substances/agents such as recreational drugs and addictive medications. Examples of addictive substances/agents include, but are not limited to, alcohol, e.g., ethyl alcohol, gamma hydroxybutyrate (GHB), caffeine, nicotine, cannabis (marijuana) and cannabis derivatives, opiates and other morphine-like opioid agonists such as heroin, phencyclidine and phencyclidine-like compounds, sedative hypnotics such as benzodiazepines, methaqualone, mecloqualone, etaqualone and barbiturates and psychostimulants such as cocaine, amphetamines and amphetamine-related drugs such as dextroamphetamine and methylamphetamine. Examples of addictive medications include, e.g., benzodiazepines, barbiturates, and pain medications including alfentanil, allylprodine, alphaprodine, anileridine benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene fentanyl, heroin, hydrocodone, hydromorphone, hydroxypethidine, isomethadone, ketobemidone, levallorphan, levorphanol, levophenacylmorphan, lofenitanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, OXYCONTIN®, oxymorphone, papaveretum, pentazocine, phenadoxone, phenomorphan, phenazocine, phenoperidine, piminodine, piritramide, propheptazine, promedol, properidine, propiram, propoxyphene sufentanil, tramadol, and tilidine. In some embodiments, the disease or disorder is alcohol use disorder. In some embodiments, the disease or disorder is nicotine use (e.g., smoking) disorder, and the therapy is used for e.g., smoking cessation.

In some embodiments, the disease or disorder is an eating disorder. As used herein, the term “eating disorder” refers to any of a range of psychological disorders characterized by abnormal or disturbed eating habits. Non-limiting examples of eating disorders include pica, anorexia nervosa, bulimia nervosa, rumination disorder, avoidant/restrictive food intake disorder, binge-eating disorder, other specified feeding or eating disorder, unspecified feeding or eating disorder, or combinations thereof. In some embodiments, the eating disorder is pica, anorexia nervosa, bulimia nervosa, rumination disorder, avoidant/restrictive food intake disorder, binge-eating disorder, or combinations thereof. In some embodiments, the methods disclosed herein treat chronic eating disorders. As used herein, a “chronic” eating disorder is recurring. In some embodiments, at least one sign or symptom of an eating disorder is improved following administration of a compound disclosed herein. In some embodiments, a sign or symptom of an eating disorder is measured according to a diary assessment, an assessment by a clinician or caregiver, or a clinical scale. Non-limiting examples of clinical scales, diary assessments, and assessments by a clinician or caregiver include: the Mini International Neuropsychiatric Interview (MINI), the McLean Screening Instrument for Borderline Personality Disorder (MSI-BPD), the Eating Disorder Examination (EDE), the Eating Disorder Questionnaire (EDE-Q), the Eating Disorder Examination Questionnaire Short Form (EDE-QS), the Physical Appearance State and Trait Anxiety Scale-State and Trait version (PASTAS), Spielberger State-Trait Anxiety Inventory (STAI), Eating Disorder Readiness Ruler (ED-RR), Visual Analogue Rating Scales (VAS), the Montgomery-Åsberg Depression Ratings Scale (MADRS), Yale-Brown Cornell Eating Disorder Scale (YBC-EDS), Yale-Brown Cornell Eating Disorder Scale Self Report (YBC-EDS-SRQ), the Body Image State Scale (BISS), Clinical impairment assessment (CIA) questionnaire, the Eating Disorder Inventory (EDI) (e.g. version 3: EDI-3), the Five Dimension Altered States of Consciousness Questionnaire (5D-ASC), the Columbia-Suicide Severity Rating Scale (C-SSRS), the Life Changes Inventory (LCI), and combinations thereof. In some embodiments, treating according to the methods of the disclosure results in an improvement in an eating disorder compared to pre-treatment of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, according to any one of the diary assessments, assessments by a clinical or caregiver, or clinical scales, described herein or known in the art.

In some embodiments, the disease or disorder is a fluency disorder. As used herein, a “fluency disorder” is an interruption in the flow of speaking characterized by atypical rate, rhythm, and disfluencies (e.g., repetitions of sounds, syllables, words, and phrases; sound prolongations; and blocks), which may also be accompanied by excessive tension, speaking avoidance, struggle behaviors, and secondary mannerisms. People with fluency disorders also frequently experience psychological, emotional, social, and functional impacts as a result of their communication disorder. Childhood-onset fluency disorder (stuttering), the most common fluency disorder, is an interruption in the flow of speaking characterized by specific types of disfluencies, including (i) repetitions of sounds, syllables, and monosyllabic words; (ii) prolongations of consonants when it isn't for emphasis; and (iii) blocks (i.e., inaudible or silent fixation or inability to initiate sounds). These disfluencies can affect the rate and rhythm of speech and may be accompanied by negative reactions to speaking; avoidance behaviors (i.e., avoidance of sounds, words, people, or situations that involve speaking); escape behaviors, such as secondary mannerisms (e.g., eye blinking and head nodding or other movements of the extremities, body, or face); and physical tension. Children and adults who stutter also frequently experience psychological, emotional, social, and functional consequences from their stuttering, including anxiety (e.g., social anxiety), a sense of loss of control, and negative thoughts or feelings about themselves or about communication. Childhood-onset fluency disorder (stuttering) can co-occur with other disorders, such as attention-deficit/hyperactivity disorder, autism spectrum disorder, intellectual disability, language or learning disability, seizure disorders, anxiety disorders (e.g., social anxiety disorder), speech sound disorders, and other developmental disorders.

In some embodiments, the disclosure provides for the treatment/management of different kinds of pain, including but not limited to cancer pain, e.g., refractory cancer pain; neuropathic pain; postoperative pain; opioid-induced hyperalgesia and opioid-related tolerance; neurologic pain; postoperative/post-surgical pain; complex regional pain syndrome (CRPS); shock; limb amputation; severe chemical or thermal burn injury; sprains, ligament tears, fractures, wounds and other tissue injuries; dental surgery, procedures and maladies; labor and delivery; during physical therapy; radiation poisoning; acquired immunodeficiency syndrome (AIDS); epidural (or peridural) fibrosis; orthopedic pain; back pain; failed back surgery and failed laminectomy; sciatica; painful sickle cell crisis; arthritis; autoimmune disease; intractable bladder pain; pain associated with certain viruses, e.g., shingles pain or herpes pain; acute nausea, e.g., pain that may be causing the nausea or the abdominal pain that frequently accompanies sever nausea; migraine, e.g., with aura; and other conditions including depression (e.g., acute depression or chronic depression), depression along with pain, alcohol dependence, acute agitation, refractory asthma, acute asthma (e.g., unrelated pain conditions can induce asthma), epilepsy, acute brain injury and stroke, Alzheimer's disease and other disorders. The pain may be persistent or chronic pain that lasts for weeks to years, in some cases even though the injury or illness that caused the pain has healed or gone away, and in some cases despite previous medication and/or treatment. In some embodiments, the pain is chronic pain. In addition, the disclosure includes the treatment/management of any combination of these types of pain or conditions.

In some embodiments, the pain treated/managed is acute breakthrough pain or pain related to wind-up that can occur in a chronic pain condition. In some embodiments, the pain treated/managed is cancer pain, e.g., refractory cancer pain. In some embodiments, the pain treated/managed is post-surgical pain. In some embodiments, the pain treated/managed is orthopedic pain. In some embodiments, the pain treated/managed is back pain. In some embodiments, the pain treated/managed is neuropathic pain. In some embodiments, the pain treated/managed is dental pain. In some embodiments, the condition treated/managed is depression. In some embodiments, the pain treated/managed is chronic pain in opioid-tolerant patients.

In some embodiments, the disease or disorder is arthritis. Types of arthritis include osteoarthritis, rheumatoid arthritis, childhood arthritis, fibromyalgia, gout, and lupus. In some embodiments, the disease or disorder is osteoarthritis. In some embodiments, the disease or disorder is rheumatoid arthritis. In some embodiments, the disease or disorder is childhood arthritis. In some embodiments, the disease or disorder is gout. In some embodiments, the disease or disorder is lupus. In some embodiments, the disease or disorder is fibromyalgia. Fibromyalgia is a disorder characterized by widespread musculoskeletal pain accompanied by fatigue, sleep, memory and mood issues. Fibromyalgia is believed to amplify painful sensations by affecting brain and spinal cord processes involving painful and nonpainful signaling. Symptoms often begin after an event, such as physical trauma, surgery, infection or significant psychological stress. In other cases, symptoms gradually accumulate over time with no single triggering event. Women are more likely to develop fibromyalgia than are men. Many people who have fibromyalgia also have tension headaches, temporomandibular joint (TMJ) disorders, irritable bowel syndrome, anxiety and depression.

Non-limiting examples of PD clinical scales, diary assessments, and assessments by a clinician or caregiver which can be used to assess the duration of action, psychedelic state, mental state, mood, drug effect, etc. of the patient across one or more indications include, but are not limited to, Mini International Neuropsychiatric Interview (MINI), Mystical Experience Questionnaire (MEQ30), the 5-Dimensional Altered States of Consciousness Rating Scale (5D-ASC), the Hallucinogen Rating Scale (HRS), the visual analogue scale (VAS) ratings of ‘Any drug effect’, a 5-item Persisting Effects Questionnaire (PEQ) assessing the meaningfulness, spirituality and psychological insights and challenges of the experience and overall drug effect, the NeuroCart test battery (e.g., saccadic eye movements, smooth pursuit eye movements, adaptive tracking, body sway, pupil size ratio, Visual analogs Scales Bond and Lader (VAS B&L) and Bowdle (VAS Bowdle)), pharmaco electroencephalogram (EEG), State-Trait Anxiety Inventory (STAI), The Dutch Temperament and Character Inventory (TCI), The Dutch Personality Questionnaire-2-Revised (NPV-2-R), The Profile of Mood States (POMS), and the Real-Time Intensity scale.

The Mini International Neuropsychiatric Interview (MINI) (for example, version 7.0.2) is a diagnostic interview instrument for psychiatric disorders in the Diagnostic and Statistical Manual of Mental Disorders (DSM-5) and the International Classification of Diseases-10. In some embodiments, treating according to the methods of the disclosure results in an improvement in a psychiatric disorder (e.g., depression, anxiety, etc.) compared to pre-treatment of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, according to a Mini International Neuropsychiatric Interview assessment. In some embodiments, the subject has a lower MINI 7.0.2 score compared with the subject's MINI 7.0.2 score prior to treatment.

The Mystical Experiences Questionnaire was first developed during an online survey on psilocybin-containing mushrooms and validated using data from experimental studies with controlled doses of psilocybin. The revised version contains 30 items (MEQ30) regarding subjective drug effects and is completed retrospectively. Effects are scored in total and on four subdomains (mystical, positive mood, space/time, ineffability), based on a percentage of maximum possible score. A Mystical Experience Questionnaire (MEQ30) score of greater than 60%, the criteria for a complete mystical experience, has been shown to be a mediator of symptom improvement in previous psychedelic clinical trials. In some embodiments, the methods herein provide the patient with a Mystical Experience Questionnaire (MEQ30) score of at least 60%, at least 65%, at least 70%, at least 75%, at least 80%, at least 85%, at least 90%, at least 95%. In some embodiments, at least 50%, at least 55%, at least 60%, at least 65%, at least 70%, at least 75%, at least 80%, at least 90% of subjects treated with the methods described herein have a Mystical Experience Questionnaire (MEQ30) score of at least 60%, at least 65%, at least 70%, at least 75%, at least 80%, at least 85%, at least 90%, at least 95%.

The 5-Dimensional Altered States of Consciousness Rating Scale (5D-ASC) measures altered states of consciousness and contains 94 items (visual analog scales). The 5D-ASC scale measures alterations in mood, perception, experience of self in relation to environment, and thought disorder. The instrument consists of five subscales/dimensions and 11 lower-order scales. The 5D-ASC dimension “Oceanic Boundlessness” (27 items) measures derealization and depersonalization associated with positive emotional states, ranging from heightened mood to euphoric exaltation. The corresponding lower-order scales include “experience of unity,” “spiritual experience,” “blissful state,” and “insightfulness.” The dimension “Anxious Ego Dissolution” (21 items) summarizes ego disintegration and loss of self-control phenomena associated with anxiety. The corresponding lower-order scales include “disembodiment,” “impaired control of cognition,” and “anxiety.” The dimension “Visionary Restructuralization” (18 items) consists of the lower-order scales “complex imagery,” “elementary imagery,” “audio-visual synesthesia,” and “changed meaning of percepts.” Two additional dimensions describe “Auditory Alterations” (15 items) and “Reduction of Vigilance” (12 items). Scoring is based on a percentage of maximum possible score. The scale is well-validated and widely used to characterize the subjective effects of various psychedelic drugs. In some embodiments, the 5D-ASC is used to measure changes in the subjects' subjective psychedelic experience. In some embodiments, the methods herein provide the patient with a 5D-ASC score (e.g., on the oceanic boundlessness subscale) of at least 60%, at least 65%, at least 70%, at least 75%, at least 80%, at least 85%, at least 90%, at least 95%. In some embodiments, at least 50%, at least 55%, at least 60%, at least 65%, at least 70%, at least 75%, at least 80%, at least 90% of subjects treated with the methods described herein have a 5D-ASC score (e.g., on the oceanic boundlessness subscale) of at least 60%, at least 65%, at least 70%, at least 75%, at least 80%, at least 85%, at least 90%, at least 95%.

The Hallucinogen Rating Scale (HRS) is a questionnaire with up to 100 items, and the questionnaire is designed to assess the subjective effects of hallucinogenic substances. Participants rate responses to the majority of questions on a 5-point intensity scale: 0=not at all; 1=slightly; 2=moderately; 3-quite a bit; and 4-extremely. Some questions have a slightly modified scale, and one question asks to rate the amount of time between when the drug was administered and feeling an effect from: no effect, 0-5 minutes, 5-15 minutes, 15-30 minutes, 30-60 minutes, or more than one hour. Questions are grouped into factors or domains including: 1) somaesthesia, 2) affect, 3) perception, 4) cognition, 5) volition, and 6) intensity. In some embodiments, the methods herein provide the patient with a Hallucinogen Rating Scale (HRS) score (e.g., on the intensity subscale) of at least 1.8, at least 2.0, at least 2.2, at least 2.4, at least 2.6, at least 2.8, at least 3.0, at least 3.2, at least 3.4, at least 3.6, at least 3.8, or 4.0. In some embodiments, at least 50%, at least 55%, at least 60%, at least 65%, at least 70%, at least 75%, at least 80%, at least 90% of subjects treated with the methods described herein have a score on the Intensity subscale of the HRS of greater than >2.79, for example, a score of at least 2.8, at least 3.0, at least 3.2, at least 3.4, at least 3.6, at least 3.8, or 4.0.

The visual analogue scale (VAS) is a psychometric response scale that can be used in questionnaires. It is a measurement instrument for subjective characteristics or attitudes that cannot be directly measured. When responding to a VAS item, respondents specify their level of agreement to a statement by indicating a position along a continuous line between two end points, usually a horizontal 100-mm line marked from “not at all,” “definitely not,” or the like, on the left to “extremely,” “definitely so,” or the like, on the right. The Bond and Lader VAS involves the subject indicating (with vertical marks) on sixteen horizontal 100-mm visual analogue scales how he/she feels. From these measurements, three main factors are calculated as described by Bond and Lader: alertness (from nine scores), contentedness (often called mood; from five scores), and calmness (from two scores). The Bowdle VAS and Drug Rating VAS provides the subject with items of the drug rating questionnaire: feel drug, like drug, and dislike drug. The VAS any drug effect involves the subject indicating (with vertical marks) on horizontal 100-mm visual analogue scales with the any drug effects item (“I can feel any drug effect”). In some embodiments, the subject has a maximum VAS score (e.g., Bond and Lader VAS, Bowdle VAS and Drug Rating VAS, and/or VAS any drug effect) of ≥60 mm after treatment. In some embodiments, the subject has a VAS score (e.g., Bond and Lader VAS, Bowdle VAS and Drug Rating VAS, and/or VAS any drug effect) of greater than or equal to 60 mm, 65 mm, 70 mm, 75 mm, 80 mm, 85 mm, 90 mm, or 95 mm, after treatment. In some embodiments, at least 50%, at least 55%, at least 60%, at least 65%, at least 70%, at least 75%, at least 80%, at least 85%, at least 90% of subjects treated with the methods described herein report a maximum VAS score (e.g., Bond and Lader VAS, Bowdle VAS and Drug Rating VAS, and/or VAS any drug effect) of greater than or equal to 60 mm, 65 mm, 70 mm, 75 mm, 80 mm, 85 mm, 90 mm, or 95 mm following treatment. In some embodiments, the subject has a maximum VAS any drug effect score of ≥60 mm after treatment. In some embodiments, the subject has a VAS any drug effect score of greater than or equal to 60 mm, 65 mm, 70 mm, 75 mm, 80 mm, 85 mm, 90 mm, or 95 mm, after treatment. In some embodiments, at least 50%, at least 55%, at least 60%, at least 65%, at least 70%, at least 75%, at least 80% of subjects treated with the methods described herein report a maximum VAS any drug effect score of greater than or equal to 60 mm, 65 mm, 70 mm, 75 mm, 80 mm, 85 mm, 90 mm, or 95 mm following treatment.

The 5-item Persisting Effects Questionnaire (PEQ) is a 5-item questionnaire that assesses the meaningfulness, spiritual significance, psychological insightfulness, and how psychologically challenging a participant experience was during the medicine session. Scores are assessed on a scale from 0 (not at all) to 5 (extremely). Higher scores (under consideration of reverse-scored items) indicate stronger persisting treatment effects. In some embodiments, the patient reports their experience as the most or among the top five most meaningful experiences of their life according to the Persisting Effects Questionnaire (PEQ). In some embodiments, the patient reports their experience as the most or among the top five most psychologically insightful experiences of their life, according to the Persisting Effects Questionnaire (PEQ).

The Profile of Mood States (POMS) measures six identifiable mood- or affective states: Tension-Anxiety, Depression-Rejection, Anger-Hostility, Vigor-Activity, and Fatigue-Inertia. In the original POMS, these states are addressed through 65 five-point adjective rating scales, and a sixth dimension (confusion-bewilderment) is added that does not appear in the abbreviated POMS wherein 32 questions are used to assess different periods (usually past two hours, sometimes past week). The scales use a five-point score system, ranging from 0 (not at all) to 4 (extremely). The scores of the different states are calculated using a scoring algorithm.

In the Real-Time Intensity scale, subjects are asked to verbally rate the psychological intensity of their experience on a scale from 0-4 (0=not at all; 1=slightly; 2=moderately; 3 very much; 4=extremely) on three levels: visual intensity, bodily intensity, and emotional/metacognitive intensity. Their answers are recorded during the drug administration (during the experience). In some embodiments, the methods herein provide the patient with a Real-Time Intensity scale score (e.g., on the emotional/metacognitive subscale) of at least 1.8, at least 2.0, at least 2.2, at least 2.4, at least 2.6, at least 2.8, at least 3.0, at least 3.2, at least 3.4, at least 3.6, at least 3.8, or 4.0. In some embodiments, at least 50%, at least 55%, at least 60%, at least 65%, at least 70%, at least 75%, at least 80%, at least 90% of subjects treated with the methods described herein have a score on the Real-Time Intensity scale of greater than >2.79, for example, a score of at least 2.8, at least 3.0, at least 3.2, at least 3.4, at least 3.6, at least 3.8, or 4.0.

In some embodiments, a first dose of about 8-16 mg, or about 8-14 mg, or about 8-12 mg, or about 12-16 mg, or about 12 mg, or about 16 mg of a compound of Formula (I-3), or a pharmaceutically acceptable salt, polymorph, or solvate thereof and a suitable assessment (e.g., a VAS any drug effect questionnaire, the Montgomery-Åsberg Depression Ratings Scale (MADRS), or both) are used to select subjects that are suitable candidates for further treatment. For example, in some embodiments, the methods comprise administering a first dose (e.g., about 8-16 mg or about 8-14 mg or about 8-12 mg or about 12-16 mg, or about 12 mg, or about 16 mg) of a compound of Formula (I-3), or a pharmaceutically acceptable salt thereof, or a solvate or polymorph thereof to a subject, followed by administering a suitable assessment (e.g., VAS any drug effect questionnaire, the Montgomery-Åsberg Depression Ratings Scale (MADRS), or both) to the subject (e.g., 4 to 10 hours postdose), and if the subject has a maximum VAS any drug effect score of greater than or equal to 60 mm, 65 mm, 70 mm, 75 mm, 80 mm, 85 mm, 90 mm, or 95 mm, and/or is deemed to be a responder based on a reduction in MADRS score (e.g., a reduction of MADRS score from baseline of at least about 50%, 55%, 60%, 65%, 70%, 75%, etc.) following the first dose, then administering to those subjects a second dose of a compound of Formula (I-3), or a pharmaceutically acceptable salt, polymorph, or solvate thereof, wherein the second dose is greater than or equal to the first dose. In some embodiments, the first dose is about 8 mg of the compound of Formula (I-3), or a pharmaceutically acceptable salt, polymorph, or solvate thereof, and the second dose is about 10-14 mg of the compound of Formula (I-3), or a pharmaceutically acceptable salt, polymorph, or solvate thereof. In some embodiments, the first dose is about 10 mg of the compound of Formula (I-3), or a pharmaceutically acceptable salt, polymorph, or solvate thereof, and the second dose is about 12-14 mg of the compound of Formula (I-3), or a pharmaceutically acceptable salt, polymorph, or solvate thereof. In some embodiments, the first dose is about 8-12 mg of the compound of Formula (I-3), or a pharmaceutically acceptable salt, polymorph, or solvate thereof, and the second dose is about 14-16 mg of the compound of Formula (I-3), or a pharmaceutically acceptable salt, polymorph, or solvate thereof. In some embodiments, the first dose is about 8-16 mg of the compound of Formula (I-3), or a pharmaceutically acceptable salt, polymorph, or solvate thereof, and the second dose is about 8-16 mg of the compound of Formula (I-3), or a pharmaceutically acceptable salt, polymorph, or solvate thereof. In some embodiments, the first dose is about 12 mg to about 16 mg of the compound of Formula (I-3), or a pharmaceutically acceptable salt, polymorph, or solvate thereof, and the second dose is about 12 mg to about 16 mg of the compound of Formula (I-3), or a pharmaceutically acceptable salt, polymorph, or solvate thereof. In some embodiments, the first dose is about 12 mg of the compound of Formula (I-3), or a pharmaceutically acceptable salt, polymorph, or solvate thereof, and the second dose is about 12 mg of the compound of Formula (I-3), or a pharmaceutically acceptable salt, polymorph, or solvate thereof. In some embodiments, the first dose is about 16 mg of the compound of Formula (I-3), or a pharmaceutically acceptable salt, polymorph, or solvate thereof, and the second dose is about 16 mg of the compound of Formula (I-3), or a pharmaceutically acceptable salt, polymorph, or solvate thereof.

In some embodiments, a first dose of a compound of Formula (I-3), or a pharmaceutically acceptable salt, polymorph, or solvate thereof and a suitable assessment (e.g., a VAS any drug effect questionnaire, the Montgomery-Åsberg Depression Ratings Scale (MADRS) when the disorder is a depressive disorder, or both) are used to select subjects that are suitable candidates for an increased dosage amount or regimen. For example, in some embodiments, the methods comprise administering a first dose (e.g., about 8-12 mg or about 8-10 mg) of a compound of Formula (I-3), or a pharmaceutically acceptable salt thereof, or a solvate or polymorph thereof to a subject, followed by administering a suitable assessment (e.g., VAS any drug effect questionnaire, the Montgomery-Åsberg Depression Ratings Scale (MADRS), or both) to the subject (e.g., 4 to 10 hours postdose), and if the subject has a maximum VAS any drug effect score of less than 60 mm, less than 55 mm, less than 50 mm, less than 45 mm, less than 40 mm, less than 35 mm, less than 30 mm, or less than 25 mm, and/or a reduction of MADRS score from prior to treatment (baseline) of less than about 50%, such as about 5%, about 10%, about 15%, about 20%, about 25%, about 30%, about 35%, about 40%, about 45%, or no change in MADRS score following the first dose, then administering to those subjects a second dose of a compound of Formula (I-3), or a pharmaceutically acceptable salt, polymorph, or solvate thereof, wherein the second dose is greater than the first dose. In some embodiments, the first dose is about 8 mg of the compound of Formula (I-3), or a pharmaceutically acceptable salt, polymorph, or solvate thereof, and the second dose is about 10-16 mg, about 10-14 mg, or about 12-16 mg of the compound of Formula (I-3), or a pharmaceutically acceptable salt, polymorph, or solvate thereof. In some embodiments, the first dose is about 10 mg of the compound of Formula (I-3), or a pharmaceutically acceptable salt, polymorph, or solvate thereof, and the second dose is about 12-16 mg, or about 12-14 mg, or about 14-16 mg of the compound of Formula (I-3), or a pharmaceutically acceptable salt, polymorph, or solvate thereof. In some embodiments, the first dose is about 8-12 mg of the compound of Formula (I-3), or a pharmaceutically acceptable salt, polymorph, or solvate thereof, and the second dose is about 14-16 mg of the compound of Formula (I-3), or a pharmaceutically acceptable salt, polymorph, or solvate thereof. In some embodiments, the first dose is about 10-12 mg of the compound of Formula (I-3), or a pharmaceutically acceptable salt, polymorph, or solvate thereof, and the second dose is about 14-16 mg of the compound of Formula (I-3), or a pharmaceutically acceptable salt, polymorph, or solvate thereof. In some embodiments, the first dose is about 10 mg of the compound of Formula (I-3), or a pharmaceutically acceptable salt, polymorph, or solvate thereof, and the second dose is about 16 mg of the compound of Formula (I-3), or a pharmaceutically acceptable salt, polymorph, or solvate thereof. In some embodiments, the first dose is about 12 mg of the compound of Formula (I-3), or a pharmaceutically acceptable salt, polymorph, or solvate thereof, and the second dose is about 16 mg of the compound of Formula (I-3), or a pharmaceutically acceptable salt, polymorph, or solvate thereof.

In some embodiments, the methods provide a neocortical serotonin 5-HT_{2A} receptor occupancy of at least 55%, at least 60%, at least 65%, at least 70%, at least 75%, at least 80%, at least 85%, at least 90%, at least 95%, as determined by positron emission tomography according to Madsen M K, Fisher P M, Burmester D, et al (2019). Psychedelic effects of psilocybin correlate with serotonin 2A receptor occupancy and plasma psilocin levels. Neuropsychopharmacology, 44:1328-34.

In some embodiments, the compound of Formula (I-3), or a pharmaceutically acceptable salt, polymorph, or solvate thereof, may be used as a standalone therapy.

Adjuvant/Combination Therapy In some embodiments, the compound of Formula (I-3), or a pharmaceutically acceptable salt, polymorph, or solvate thereof, may be used in adjuvant/combination therapy. In some embodiments, the subject (e.g., with MDD) is administered the compound of Formula (I-3), or a pharmaceutically acceptable salt, polymorph, or solvate thereof, and at least one additional therapy and/or therapeutic. Administration of the compound of Formula (I-3), or a pharmaceutically acceptable salt, polymorph, or solvate thereof, and at least one additional therapy and/or therapeutic may be performed simultaneously, sequentially, or separately. In some embodiments, administration of an additional therapy and/or therapeutic is prior to administration of the compound of Formula (I-3), or a pharmaceutically acceptable salt, polymorph, or solvate thereof. In some embodiments, administration of an additional therapy and/or therapeutic is after administration of the compound of Formula (I-3), or a pharmaceutically acceptable salt, polymorph, or solvate thereof. In some embodiments, administration of an additional therapy and/or therapeutic is concurrent with administration of the compound of Formula (I-3), or a pharmaceutically acceptable salt, polymorph, or solvate thereof. In some embodiments, administration of an additional therapy and/or therapeutic is prior to, during, and/or after administration of the compound of Formula (I-3), or a pharmaceutically acceptable salt, polymorph, or solvate thereof. In some embodiments, administration of the compound of Formula (I-3), or a pharmaceutically acceptable salt, polymorph, or solvate thereof, as part of combination or adjunctive therapy, may be accompanied by psychotherapy before, during, and/or after each dose.

In the adjunctive or combination therapy described herein, the compound of Formula (I-3), or a pharmaceutically acceptable salt, polymorph, or solvate thereof, may be administered using a different dosing schedule than the additional therapy and/or therapeutic. For example, the subject may be taking an antidepressant medication on a daily schedule (e.g., daily oral dosing), while the compound of Formula (I-3), or a pharmaceutically acceptable salt, polymorph, or solvate thereof, may be administered using an intermittent dosing schedule as described herein, e.g., one dose in a treatment course or two doses administered three weeks apart (±3 days) in a treatment course. In some embodiments, adjunctive therapy or combination therapy provides detectable plasma levels of the compound of Formula (I-3) and the at least one additional therapeutic (e.g., antidepressant medication) at the same time. For example, a subject taking an antidepressant medication on a daily schedule (e.g., daily oral dosing) who is administered the compound of Formula (I-3), or a pharmaceutically acceptable salt, polymorph, or solvate thereof, may have detectable plasma levels of each agent shortly after dosing of the compound of Formula (I-3), or a pharmaceutically acceptable salt, polymorph, or solvate thereof. In some embodiments, adjunctive therapy or combination therapy does not lead to detectable plasma levels of the compound of Formula (I-3) and the at least one additional therapeutic (e.g., antidepressant medication) at the same time.

In some embodiments, the additional therapeutic is an antidepressant medication, an anticonvulsant, lisdexamfetamine dimesylate, an antipsychotic, an anxiolytic, an anti-inflammatory drug, a benzodiazepine, an N-methyl-D-aspartate (NMDA) receptor antagonist, an analgesic drug, a cardiovascular drug, an opioid antagonist, or combinations thereof.

In some embodiments, the additional therapeutic is an antidepressant medication. In some embodiments, the compound of Formula (I-3), or a pharmaceutically acceptable salt, polymorph, or solvate thereof, is used in adjuvant therapy for patients already taking an antidepressant medication (e.g., SSRIs, SNRIs, etc.). For clarity, when referencing combination or adjunctive therapy involving an “antidepressant medication” (or simply an “antidepressant”) the compound of Formula (I-3), or a pharmaceutically acceptable salt, polymorph, or solvate thereof, is considered herein to be separate and distinct from the antidepressant medication, despite both having antidepressant properties. Instead, “antidepressant medication” (or “antidepressant”) refers to non-psychedelic agents, with most regulator-approved antidepressant medications being monoamine antidepressant agents characterized by a mode of action which addresses one or more imbalance (usually a deficiency) of neurotransmitters serotonin, norepinephrine and dopamine. In some embodiments, the antidepressant medication is an approved pharmacological treatment for a depressive disorder in at least one jurisdiction, such as the United States of America, the European Union, the United Kingdom, Australia, New Zealand, and Japan. In some embodiments, an antidepressant medication indirectly affects a neurotransmitter receptor, e.g., via interactions affecting the reactivity of other molecules at a neurotransmitter receptor. In some embodiments, the antidepressant medication is an agonist. In some embodiments, the antidepressant medication is an antagonist. In some embodiments, the antidepressant medication is a tricyclic antidepressant (“TCA”), selective serotonin reuptake inhibitor (“SSRI”), serotonin reuptake inhibitor (“SRI”), serotonin and noradrenaline reuptake inhibitor (“SNRI”), dopamine reuptake inhibitor (“DRI”), noradrenaline reuptake inhibitor (“NRU”), dopamine, serotonin, and noradrenaline reuptake inhibitor (“DSNRI”), a monoamine oxidase inhibitor (“MAOI”), including a reversible inhibitor of monoamine oxidase type A (RIMA), or combinations thereof. In some embodiments, the antidepressant medication is a TCA. In some embodiments, the TCA is imipramine or clomipramine. In some embodiments, the antidepressant medication is a serotonin reuptake inhibitor (“SRI”). In some embodiments, the antidepressant medication is a selective serotonin reuptake inhibitor (SSRI). In some embodiments, the SSRI is citalopram, escitalopram, paroxetine, sertraline, fluvoxamine, fluoxetine, or combinations thereof. In some embodiments, the antidepressant medication is a serotonin and noradrenaline reuptake inhibitor (SNRI). In some embodiments, the SNRI is venlafaxine, desvenlafaxine, duloxetine, levomilnacipran, or combinations thereof. In some embodiments, the antidepressant effects are augmented with the use of pregabalin as an additional therapeutic. In some embodiments, the antidepressant medication acts (either directly or indirectly) at more than one type of neurotransmitter receptor. In some embodiments, the antidepressant medication is chosen from buproprion, citalopram, duloxetine, escitalopram, fluoxetine, fluvoxamine, milnacipran, mirtazapine, paroxetine, reboxetine, sertraline, venlafaxine, dextromethorphan, cariprazine, vortioxetine, vilazodone, and levomilnacipram. Antidepressant medications such as SSRIs are usually taken orally, typically in tablet form, and usually need to be taken daily. In some embodiments, the compound of Formula (I-3), or a pharmaceutically acceptable salt, polymorph, or solvate thereof, is used as adjuvant therapy for patients taking a stable dose of antidepressant medication, such as patients on background antidepressant medication(s) including SSRIs and SNRIs. In some embodiments, the compound of Formula (I-3), or a pharmaceutically acceptable salt, polymorph, or solvate thereof, is used as adjuvant therapy for patients taking a stable dose of antidepressant medication with an inadequate response to that antidepressant medication. In some embodiments, the subject is taking an antidepressant medication monotherapy. In some embodiments, the subject is taking a combination of antidepressant medications. In some embodiments, the subject is taking an antidepressant medication such as citalopram, escitalopram, paroxetine, sertraline, fluoxetine, venlafaxine, desvenlafaxine, duloxetine, levomilnacipran, buproprion, vortioxetine, vilazodone, or combinations thereof.

In some embodiments, the additional therapeutic is an anticonvulsant. In some embodiments, the anticonvulsant is gabapentin, carbamazepine, ethosuximide, lamotrigin, felbamate, topiramate, zonisamide, tiagabine, oxcarbazepine, levetiracetam, divalproex sodium, phenytoin, fosphenytoin. In some embodiments, the anticonvulsant is topiramate.

In some embodiments, the additional therapeutic is an antipsychotic. In some embodiments, the antipsychotic is a phenothiazine, butryophenone, thioxanthene, clozapine, risperidone, olanzapine, or sertindole, quetiapine, aripiprazole, zotepine, perospirone, a neurokinin-3 antagonist, such as osanetant and talnetant, rimonabant, or a combination thereof.

In some embodiments, the additional therapeutic is an anxiolytic. In some embodiments, an anxiolytic is chosen from alprazolam, an alpha blocker, an antihistamine, a barbiturate, a beta blocker, bromazepam, a carbamate, chlordiazepoxide, clonazepam, clorazepate, diazepam, flurazepam, lorazepam, an opioid, oxazepam, temazepam, or triazolam.

In some embodiments, the additional therapeutic is an anti-inflammatory drug. In some embodiments, the anti-inflammatory drug is a nonsteroidal anti-inflammatory drugs (NSAIDS), steroid, acetaminophen (COX-3 inhibitors), 5-lipoxygenase inhibitor, leukotriene receptor antagonist, leukotriene A4 hydrolase inhibitor, angiotensin converting enzyme antagonist, beta blocker, antihistaminic, histamine 2 receptor antagonist, phosphodiesterase-4 antagonist, cytokine antagonist, CD44 antagonist, antineoplastic agent, 3-hydroxy-3-methylglutaryl coenzyme A inhibitor (statins), estrogen, androgen, antiplatelet agent, antidepressant, Helicobacter pylori inhibitors, proton pump inhibitor, thiazolidinedione, dual-action compounds, or combination thereof.

In some embodiments, the additional therapeutic is a benzodiazepine. In some embodiments, the benzodiazepine is diazepam or alprazolam.

In some embodiments, the additional therapeutic is a N-methyl-D-aspartate (NMDA) receptor antagonist. In some embodiments, the NMDA receptor antagonist is ketamine. In some embodiments, the NMDA receptor antagonist is nitrous oxide.

In some embodiments, the additional therapeutic is an analgesic. In some embodiments, the analgesic is acetaminophen. In some embodiments, the analgesic is a nonsteroidal anti-inflammatory drug (NSAID), such as aspirin, ibuprofen and naproxen. In some embodiments, the analgesic is a COX-2 inhibitor, such as rofecoxib, celecoxib, parecoxib, and etoricoxib. In some embodiments, the analgesic is an opioid.

In some embodiments, the additional therapeutic is a cardiovascular drug. Non-limiting examples of cardiovascular drugs include digoxin or (3β,5β,12β)-3-[(O-2,6-dideoxy-β-D-ribo-hexopyranosyl-(1→4)-O-2,6-dideoxy-β-D-ribo-hexopyranosyl-(1→4)-2,6-dideoxy-β-D-ribohexopyranosyl)oxy]-12,14-dihydroxy-card-20(22)-enolide, lisinopril, captopril, ramipril, trandolapril, benazepril, cilazapril, enalapril, moexipril, perindopril, quinapril, fludrocortisone, enalaprilate, quinapril, perindopril, apixaban, dabigatran, edoxaban, heparin, rivaroxaban, warfarin, aspirin, clopidogrel, dipyridamole, prasugrel, ticagrelor, azilsartan, candesartan, eprosartan, irbesartan, losartan, olmesartan, telmisartan, valsartanscaubitril, acebutolol, atenolol, betaxolol, bisoprolol, metoprolol, nadolol, propranolol, sotalol, amlodipine, diltiazem, felodipine, nifedipine, nimodipine, nisolidipine, verapamil, statins, nicotinic acids, diuretics, vasodilators, and combinations thereof.

In some embodiments, the additional therapeutic is an opioid antagonist. Non-limiting examples of opioid antagonists include naloxone, naltrexone, nalmefene, nalorphine, nalrphine dinicotinate, levallrphan, samidorphan, nalodeine, alvimopan, methylnaltrexone, naloxegol, 6-naltrexol, axelopran, bevenopran, methylsamidorphan, naldemedine, buprenorphine, decozine, butorphanol, levorphanol, nalbuphine, pentazocine, and phenazocine.

In some embodiments, the additional therapeutic is a serotonin receptor modulator. Non-limiting examples of serotonin receptor modulators include glemanserin (MDL-11,939), eplivanserin (SR-46,349), ketanserin, ritanserin, altanserin, acepromazine, mianserin, mirtazapine, quetiapine, SB204741, SB206553, SB242084, LY272015, SB243213, blonanserin, SB200646, RS102221, nefazodone, volinanserin (MDL-100,907), pimavanserin (ACO-103), nelotanserin, lorcaserin, flibanserin, and roluperiodone.

Some medications are preferably not used in combination with the compound of Formula (I-3), or a pharmaceutically acceptable salt, polymorph, or solvate thereof, for example, those medications which are contraindicated, those which may potentially augment subjective effects to too great an extent, or those which may potentially blunt desirable psychedelic effects. Examples of medications which, in preferred embodiments, are not used in combination with the compound of Formula (I-3), or a pharmaceutically acceptable salt, polymorph, or solvate thereof, include, but are not limited to, a monoamine oxidase inhibitor, a tricyclic antidepressant (“TCA”), and a serotonin receptor modulator such as a 5-HT_{2A} blocker. In some embodiments, the subject is not taking or administered a monoamine oxidase inhibitor (MAOI), in particular a monoamine oxidase type A or type B (MAO-A/B) inhibitor. In some embodiments, the subject is not taking or administered a tricyclic antidepressant (“TCA”). In some embodiments, the subject is not taking or administered a serotonin receptor modulator. In some embodiments, the subject is not taking or administered a 5-HT_{2A} blocker, such as mirtazapine.

In some embodiments, the subject is administered at least one therapy in addition to administration of a compound of Formula (I-3), or a pharmaceutically acceptable salt, polymorph, or solvate thereof. Non-limiting examples of therapies include transcranial magnetic stimulation, cognitive behavioral therapy, interpersonal psychotherapy or psychedelic-assisted psychotherapy (PAP), dialectical behavior therapy, mindfulness techniques, or acceptance, commitment therapy, or combinations thereof. In some embodiments, the subject is administered a six-domain framework for psychedelic-assisted psychotherapy referred to as EMBARK therapy.

In some embodiments, a method of treating a subject in need thereof comprises administering to the subject a therapeutically effective amount of a compound of Formula (I-3), or a pharmaceutically acceptable salt, polymorph, or solvate thereof, in a controlled environment, wherein the subject is provided with psychological support.

In some embodiments, a method of treating a subject in need thereof comprises at least one of the following:
(i) administering to the subject a therapeutically effective amount of the compound of Formula (I-3), or a pharmaceutically acceptable salt, polymorph, or solvate thereof, in a controlled environment, wherein the subject is provided with psychological support;
(ii) having the subject participate in one or more pre-administration psychological support session(s); and/or
(iii) having the subject participate in one or more post-administration psychological support session(s).

In some embodiments, after administration, the subject may not feel the effects of the drug for about 5 minutes, about 10 minutes, about 15 minutes, about 30 minutes, about 45 minutes, about 60 minutes, or about 90 minutes, or any range therebetween. This period after administration and before the onset of effects will be referred to herein as the initial stage of the treatment session. The time marked by the onset of the drug's effects will be referred to herein as the early stage of the treatment session.

In some embodiments, the subject will experience peak effects at about 0.15 hours, about 0.25 hours, about 0.5 hours, about 0.75 hours, about 1.0 hours, about 1.25 hours, about 1.5 hours, about 1.75 hours, about 2 hours, about 2.25 hours, about 2.5 hours, about 2.75 hours, about 3 hours, about 3.25 hours, about 3.5 hours after administration, or any range therebetween. The time period marked by the peak drug experience will be referred to herein as the peak stage of the treatment session.

In some embodiments, the effects of the compound of Formula (I-3) may substantially wear off from about 1 hour, about 2 hours, about 3 hours, about 4 hours, about 5 hours after administration, or any range therebetween. This time period will be referred to as the late stage of the treatment session.

In some embodiments, the subject's ability to reach a non-dual state (e.g., a mystical experience), or a sense of unity, boundlessness, ego-dissolution or transcendence correlates with positive clinical outcome. Each of these terms may be commonly defined as the breakdown of the usual relationship between self and other, whereby the subject might feel a oneness and increased sense of connectedness to the surrounding environment and/or the world at large. In some embodiments, low levels of emotional arousal-which could indicate avoidance, lack of involvement or intellectualization-might, in some embodiments, be correlated with little or no improvement in treatment outcomes.

Factors that may influence the subjective experience may include, for example, (i) dose, (ii) the mindset of the participant prior to the session, (iii) the setting of the session, (iv) the subject's ability to focus and stay with the experience, and/or (v) the subject's prior experience with psychedelics. These, and other factors, will be described in more detail below, along with ways to maximize therapeutic benefit of the treatment session.

### Pre-Administration Psychological Support Sessions (“Preparation Sessions”)

In some embodiments, the subject participates in at least one psychological support session before administration begins (“pre-administration psychological support session” or “preparation session”). In some embodiments, a pre-administration psychological support session may be held about 1 month prior to the administration. In some embodiments, a pre-administration psychological support session may be held about 2 weeks prior to the administration. In some embodiments, a pre-administration psychological support session may be held about 1 week prior to the administration. In some embodiments, a pre-administration psychological support session may be held about 3 days prior to the administration. In some embodiments, a pre-administration psychological support session may be held about 1 day prior to the administration. In some embodiments, a pre-administration psychological support session may be held on the same day as and prior to administration. In some embodiments, a pre-administration psychological support session may be held in any range in between about 1 month prior to the administration to the same day as administration.

In some embodiments, the subject may participate in one, two, three, four, five, six, seven, or eight pre-administration psychological support sessions. In some embodiments, the subject may participate in at least two pre-administration psychological support sessions. In some embodiments, the subject may participate in at least three pre-administration psychological support sessions. In some embodiments, the subject may participate in pre-administration psychological support sessions at least once per week, for at least two or three weeks prior to the administration session. In some embodiments, the subject may additionally participate in a pre-administration psychological support session the day before the administration session.

The pre-administration psychological support sessions may be individual sessions, wherein a subject meets one-on-one with a therapist. In some embodiments, the psychological support sessions may be group sessions, wherein more than one subject meets with a single therapist, or more than one therapist. In some embodiments, one or more of the subject's family members or friends may be present at the pre-administration psychological support session(s).

In some embodiments, the goals of the pre-administration session may include (i) establishing therapeutic alliance between subject and therapist; (ii) answering the subject's questions and addressing any concerns; and/or (iii) demonstrating and practicing the skills of self-directed inquiry and experiential processing. In some embodiments, the pre-administration psychological support sessions focus on discussion of possible psychedelic effects, and/or preparing subjects for the dosing session by practicing relevant therapeutic techniques to reduce avoidance and anxiety, eliciting relevant therapeutic goals, building rapport, and/or establishing therapeutic alliance. During the psychological support session, skills of self-directed inquiry and experiential processing may be demonstrated and/or practiced. In some embodiments, breathing exercises meant to promote calm and/or ease anxiety may be demonstrated and/or practiced. In some embodiments, the breathing exercise comprise instructing the subject to focus on their breath and/or sensations associated with the breath throughout the body. For example, the subject may be instructed to breathe in for a count of four, to hold their breath for a moment, and then to breathe out for a count of eight. In some embodiments, the therapist and subject may discuss the most helpful ways to support in case of emotional distress during the treatment session. In some embodiments, the subject is given access (e.g., online access) to materials concerning the safety and mechanism of action of the drug.

In some embodiments, the pre-administration psychological support sessions will serve to establish a therapeutic goal for the treatment session. In some embodiments, the subject suggests the therapeutic goal for herself or himself. In some embodiments, the therapist suggests the therapeutic goal to the subject. In some embodiments, the subject is reminded of the therapeutic goal during the pre-administration psychological support session.

In some embodiments, the therapists are trained to counsel the subject before, during, and/or after the treatment sessions. In some embodiments, the therapist will have mental health training. In some embodiments, the therapist will be a clinical psychologist, a psychiatrist, a social worker, a doctor or a nurse. In some embodiments, the therapist will meet the following criteria: (i) demonstrate independent clinical experience with direct subject care in areas that require counselling and psychotherapeutic skills; (ii) current unrestricted professional license and/or good professional standing with no history of suspension, professional misconduct or disciplinary actions; and/or (iii) high level of openness to learning new approaches and receiving feedback.

### Psychological Support During Treatment Sessions (“Medicine Sessions”)

During the treatment session, the subject may be supervised by one or more trained therapists. The therapist supervising the subject during the treatment session may be the same therapist from the subject's pre-administration psychological support session(s), or may be a different therapist. The therapist(s) may provide psychological support to the subject as necessary. As used herein, the term “psychological support” refers to any measure(s) taken by the therapist during the subject's treatment session to ensure the safety of the subject and maximize the clinical effectiveness of the treatment session. For example, the psychological support may be anything done by the therapist to (1) to ensure psychological safety of the subject; (2) to allow the subject's subjective experience to unfold naturally within the boundaries of the therapeutic intention set at the preparation; (3) to maintain participant's attention and awareness on the experience of the present moment thus allowing exposure and processing of the challenging emotional states and personal memories; and/or (4) to generate insights and solutions for the resolution of challenging personal situations, conflicts and traumatic experiences. In some embodiments, support can be in the form of therapeutic touch, verbal reassurance, guided imagery and/or relaxation or breathing exercises. In some embodiments, the support may comprise reminders, encouragement, or active guiding. Typically, only one technique is applied at a time to allow for minimal intervention and interference with the subject's unique process.

In some embodiments, the main therapeutic goals of the therapist during the treatment session are to (i) minimize extreme anxiety, and (ii) provide appropriate support that enables the skills and processes of self-directed inquiry and experiential processing. In some embodiments, the therapist demonstrates genuine presence, patience, curiosity, and/or openness during the treatment session. “Presence” refers to being totally available and present with the subject during all stages of the treatment session, and exuding calmness at all times. “Curiosity” refers to interest and willingness to understand the subject's experience, without making assumptions. “Patience” means that the therapist facilitates the participant taking as much time as needed to explore their experiences without controlling the natural urge to help or direct the experience. “Openness” is the ability of the therapist to remain cognitively and experientially open, including a capacity to be curious about how the subject's mind may uniquely choreograph the unfolding content of a session. This includes welcoming all emotions and expressions that might occur.

In some embodiments, the psychological support may comprise curious questioning. In this technique, brief, but detailed, questioning of subjects is used to help the subjects shift and sustain their attention towards different levels of cognition and emotions (“How does that make you feel?”). Due to the applicability across a range of mental states and within various settings, the technique of curious questioning can typically be used safely and consistently during the treatment session, regardless of the quality or intensity of the experience of each subject.

In some embodiments, the level of psychological support will vary during the various stages of the subject's treatment experience (e.g., the initial stage, the early stage, the peak stage, and the late stage). In some embodiments, the type of psychological support will vary during the various stages of the subject's treatment experience (e.g., the initial stage, the early stage, the peak stage, and the late stage). Because non-dual, ego-dissolution or “unitive” experiences have been shown to positively correlate with the magnitude and durability of the clinical response, the therapist will, in some embodiments, attend to such states with particular care.

In some embodiments, a subject may experience of a compromised sense of self during the subject's treatment experience. In some embodiments, this is interpreted from a psychoanalytic perspective as a disruption of ego-boundaries, which results in a blurring of the distinction between self-representation and object-representation, and precludes the synthesis of self-representations into a coherent whole. In some embodiments, non-dual, ego-dissolution or “unitive” experiences refer to an altered state of consciousness in which there is a reduction in the self-referential awareness that defines normal waking consciousness, resulting in a compromised sense of “self” and instead only a undivided background awareness, often characterized by a sense of unity or “oneness” that exceeds sensory or cognitive apprehension. In some embodiments, a non-dual experience is state of consciousness in which the subject-object dichotomy in normal waking consciousness is substituted for a unified background awareness that is centerless and undivided. In some embodiments, an ego dissolution experience is a spontaneously occurring state of consciousness where there is a reduction in the self-referential awareness that defines normal waking consciousness, resulting in a compromised sense of “self”. In some embodiments, a unitive experience is an experience characterized by a sense of unity or “oneness” that exceeds sensory or cognitive apprehension.

At the initial and early stage of the treatment session, psychological support may be used to reduce severe and/or prolonged anxiety. Anxiety prior to or during the onset of effects may not be uncommon, and the therapists may be specially trained to recognize and actively manage subjects through such periods of anxiety until the subject is comfortable enough to continue on their own. In some embodiments, therapists validate the subject's feelings of anxiety without providing interpretations of perceptual disturbances or guiding subjects towards a particular image or memory, other than encouraging them to stay relaxed and open to the emergent experiences. For example, in some embodiments, the therapist may help alleviate anxiety using a grounding exercise. In such an exercise, the subject may be encouraged to pay attention to the sounds around them or to sensations on their skin when touching the bed/couch, ground, or other objects.

At the initial and early stage of the treatment session, the therapist may encourage the subject to lie down, practice relaxation and breathing exercises, and/or listen to calming music. In some embodiments, the therapist may remind the subject of the intention for the treatment session. For example, the therapist may ask the subject “What does feeling better or recovery feel like?” or any number of similar questions. Such reminders prior to the onset of or at the onset of effects provide an implicit direction for the subjective experience during the treatment session. In some embodiments, the therapist may remind the subject that their primary task during this session is to simply collect new and interesting experiences which can then be discussed with the therapist after the session. The therapist may remind the participant of the purpose of the therapy and the role of experiential processing, namely allowing the participant to be open and curious to whatever arises and encountering thoughts and feelings previously unknown to them. In some embodiments, the therapist emphasizes that this process inherently requires letting go and a willing passivity to the psychedelic experience.

During the acute onset of action, the subject might experience perceptual changes in visual, auditory or olfactory modes, and a range of unusual physical sensations. These experiences could be anxiety-provoking. In some embodiments, the therapist may practice reassuring “arm holding”. This is where, upon the subject's request, a therapist will place his or her hand on the subject's wrist, arm, hand, or shoulder, as a way of helping the subject feel secure during this phase. This exercise may have been previously practiced during the pre-administration psychological support session.

In some embodiments, the therapist may encourage the subject to put on an eye mask, such as a Mindfold eyeshade. In some embodiments, the therapist encourages the subject to put on the eye mask before, during, or after the onset of the effects.

In some embodiments, the therapist may encourage the subject to put on headphones and listen to music. In some embodiments, the headphones reduce outside noise (e.g., “noise-cancelling” headphones). In some embodiments, the music is calming music such as instrumental (e.g., classical) music. In some embodiments, the music comprises nature sounds and/or the sound of moving water (e.g., ocean sounds). In some embodiments, the music comprises isochronic tones. In some embodiments, the music comprises moments of silence. In some embodiments, the music is emotionally evocative. In some embodiments, the music comprises a playlist which mirrors the pharmacodynamics of a typical high-dose treatment session: the initial stage, the early stage, the peak stage, and the late stage. In some embodiments, listening to music helps the subject to focus on their internal experience.

In case of prolonged anxiety or distress, therapists may, in some embodiments, actively guide participants through such experiences without interpreting or judging the experiences or giving advice. Once participants are comfortable, the therapist may encourage them to again engage in introspection.

During the peak and late stages of the treatment session, the therapist may encourage subjects to face and explore their experience, including the challenging ones. Therapists may direct subjects to participate self-directed inquiry and experiential processing to develop a different perspective on their personal challenges and conflicts, and to generate their own solutions. Such self-generated insights are not only therapeutic because of the emotional resolution, but also empowering to subjects.

As used herein, the term “self-directed inquiry” refers to directing attention to internal states. Subjects are encouraged to be curious about experiences in the present moment, including foreground and background thoughts, emotions, and physical sensations. During the preparation and integration stages, this inquiry might mean asking specific and detailed questions to help direct attention to internal states. However, during the period of drug action, inquiry might simply mean an attitude of openness to inner experiences.

As used herein, “experiential processing” refers to a participant's ability to maintain full attention on the experiences that come into awareness through self-directed enquiry. This includes a willingness and ability to be with and/or move ‘in and through’ even uncomfortable or challenging thoughts, feelings, sensations or emotions, until discomfort is diminished or resolved.

In some embodiments, the therapist will employ a transdiagnostic therapy. In some embodiments, the transdiagnostic therapy is a Method of Levels (MOL) therapy. In still further embodiments, the MOL therapy comprises Self-Directed Enquiry and Experiential Processing. Typically, MOL uses brief, but detailed, curious questioning to help subjects shift and sustain their attention towards different levels of cognition and emotions. The emphasis within MOL is on identifying and working with a subject's underlying distress as opposed to just their symptoms. Such MOL related methods and techniques can include: (1) Self-directed enquiry-directing attention to internal states. Participants are encouraged to be curious about experiences in the present moment, including foreground and background thoughts, emotions, and physical sensations; during the preparation and integration stages, such enquiry can mean asking specific and detailed questions to help direct attention to internal states, although for some embodiments, during the period of drug action, enquiry can refer to an attitude of openness to inner experiences; and (2) Experiential processing-sustained focus on the experience; which refers to a participant's ability to maintain full attention on the experiences that come into awareness through self-directed enquiry. This includes a willingness and ability to be with and/or move ‘in and through’ even uncomfortable or challenging thoughts, feelings, sensations or emotions, until discomfort is diminished or resolved.

In some embodiments, the psychological support comprises mindfulness-based therapy or cognitive behavioral therapy (CBT). In some embodiments, the psychological support is informed by a functional theory of human behavior called Perceptual Control Theory.

Occasionally, the subject will try to avoid emerging experiences or distract him/herself while trying to regain cognitive control over the unusual state of their mind. Such distractions may take different forms. For example, the subject might want to engage in a conversation or prematurely describe in detail their experience, visions or insights. When this occurs, the therapist may aim to remain as silent as possible, thereby enabling the subject and his/her inner experience to direct the course of the treatment session. In some embodiments, the therapist may use active listening skills paired with prompts to encourage the subject to continue focusing attention on present experiences, particularly if the participant engages the therapist in conversation. In another example, a subject might ask to go to the bathroom or have a drink of water. The sudden and urgent character of such requests might suggest that they are really trying to avoid emerging material. In such cases, the therapist may encourage the subject to stay with the experience by simply redirecting their attention. For example, the therapist may say something like, “We will take a bathroom break at the end of this piece of music” or “I will get you water in a little while. Why don't you put the eye shades back on and relax for a few minutes?” If the subject is trying to avoid a difficult experience, they might listen to the suggestion and relax.

In some embodiments, spontaneous movement such as shaking, stretching or dancing while engaging with the experience is accepted and often encouraged, unless the movement seems to be a way to distract oneself from the experience. In some embodiments, if the subject continues to move around a lot, reminders to periodically return to a lying down position and to actively focus inwards may be provided.

The therapist is not required to understand, support or even have an opinion about the nature or content of the subject's experiences, but the therapist may validate them and convey openness toward the subject's own view of them without dismissing or pathologizing any experience based on its unusual content. These experiences may provide the subject with a perspective that goes beyond identification with their personal narrative. In some embodiments, the therapist will validate one or more of the subject's experiences. In some embodiments, validation of the experiences simply means acknowledging the courage of opening up to the experience and the possibility that any experience will serve the intention of the session.

In some embodiments, a therapist provides psychological support for approximately 1 hour, 2 hours, 3 hours, 4 hours, 5 hours, 6 hours, 7 hours, 8 hours, or any range therebetween such as 4-8 hours or 2-4 hours immediately after administration. In some embodiments, the therapist uses guided imagery and/or breathing exercises to calm the subject and/or focus the subject's attention. In some embodiments, the therapist holds the hand, arm, or shoulder of the subject. In some embodiments, the therapist counsels the subject to do one or more of the following: (1) to accept feelings of anxiety, (2) to allow the experience to unfold naturally, (3) to avoid psychologically resisting the experience, (4) to relax, and/or (5) to explore the subject's own mental space.

In some embodiments, the therapist avoids initiating conversation with the subject, but responds if the subject initiates conversation. Typically, active intervention is kept to a minimum during the treatment experience. In some embodiments, the subject is encouraged to explore their own mental space, and simple guided imagery may be used to assist relaxation. “Guided imagery” refers to an exercise wherein the subject is asked to imagine a scene (e.g., “Invite a scene, perhaps a landscape, and tell me where you find yourself”; “Imagine a place that feels safe to you.”).

### Post-Administration Psychological Support Session (“Integration Session”)

In some embodiments, subjects may be encouraged to engage in post-administration integration sessions with their therapist. Integration is a process that involves processing, or embodying, a psychedelic experience within a therapeutic context. The process initially begins by the subject verbalizing and reflecting upon any experience from the treatment session, and discussing it openly with their therapist. Successful integration of a therapy experience accommodates for emotional changes and comprises of translating experiences into new insights, perspectives, and subsequently new behaviors that can be used to benefit the subject's quality of life. New perspectives might in turn influence the participant's current knowledge or values and lead to new ways of relating to cognitions, emotions, behaviors and physical experiences.

In some embodiments, the goals and supportive methods used by the therapist throughout integration sessions should remain consistent, regardless of the intensity or content of the subjective experience explored by the subject. That said, the methods of support used by the therapist should accommodate for the full range of experiences a subject might have faced.

The integration process is not one that should be limited to the sessions with the therapist, and is a process that will likely continue to unfold beyond the visits in clinic. The therapist might encourage the participant to use methods such as spending time in nature, exercise, or creative expression to help facilitate the process further. The subject might also be encouraged to discuss experiences with their friends, family, and/or support network. The role of the integration sessions is not to cover and work on every experience, but to empower the participant by building their capacity to experientially process information safely. This enables the participant to continue self-directed integration, even outside of study visits.

In some embodiments, the subject participates in at least one psychological support session after administration (“post-administration psychological support session” or “integration session”). In some embodiments, a post-administration psychological support session may be held on the same day as the treatment session, after the effects of the drug (e.g., a compound of Formula (I-3)) have substantially worn off. In some embodiments, a post-administration psychological support session may be held the day after the treatment session. In some embodiments, a post-administration psychological support session may be held two days after the treatment session. In some embodiments, a post-administration psychological support session may be held three days after the treatment session. In some embodiments, a post-administration psychological support session may be held about one week after the treatment session. In some embodiments, a post-administration psychological support session may be held about two weeks after the treatment session. In some embodiments, a post-administration psychological support session may be held about one month after the treatment session. In some embodiments, a post-administration psychological support session may be held about three months after the treatment session. In some embodiments, a post-administration psychological support session may be held about six months after the treatment session. In some embodiments, a post-administration psychological support session may be held about twelve months after the treatment session. In some embodiments, post-administration psychological support session may be held in a time range in between any of the preceding times.

In some embodiments, the subject may participate in one, two, three, four, five, six, seven, or eight post-administration psychological support sessions. In some embodiments, the subject may participate in at least two, or at least three post-administration psychological support sessions.

The post-administration psychological support sessions may be individual sessions, wherein a subject meets one-on-one with a therapist. In some embodiments, the psychological support sessions may be group sessions, wherein more than one subject meets with a single therapist, or more than one therapist. In some embodiments, one or more of the subject's family members or friends may be present at the post-administration psychological support session(s).

In some embodiments, the post-administration psychological support session may focus on integration of the treatment experience. Integration may involve processing a psychedelic experience in a therapeutic context. Integration may comprise psychological and somatic processing of the experience and a successful assimilation of insights into the subject's life for the purpose of growth, healing and/or well-being. During an integration session, a subject may be encouraged to talk about and reflect upon their experiences during the treatment session. In some embodiments, integration may comprise an external expression of the treatment experience, such as choice of words, tone of voice, gestures, and/or particular physical activities (yoga, exercise, bodywork, etc.). In some embodiments, integration comprises creatively expressing any insights or experiences gained during a treatment experience, for example through poetry, art, music/singing, dance, writing or drawing. In some embodiments, the subject may be encouraged to reflect on both the thoughts and the feelings that he or she underwent during the treatment session, as well as to express those ideas and emotions into a concrete form that can serve as a tool for continuing to remember and integrate those lessons into the future. In some embodiments, the subject may be encouraged to acknowledge and connect with the range of the emotional cognitive and physical experiences of the treatment session and relate them to current experiences in their life situation. This may be accomplished, for example, by discussing them initially with their therapist, and perhaps later with their family, friends, and support circle. Integration helps accommodate changes in emotional states as new insights are generated and integrated. When further explored through oscillating attention between foreground and background thoughts and emotions, such insights may lead to natural and effortless changes in perspectives or behaviors. In some embodiments, the integration process is not limited to initial integration meetings with the therapist, but continues to unfold spontaneously through a participant's own processing and actions in every day life.

In the case of a low-intensity experience, the integration process might focus on the mental content that emerged during the hours of relaxation and introspection. This might also include reactions to what might have been an unremarkable experience, such as feeling of disappointment, anger, relief, etc.

### Embark

In some embodiments, the psychological support administered to the subject is a six-domain framework for psychedelic-assisted psychotherapy (PAP) referred to as EMBARK, which is a transdiagnostic, trans-drug model. The six clinical domains of EMBARK are: (1) Existential-Spiritual, (2) Mindfulness, (3) Body Aware, (4) Affective-Cognitive, (5) Relational, and (6) Keeping Momentum.

(1) Existential-Spiritual. Psychedelic medicines are well known to catalyze profound encounters with mystical or spiritual content and existential concerns, such as mortality, alienation, or questions of life meaning. Several PAP clinical trials have found that participants often report profound experiences of an existential or spiritual nature that may hold enduring significance for them. The potency of participants' mystical experiences has been found to correlate with a range of treatment benefits, including reductions in symptoms of depression and treatment-resistant depression, increased motivation to stop problematic cocaine use, decreases in cancer-related depression and anxiety, greater success in nicotine cessation, and other positive changes in psychological functioning. Despite calls for further elucidation of specific therapeutic mechanisms in this domain, existential and spiritual elements of PAP warrant recognition as potential sources of treatment benefit. The current state of knowledge suggests that anti-depressive outcomes may be facilitated simply by having a mystical experience during a medicine session. However, providing support for a participant's post-medicine spiritual self-development may contribute additional benefit, as suggested by prior PAP research and non-psychedelic findings of negative correlations between spirituality and depressive symptoms. The role of EMBARK therapists, treating for example MDD, by way of this domain is thus to create the conditions for mystical or spiritual phenomena to potentially arise and to support participants in using them as an impetus for spiritual growth. In the preparation phase, therapists assess for any intrinsic motivation a participant may have to bring existential-spiritual elements into their treatment and work with them in developing this motivation into their intentions for the medicine session. For the medicine session, therapists prepare the physical treatment space in a way that demonstrates respect for the subjective sense of sacredness that may arise for the participant and open the session with a brief, collaboratively designed ritual. If phenomena in this domain arise during the medicine session, therapists are invited to use supportive psychotherapy or evidence-based interventions of their preference during the integration phase to explore the participant's experience, support them in developing a sense of what actions it could motivate, and co-create plans for life changes and/or further spiritual self-development. Examples of approaches with evidentiary bases to use in this process include meaning-oriented psychotherapies, Logotherapy, or Spiritual Guidance, which is derived from the evidence-based approach of motivational interviewing (MI), despite not being an Evidence-Based Therapy (EBT) itself. Throughout all phases, therapists are taught to attend to their own biases and beliefs in order to avoid imposing their own understanding or interpretation on a participant's experience in this domain.

(2) Mindfulness. This domain refers to treatment events that result in the participant becoming more capable of recognizing symptomatic internal states and responding to them with a greater capacity for self-compassion and self-regulation. Mindfulness in EMBARK has significant conceptual overlap with the notion of “psychological flexibility” that the ACE Model derives from Acceptance and Commitment Therapy (ACT). In previous PAP trials, participants have often experienced various forms of disruption of their habitual self, a sense of “mental freedom,” or an increased feeling of sovereignty in how one relates to the workings of their own mind. Overall, the “M” domain represents a place in EMBARK for preparing the participant to attend to their thoughts and feelings with compassion during a medicine session and for working with increases in psychological flexibility and other metacognitive shifts during integration. In the treatment of MDD, mindfulness has been found helpful in disrupting ruminative thought patterns, enabling more cognitive flexibility, and fostering a more compassionate stance toward oneself. To support these outcomes, EMBARK therapists begin a course of PAP treatment for MDD by teaching basic mindfulness skills to the participant. This teaching is only meant to ensure that the participant knows how to attend to their internal experience during the medicine session, which is when a more enduringly increased capacity for mindfulness may arise. During the integration phase, therapists help to anchor whatever aspect of mindfulness arose for the participant through the use of indication-specific mindfulness practices. Therapists may also help the participant integrate a new feeling of self-compassion or support them in using the momentary abatement of ruminative thoughts as an opportunity to develop mindfulness-based skills that may prevent a recurrence. Therapists can use their own mindfulness-based interventions within the bounds of the guidelines provided, or they can use suggested interventions drawn from Rumination-Focused Cognitive-Behavioral Therapy (RF-CBT) Mindfulness-Based Cognitive Therapy (MBCT) included in the treatment manual. At all times, therapists are taught to work with the participant in this domain within a trauma-informed approach to mindfulness and to avoid imposing one's own beliefs or biases onto the participant's experience.

(3) Body Aware. PAP participants have reported that embodied phenomena are a notable part of their experience of a medicine session. There are few EBTs that provide support in conceptualizing or responding to these phenomena, though innovative somatic psychotherapy approaches have offered suggestions. EMBARK therapists are prepared to respond to embodied treatment events using the most widely accepted elements of these novel somatic approaches, such as “pendulation,” or the process of helping a participant alternate between active, embodied engagement with trauma material and self-soothing. EMBARK also encourages integration of somatic elements from more established EBTs, such as somatic awareness training exercises and self-regulation skills from Mindfulness-Based Relapse Prevention (MBRP) or Dialectical Behavioral Therapy (DBT) to support therapeutic outcomes in this domain. The techniques used by EMBARK therapists in this domain do not require them to work with the body in an intensive, hands-on way and are thus not prohibitively far beyond their standard psychotherapeutic training. In preparation, therapists train the participant in basic somatic awareness so that they can attend to and thereby facilitate pro-therapeutic bodily phenomena during the medicine session. Once the medicine is administered, the therapists' role is to guide the participant back toward an awareness of their body when clinically indicated and help them remain within their zone of optimal arousal through the use of the self-soothing interventions or therapist-participant touch-based interventions. There remains a lack of knowledge or consensus around the efficacy, safety, or necessity of therapist-participant touch in PAP and thus EMBARK therapists are instructed to prioritize non-touch interventions and limit their touch-based interventions to basic supportive touch that minimizes points of contact between parties, like handholding or placing a hand on a participant's shoulder to convey support or offer grounding. More intensive forms of touch (e.g., full-body embraces) are omitted until further research establishes them as safe, effective interventions. Therapists also rigorously assess for the participant's consent to touch-based interventions during preparation and proactively give participants a chance to reject any form of touch during preparation, immediately before the provision of touch in the medicine session, and at any time during the touch, so as not to move beyond the participant's espoused level of comfort. The primary distinction between the Body aware and Mindfulness domains is found less in their associated interventions than in participants' subjective experiences of how benefits arise and the integration goals that best support these benefits. Body aware treatment goals follow from medicine session events that a participant locates in their body and involve integration practices that work with shifts in somatic awareness. Treatment goals in the Mindfulness domain involve benefits that adhere more closely to the core concepts of psychological flexibility and other metacognitive shifts and are sustained with practices that build upon a participant's revised relationship with the contents of their mind. In the treatment of MDD, somatic phenomena are hypothesized to contribute to therapeutic outcomes in two ways. Depressive symptoms are notable for their disturbance of embodiment (e.g., fatigue or energy loss, disruptions of sleep and appetite, weight gain or loss). It has been suggested that disruptions of one's lived experience of their body are the most fundamental and cross-culturally consistent MDD phenomena. As such, the experiences of enlivenment and sensory embodiment that some PAP participants describe may form the basis for a relationship with one's body that opposes the recurrence of depressive symptoms. If such an experience arises for a participant, therapists may work with them to build and strengthen this new relationship. Additionally, it is likely that treating MDD will often involve working with trauma. The comorbidity and symptomology of MDD and trauma has been posited as evidence that many cases of MDD can be meaningfully thought of as a subtype of PTSD characterized by an internalizing response to trauma. Although classic psychedelics have not yet been used to address trauma in a completed clinical trial, it has been suggested that the disinhibiting effect of psilocybin on frontal-limbic neural circuits of emotional regulation are similar to those of MDMA, which has more established efficacy in treating trauma. Classic psychedelics may thus have facility in trauma treatment by way of similar disinhibitory mechanisms. It is likely that PAP treatment of MDD with a classic psychedelic will sometimes facilitate the appearance of trauma symptoms, possibly in the form of the somatic phenomena described earlier (nausea, shaking, etc.). When this occurs, a skillful therapeutic response may facilitate lasting resolution.

(4) Affective-Cognitive. During a medicine session, some PAP participants experience dramatic shifts in their emotions and cognition. Participants have often reported that they experience a degree of emotionality that is ordinarily unavailable to them, including feelings of bliss, love, despair, fear, and grief, often in the context of a healing catharsis. They may also find themselves confronting maladaptive self-beliefs with a directness they would normally avoid. Engaging non-defensively with these emotions and beliefs during a medicine session has become a widely accepted practice among PAP models. This practice mirrors that found in several non-psychedelic EBTs, such as ACT's emphasis on acceptance and Emotion-Focused Therapy's (EFT) emphasis on entering into maladaptive states in service of transformation. This domain serves as a space within EMBARK for incorporating approach-oriented practices for working with emotions and self-beliefs in way that best addresses the indication being studied. For example, the EMBARK approach to MDD in this domain begins with the notion that many depressed individuals have developed a habitual response to challenging feelings that entails dimming their awareness of them through a kind of automatic, unconscious avoidance and characteristic depressive experience of feeling numb and withdrawn. Participants in PAP medicine sessions have often experienced a greater facility in reconnecting with this avoided material and have implicated these experiences in their positive treatment outcomes. In the preparation phase of MDD treatment, EMBARK therapists are tasked with helping participants understand the importance of adopting an approach orientation during the medicine session. They also help the participants develop one or more self-soothing techniques. During the medicine session, the therapists' role is to remind the participant to welcome challenging content if needed and to help them utilize the previously learned self-soothing techniques when necessary. To integrate these experiences, EMBARK therapists help participants use experiences of approaching challenging material as the basis for updating maladaptive core beliefs about oneself or the world and cultivating an enduring attitude of greater acceptance in their emotional life. All of these interventions are taught and employed in a way that is respectful of participant autonomy, the principles of trauma-informed care, and cultural differences in relating to and expressing one's emotions.

(5) Relational. Practitioner-participant interactions are often significant events in medicine sessions that have yet to be adequately characterized by other PAP models. These interactions may be home to amplified versions of dynamics that are typically found in any relational form of psychotherapy, including transferences, projection, or reenactments of traumatizing dynamics or events. As in talk therapy, the appearance of these phenomena represents an opportunity for clinical gain if handled skillfully or rupture and harm if handled poorly. EMBARK therapists are thus trained to work ethically and efficaciously in this domain through the didactic and experiential processes discussed later under the Ethically rigorous care cornerstone. In the treatment of MDD, for example, these relational events are framed as potential moments of relational repatterning that may reduce depressive symptoms. A core element of depression is social isolation, both actual and felt. This isolation may derive from patterns or beliefs learned in early life relationships, such as a sense that one is unacceptable, deserves to be alone, or may lose love if they express themselves freely in the presence of another person. The altered relational dynamics of a PAP medicine session may provide opportunities for relational repatterning that supplants these maladaptive beliefs. EMBARK therapists are also prepared to support relational benefits that may arise for the participant outside of their interactions with the therapists, such as an internally felt sense of social connectedness or emotional empathy or an autobiographical review process that examines past and present relationships in the participant's life. If these shifts in social cognition occur and are skillfully worked with in the integration phase, a depressed participant can be helped to use them as the grounds for a less isolative social life.

(6) Keeping Momentum. A course of PAP treatment is brief, and its most enduring benefits have been conceptualized as those that continue to unfold well beyond the final session. PAP participants often emerge from a medicine session with a sharp uptick in the sense of motivation, self-efficacy, and commitment to making pro-therapeutic changes to their behavior or life context. Some may also develop a clarified sense of their deeply held values, which may form the basis for beneficial post-treatment actions. At the neural level, it has been suggested that the plasticity brought about by many psychedelic medicines may signal the reopening of a social reward critical learning period for weeks after administration, suggesting that pro-therapeutic changes may take root during a time period that extends beyond the end of what is typically considered integration. The EMBARK approach recognizes this unique opportunity. Therapists are trained to support a participant's movement from the setting of intentions to the planning of concrete actions by attending to importance of post-treatment changes throughout all stages of PAP treatment. For MDD or any other indication under study, these changes are expected to look very different for each participant. EMBARK therapists are trained to apply a broad lens to what helpful post-treatment change might look like. For some participants, the most supportive change may be at the level of personal behaviors, such as problem drinking or procrastination. For others, change may be warranted in their personal contexts, such as relationships or work environments. Some participants may find additional benefit in taking aim at collective concerns that have a bearing on their life, such as structural racism or exploitative work conditions, through participation in collective organizing. The EMBARK approach recognizes the potential of both individual and collective forms of change in the service of enhancing a participant's psychological, spiritual, and social wellbeing.

The EMBARK model adheres to the three-phase PAP treatment design that has been used in all clinical trials published to date. It involves non-drug preparation sessions prior to administration (Pre-Administration Psychological Support Sessions), medicine sessions in which the psychedelic medicine is administered (Psychological Support During Treatment Sessions), and non-drug integration sessions after the date of administration (Post-Administration Psychological Support Sessions).

For the sessions within each phase, therapists are given a set of general tasks, as well as domain-specific tasks for each of the six domains. For the preparation and medicine phases, these two sets of tasks are woven together into suggested agendas for each session, which therapists can apply with flexibility and responsiveness to the needs of a specific participant. In the integration phase, therapists and participants collaboratively choose which integration goals they will pursue based on their pertinence to what arose in the participant's medicine session. Each indication-specific EMBARK treatment manual provides guidance on which integration goals to consider with the participant based on the specific treatment events that arose during the medicine session. The way in which EMBARK's eclectic, six-domain approach comes together into a unified approach across the three phases of treatment is described below.

Preparation Sessions. All EMBARK protocols developed so far have included three preparation sessions leading up to each medicine session, though this may change if called for by the indication under study. The therapists' general aims for this phase have included building rapport and trust, learning about the participant's experience of their mental health challenges, explaining basic elements of PAP treatment and what to expect from the medicine session, providing preparatory instructions about diet and aftercare, and responding to participant questions about PAP treatment. Therapists are also given domain-specific tasks in the preparation phase, which may vary across protocols in response to the specific proposed mechanisms of change for the clinical indication under study. These tasks lay the groundwork for the participant to receive benefit within whichever of the six domains become important for them during the medicine and integration phases. Every participant is prepared for potential benefit across all six domains because EMBARK therapists make no determination before the medicine session about which domains may or may not become relevant later in treatment, despite any prediction or wish held by them or the participant during this phase. Even if a participant frames their disease or disorder (e.g., MDD) in, for example, primarily spiritual terms and expresses a desire to address their symptoms within a spiritual framing, it is still very possible that treatment benefits will occur for them in other domains, in addition to or instead of the “E” domain, if they are properly prepared. During preparation, the participant's lived experience of their symptoms and their functional significance are assessed, but no attempt is made by therapists to use this information to focus the participant's treatment on a specific subset of psychedelic-induced phenomena during the medicine session. However, this information is taken into consideration later during the integration phase when deciding what integration goals would be most supportive. Since it is unknown during the preparation phase which domains and domain-associated mechanisms of change will be most relevant to the specific participant, therapists' role in this phase tends to be more standardized than it is in later phases. However, while preparation tasks are fixed in their intent, therapists are invited to bring in interventions from their preferred clinical orientation(s), as long as they conform to the guidelines that ensure that the intended utility of each task is conferred to the participant. For example, therapists tasked with teaching basic mindfulness skills to a participant may do so using mindfulness-based tools from ACT, DBT, other mindfulness-based EBTs, or a meditative spiritual tradition, provided that the tool meets the following criteria set forth in the EMBARK manual: (1) it invites the participant to cultivate a receptive, attentive state, (2) does not contain elements that could potentially clash with a participant's religious beliefs, (3) and abides by trauma-informed practices detailed in the manual. The manual also provides example interventions for therapists who do not have relevant expertise to bring in for any given task. Ethical considerations based on the four care cornerstones are woven throughout the guidelines set forth for therapist interventions. For example, the guidelines for the discussion of therapist-participant dynamics requires that it include an exploration of cultural dynamics, in line with the cornerstone of culturally competent care.

Medicine Sessions. At the outset of a medicine session, the therapists maintain an agnosticism about which benefits and which domains will ultimately become most salient for a participant, much as they did in the preparation phase. The pre-dosing therapist tasks thus serve a similar purpose to the tasks in the preparation phase in that they set the stage for phenomena in any domain to arise and bring the potential for benefit. These stage-setting tasks are woven together into a suggested pre-dosing agenda for the medicine phase that includes a collaborative ritual (E), a check-in about intentions (K), and a brief somatic awareness practice (B). The six EMBARK domains thus continue to serve therapists as a conceptual frame for laying the groundwork for a broad variety of pro-therapeutic outcomes. However, once a psychedelic medicine is administered, the therapists' role becomes more responsive to the specific situation and less about a domain-agnostic approach to preparation. At this point, the course of a participant's treatment starts to come into focus, and specific EMBARK domains present themselves as more pertinent to the participant's experience of the medicine. EMBARK's six-domain framework comes to serve therapists in a new way as a practical rubric for helping them to characterize in-session events and determine what domain-specific interventions these events might call for. For example, if they observe the participant experiencing what they identify as a somatic trauma process, they can recall the training they received in the Body aware domain, and apply the interventions associated with it. Support in identifying and responding to these events is provided in the EMBARK training program and in each indication-specific EMBARK manual.

The value of organizing the preparation work by domains becomes clear during this phase, as many of the responsive interventions applied in a particular domain will draw upon work that was conducted with the participant during the preparation phase in that same domain. For example, a reminder to “move toward” challenging emotional material will draw from preparatory psychoeducation provided in the Affective-cognitive domain, or a reminder to use a somatic self-soothing technique in response to intense somatic events will rely on what was taught during preparation in the Body aware domain. Therapists' incorporation of their own favored interventions during the preparation phase also ensures that they will be working squarely within their competence during this more unpredictable phase as well.

Integration Sessions. At this point, the therapists and participant are likely to have a strong sense of the most beneficial direction for a participant's continued therapeutic progress. During the first debrief session, all parties collaboratively determine what treatment goals might be best to work toward in integration. This process entails (1) debriefing and supporting the participant's sense of what transpired in the medicine session, (2) relating material that arose in the medicine session to their symptoms and treatment goals, (3) collaboratively identifying new attitudes, beliefs, behaviors, values, or other subjective shifts that may contribute to symptom reduction, and (4) planning post-treatment changes that may support and sustain this outcome. A set of suggested integration goals are provided in each indication-specific manual, along with guidelines and suggestions for working toward each of these goals. These goals are organized by domain to provide continuity with events that arose in the medicine session. Together, therapists and participants choose a subset of these goals, or develop their own, as long as they are based on a clear clinical rationale. This selection process is guided primarily by what transpired in the medicine session using a tool provided in each EMBARK manual. For instance, a participant who had an experience that they identify as spiritual may benefit from support in advancing their spiritual self-development or spiritual practices (E), or a participant who had a strong emotional opening might be best served by continued processing and reflection that may lead to revised core beliefs or a sustained movement away from emotional avoidance (A). However, the selection process is also informed by the participant's previously stated intentions for treatment and the functional meanings of their symptoms. For instance, if a depressed participant initially framed their suffering in terms of loneliness and set an intention of understanding their isolation, it might benefit them to consider goals in the Relational domain, even if nothing observable transpired in that domain during the medicine session. All integration goals are framed in terms of three possible spheres of change: individual behavior, personal context, and broader context. Individual behavior changes may include stopping a maladaptive behavior, changing an old behavior, or adopting a new practice. Personal context changes may include updates to social, vocational, or physical contexts that support treatment benefit, such as moving away from a social circle that encourages problem drinking or moving into a vocational field more congruent with one's revised personal values. The broader context refers to structural, cultural, or economic conditions that have real, mental health consequences for the individual. Participants who decide that change at this level would be supportive for them may benefit from taking collective action (e.g., community activism, labor organizing) that addresses broader conditions in a way that feels congruent with their revised values or sense of self and/or serve as a form of socialization or behavioral activation. This may have acute benefit for them while also contributing to the amelioration the conditions that had engendered or exacerbated their distress in the first place (e.g., exploitative work conditions). Participants and therapists determine together which of the three spheres might be appropriate foci for post-treatment changes that support participant wellbeing. The number of integration sessions is left to the discretion of the group employing EMBARK, though all EMBARK manuals written to date have included a standard of three integration sessions per medicine session. It is suggested that, whatever the intended number may be, therapists and participants both use their judgment to determine if additional sessions would be supportive of the participant's wellbeing and to undergo these sessions whenever possible. Otherwise, therapists working within the EMBARK approach are required to be prepared to make appropriate outside referrals if a participant requires ongoing therapeutic support. While EMBARK was designed to support short-term PAP interventions in clinical trial settings, aftercare may also be considered or needed to properly support the patient after their participation in the trial or other treatment settings.

### Psychological Support Provided Remotely

In some embodiments, psychological support may be provided remotely to a subject. For example, a therapist providing psychological support may not be in the same room, the same building, or in the same facility as a subject. Remote psychological support may be provided, for example by telephone (i.e., by voice call), by video call or video conference, by text, or by email.

In some embodiments, a pre-administration therapy session is conducted remotely. In some embodiments, a post-administration therapy session (e.g., an integration session) is conducted remotely.

In some embodiments, psychological support is provided remotely during the subject's treatment session. For example, in some embodiments, the subject takes the compound of Formula (I-3), or a pharmaceutically acceptable salt, polymorph, or solvate thereof, in his or her own home, and a therapist provides psychological support by voice call, video call, text, email, etc., for the hours (e.g., 1-8 hours, 2-8 hours, 4-8 hours, 1-2 hours, 1-3 hours, etc.) after the subject has taken the drug. In some embodiments, the subject takes the compound of Formula (I-3), or a pharmaceutically acceptable salt, polymorph, or solvate thereof, in an administration facility as described herein, and the therapist provides psychological support to the subject a therapist provides psychological support by voice call, video call, text, email, etc., for the hours (e.g., 1-8 hours, 2-8 hours, 4-8 hours, 1-2 hours, 1-3 hours, etc.) after the subject has taken the drug.

In some embodiments, remote psychological support is provided to the subject using a digital or electronic system. In some embodiments, the digital or electronic system may comprise one or more of the following features: 1) The digital or electronic system securely connects patients with one or more therapists or physicians for “virtual visits.” These virtual visits may be introductory or routine; 2) The digital or electronic system allows a subject to qualify, prequalify, or register for a clinical trial, or a psychological support session; 3) The digital or electronic system is configured to help therapists and/or physicians manage and interact with patients, for example, the electronic system may allow the therapist to share documents with subjects, keep notes about sessions, or schedule future sessions; 4) The digital or electronic system is configured to provide alerts for crisis intervention, for example, the digital or electronic system may allow the subject to contact the therapist if they are feeling anxiety or otherwise urgently need to talk to the therapist; 5) The digital or electronic system is configured to help prepare the subject for a visit with their therapist and/or physician, for example, the digital or electronic system may contain information regarding drug (e.g., psilocybin or a deuterated form thereof), the therapeutic protocol, etc.; 6) The digital or electronic system is configured to allow the therapist to provide psychological support during the subject's treatment session, for example, the system may comprise a video calling or chat feature; 7) The digital or electronic system is configured to allow the therapist to provide psychological support during a post-administration session (e.g., an integration session); 8) The digital or electronic system is configured to track the subject's adherence to the treatment regimen or goals; 9) The digital or electronic system is configured to assess one or more clinical endpoints in the subject, for example the system may comprise one or more questionnaires or exercises for the subject to complete, and the results may be made available to the subject's physician and/or therapist.

In some embodiments, the digital or electronic system is an “app” for use on a mobile phone or a computer. In some embodiments, the digital or electronic system is a website. In some embodiments, the digital or electronic system comprises a “chat” feature which allows communication between the subject and the therapist in real time. In some embodiments, the website comprises a video calling feature, which allows for the therapist to communicate with the subject using video communication. In some embodiments, the digital or electronic system is configured to allow a single therapist to provide psychological support to one or more subjects at or around the same time.

In some embodiments, psychological support sessions may be pre-recorded (e.g., audio or video recording) and provided to the subject for use at the subject's convenience via the digital or electronic system.

### Administration Facility, “Set and Setting”

As used herein, the term “set and setting” refers to the subject's mindset (“set”) and the physical and social environment (“setting”) in which the user has the treatment session. In some embodiments, the compound of Formula (I-3), or a pharmaceutically acceptable salt, polymorph, or solvate thereof, may be administered in a particular set and setting. In some embodiments, the set and setting is controlled, to the extent possible, to maximize therapeutic benefit of the treatment session.

In some embodiments, administration is performed in a facility specifically designed for treatment. Administration to the subject in a facility where the subject feels safe and comfortable may help ease anxiety in the subject, and may facilitate maximum clinical benefit. Administration may be performed, for example, in the subject's home or at a clinical facility.

In some embodiments, administration is performed in a facility (e.g., a room) with a substantially non-clinical appearance, for example, in a room that comprises soft furniture (e.g., plush couches, chairs, or pillows) and/or plants. In some embodiments, the room may be decorated using muted colors (e.g., greyed, dulled, or desaturated colors). In some embodiments, the light in the room is dimmed and/or light levels are kept or adjusted to be relatively low. In some embodiments, the room lighting is adjusted for intensity and/or color. In some embodiments, a virtual reality or augmented reality system (e.g., computer with visual/graphical and auditory outputs) is used. In some embodiments, the room comprises a sound system, for example a high-resolution sound system. In some embodiments, the sound system can allow for simultaneous ambient and earphone listening. In some embodiments, the subject may bring meaningful photographs or objects into the administration room.

In some embodiments, the room comprises a couch. In some embodiments, the room comprises a bed. In some embodiments, the room comprises more than one couch or bed, such as 2, 3, 4, 5, 6, 7, 8, 9, or 10 couches or beds. In some embodiments, the subject sits on or lies in the couch or bed for approximately 1 hour, 2 hours, 3 hours, 4 hours, 5 hours, 6 hours, 7 hours, 8 hours, or any range therebetween such as 4-8 hours or 2-4 hours, or a substantial fraction thereof, immediately after administration. In some embodiments, the subject listens to music for approximately 1 hour, 2 hours, 3 hours, 4 hours, 5 hours, 6 hours, 7 hours, 8 hours, or any range therebetween such as 4-8 hours or 2-4 hours, or a substantial fraction thereof, immediately after administration. In some embodiments, the subject wears an eye mask for approximately 1 hour, 2 hours, 3 hours, 4 hours, 5 hours, 6 hours, 7 hours, 8 hours, or any range therebetween such as 4-8 hours or 2-4 hours, or a substantial fraction thereof, immediately after administration. In some embodiments, the subject is provided with a weighted blanket.

In some embodiments, each subject is supervised by one therapist during the treatment session. In some embodiments, each subject is supervised by more than one therapist during the treatment session, such as two therapists, three therapists, four therapists, or five therapists. In some embodiments, one therapist may supervise multiple subjects, wherein each subject is participating in a treatment session. For example, one therapist may supervise two, three, four, five, six, seven, eight, nine, or ten subjects.

Embodiments of the disclosure include use of additional tools and/or technique(s) with dosage/administration, including various transcranial magnetic stimulation (TMS) methods and protocols, for example, prior or subsequent to one or more dosing(s), biofeedback devices, etc.

Some embodiments can be used with a digital health product or digital solution. Teachings of the disclosure include utilization of such digital health products and/or related digital biomarkers as diagnostic and/or prognostic tools for patient monitoring and management pre-treatment, during treatment, and/or post treatment. Digital biomarkers can include, but are not limited to number of and/or time of phone calls/e-mails/texts; word length in text communication; gestures used (taps, swipes, or other); gyroscope derived information e.g. orientation of the phone; acceleration of the phone; keystroke patterns; location derived information from GPS; facial expressions and/or microexpressions; voice or vocal markers; natural language processing; social media use; sleep patterns; specific words or emojis used or not used; and/or the like. For example, in one embodiment, a digital health product can be utilized to determine dosing amount and/or dosing frequency, indicator of a need for re-dosing, re-dosing amount, a warning or alert, as tracking of compliance, etc.

In some embodiments, methods of treatment can include providing a clearance time for a subject or patient, such one or more medications is not present or substantially cleared from the system of the subject/patient. For example, methods of treatment can be configured such that, upon administration, the subject is not taking other serotonergic medications such as: selective-serotonin reuptake inhibitors, selective norepinephrine reuptake inhibitors, tricyclic anti-depressants, monoamine oxidase inhibitors and/or antipsychotics. In some embodiment, the method of treatment includes treatment concurrently with one or more medications, including but not limited to selective-serotonin reuptake inhibitors, selective norepinephrine reuptake inhibitors, tricyclic antidepressants, and/or monoamine oxidase inhibitors. In some embodiments, the method includes treatment such that subjects or patients take concomitant compounds or medications, including but not limited to benzodiazepines, cannabidiol (CBD) and/or other cannabinoids (e.g., THC (tetrahydrocannabinol); THCA (tetrahydrocannabinolic acid); CBD (cannabidiol); CBDA (cannabidiolic acid); CBN (cannabinol); CBG (cannabigerol); CBC (cannabichromene); CBL (cannabicyclol); CBV (cannabivarin); THCV (tetrahydrocannabivarin); CBDV (cannabidivarin); CBCV (cannabichromevarin); CBGV (cannabigerovarin); CBGM (cannabigerol monomethyl ether); CBE (cannabielsoin); CBT (cannabicitran); and/or the like), magnesium, levomefolic acid, e.g., for a period of time prior to, just prior to, and/or at the same time as receiving the compound of Formula (I-3), or a pharmaceutically acceptable salt, polymorph, or solvate thereof. In some embodiments, the methods of the present disclosure allow the patient being treated to remain on any ongoing antidepressant medications (e.g., SSRIs, SNRIs, etc.) throughout the treatment course. For example, the patient to be treated with the compound of Formula (I-3), or a pharmaceutically acceptable salt, polymorph, or solvate thereof, may be on a stable dose of an antidepressant medication, e.g., an SSRI such as escitalopram.

In some embodiments, the method includes treatment such that a subject has not taken one or more medications, particularly has not taken one or more serotonergic medications for at least 2 days, at least, 3 days, at least 4 days, at least 5 days, at least six days, at least 1 week, at least 2, 3, or 4 weeks before administration of the compound of Formula (I-3), or a pharmaceutically acceptable salt, polymorph, or solvate thereof. In some embodiments, the method includes treatment such that a subject has taken one or more medications, particularly has taken one or more serotonergic medications for at least 2 days, at least, 3 days, at least 4 days, at least 5 days, at least six days, at least 1 week, at least 2, 3, or 4 weeks before administration of the compound of Formula (I-3), or a pharmaceutically acceptable salt, polymorph, or solvate thereof.

Embodiments of the disclosure include methods utilizing a digital biomarker, for example, as a diagnostic and/or prognostic tool for patient management pre-, during and/or post treatment, wherein the digital biomarker is one or more biomarkers associated with executive function, cognitive control, working memory, processing speed, and/or emotional valence.

In some embodiments, the digital biomarker is identified from patterns in smartphone use such as swipes, taps, and other touchscreen activities, and can be scientifically validated to provide measurements of subject status, such as cognition and mood, including, by way of non-limiting example, as disclosed in one or more of the following, each of which is herein expressly incorporated by reference for all purposes: US20170086727, US20170258382, US20170258383, US20170287348, U.S. Ser. No. 10/148,534, U.S. Pat. No. 9,737,759, and/or U.S. Ser. No. 10/231,651.

Biomarkers which may serve as a diagnostic and/or prognostic tool for patient management pre, during and/or post treatment may be identified using one or more of: number of and/or time of phone calls/e-mails/texts; word length in text communication; gestures used (taps, swipes, or other); gyroscope derived information e.g. orientation of the phone; acceleration of the phone; keystroke patterns; location derived information from GPS; facial expressions and/or microexpressions; voice or vocal markers; natural language processing; social media use; sleep patterns; specific words or emojis used or not used; and/or the like. In some embodiments, health components and/or connected biomonitors and/or smart devices/wearables can be utilized to collect information to be used in diagnostic and/or prognostic outputs. For example, in some embodiments, a heart rate monitor or similar device can collect a subject's data and heart rate variability (for example only, as disclosed in U.S. Pat. No. 10,058,253, the entirety of which is herein incorporated by reference) can be used to assess/determine a metric relating to the subject's current emotional state, relative change in emotional state, etc., which can be used in determining a new or follow-on treatment plan, adjusting a treatment plan, etc.

In accordance with a further aspect of the disclosure there is provided a method of assessing a subject pre, during and/or post treatment of a central nervous system disorder to determine whether to provide treatment or a further treatment comprising monitoring one or more biomarkers associated with executive function, cognitive control, working memory, processing speed, and emotional valence, and determining the treatment based on an outcome. The method can further comprise the step of administering the compound of Formula (I-3), or a pharmaceutically acceptable salt, polymorph, or solvate thereof, for a first or a subsequent time.

In some embodiments, the biomarker is identified from patterns in smartphone use such as swipes, taps, and other touchscreen activities, and are scientifically validated to provide measurements of cognition and mood. For example, in some instances, the pattern is identified using one or more of: number of and/or time of phone calls/e-mails/texts; word length in text communication; gestures used (taps, swipes, or other); gyroscope derived information e.g. orientation of the phone; acceleration of the phone; keystroke patterns; location derived information from GPS; facial expressions and/or microexpressions; voice or vocal markers; natural language processing; social media use; sleep patterns; specific words or emojis used or not used; and/or the like.

Embodiments include a method of assessing a subject pre, during and/or post treatment of a central nervous system disorder to determine whether to provide treatment or a further treatment with the compound of Formula (I-3), or a pharmaceutically acceptable salt, polymorph, or solvate thereof, comprising monitoring one or more biomarkers associated with executive function, cognitive control, working memory, processing speed, and emotional valence, and determining the treatment based on an outcome; the method can further comprise administering the compound of Formula (I-3), or a pharmaceutically acceptable salt, polymorph, or solvate thereof, for a first or a subsequent time.

In some embodiments, the disclosure provides for treating 2 or more subjects, the method comprising administering to each subject a therapeutically effective amount of the compound of Formula (I-3), or a pharmaceutically acceptable salt, polymorph, or solvate thereof, at the same time or substantially the same time (e.g., dosed within several minutes of each other, within 5, 10, 15, 20, 25, or 30 min of each other), wherein each subject is aware of the other subject also receiving treatment. In some embodiments, the subjects are in the same room. In some embodiments, the subjects are in different rooms.

In some embodiments, the disclosure provides a method of treating a subject, the method comprising administering to the subject a therapeutically effective amount of the compound of Formula (I-3), or a pharmaceutically acceptable salt, polymorph, or solvate thereof, and providing a virtual reality/immersive reality digital tool. In some embodiments, the light in the room is dimmed and/or light levels are kept or adjusted to be relatively low. In some embodiments, darkened glasses or eye shades are provided. In some embodiments, the room lighting is adjusted for intensity and/or color. In some embodiments, a virtual reality or augmented reality system (e.g., computer with visual/graphical and auditory outputs) is used.

### Subjects

In some embodiments, the subject is a male. In some embodiments, the subject is a female. In some embodiments, the female subject is pregnant or post-partum. In some embodiments, the subject is attempting to reduce or eliminate their use of a pharmaceutical agent, such as an antidepressant or an anti-epileptic drug. In some embodiments, the subject is attempting to reduce or eliminate their use of the pharmaceutical agent before becoming pregnant, having surgery or other medical procedure, or starting to use a different pharmaceutical agent.

The subject may be a geriatric subject, a pediatric subject, a teenage subject, a young adult subject, or a middle aged subject. In some embodiments, the subject is less than about 18 years of age. In some embodiments, the subject is at least about 18 years of age. In some embodiments, the subject is at least about 21 years of age. In some embodiments, the subject is about 5-10, about 10-15, about 15-20, about 20-25, about 25-30, about 30-35, about 35-40, about 40-45, about 45-50, about 50-55, about 55-60, about 60-65, about 65-70, about 70-75, about 75-80, about 85-90, about 90-95, or about 95-100 years of age, or any range between any of the aforementioned ages.

The subject may have a chronic disease or a terminal disease. In some embodiments, the subject may have a life-altering disease or condition (such as the loss of a limb or onset of blindness).

The subject may have recently been diagnosed with a disease, disorder, or condition. For example, the subject may have been diagnosed within 1 month, within 3 months, within 6 months, or within 1 year. In some embodiments, the subject may have been living with a disease, disorder, or condition for an extended period time, such as at least 6 months, at least 1 year, at least 3 years, at least 5 years, or at least 10 years.

In some embodiments, the subject may be a cancer patient, such as a Stage 4 or terminal cancer patient. In some embodiments, the subject may have been determined to have a limited time to live, such as less than 1 year, less than 6 months, or less than 3 months.

The subject may have previously taken a psychedelic drug, or may have never previously taken a psychedelic drug. For example, the subject may or may not have previously taken psilocybin, a psilocybin mushroom (“magic mushroom”), LSD (lysergic acid diethylamide or acid), mescaline, or DMT (N,N-dimethyltryptamine).

In some embodiments, the subject may have previously taken one or more serotonergic antidepressants (e.g., selective serotonin reuptake inhibitors (SSRIs), SNRIs, etc.). In some embodiments, the subject has never previously taken a serotonergic antidepressant. In some embodiments, the subject has not taken any serotonergic antidepressants for at least 2 weeks, at least 4 weeks, or at least 6 weeks prior to receiving treatment with the compound of Formula (I-3), or a pharmaceutically acceptable salt, polymorph, or solvate thereof. In some embodiments, the subject treated with the methods herein is taking an antidepressant medication such as a serotonergic antidepressant as part of ongoing treatment. For example, the subject to be treated herein may be taking a stable chronic dose of antidepressant medication(s). In some embodiments, the subject treated with the methods herein is taking a sedative or hypnotic as part of ongoing treatment.

In some embodiments, the subject may have previously received electroconvulsive therapy (ECT). In some embodiments, the subject has not received any ECT for at least 2 weeks, at least 4 weeks, or at least 6 weeks prior to receiving treatment with the compound of Formula (I-3), or a pharmaceutically acceptable salt, polymorph, or solvate thereof.

The subject may have a medical condition that prevents the subject from receiving a particular medical therapy (such as an SSRI or ECT). In some embodiments, the subject may have previously had an adverse reaction to a particular medical therapy (such as an SSRI or ECT). In some embodiments, a prior medical therapy (such as an SSRI or ECT) was not effective in treating a disease, disorder, or condition (e.g., MDD) in the subject.

In some embodiments, the subject has reduced monoamine oxidase type A activity, such as a genetic factor that is associated with reduced monoamine oxidase type A activity, e.g., a genetic mutation or genetic polymorphism of the monoamine oxidase type A gene or gene promoter that is associated with reduced monoamine oxidase type A activity. In some embodiments, the subject has reduced monoamine oxidase type A activity due to recent exposure to a monoamine oxidase inhibitor (MAOI). In some embodiments, the subject does not have reduced monoamine oxidase type A activity, for example the subject does not have a genetic factor that is associated with reduced monoamine oxidase type A activity, e.g., does not have a genetic mutation or genetic polymorphism of the monoamine oxidase type A gene or gene promoter that is associated with reduced monoamine oxidase type A activity. In some embodiments, the subject does not have reduced monoamine oxidase type A activity as a consequence of being recently exposed to a monoamine oxidase inhibitor (MAOI).

Also disclosed herein is a method for decreasing time of therapeutic onset relative to a psilocybin-based drug comprising administering a therapeutically effective amount of a compound/composition as disclosed herein (e.g., the compound of Formula (I-3), or a pharmaceutically acceptable salt, polymorph, or solvate thereof) to a patient in need thereof.

Also disclosed herein is a method of reducing psychedelic side effects relative to a psilocybin-based drug comprising administering a therapeutically effective amount of a compound/composition as disclosed herein (e.g., the compound of Formula (I-3), or a pharmaceutically acceptable salt, polymorph, or solvate thereof) to a patient in need thereof.

The terms “hallucinogenic side effects” and “psychedelic side effects” are used in the present disclosure interchangeably to refer to unwanted and/or unintended secondary effects caused by the administration of a medicament to an individual resulting in subjective experiences being qualitatively different from those of ordinary consciousness. These experiences can include derealization, depersonalization, hallucinations and/or sensory distortions in the visual, auditory, olfactory, tactile, proprioceptive and/or interoceptive spheres and/or any other perceptual modifications, and/or any other substantial subjective changes in cognition, memory, emotion and consciousness.

In some embodiments, the administration of the compound/composition (e.g. the compound of Formula (I-3), or a pharmaceutically acceptable salt, polymorph, or solvate thereof) causes no hallucinogenic and/or psychedelic side effects and/or less hallucinogenic and/or psychedelic side effects relative to a psilocybin-based drug. In some embodiments, the administration of the compound/composition (e.g. the compound of Formula (I-3), or a pharmaceutically acceptable salt, polymorph, or solvate thereof), alleviates, reduces, removes, and/or eliminates the hallucinogenic and/or psychedelic side effects caused by a psilocybin-based drug.

Also disclosed herein is a method of reducing dose related side-effects, e.g., nausea, relative to treatment with a psilocybin-based drug, comprising administering a therapeutically effective amount of a compound/composition as disclosed herein (e.g., the compound of Formula (I-3), or a pharmaceutically acceptable salt, polymorph, or solvate thereof) to a subject in need thereof. The compound/composition as disclosed herein (e.g. the compound of Formula (I-3), or a pharmaceutically acceptable salt, polymorph, or solvate thereof), has better brain penetration (i.e., a higher brain: plasma ratio) than that obtained from administration of psilocybin. As a result, the effective dosing for the compounds of the present disclosure can be lowered, thereby reducing dose related side effects such as nausea.

Also disclosed herein is a method of decreasing duration of effect relative to a psilocybin-based drug comprising administering a therapeutically effective amount of a compound/composition as disclosed herein (e.g., the compound of Formula (I-3), or a pharmaceutically acceptable salt, polymorph, or solvate thereof) to a patient in need thereof.

Generally, a duration of effect for a psilocybin-based drug is about 6-8 hours. In some embodiments, the duration of effect of the compound of Formula (I-3) is less than the duration of effect for a psilocybin-based drug. In some embodiments, the duration of effect of the compound of Formula (I-3) is 7 hours, 6 hours, 5 hours, 4 hours, 3 hours, 2 hours, 1 hour or less, or 50 minutes, 40 minutes, 30 minutes, 20 minutes, 10 minutes, 5 minutes or less. In some embodiments, the duration of effect of the compound of Formula (I-3) is less than the duration of effect of a psilocybin-based drug by 7 hours, 6 hours, 5 hours, 4 hours, 3 hours, 2 hours, 1 hour or less, or 50 minutes, 40 minutes, 30 minutes, 20 minutes, 10 minutes, 5 minutes or less.

The present disclosure also provides a kit comprising a pharmaceutical composition according to any embodiment described herein (e.g., a capsule comprising the compound of Formula (I-3), or a pharmaceutically acceptable salt, polymorph, or solvate thereof), and instructions for use in the treatment of a disease, disorder, or condition according to any embodiment described herein, e.g., instructions for use in the treatment of a depressive disorder (e.g., MDD), a substance use disorder, an anxiety disorder (e.g., OCD, PTSD, etc.), an eating disorder, pain (e.g., fibromyalgia), a headache disorder, etc. In some embodiments, the kit comprises two or more components that can be combined to form a pharmaceutical composition suitable for dosing, and instructions for use in the treatment of a disease, disorder, or condition according to any embodiment described herein. For example, kits may be provided which include a first component (e.g., a compound of Formula (I-3), or a pharmaceutically acceptable salt, polymorph, or solvate thereof) and a second component (e.g., a pharmaceutically acceptable aqueous medium), with instructions to combine the first component with the second component to form an oral liquid dosage form, and instructions for administering orally to the subject the oral liquid dosage form for the treatment of a disease, disorder, or condition according to any embodiment described herein, e.g., instructions for use in the treatment of a depressive disorder (e.g., MDD), a substance use disorder, an anxiety disorder (e.g., OCD, PTSD, etc.), an eating disorder, pain (e.g., fibromyalgia), a headache disorder, etc.

### Examples

### I. Compounds and Pharmaceutical Compositions of the Invention, and Analytical Methods to Describe Same

Psilocin-d₁₀ and pharmaceutically acceptable salts thereof according to any embodiment described herein can be prepared according to the methods described in WO2022195011 and WO2023078604.

Additionally, the analytical methods to describe Differential scanning calorimetry (DSC), X-ray powder diffraction (XRPD), Gravimetric Vapor Sorption (GVS)/Dynamic vapor sorption (DVS), Nuclear Magnetic Resonance (NMR), Thermogravimetric Analysis (TGA), Ultra Performance Liquid Chromatography (UPLC), High Resolution Mass Spectrometry (HRMS) and MS/MS are also described in WO2022195011 and WO2023078604.

### II. In Vivo Studies

### Predicted Dose Requirements

### Pre-Clinical Pharmacokinetics (PK)

To determine whether there are differences between psilocin after psilocybin and psilocin-d₁₀ pharmacokinetics to use for clinical pharmacokinetic simulations, pre-clinical data from male dogs and rats were studied and PK parameters determined after oral administration of psilocybin, psilocin, and psilocin-d₁₀. Due to differences in the molecular weight between the administered compounds and the analytes measured, results were converted to molar and normalized around a fixed dose to allow comparison of cohorts.

Calculations used for conversion of dose and concentration data were as follows:

$$Psilocybin :$$

$$Dose μmol / kg = dose mg / kg * 1000 / 284.25 μg / μmol$$

$$Concentration μmol / L = concentration ng * mL ( μg / L ) / 284.25 μ g / μmol$$

$$Psilocin :$$

$$Dose μmol / kg = dose mg / kg * 1000 / 204.27 μg / μmol$$

$$Concentration μmol / L = concentration ng * mL ( μg / L ) / 204.27 μ g / μmol$$

$$Psilocin - {d}_{10} :$$

$$Dose μmol / kg = dose mg / kg * 1000 / 214.33 μg / μmol$$

$$Concentration μmol / L = concentration ng * mL ( μg / L ) / 21 4.33 μ g / μmol$$

In Sprague Dawley rat studies, in comparison to psilocin after psilocybin, psilocin-d₁₀ yielded a lower oral exposure (112% reduction in AUC₀₋ₜ), a lower oral concentration (16% reduction in Cₘₐₓ), a reduced bioavailability (F) from 16% to 12% across all cohorts with comparable absorption kinetics (tₘₐₓ).

After oral administration of psilocybin or psilocin-d₁₀ at equivalent doses to male Beagle dogs, psilocin-d₁₀ produced a 38% higher exposure (AUC) and a 31% higher concentration (Cₘₐₓ) compared to psilocin after psilocybin. Oral bioavailabilities for psilocin-d₁₀ and psilocin after administration of psilocybin were 59% and 63%, respectively, with similar corresponding absorption kinetics (tₘₐₓ) at 0.5 hr.

A comparison of PK properties between psilocin and psilocin-d₁₀ was also determined in rats and dogs. Psilocin-d₁₀ was shown to have similar PK properties to psilocin when administered orally to rats, but with approximately 60% greater oral bioavailability (12.0% versus 7.5%). In dogs, oral availability of psilocin-d₁₀ was found to be much higher than in rats at 91.3% and 58.6%, female and male, respectively. Overall, psilocin-d₁₀ PK properties were similar to those of psilocin with about a 30% difference in systemic exposure in both rats and dogs.

Therefore, based on the pre-clinical reports it was concluded that psilocin-d₁₀ ranges between a decrease of 112% to an increase of 38% in AUC in comparison to oral psilocin after psilocybin and that psilocin-d₁₀ has similar PK properties as psilocin. There were inconclusive results between rats and dogs regarding changes in psilocin-d₁₀ exposure compared to psilocybin.

With the understanding that psilocin-d₁₀ preclinical PK properties are similar to those of psilocin and that psilocybin is rapidly converted to psilocin, existing clinical psilocin after psilocybin population pharmacokinetic models were deemed as base models. For psilocin-d₁₀ simulations, relative exposure boundaries of psilocin-d₁₀ were set by the preclinical studies ranging from −50% up to +60%, assuming psilocin and psilocin-d₁₀ absorption kinetics (tₘₐₓ) were similar.

Clinical Pharmacokinetics (PK) of Psilocin after Psilocybin

Psilocybin has been administered (oral and intravenous) to healthy subjects in previous literature reports and pharmacokinetic data has been collected. The previous literature reports from which data were studied/simulated herein are presented below:
Brown et al. 2017 (Brown R T, Nicholas C R, Cozzi N V, et al. (2017). Pharmacokinetics of escalating doses of oral psilocybin in healthy adults. Clin Pharmacokinet, 56(12), 1543-1554 (Clinical Trials Identifier NCT02163707)—This study involved three dosing levels (0.3, 0.45, and 0.6 mg/kg, equivalent to 25, 37.5 and 50 mg) of psilocybin, on which a population pharmacokinetic model was developed and a highest dose (0.6 mg/kg) identified that was given to humans in a clinical trial. The study involved a standardized breakfast time (time before dose is unknown), after which subjects walked 500 m to the study room, in which subjects were subjected to 15 minutes of centering meditation. Oral administration of an opaque methylcellulose capsule of psilocybin was then administered with 360 mL of water. No psilocybin was detected in plasma arguing for the rapid luminal and first pass formation. No weight relationship with clearance (CL) was identified in this study. It is noted that the reported AUC was incorrect compared to manual calculation and new analysis of the identical dataset in Dahmane E, Hutson P R, Gobburu J V S (2021). Exposure-response analysis to assess the concentration-QTc relationship of psilocybin/psilocin. Clin Pharmacol Dev 10(1):78-85;
Hasler et al. 1997 (Hasler F, Bourquin D, Brenneisen R, et al. (1997). Determination of psilocin and 4-hydroxyindole-3-acetic acid in plasma by HPLC-ECD and pharmacokinetic profiles of oral and intravenous psilocybin in man. Pharm Acta Helv, 72(3), 175-184)—this was a partial cross-over study with oral (between 10-20 mg) and intravenous (1 mg) administration of psilocybin. Psilocybin was administered after an overnight fast. Estimates of absolute bioavailability of psilocin after psilocybin (recalculated from publication) are as follows:

$${AUC}_{inf} after 1 mg intravenous dosing = 240 ng * min / mL$$

$$Cross over subjects only \text{:} 55 \% ( N = 3 ) ( individual values of 29.3 \% , 60.5 \% and 75.1 \% )$$

$$Summary statistics \text{:} 56.4 \% \pm 18.5 \% ( N = 6 ) ;$$

Holze et al. 2022 (Holze F, Ley L, Müller F, et al (2022). Direct comparison of the acute effects of lysergic acid diethylamide and psilocybin in a double-blind placebo-controlled study in healthy subjects. Neuropsychopharmacology, 47(6): 1180-87)—this was an extensive pharmacokinetic and pharmacodynamic study with LSD and two doses of psilocybin; oral doses (capsules) of 15 mg and 30 mg psilocybin dihydrate→13.83 mg and 27.66 mg psilocybin, respectively. The study was performed on 28 healthy subjects (14 Male/14 Female), which were dosed at 9:00 am, and a standardized breakfast (2 croissants) was served before substance administration.

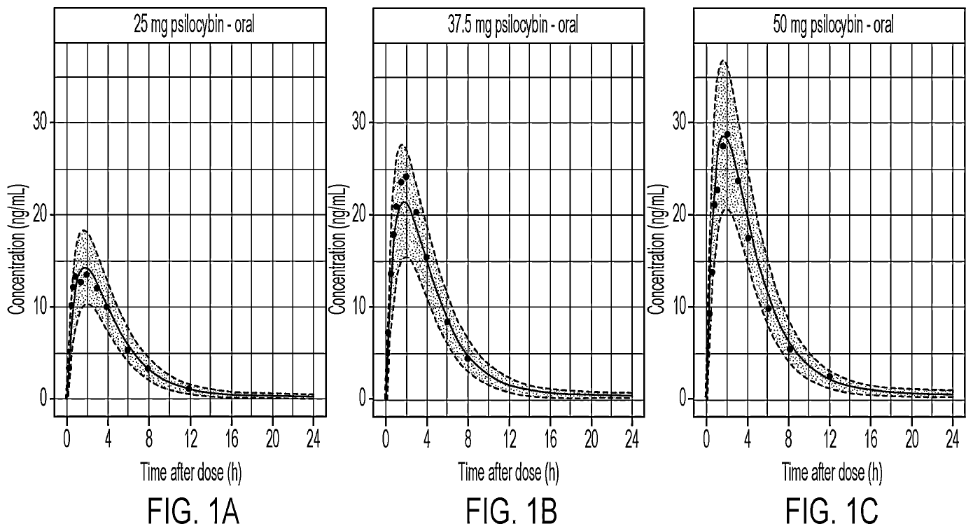

Simulations were performed herein on Brown et al. 2017 (N=12), in which body weight doses were converted to fixed doses, which were tested as non-significantly different in Brown et al. The model was recreated and used for simulation of the Brown et al. dosing levels. FIGS. 1A-1C show the Brown et al. 2017 simulations for concentration (ng/ml) versus time after dose (h) for 25 mg (FIG. 1A), 37.5 mg (FIG. 1B), and 50 mg (FIG. 1C) oral psilocybin, indicating correct reproduction of the population PK model and estimated model parameters. The model simulations correspond with published data for all dosing levels, with a slight underprediction of the 37.5 mg (0.45 mg/kg) dose.

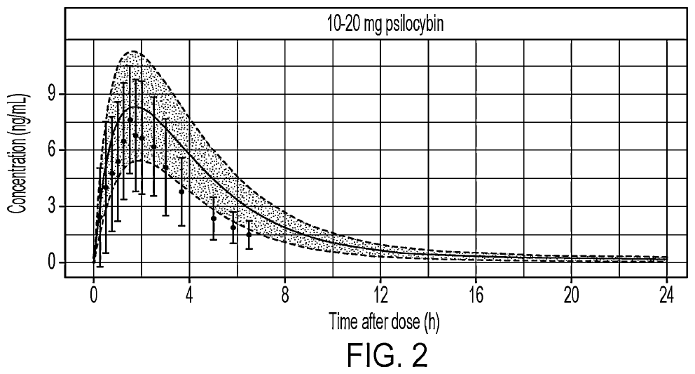

Individual oral doses of psilocybin administered by Hasler et al. 1997 (N=6) (ranging from 10-20 mg) were simulated herein as shown by the concentration (ng/mL) versus time after dose (h) plot of FIG. 2. The model (Brown et al. 2017) overlaps with Hasler et al. 1997 concentrations up to three hours postdose. In general, there was an overprediction of data, perhaps due to the overnight fast causing a lowering of the bioavailability. The variability (based on standard deviation) is comparable between studies.

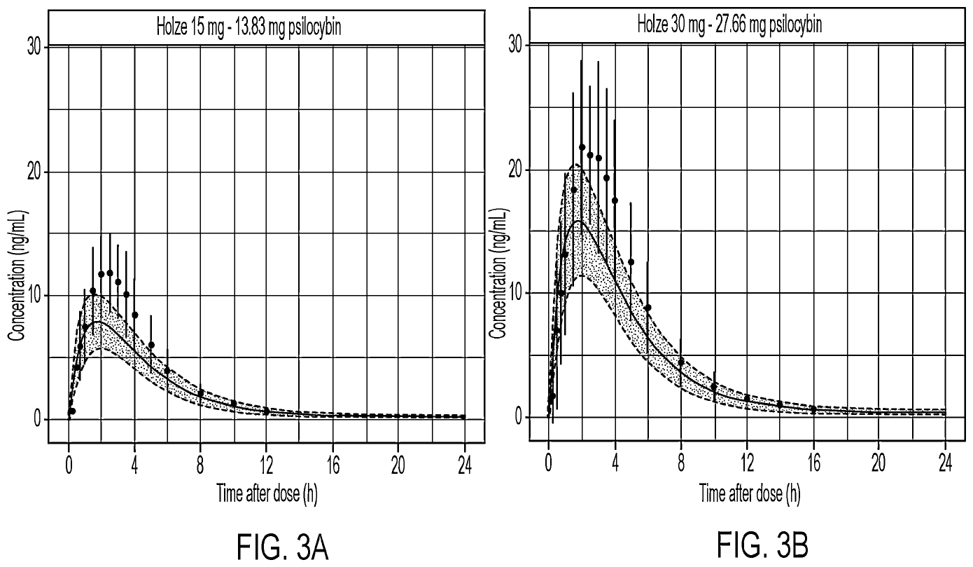

Oral doses of psilocybin (13.83 mg and 27.66 mg) administered by Holze et al. 2022 (N=28) were simulated herein as shown by the concentration (ng/ml) versus time after dose (h) plot of FIGS. 3A-3B. The model (Brown et al. 2017) underpredicts the Holze et al. 2022 data. The absorption profile is similar with comparable variability. Dosing in a fed state might have increased bioavailability. The variability (based on standard deviation) is comparable between studies.

In terms of bioavailability, a bioavailability (F: Fraction) comparison between these studies is presented in Table 3.

TABLE 3

| | Calculated F based | % relative | |
|---|---|---|---|
| | on reference AUC_{inf} | change | |
| | for intravenous* | from | |
| | Mean⁺ (SD) | Brown | |
| Study | [Mean per cohort] | et al. | Notes |
| Brown et al. | 83.7% | — | Reference population PK |
| 2017 | [83%, 90%, 78%] | | model |
| | | | Breakfast prior to dosing |
| Hasler et al. | 56.4% (18.48%) | −32.5% | Overnight fast |
| 1997 | | | |
| Holze et al. | 114.3% | +36.6% | Breakfast prior to dosing |
| 2022 | [110.3%, 118.4%] | | Highest observed F after |
| | | | oral dosing of psilocybin |
| | | | Higher AUC/mg |
| | | | compared to intravenous |
| | | | dose |

*Reference AUC_{inf} after 1 mg intravenous psilocybin Hasler et al. 1997 = 240 ng*min/mL
⁺Mean per cohort is given, if applicable

Manual calculation was required due to incorrect reporting in multiple publications. As can be seen, Holze et al. 2022 shows F above 100%. Reference Hasler et al. 1997 for AUC after intravenous administration is based on limited (N=6) sample size and may/will have a high level of uncertainty. Holze et al. 2022 is expected to approach a 100% F. Simulations will use a % relative change compared to Brown et al. 2017.

Based on these clinical PK literature reports and the simulations thereof presented here, it can be concluded that high quality data is available for psilocin after psilocybin administration, and a population PK model is able to simulate new dosing levels of different studies. Moreover, it can be concluded that inter-individual variability between studies was comparable, and that moderate variability in bioavailability was observed between studies, which may potentially be linked to food intake prior to dosing. Accordingly, the following assumptions were taken into account during simulations of psilocin-d₁₀: existing psilocin after psilocybin population pharmacokinetic models can be used as a base model for psilocin-d₁₀ simulations, and as psilocin-d₁₀ was given with orange juice instead of a tablet and in a fasted state, absorption may be faster but bioavailability may be either increased or decreased.

Clinical Pharmacodynamics (PD) of Psilocin after Psilocybin

To evaluate potential psychedelic effects at various doses of psilocin-d₁₀, data were summarized/modeled from published literature for psilocin after psilocybin administration. If only a psilocybin dose was given and no concentrations were available, the corresponding typical Cₘₐₓ values were simulated based on the Brown et al. 2017 population PK model.

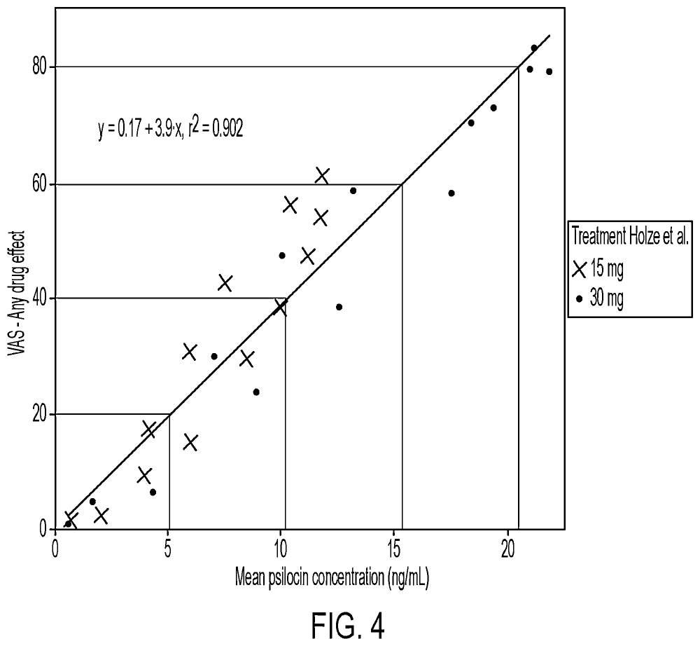

First, the VAS—Any drug effect score was modeled based on data from Holze et al. 2022 using a linear effect relationship to determine psilocin plasma concentrations in which a VAS-Any drug effect score of 20, 40, 60, and 80 mm was reached. This relationship is presented graphically in FIG. 4, based on the extraction of mean PK and PD data from the figures in Holze et al. 2022. Here, a linear concentration-effect relationship was identified with no evidence of hysteresis. As is evident from this modeled linear concentration-effect relationship according to Holze et al. 2022 (y=0.17+3.9x), a psilocin plasma concentration of about 15.34 ng/ml is the predicted amount required to achieve VAS—Any drug effect scores above 60 mm, a psilocin plasma concentration of about 17.9 ng/ml is the predicted amount required to achieve VAS—Any drug effect scores above 70 mm, and a psilocin plasma concentration of about 20.47 ng/ml is the predicted amount required to achieve a VAS—Any drug effect score of 80 mm. For comparison, Brown et al. 2017 reports that ingestion of 25 mg of psilocybin provides a mean Cₘₐₓ of about 15 ng/ml of psilocin.

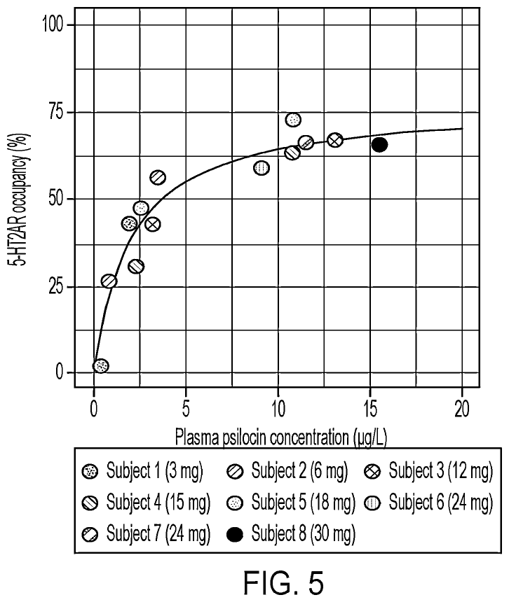

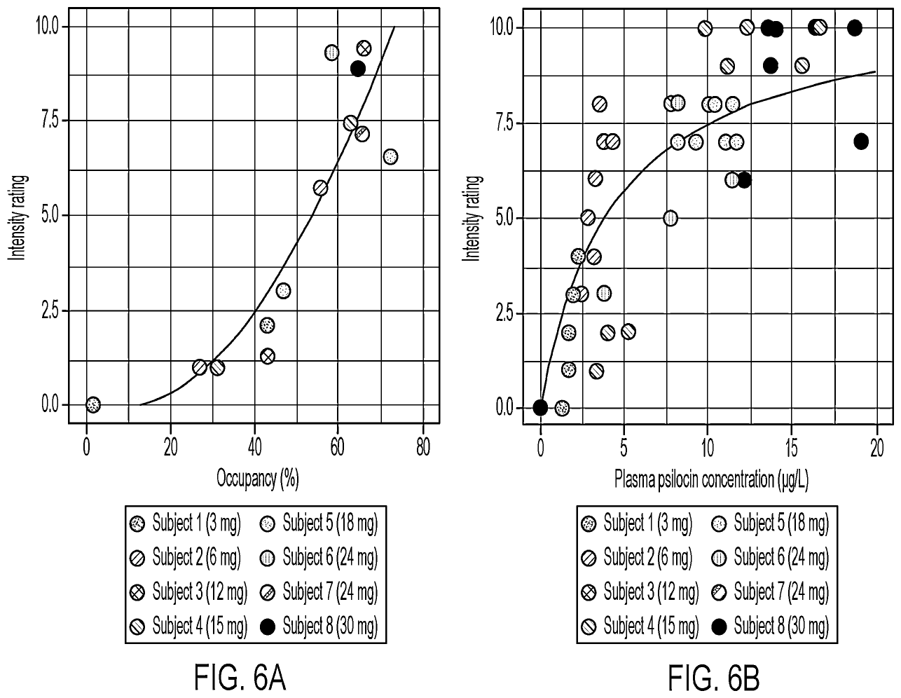

This modeled relationship between psilocin plasma concentration and VAS—Any drug effect score is also in agreement with reported literature studies relating plasma psilocin concentrations (μg/L) to neocortical serotonin 5-HT_{2A} receptor occupancy % and subjective intensity of the psychedelic experience (see Madsen M K, Fisher P M, Burmester D, et al (2019). Psychedelic effects of psilocybin correlate with serotonin 2A receptor occupancy and plasma psilocin levels. Neuropsychopharmacology, 44:1328-34), albeit the Madsen et al. study uses a VAS scoring system related specifically to psychedelic effects. The psilocin concentrations needed to reach the plateau in receptor occupancy and the required VAS scores (psychedelic effects) can be seen graphically in FIG. 5 and FIGS. 6A-6B, which are reproduced from Madsen et al. 2019 (FIGS. 3 and 4, respectively, in Madsen et al. 2019).

Pharmacodynamic effects, including VAS—Any drug effect, MEQ30, and 5D-ASC, over a range of reported/simulated psilocin Cₘₐₓ values after psilocybin are depicted in Table 4. This PK/PD data reiterates that a psilocin plasma concentration of about 15 ng/ml (equating to about a 25 mg oral dose of psilocybin) provides a VAS—Any drug effect score of 60, while higher oral doses providing about 20 ng/mL psilocin are required to achieve VAS—Any drug effect scores of 80. These concentration thresholds are used in subsequent simulated concentration (ng/ml) time after dose (h) profile plots of psilocin-d₁₀. Given the demonstrated clinical efficacy of 25 mg psilocybin (Goodwin et al., Single-Dose Psilocybin for a Treatment-Resistant Episode of Major Depression. N Engl J Med. 2022 Nov. 3; 387(18):1637-1648), a 60+ mm score on the VAS—Any drug effect is considered herein to be clinically meaningful.

TABLE 4
PK/PD data reported/simulated for psilocin after psilocybin

| Cₘₐₓ (ng/mL) | Description | | Reference |
|---|---|---|---|
| 1.83 | No change list of complaints + 5D-ASC | | Studerus et al. 2011^{a} |
| 5.08 | VAS-Any drug effect: | 20 | Holze et al. 2022 |
| 10.2 | VAS-Any drug effect: | 40 | Holze et al. 2022 |
| 13 | 5D-ASC total: | 19% | Holze et al. 2022 |
| 14.53 | MEQ30 total: | 66%+ | Griffiths et al. 2018^{b} and |
| | | | Holze et al. 2022 |
| 15.34 | VAS-Any drug effect: | 60 | Griffiths et al. 2018^{b} and |
| | | | Holze et al. 2022 |
| 20.47 | VAS-Any drug effect: | 80 | Holze et al. 2022 |
| 25 | 5D-ASC total score: | 29% | Holze et al. 2022 |
| 343 | Rat Cₘₐₓ at 40 | mg/kg | Internal dose range finding study |
| 51.7 | Dog Cₘₐₓ at 0.5 | mg/kg | Internal 4-week GLP |
| | | | toxicology study |

^{a}Studerus E, Kometer M, Hasler F, Vollenweider FX (2011). Acute, subacute and long-term subjective effects of psilocybin in healthy humans: a pooled analysis of experimental studies. J Psychopharmacol, 25(11):1434-52
^{b}Griffiths RR, Johnson MW, Richards WA, et al. (2018). Psilocybin-occasioned mystical-type experience in combination with meditation and other spiritual practices produces enduring positive changes in psychological functioning and in trait measures of prosocial attitudes and behaviors. J Psychopharmacol, 32(1), 49-69

### Pharmacokinetic (PK)/Pharmacodynamic (PD) Simulations of Psilocin-d₁₀

After the threshold plasma psilocin concentration causing psychedelic effects in humans was defined based on literature studies/modeling related to psilocybin administration, the objectives were to i) predict psilocin-d₁₀ concentration in humans by modifying existing population PK models of psilocybin, based on the pre-clinical psilocin-d₁₀ data, ii) simulate PK profiles of psilocin-d₁₀ to support phase 1 dose selection while investigating the impact of assumptions on absorption rate, bioavailability, and inter-individual variability, and iii) simulate multiple psilocin-d₁₀ dosing levels, assuming linear PK, covering subtherapeutic, therapeutic, and supratherapeutic concentrations to support dose escalation design.

The existing population PK model was modified to be used for psilocin-d₁₀ PK simulations with different assumptions. The following four simulation scenarios outlined in Table 5 were utilized to predict exposures of psilocin-d₁₀ compared to the Brown et al. 2017 population PK model.

TABLE 5

| Scenario | Change compared to Brown | |
|---|---|---|
| Description | et al. 2017 | Rationale |
| Scenario 1- | No change compared to Brown | Psilocin-d₁₀ absorption and |
| Identical | et al. 2017 | bioavailability behaves identical |
| | | to psilocin after psilocybin |
| | | administration in Brown et al. |
| | | 2017 |
| Scenario 2- | Increased absorption rate constant | Psilocin-d₁₀ behaves identical |
| Faster absorption | (from 0.367/h to 1/h), same | to psilocin after psilocybin but |
| | bioavailability | with a faster absorption |
| | | rate due to orange juice |
| | | administration |
| Scenario 3- | Increased absorption rate constant | Safety scenario where the |
| Faster absorption | (from 0.367/h to 1/h) and | highest Cₘₐₓ concentrations |
| and | AUC increase matches highest | are reached with a faster |
| Increased F | pre-clinical dog data (+60% | absorption compared to |
| | increase in F relative to psilocybin | literature and higher |
| | from Brown et al. 2017) | bioavailability |
| Scenario 4- | Reduced bioavailability based on | Fasted administration might |
| Decreased F | pre-clinical rat data and Hasler | reduce the bioavailability, |
| | et al. 1997 fasted | with comparable |
| | administration (−50%) | absorption kinetics |

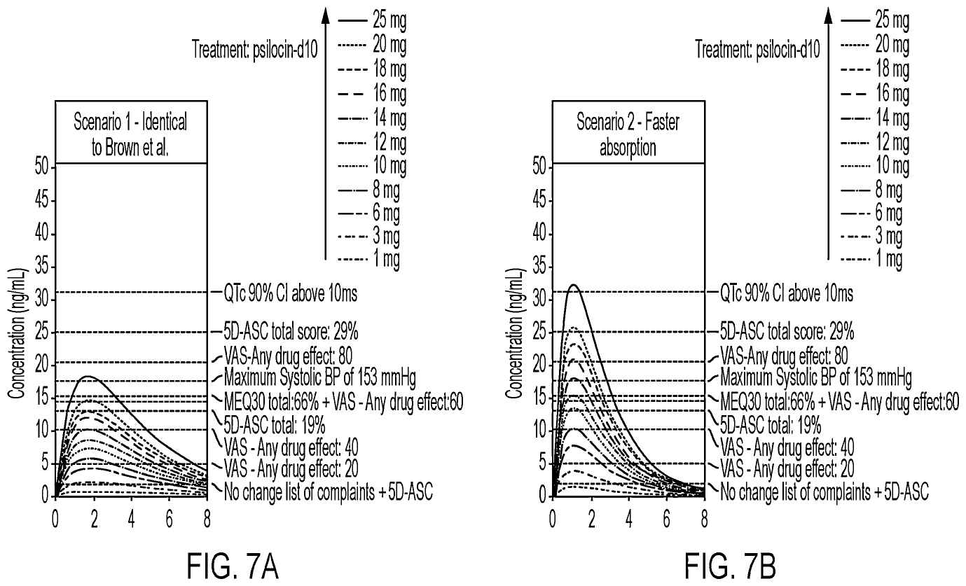

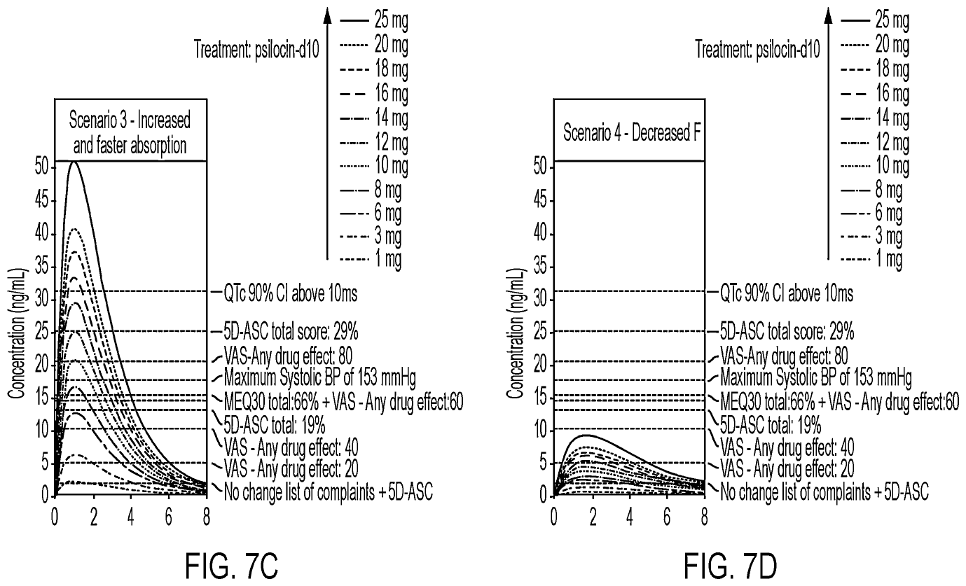

Scenarios 1 through 4 were used to simulate four psilocin-d₁₀ concentration (ng/ml) time after dose (h) profiles, as shown in FIGS. 7A-7D. These simulated profiles present the median of the simulated population (N=1000 per treatment), and were benchmarked with various PD thresholds including VAS—Any drug effect, MEQ30, and 5D-ASC, based on available data. The psilocin-d₁₀ concentrations are presented as psilocin concentrations for comparison with other figures and can be converted to psilocin-d₁₀ concentrations by multiplication of 1.049.

As is clear from these psilocin-d₁₀ PK simulations covering sub-therapeutic, therapeutic, and supratherapeutic levels, 1 mg and 3 mg were assumed to result in sub-therapeutic concentrations based on all explored scenarios. Further, the investigated psilocin-d₁₀ dosing range of initial cohorts (up to 14 mg psilocin-d₁₀ in Scenario 3) remains below the investigated psilocin concentrations after psilocybin previously investigated (0.6 mg/kg, Brown et al 2017).

Based on these simulations with the available data, study psilocin-d₁₀ administration was anticipated to follow PK simulation Scenario 2 (psilocin-d₁₀ behaves identical to psilocin after psilocybin but with a faster absorption rate due to orange juice administration). Following this assumption, 12 mg of psilocin-d₁₀ was believed to be the threshold dose where clinically meaningful PD effects might be seen, albeit a suboptimal dose, and doses of 20 mg psilocin-d₁₀ or higher would be required if psilocin-d₁₀ absorption and bioavailability behaves the same as psilocin after psilocybin administration in Brown et al. 2017 (Scenario 1). Accordingly, prior to study commencement, it was assumed that no clinically meaningful PD effects would be realized until oral psilocin-d₁₀ doses of at least 12 mg (and even then, suboptimal), but more likely that doses of at least 20 mg would be needed. These assumptions led to a study design with doses below 12 mg in healthy volunteers and starting (assumed to be suboptimal) doses of 12 mg in MDD patients.

Clinical Pharmacokinetic (PK)/Pharmacodynamic (PD) Study with Psilocin-d₁₀

### Trial Design.

Phase I/IIa, Randomized, Double-Blind, Placebo-Controlled Study which assesses the safety, tolerability, pharmacokinetics, and pharmacodynamics of multiple ascending oral doses of psilocin-d₁₀ in participants with and without MDD (ClinicalTrials.gov Identifier: NCT05385783). All subjects receiving study medication in Cohorts 1-6 received oral psilocin-d₁₀ powder in solution (0.1M citric acid+Tang orange drink, 30 mL dose in a dose cup) and subjects receiving placebo received a similar volume of Tang orange drink without the psilocin-d₁₀ study drug. All subjects receiving study medication in the relative bioavailability (BA) cohort received powder-filled capsules containing psilocin-d₁₀ benzenesulfonate salt by ingestion with approximately 240 mL room temperature water, or oral psilocin-d₁₀ powder in solution (0.1M citric acid+Tang orange drink, 30 mL dose in a dose cup) in a cross-over design. Psilocin-d₁₀ for the powder in solution dosage form was manufactured by Patheon Pharma Services, with an isotopic purity of 79.89%. Psilocin-d₁₀ benzenesulfonate salt for the powder-filled capsule dosage form was manufactured by Patheon Pharma Services, with an isotopic purity of 85.20%.

### Population, Treatment Groups, and Number of Subjects.

Cohorts 1, 4, 5, and 6 of the study was comprised of participants with MDD. Cohorts 2, 3, and the relative BA cohort of the study were comprised of normal healthy volunteers (NHV). Participants with MDD had a diagnosis of moderate to severe MDD as defined by the DSM, 5th edition (DSM-5) and scored ≥21 on the Montgomery-Åsberg Depression Scale (MADRS). MDD participants had been on a stable dose of antidepressant medication for at least one month prior to screening with an inadequate response to that antidepressant medication as judged by the Investigator, and continued to receive their prescribed antidepressant throughout the course of the study. Participants did not meet the criteria for treatment-resistant depression.

Cohort 1. For Cohort 1, 2 sentinel subjects were randomized and dosed, including 1 subject who received a psilocin-d₁₀ 1 mg oral dose and 1 subject who received a placebo dose. After the sentinel subjects were dosed, an additional 2 subjects were dosed, including a replacement subject for one of the sentinel subjects who withdrew consent and one additional subject. In total, four Cohort 1 subjects received a first dose of study medication on Day 1, and 3 subjects received a second dose of study medication on Day 22. For the first dose, 2 subjects received an active psilocin-d₁₀ 1 mg dose and 2 subjects received placebo. For the second dose, 3 subjects received an active psilocin-d₁₀ 1 mg dose.

Cohort 2. For Cohort 2, 4 subjects were randomized and dosed on Day 1 (2 subjects on one day, 2 subjects on the next day), including 3 subjects who received a psilocin-d₁₀ 3 mg oral dose and 1 subject who received a placebo dose. These same 4 subjects were again dosed on Day 8 (2 subjects on one day, 2 subjects on the next day), with all 4 subjects receiving a psilocin-d₁₀ 8 mg oral dose (note the actual washout was 11 days between doses).

Cohort 3. For Cohort 3 Day 1, 4 subjects were randomized and dosed on Day 1 (2 subjects on one day, 2 subjects on the next day), including 3 subjects who received a psilocin-d₁₀ 8 mg oral dose and 1 subject who received a placebo dose. One subject vomited 10 minutes after the study medication dose but continued in the study and had a full PK sampling obtained from 0 to 24 hours. Another subject was dosed and left the study after the 2-hour PK sampling and has limited PK samples available for analysis from 0 to 2 hours—this subject's PK data has thus been excluded from the analysis presented hereinafter. For Cohort 3 Day 8, 3 subjects were dosed on Day 8 with a psilocin-d₁₀ 10 mg dose (2 sentinel subjects were dosed on one day, 1 non-sentinel subject was dosed on the next day).

Cohorts 4 and 5. For Cohorts 4 and 5, 12 participants each (24 participants total) were randomized in a 3:1 ratio to receive 12 mg of psilocin-d₁₀ or matched placebo in a single dosing session on Day 1. Following a 21-day interval, all participants received a dose of 12 mg psilocin-d₁₀ on Day 22 (second dose for participants originally randomized to psilocin-d₁₀, first dose for participants randomized to placebo), and were followed to Day 42. Three participants in the psilocin-d₁₀ treatment group did not have Day 21 endpoints due to study discontinuation and withdrawal of consent unrelated to treatment. One participant in the 12 mg psilocin-d₁₀ group did not receive a second dose of psilocin-d₁₀ due to needing more time for integration prior to receiving a second dose of study treatment.

Cohort 6. For Cohort 6, 12 participants were randomized in a 3:1 ratio to receive 16 mg of psilocin-d₁₀ or matched placebo in a single dosing session on Day 1. Following a 21-day interval, all participants received a dose of 16 mg psilocin-d₁₀ on Day 22 (second dose for participants originally randomized to psilocin-d₁₀, first dose for participants randomized to placebo), and were followed to Day 42. Given the availability of data and considering that the secondary endpoint was a change in baseline to Day 21 in MADRS, efficacy data presented herein considers the data cut off of Day 21 postdose. One participant had to be withdrawn from the study due to pregnancy.

Relative BA cohort. For the relative BA cohort, 8 NHV participants received 10 mg doses of the oral powder in solution orange drink and of the powder-filled capsule on Day 1 and Day 8, in a random crossover manner. One subject terminated early after dose 1 (powder-filled capsule) and did not receive dose 2. One subject terminated early after dose 1 (powder in solution) and did not receive dose 2. One subject received both formulations but had only PK samples BLOQ after vomiting for the capsule-formulation. To have 6 matched pairs with data for both capsule and liquid, one subject was replaced.

All other withdrawals were due to scheduling conflicts with the remaining study visits.

The primary efficacy endpoint defined in the statistical analysis plan (SAP) is the change from baseline to Day 21 of the total Montgomery-Åsberg Depression Rating Scale (MADRS) score compared to placebo, therefore efficacy data presented herein considers the data cut off of Day 21 postdose. Mean total score and change from baseline on Montgomery-Åsberg Depression Rating Scale (MADRS) on days 1, 10, 17, 22, 31, 38, and 42 are presented as secondary efficacy endpoints.

Plasma samples (9 samples per subject from 0 to 24 hours postdose for each dose, except for 1 subject in Cohort 3 Day 8 who had 8 samples from 0 to 24 hours postdose with a missed 4 hour postdose sample) were analyzed by Pyxant Labs for the analyte psilocin-d₁₀ and results were reported to the unblinded pharmacokineticist. The lower limit of quantitation (LLOQ) of the assay was 0.00500 ng/ml (5.00 μg/mL). Measurable plasma psilocin-d₁₀ concentrations were reported for the subjects receiving an active psilocin-d₁₀ dose.

The Subject in Cohort 3 Day 8 who had a missed Day 8, 4-hour postdose PK sample distorted the mean psilocin-d₁₀ plasma concentration-time curve for the 10 mg dose group when this was initially plotted by the pharmacokineticist. This subject had an otherwise well characterized plasma-concentration time profile at other timepoints and had the highest plasma concentrations at other timepoints for the 3 subjects receiving 10 mg. To allow for examination of the 10 mg dose summary plot without this distortion, the missing 4-hour concentration for this subject was estimated and set at ˜20% less than the previous 2-hour concentration of 18700 μg/mL. The ˜20% reduction estimate between the 2-hour and 4-hour timepoint was calculated based on the slope of the curves from previously collected data. An estimated psilocin-d₁₀ concentration of 14900 μg/mL was thus used for the 4-hour value for summary statistics, graphical displays, and noncompartmental analysis (NCA) PK analysis purposes.

### Results—Pharmacokinetics and VAS—Any Drug Effect

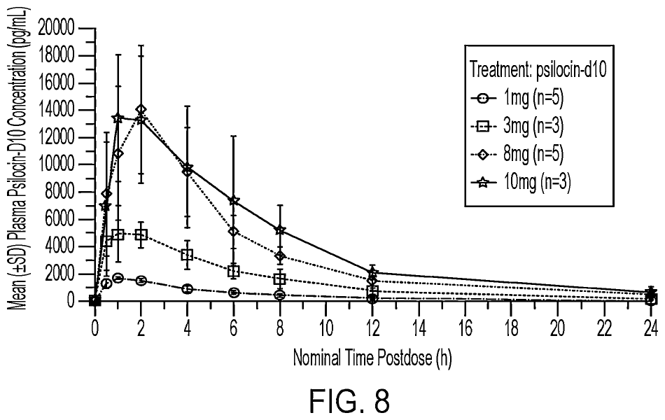

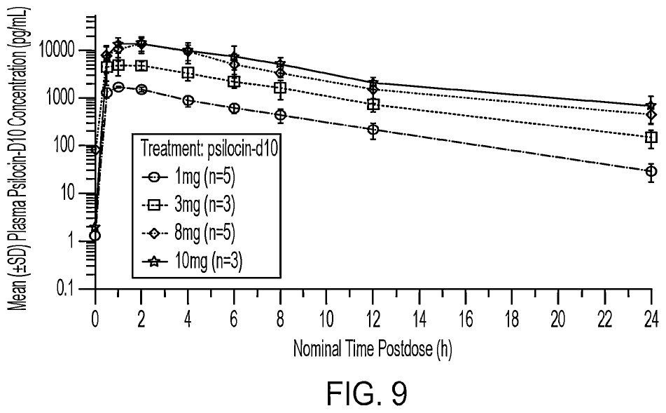

Summary plasma psilocin-d₁₀ concentration-time plots for Cohorts 1-3 are displayed in FIG. 8 (linear scale) and FIG. 9 (semi-log scale) by psilocin-d₁₀ 1, 3, 8, and 10 mg doses (1 mg on Cohort 1, Day 1 and Day 22 were pooled; 3 mg on Cohort 2, Day 1; 8 mg on Cohort 2, Day 8 and Cohort 3, Day 1 were pooled; 10 mg on Cohort 3, Day 8). For this set of figures, one subject in Cohort 3, Day 1 who only had PK sampling from 0 to 2 hours postdose and another subject in Cohort 3, Day 1 who vomited within 10 minutes of dosing were excluded from the 8 mg pooled dose group. Overall, plasma psilocin-d₁₀ concentrations appeared to increase in an approximately dose proportional manner for psilocin-d₁₀ 1, 3, 8, and 10 mg doses.

A noncompartmental analysis was performed using WinNonlin Version 8.3.5 (Certara, Princeton, New Jersey, USA) using the linear up/log down method with actual PK sample collection times postdose.

Key PK parameter results for Cohorts 1-6 with psilocin-d₁₀ 1, 3, 8, 10, 12, and 16 mg doses with days combined for a given dose (i.e., Cohort 1, 1 mg Day 1 and Day 22 were pooled, Cohort 2, 8 mg Day 8, and Cohort 3, 8 mg Day 1 were pooled, Cohort 4, 12 mg Day 1 and Day 22 were pooled, Cohort 5, 12 mg Day 1 and Day 22 were pooled, and Cohort 6, 16 mg Day 1 and Day 22 were pooled) are presented in Table 6.

TABLE 6

| | | MDD | | | | | | | |
|---|---|---|---|---|---|---|---|---|---|
| | | Participants | | NHV Participants | | | | | |
| | | Cohort 1 | | | | Cohorts 2 and 3 | | | |
| | | psilocin-d₁₀ | | Cohort 2 | | psilocin-d₁₀ | | Cohort 3 | |
| | | 1 mg | | psilocin-d₁₀ | | 8 mg | | psilocin-d₁₀ | |
| PK | | (Day 1 and | | 3 mg | | (Day 1 and | | 10 mg | |
| Parameter | Statistic | Day 22) | | (Day 1) | | Day 8)* | | (Day 8) | |
| Cₘₐₓ | n | 5 | | 3 | | 5 | | 3 | |
| (ng/mL) | Mean (SD) | 1.69 | (0.072) | 5.91 | (1.18) | 15.00 | (4.85) | 13.43 | (4.65) |
| | Median | 1.70 | | 5.87 | | 17.10 | | 10.90 | |
| | Min., Max. | 1.61, 1.79 | | 4.75, 7.11 | | 8.51, 20.40 | | 10.60, 18.80 | |
| | CV % | 4.23 | | 19.97 | | 32.35 | | 34.62 | |
| Tₘₐₓ (h) | n | 5 | | 3 | | 5 | | 3 | |
| | Median | 1.00 | | 2.00 | | 2.00 | | 1.00 | |
| | Min., Max. | 0.97, 1.03 | | 1.00, 2.03 | | 1.02, 2.00 | | 1.00, 1.03 | |
| AUC₀₋₂₄ | n | 5 | | 3 | | 5 | | 3 | |
| (h*ng/ml) | Mean (SD) | 10.00 | (1.92) | 34.54 | (4.38) | 83.98 | (21.46) | 100.92 | (39.78) |
| | Min., Max. | 8.09, 12.10 | | 30.37, 39.11 | | 52.36, 104.26 | | 66.90, 144.7 | |
| | CV % | 19.24 | | 12.69 | | 25.55 | | 39.42 | |
| AUCₗₐₛₜ | n | 5 | | 3 | | 5 | | 3 | |
| (h*ng/mL) | Mean (SD) | 10.01 | (1.94) | 34.54 | (4.38) | 83.98 | (21.45) | 100.95 | (39.84) |
| | Min., Max. | 8.09, 12.11 | | 30.37, 39.11 | | 52.37, 104.24 | | 66.88, 144.8 | |
| | CV% | 19.34 | | 12.69 | | 24.39 | | 39.46 | |
| AUC_{0-inf} | n | 5 | | 3 | | 5 | | 3 | |
| (h*ng/mL) | Mean (SD) | 10.19 | (2.01) | 35.60 | (4.60) | 87.77 | (23.11) | 106.16 | (42.54) |
| | Min., Max. | 8.24, 12.41 | | 30.88, 40.07 | | 53.49, 109.46 | | 68.46, 152.3 | |
| | CV % | 19.71 | | 12.91 | | 26.33 | | 40.08 | |
| T_{1/2} (h) | n | 5 | | 3 | | 5 | | 3 | |
| | Mean (SD) | 4.18 | (0.43) | 4.76 | (0.92) | 5.51 | (1.17) | 5.12 | (0.78) |
| | Min., Max. | 3.70, 4.63 | | 4.21, 5.82 | | 4.11, 6.68 | | 4.37, 5.93 | |
| | CV% | 10.28 | | 19.40 | | 21.19 | | 15.26 | |
| CL/F | n | 5 | | 3 | | 5 | | 3 | |
| (L/h) | Mean (SD) | 101.08 | (18.94) | 85.22 | (11.21) | 97.56 | (31.21) | 104.7 | (40.25) |
| | Min., Max. | 80.59, 121.29 | | 74.88, 97.13 | | 73.09, 149.57 | | 65.7, 146.1 | |
| | CV % | 18.74 | | 13.15 | | 31.99 | | 38.45 | |
| Vz/F (L) | n | 5 | | 3 | | 5 | | 3 | |
| | Mean (SD) | 607.8 | (125.0) | 584.1 | (124.6) | 743.9 | (137.5) | 758.3 | (241.9) |
| | Min., Max. | 478.7, 808.3 | | 454.5, 703.1 | | 613.7, 892.5 | | 480.2, 920.0 | |
| | CV % | 20.57 | | 21.33 | | 18.49 | | 31.90 | |
| | | MDD Participants | | | | | | | |
| | | Cohort 4 | | Cohort 5 | | Cohorts 4 and 5 | | Cohort 6 | |
| | | psilocin-d₁₀ | | psilocin-d₁₀ | | psilocin-d₁₀ | | psilocin-d₁₀ | |
| | | 12 mg | | 12 mg | | 12 mg | | 16 mg | |
| PK | | (Day 1 and | | (Day 1 and | | (Day 1 and | | (Day 1 and | |
| Parameter | Statistic | Day 22) | | Day 22) | | Day 22) | | Day 22)** | |
| Cₘₐₓ | n | 19 | | 20 | | 39 | | 21 | |
| (ng/mL) | Mean (SD) | 19.66 | (8.27) | 16.91 | (6.11) | 18.25 | (7.28) | 19.80 | (9.02) |
| | Median | 18.60 | | 14.75 | | 16.20 | | 17.40 | |
| | Min., Max. | 8.16, 38.50 | | 9.82, 29.60 | | 8.16, 38.50 | | 5.85, 37.50 | |
| | CV % | 42.06 | | 36.15 | | 39.89 | | 45.56 | |
| Tₘₐₓ (h) | n | 19 | | 20 | | 39 | | 21 | |
| | Median | 1.03 | | 1.03 | | 1.03 | | 1.03 | |
| | Min., Max. | 1.00, 5.98 | | 0.97, 6.20 | | 0.97, 6.20 | | 1.02, 8.08 | |
| AUC₀₋₂₄ | Mean (SD) | 19 | | 20 | | 39 | | 20 | |
| (h*ng/ml) | n | 154.82 | (61.91) | 137.12 | (50.31) | 145.74 | (56.23) | 154.76 | (61.37) |
| | Min., Max. | 70.67, 320.71 | | 90.28, 259.95 | | 70.67, 320.71 | | 44.70, 250.73 | |
| | CV % | 39.99 | | 36.69 | | 38.58 | | 32.37 | |
| AUCₗₐₛₜ | n | 19 | | 20 | | 39 | | 21 | |
| (h*ng/ml) | Mean (SD) | 154.02 | (62.97) | 137.19 | (50.38) | 145.39 | (56.75) | 151.31 | (60.92) |
| | Min., Max. | 70.67, 320.97 | | 90.30, 260.35 | | 70.67, 320.97 | | 44.83, 251.23 | |
| | CV % | 40.88 | | 36.73 | | 39.03 | | 40.26 | |
| AUC_{0-inf} | n | 19 | | 20 | | 39 | | 19 | |
| (h*ng/ml) | Mean (SD) | 165.45 | (70.05) | 144.13 | (54.63) | 154.51 | (62.71) | 161.33 | (66.32) |
| | Min., Max. | 73.17, 355.27 | | 91.04, 289.77 | | 73.17, 355.27 | | 45.94, 268.19 | |
| | CV % | 43.34 | | 37.90 | | 40.59 | | 41.11 | |
| T_{1/2} (h) | n | 19 | | 20 | | 39 | | 19 | |
| | Mean (SD) | 5.62 | (0.87) | 5.08 | (1.15) | 5.35 | (1.05) | 5.57 | (1.23) |
| | Min., Max. | 4.00, 7.48 | | 3.29, 7.57 | | 3.29, 7.57 | | 3.68, 8.21 | |
| | CV % | 15.55 | | 22.63 | | 19.60 | | 22.02 | |
| CL/F | n | 19 | | 20 | | 39 | | 19 | |
| (L/h) | Mean (SD) | 85.32 | (35.41) | 91.95 | (25.38) | 88.72 | (30.45) | 128.11 | (83.50) |
| | Min., Max. | 33.78, 163.99 | | 41.41, 131.81 | | 33.78, 163.99 | | 59.66, 348.3 | |
| | CV% | 41.51 | | 27.61 | | 34.33 | | 865.18 | |
| Vz/F (L) | n | 19 | | 20 | | 39 | | 19 | |
| | Mean (SD) | 667.83 | (242.48) | 665.52 | (217.27) | 666.65 | (226.84) | 985.5 | (578.7) |

| | | | | |
|---|---|---|---|---|
| Min., Max. | 364.30, 1162.32 | 339.85, 1027.34 | 339.85, 1162.32 | 477.8, 2326 |
| CV % | 36.31 | 32.65 | 34.03 | 58.72 |

*Includes all 4 subjects from Cohort 2, Day 8 and only 1 subject from Cohort 3, Day 1 who received 8 mg dose (excludes two subjects from Cohort 3, Day 1; 1 subject who only had 0-2 h PK sampling and another subject in Cohort 3, Day 1 who vomited within 10 minutes of dosing)
**data from all subjects, including 3 subjects with lower exposure due to emesis

Mean plasma psilocin-d₁₀ Cₘₐₓ was slightly more than 3-fold higher with the 3 mg dose (3.5-fold higher) compared to the 1 mg dose. Mean Cₘₐₓ for the 8 mg dose was slightly less than dose proportional (2.45-fold higher for a 2.67-fold increase in dose) compared to the 3 mg dose and greater than dose proportional (8.6-fold higher for an 8-fold increase in dose) compared to the 1 mg dose. Mean plasma psilocin-d₁₀ Cₘₐₓ was ˜10% lower for the 10 mg dose cohort compared to the 8 mg dose cohort and levels remained approximately the same from 1 to 2 hours following the 10 mg dose followed by a decline during the elimination phase. Median Tₘₐₓ was 1.00 hours for the 1 mg and 10 mg doses and 2.00 hours for the 3 and 8 mg doses.

Mean plasma psilocin-d₁₀ AUC₀₋₂₄, AUCₗₐₛₜ and AUC_{0-inf} were slightly more than 3-fold higher for the 3 mg dose (3.4 to 3.5-fold higher) compared to the 1 mg dose. Mean AUC₀₋₂₄, AUCₗₐₛₜ and AUC_{0-inf} for the 8 mg dose were slightly less than dose proportional (2.3 to 2-4-fold higher for a 2.67-fold increase in dose) compared to the 3 mg dose and approximately dose proportional (8.1 to 8.3-fold higher for an 8-fold increase in dose) compared to the 1 mg dose. Mean plasma psilocin-d₁₀ AUC₀₋₂₄, AUCₗₐₛₜ and AUC_{0-inf} were ˜20% higher for the 10 mg dose compared to the 8 mg dose.

Elimination half-life was 4.18 to 5.41 hours, on average, for the psilocin-d₁₀ 1, 3 and 8 mg doses. Mean elimination half-life (T_{1/2}) was 5.12 hours for the psilocin-d₁₀ 10 mg dose, which was generally consistent with mean T_{1/2} values for the other psilocin-d₁₀ doses. Apparent volume of distribution (Vz/F) was 584.1 to about 760.8 L, on average, indicating the drug is extensively distributed throughout body tissues. Mean clearance (CL/F) was 85.22 to 104.7 L/h, on average, which is consistent with a moderately high clearance drug.

Mean plasma psilocin-d₁₀ Cₘₐₓ for the 12 mg dose was slightly less than dose proportional to the 8 mg dose (˜1.2-fold increase in Cₘₐₓ for a 1.5-fold increase in dose). Median Tₘₐₓ was 1.03 hours (note that PK sampling immediately postdose was limited to the 1 hour timepoint with respect to determining Tₘₐₓ, then the next PK sample was at 6 hours). Mean plasma psilocin-d₁₀ AUC₀₋₂₄, AUCₗₐₛₜ and AUC_{0-inf} increased in a slightly greater than dose proportional manner compared to the 8 mg dose (˜1.7 to 1.8-fold increase in AUC for a 1.5-fold increase in dose). Mean elimination half-life (T_{1/2}) was 5.35 hours for the psilocin-d₁₀ 12 mg dose, which was generally similar to mean T_{1/2} values for the other psilocin-d₁₀ doses. Mean volume of distribution (Vz/F) was ˜667 L for the 12 mg dose, which was generally similar to prior dose Vz/F values and indicates that the drug is extensively distributed throughout body tissues. Mean clearance (CL/F) was ˜89 L/h which was generally similar to prior dose clearance values and indicative of a moderately high clearance of drug.

Mean plasma psilocin-d₁₀ Cₘₐₓ for the 16 mg dose (18 doses, no emesis) was less than dose proportional compared to the 12 mg dose (˜1.1-fold increase in Cₘₐₓ for a 1.33-fold increase in dose). Median Tₘₐₓ was 1.03 hours (note that PK sampling immediately postdose was limited to the 1 hour timepoint with respect to determining Tₘₐₓ, then the next PK sample was at 6 hours). Mean plasma psilocin-d₁₀ AUC₀₋₂₄, AUCₗₐₛₜ and AUC_{0-inf} for the 16 mg dose (18 doses, no emesis) was less than dose proportional compared to the 12 mg dose (˜1.1-fold increase in AUC). Mean elimination half-life (T_{1/2}) was 5.58 hours for the psilocin-d₁₀ 16 mg dose, which was generally similar to mean T_{1/2} values for the other psilocin-d₁₀ doses. Mean volume of distribution (Vz/F) was ˜834.5 L (18 doses, no emesis) for the 16 mg dose which was generally similar to prior dose Vz/F values and indicates that the drug is extensively distributed throughout body tissues. Mean clearance (CL/F) was ˜107.2 L/h (18 doses, no emesis), which was generally similar to prior dose clearance values and indicative of a moderately high clearance drug.

Table 7 shows a comparison between the predicted psilocin-d₁₀ Cₘₐₓ and AUC values using PK simulation Scenario 2 (accounting for a faster absorption rate compared to Brown et al. 2017 due to orange juice administration) versus the observed psilocin-d₁₀ mean Cₘₐₓ and AUC₀₋₂₄ values obtained across dose levels in Cohorts 1 to 6.

TABLE 7
Predicted versus observed Cₘₐₓ and AUC₀₋₂₄ PK parameters by psilocin-dio dose

| Dose level | Predicted Cₘₐₓ¹ | | Observed Mean | Predicted AUC^{1,2} | | Observed Mean |
|---|---|---|---|---|---|---|
| (Cohorts | (Scenario 2) | | Cₘₐₓ | (Scenario 2) | | AUC₀₋₂₄ |
| 1-6) | (ng/ml) | | (ng/mL) | (h*ng/ml) | | (h*ng/ml) |
| 1 mg | 1.34/1.29 = | 0.42 | 1.69 | 4.46/4.35 = | 1.05 | 10.00 |
| | (0.74-2.07) | | (N = 5) | (2.97-6.29) | | (N = 5) |
| 3 mg | 4.05/3.92 = | 1.32 | 5.91 | 13.49/13.15 = | 3.23 | 34.54 |
| | (2.19-6.39) | | (N = 3) | (8.8-19.1) | | (N = 5) |
| 8 mg | 10.88/10.59 = | 3.61 | 15.003 | 36.19/35.37 = | 9.24 | 83.983 |
| | (5.73-17.32) | | (N = 5) | (23.15-52.92) | | (N = 5) |
| 10 mg | 13.80/13.46 = | 4.28 | 13.43 | 45.11/43.77 = | 10.78 | 100.92 |
| | (7.68-21.49) | | (N = 3) | (29.96-64.59) | | (N = 3) |
| 12 mg | 16.34/15.67 = | 5.18 | 18.25 | 54.51/53.26 = | 13.09 | 145.74 |
| | (8.99-25.62) | | (N = 39) | (36.01-77.17) | | (N = 39) |
| 16 mg | 21.91/20.95 = | 6.57 | 20.734 | 71.61/69.84 = | 17.47 | 165.195 |

| | | | |
|---|---|---|---|
| (12.05-33.98) | (N = 18) | (45.48-102.58) | (N = 18) |

¹Predicted mean/median ± standard deviation (5%-95% distribution of the population) are reported.
²AUC₀₋₂₄ is reported instead of AUCinf because 24h concentrations are low.
³Mean Cₘₐₓ and AUC₀₋₂₄ when one subject who only had 0-2 h PK sampling and another subject who vomited are excluded from PK parameter calculations.
Predicted psilocin-d₁₀ concentrations are presented as psilocin concentrations for comparison with other figures. Can be converted to psilocin-d₁₀ concentrations by multiplication of 1.049.
N = number of subjects per dose
⁴Mean values excluding 3 subjects' psilocin-d₁₀ doses who vomited postdose.
⁵One subject did not have a 24 h postdose PK sample.

Surprisingly, the observed mean plasma Cₘₐₓ and AUC₀₋₂₄ at 8 mg dosing (e.g., 15.00 ng/ml and 83.98 h*ng/ml, respectively) were significantly higher than what was predicted in PK simulations with psilocin-d₁₀ at the same dose level (e.g., 10.88 ng/ml and 36.19 h*ng/mL, respectively). Consequently, lower doses of psilocin-d₁₀ were needed to reach threshold plasma levels than predicted from pre-clinical and clinical simulation studies. While the observed mean Cₘₐₓ values for 10, 12, and 16 mg doses were within range of predicted values, the discrepancy between observed mean and predicted AUC₀₋₂₄ continued for the 10 mg, 12 mg, and 16 mg dosing regimens; the observed mean AUC₀₋₂₄ (e.g., 100.92 h*ng/ml, 145.74 h*ng/mL, and 165.19 h*ng/ml respectively) were significantly higher than what was predicted in PK simulations with psilocin-d₁₀ at the same dose levels (e.g., 45.11 h*ng/ml, 54.51 h*ng/ml, and 71.61 h*ng/mL, respectively), indicating that lower dosing levels of psilocin-d₁₀ are needed to provide exposure thresholds than predicted from pre-clinical and clinical simulation studies. Furthermore, the AUC₀₋₂₄ values for the 16 mg dose exceeded the maximum predefined exposure level for AUC (but not Cₘₐₓ) based on the predefined maximum exposure thresholds of dog toxicology studies.

Indeed, in Cohort 2 with 4 healthy volunteers receiving 8 mg of psilocin-d₁₀ orally, the mean maximum VAS—Any drug effect score was 77.2 (versus 42.6 predicted), as shown in Table 8, with 75% of subjects reporting meaningful maximum VAS—Any drug effect readings above 60 mm. In Cohort 3 with 3 healthy volunteers receiving 10 mg of psilocin-d₁₀ orally, the mean maximum VAS—Any drug effect score was 78.3 (versus 54.0 predicted), with 66% of subjects reporting meaningful maximum VAS—Any drug effect readings above 60 mm. Of the subjects reporting meaningful VAS—Any drug effect scores in Cohorts 2 and 3, the scores were 100 mm, 97 mm, and 89 mm (in Cohort 2-8 mg), and 100 mm and 100 mm (in Cohort 3-10 mg) which were significantly higher than expected at the 8 mg dose level and at the 10 mg dose level, respectively. In Cohorts 4 and 5 with MDD participants receiving 12 mg of psilocin-d₁₀ orally, the mean maximum VAS—Any drug effect score was 80.7 (versus 63.9 predicted) with a median score of 100 mm. In Cohort 6 with MDD participants receiving 16 mg of psilocin-d₁₀ orally, the mean maximum VAS—Any drug effect score was 89.2 (versus 85.6 predicted) with a median score of 100 mm, and with 79.2% of subjects reporting meaningful maximum VAS—Any drug effect readings above 60 mm. Notably, the 12 mg and 16 mg VAS—Any drug effect scores are based on blinded data including subjects given placebo on Day 1, meaning that the VAS—Any drug effect scores for the treatment group is likely to be even higher than what is reported for 12 and 16 mg in Table 8. These observed mean maximum VAS—Any drug effect scores obtained at the 8-16 mg psilocin-d₁₀ dose levels could not have been foreseen from literature and clinical modeling studies.

TABLE 8
Predicted versus observed Cₘₐₓ and VAS-Any drug effect score from subjects
dosed with 8 mg psilocin-d₁₀ in Cohort 2, 10 mg of psilocin-d₁₀ in Cohort 3,
12 mg of psilocin-d₁₀ in Cohorts 4 and 5, or 16 mg of psilocin-d₁₀ in Cohort 6

| | | | | Observed | |
|---|---|---|---|---|---|
| | | | | Mean | |
| | Predicted | Predicted | Observed | Maximum | |
| | mean Cₘₐₓ | VAS- | Mean | VAS-Any | |
| | (Scenario 2) | Any drug | Cₘₐₓ | drug effect | |
| Dose | (ng/mL) | effect¹ | (ng/mL) | (SD) | |
| 8 mg | 10.88 | 42.6 | 16.63 | 77.2 | (36.5) |
| (Cohort 2, Day 8) | | | (N = 4) | (N = 4) | |
| 10 mg | 13.80 | 54.0 | 13.43 | 78.3 | (37.5) |
| (Cohort 3, Day 8) | | | (N = 3) | (N = 3) | |
| 12 mg | 16.34 | 63.9 | 18.25 | 80.7³ | (26.7) |
| (Cohorts 4 and 5, | | | (N = 39) | (N = 39) | |
| Day 1 or Day 22) | | | | | |
| 16 mg | 21.91 | 85.6 | 20.73² | 89.2^{2,3} | (19.1) |

| | | |
|---|---|---|
| (Cohort 6, | (N = 18) | (N = 18) |
| Day 1 or Day 22) | | |

¹Calculated from predicted mean Cₘₐₓ in Scenario 2 using modeled linear concentration-effect relationship according to Holze et al. 2022.
Predicted psilocin-d₁₀ concentrations are presented as psilocin concentrations for comparison with other figures. Can be converted to psilocin-d₁₀ concentrations by multiplication of 1.049.
²Mean values excluding 3 subjects' psilocin-d₁₀ doses who vomited postdose.
³blinded data including 3 placebo subjects.
N = number of subjects per dose

For comparison, Table 9 shows the reported/simulated mean Cₘₐₓ plasma levels of psilocin after psilocybin administration and the corresponding mean VAS—Any drug effect scores from literature reports.

TABLE 9
Reported/simulated Cₘₐₓ values for psilocin after psilocybin
administration and corresponding VAS-Any drug effect scores¹

| | | | Reported/ |
|---|---|---|---|
| | | Reported/ | Simulated |
| | | Simulated | mean |
| | Psilocin | mean Cₘₐₓ | VAS-Any |
| Dose | equivalence² | (ng/mL) | drug effect |
| 25 mg psilocybin | 18.0 mg | 15.34 | 60 |
| 30 mg psilocybin | 21.6 mg | 20.47 | 80 |

¹see Table 4
²equivalence based on molar mass

These findings that doses of 8 mg to 16 mg of psilocin-d₁₀ provide therapeutically relevant plasma concentrations and exposures, supported by the observed VAS—Any drug effect scores in the clinically meaningful range, were unexpected based on literature psilocin after psilocybin reports, pre-clinical psilocin-d₁₀ studies, and psilocin-d₁₀ clinical simulations. These unexpected findings required a reevaluation of the 12 mg and higher psilocin-d₁₀ doses planned for the MDD cohorts in light of the surprisingly high VAS—Any drug effect scores from 8 mg and 10 mg doses of psilocin-d₁₀.

### Results—Relative BA Cohort Pharmacokinetics and VAS—any Drug Effect

Administration of psilocin-d₁₀ powder-filled capsules led to a similar PK profile to the oral psilocin-d₁₀ powder in solution dosage form at the same dose level. Table 10 shows the observed psilocin-d₁₀ mean Cₘₐₓ and AUC₀₋₂₄ values obtained from the powder-filled capsules compared to the oral psilocin-d₁₀ powder in solution dosage form from the relative BA cohort, which shows there is not a significant difference in the PK behavior between these dosage forms. Additionally, subjects in the relative BA cohort receiving 10 mg of psilocin-d₁₀ in powder-filled capsules reported a mean maximum VAS—Any drug effect score of 74, which was comparable to the mean maximum VAS—Any drug effect score of 67 reported by subjects receiving 10 mg of psilocin-d₁₀ in powder in solution dosage form (relative BA cohort).

TABLE 10
PK and VAS-Any drug effect score comparison between subjects dosed
with oral powder in solution and powder-filled capsule dosage forms

| Cohort | Dosage Form | Observed | Observed Mean | Observed Mean |
|---|---|---|---|---|
| | of psilocin-d₁₀ | Mean Cₘₐₓ | AUC₀₋₂₄ | Maximum VAS- |
| | (10 mg) | (ng/ml) | (h*ng/mL) | Any drug effect |
| Relative BA cohort | Powder in | 14.87 | 127.26 | 67 |
| | solution | (N = 6) | (N = 6) | (N = 6) |
| Relative BA Cohort | Powder-filled | 14.50 | 110.17 | 74 |
| | capsules | (N = 6) | (N = 6) | (N = 6) |

These results indicate that psilocin-d₁₀ powder in solution and powder-filled capsule dosage forms are biosimilar, and their effects, with respect to both efficacy and safety, can be considered essentially the same.

### Results—Change from Baseline Total MADRS Score

The secondary objective of the study was to evaluate the efficacy of psilocin-d₁₀ in treating symptoms of MDD as measured by the change from baseline to Day 21 of the MADRS total score in the Intent-to-Treat population, with data analyzed according to the treatment group each participant was randomized to receive. Data from participants in Cohorts 4, 5, and 6 randomized to placebo was pooled for analysis purposes (N=9). Data from participants in Cohorts 4, 5, and 6 randomized to 12 mg or 16 mg psilocin-d₁₀ (N=27) was also pooled for analysis.

At 21 days post-dose, psilocin-d₁₀ treatment (12 mg and 16 mg, pooled) resulted in a mean −16.2-point reduction (standard deviation [SD]=6.99) in MADRS total score compared to a mean reduction of −2.6 (SD=2.88) in the placebo group, equating to a statistically significant-13.75 point difference in least squares (LS) means over placebo in the reduction of MADRS total score (95% CI: −18.9, −8.6; P-value<0.0001; Table 11).

On Day 22, participants randomized to the psilocin-d₁₀ group received a second dose of psilocin-d₁₀ (12 mg for Cohorts 4 and 5, or 16 mg for Cohort 6). Upon treatment with a second dose of psilocin-d₁₀, MADRS total score was further reduced from baseline to a mean of −20.9 (SD=11.18; pooled 12 mg and 16 mg dosages) at Day 42 (21 days post-second dose). This effect was consistent across the 12 mg and 16 mg psilocin-d₁₀ dosage levels (Table 12).

Participants randomized to placebo on Day 1 received a first dose of psilocin-d₁₀ (12 mg for Cohorts 4 and 5, or 16 mg for Cohort 6) on Day 22 (Placebo/active group). At 21 days post-dose (Day 42), psilocin-d₁₀ treatment resulted in a mean-16.4-point reduction from baseline (standard deviation [SD]=10.01; pooled 12 mg and 16 mg dosages) in MADRS total score (Table 11). When compared to the LS mean total MADRS score at Day 21 (after placebo treatment), a statistically significant reduction within the placebo group in LS means of −13.89 (95% CI: −22.1, −5.7; p=0.0012) was observed at Day 42 (21 days post-psilocin-d₁₀ dose). The magnitude of MADRS reduction from baseline following a single dose of psilocin-d₁₀ observed within the placebo group is consistent with that observed at Day 21 following the first dose within the psilocin-d₁₀ group (mean-16.2-point reduction; SD=6.99). Additionally, the difference in LS means between psilocin-d₁₀ (2 doses psilocin-d₁₀: Day 1 and Day 22) and placebo (1 dose psilocin-d₁₀, Day 22) at Day 42 is not statistically significant (difference in LS means=−4.56; 95% CI: −31.1, 4.0; p=0.2825) (Table 11).

TABLE 11
Change from Baseline to Day 42 of MADRS Total Score
(12 mg and 16 mg Combined)

| | | | Placebo/active | |
|---|---|---|---|---|
| | Psilocin-d₁₀- | | (pooled 12 mg | |
| | 12 and 16 mg | | and 16 mg) | |
| | (N = 27) | | (N = 9) | |
| | | Change | | Change |
| | | from | | from |
| Visit/Statistic | Observed | Baseline | Observed | Baseline |

Baseline

| | | | | |
|---|---|---|---|---|
| n | 27 | — | 9 | — |
| Mean | 32.2 | — | 31.6 | — |
| SD | 3.76 | — | 4.93 | — |
| Median | 32.0 | — | 33.0 | — |
| Min, Max | 24.0, 42.0 | — | 24.0, 38.0 | — |

Day 21

| | | | | |
|---|---|---|---|---|
| n | 24 | 24 | 9 | 9 |
| Mean | 16.1 | −16.2 | 29.0 | −2.6 |
| SD | 7.69 | 6.99 | 5.32 | 2.88 |
| Median | 16.5 | −17.0 | 31.0 | −3.0 |
| Min, Max | 2.0, 35.0 | -32.0, -3.0 | 17.0, 35.0 | −7.0, 2.0 |

Day 42

| | | | | |
|---|---|---|---|---|
| n | 22 | 22 | 9 | 9 |
| Mean | 10.7 | −20.9 | 15.1 | −16.4 |
| SD | 11.04 | 11.18 | 8.64 | 10.01 |
| Median | 7.0 | −24.5 | 16.0 | −14.0 |
| Min, Max | 0.0, 35.0 | −35.0, 7.0 | 3.0, 29.0 | −32.0, −4.0 |

ANOVA Analysis

| | | | | |
|---|---|---|---|---|
| LS Mean (Std Err) | −16.33 | (1.30) | −2.58 | (2.12) |

| | | |
|---|---|---|
| for Day 21 | | |
| Difference in LS | −13.75 | (2.49) |

| | |
|---|---|
| Means (Std Err) | |
| 95% CI | (−18.9, −8.6) |
| P-value | <0.0001 |

ANOVA Analysis [2]

| | | | | |
|---|---|---|---|---|
| LS Mean (Std Err) | −21.04 | (2.24) | −16.48 | (3.50) |

| | | |
|---|---|---|
| for Day 42 | | |
| Difference in LS | −4.56 | (4.15) |

| | |
|---|---|
| Means (Std Err) | |
| 95% CI | (-13.1, 4.0) |
| P-value | 0.2825 |

ANOVA Analysis [3]

| | | |
|---|---|---|
| LS Mean (Std Err) | −2.65 | (2.89) |
| for Day 21 | | |
| LS Mean (Std Err) | −16.54 | (2.89) |
| for Day 42 | | |
| Difference in LS | −13.89 | (4.08) |

| | |
|---|---|
| Means (Std Err) | |
| 95% CI | (−22.1, −5.7) |
| P-value | 0.0012 |

### Results—Total MADRS Score, Comparison of Dosage Levels

Data from the study was also analyzed according to dosage level of psilocin-d₁₀. Data from participants in Cohorts 4, 5, and 6 randomized to placebo was pooled for analysis purposes (N=9). Data from participants in Cohorts 4 and 5 (treated with 12 mg psilocin-d₁₀) was also pooled for analysis (N=18).

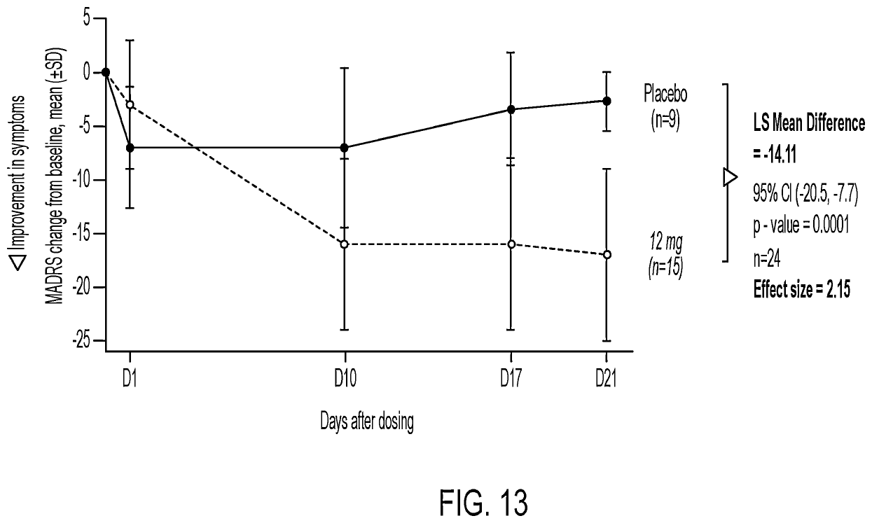

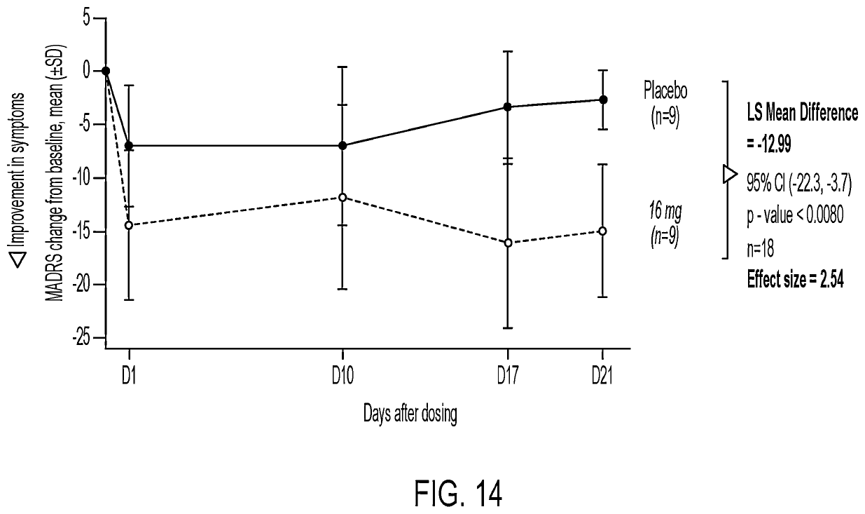

At 21 days post-dose, 12 mg psilocin-d₁₀ treatment resulted in a mean-17.0-point reduction (SD=7.51) in MADRS total score compared to a mean reduction of −2.6 (SD=2.88) in the placebo group, equating to a statistically significant-14.11 point difference in least squares (LS) means compared to placebo in the reduction of MADRS total score (95% CI: −20.5, −7.7; P-value=0.0001; FIG. 13; Table 12). Similarly, 16 mg psilocin-d₁₀ treatment resulted in a mean-14.9-point reduction (SD=6.21) in total MADRS score which represented a −12.99 point difference in least squares (LS) means compared to placebo in MADRS total score (95% CI: −22.3, −3.7; P-value=0.0080; FIG. 14; Table 12). The difference in LS means between 12 mg and 16 mg psilocin-d₁₀ at Day 21 was not statistically significant (difference LS means=−1.09; 95% CI: −12.2, 10.1, p=0.8421).

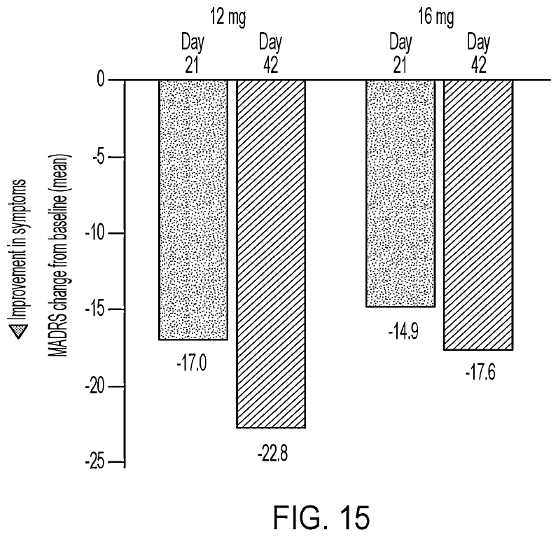

Following treatment with a second dose of psilocin-d₁₀ on Day 22, the 12 mg psilocin-d₁₀ group demonstrated a further numerical reduction in mean MADRS score at Day 42 (mean=−22.8; SD=10.79) when compared to Day 21 (mean=−17.0; SD=7.51) (FIG. 15; Table 12). A similar reduction was also observed within the 16 mg psilocin-d₁₀ group at Day 42 (mean=−17.6; SD=11.80) following a second dose of psilocin-d₁₀ when compared to data at Day 21 (mean=−14.9; SD=6.21) (FIG. 15; Table 12). The difference in LS means between 12 mg and 16 mg psilocin-d₁₀ dosage levels was not statistically significant at Day 42 (difference in LS means=3.58; 95% CI: −15.3, 22.5; p=0.6999) (Table 12).

Participants randomized to placebo received a first dose of psilocin-d₁₀ (12 mg and 16 mg pooled) on Day 22 which resulted in a mean-16.4-point reduction from baseline (SD=10.01) in MADRS total score at Day 42. The difference in LS means between 12 mg psilocin-d₁₀ (2 doses psilocin-d₁₀: Day 1 and Day 22) and placebo (1 dose psilocin-do, Day 22) at Day 42 is not statistically significant (difference in LS means=−3.34; 95% CI: −14.2, 7.5; p=0.5299) (Table 12). The difference in LS means between 16 mg psilocin-d₁₀ (2 doses psilocin-d₁₀: Day 1 and Day 22) and placebo (1 dose psilocin-d₁₀, Day 22) at Day 42 is also not statistically significant (16 mg psilocin-d₁₀ vs placebo: difference in LS means=−7.79; 95% CI: −22.8, 7.2; p=0.2931).

TABLE 12
Change from Baseline to Day 42 of MADRS Total Score (Data by Dosage Levels)

| | Cohorts 4 and 5 | | Cohort 6 | | Placebo/active | |
|---|---|---|---|---|---|---|
| | (12 mg Psilocin-d₁₀) | | (16 mg Psilocin-d₁₀) | | (pooled 12 mg and 16 mg) | |
| | (N = 18) | | (N = 9) | | (N = 9) | |
| | | Change from | | Change from | | Change from |
| Visit/Statistic | Observed | Baseline | Observed | Baseline | Observed | Baseline |

Baseline

| | | | | | | |
|---|---|---|---|---|---|---|
| n | 18 | — | 9 | — | 9 | — |
| Mean | 32.6 | — | 31.4 | — | 31.6 | — |
| SD | 3.66 | — | 4.03 | — | 4.93 | — |
| Median | 32.0 | — | 32.0 | — | 33.0 | — |
| Min, Max | 28.0, 42.0 | — | 24.0, 38.0 | — | 24.0, 38.0 | — |

Day 21

| | | | | | | |
|---|---|---|---|---|---|---|
| n | 15 | 15 | 9 | 9 | 9 | 9 |
| Mean | 15.9 | −17.0 | 16.6 | −14.9 | 29.0 | −2.6 |
| SD | 8.68 | 7.51 | 6.15 | 6.21 | 5.32 | 2.88 |
| Median | 14.0 | −17.0 | 18.0 | −17.0 | 31.0 | −3.0 |
| Min, Max | 2.0, 35.0 | −32.0, −7.0 | 7.0, 27.0 | −24.0, −3.0 | 17.0, 35.0 | −7.0, 2.0 |

Day 42

| | | | | | | |
|---|---|---|---|---|---|---|
| n | 14 | 14 | 8 | 8 | 9 | 9 |
| Mean | 9.4 | −22.8 | 13.0 | −17.6 | 15.1 | −16.4 |
| SD | 12.18 | 10.79 | 9.01 | 11.80 | 8.64 | 10.01 |
| Median | 3.5 | −25.5 | 11.5 | −20.5 | 16.0 | −14.0 |
| Min, Max | 0.0, 35.0 | −35.0, −1.0 | 3.0, 31.0 | −30.0, 7.0 | 3.0, 29.0 | −32.0, −4.0 |

ANOVA Analysis at Day 21

| | | | | | |
|---|---|---|---|---|---|
| LS Mean (Std Err) | −16.70 | (2.28) | −15.61 (3.82) | −2.59 | (2.16) |

Comparison to Cohort 6

| | | |
|---|---|---|
| Difference in LS Means (Std Err) | −1.09 | (5.44) |

| | |
|---|---|
| 95% CI | (−12.2, 10.1) |
| P-value | 0.8421 |

Comparison to Placebo

| | | | |
|---|---|---|---|
| Difference in LS Means (Std Err) | −14.11 | (3.12) | −12.99 |

| | | |
|---|---|---|
| 95% CI | (−20.5, −7.7) | (−22.3, −3.7) |
| P-value | 0.0001 | 0.0080 |

ANOVA Analysis at Day 42

| | | | | | |
|---|---|---|---|---|---|
| LS Mean (Std Err) | −19.83 | (3.87) | −23.41 (6.47) | −16.48 | (3.56) |

Comparison to Cohort 6

| | | |
|---|---|---|
| Difference in LS Means (Std Err) | 3.58 | (9.18) |

| | |
|---|---|
| 95% CI | (−15.3, 22.5) |
| P-value | 0.6999 |

Comparison to Placebo

| | | |
|---|---|---|
| Difference in LS Means (Std Err) | −3.34 | (5.25) |

| | |
|---|---|
| 95% CI | (−14.2, 7.5) |
| P-value | 0.5299 |

### Results—Remission, Response, Relapse

The rates of remission, response, and relapse were also evaluated within the study. At Day 21, 50% (12 out of 24) of participants in the psilocin-d₁₀ group (12 mg and 16 mg combined) had a response to treatment (responders) (defined as ≥50% reduction from baseline MADRS) compared to 0% in the placebo group at Day 21 (Table 13). Additionally, 20.8% (5 out of 24) of participants in the psilocin-d₁₀ group (12 mg and 16 mg combined) were in remission (remitters) (defined as a MADRS score of ≤10) compared to 0% for placebo at Day 21. The same 5 participants who were in remission were also classified as responders. None of the participants relapsed at Day 21 compared to baseline.

Treatment of the psilocin-d₁₀ group with a second dose of psilocin-d₁₀ (12 mg for Cohorts 4 and 5, or 16 mg for Cohort 6) resulted in an increase in the percent of participants responding to treatment to 77.3% (17 out of 22) (Table 14). The rate of remission was also increased upon treatment with a second dose of psilocin-d₁₀ (12 mg for Cohorts 4 and 5, or 16 mg for Cohort 6) to 68.2% (15 out of 22) of participants. None of the participants relapsed at Day 42 compared to baseline.

In the placebo/psilocin-d₁₀ group, i.e., participants receiving placebo on Day 1 and psilocin-d₁₀ on Day 22 (pooled: placebo is N=9; 12 mg is N=6; 16 mg is N=3), 44.4% (4 out of 9) of participants had a response to treatment at Day 42 (Table 14). Additionally, these 4 participants were in remission following a single dose of psilocin-d₁₀ treatment. The rates of response and remission at Day 42 in participants randomized to placebo (21 days post-first dose psilocin-d₁₀) was consistent with those rates observed in the psilocin-d₁₀ group at Day 21 following the first dose of psilocin-d₁₀. None of the participants in the placebo group relapsed at Day 42 compared to baseline.

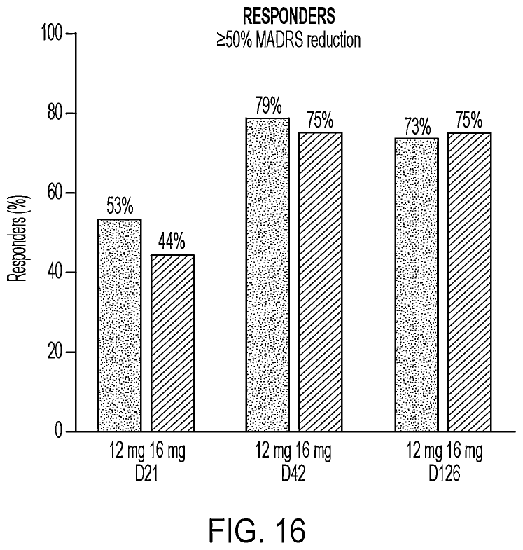

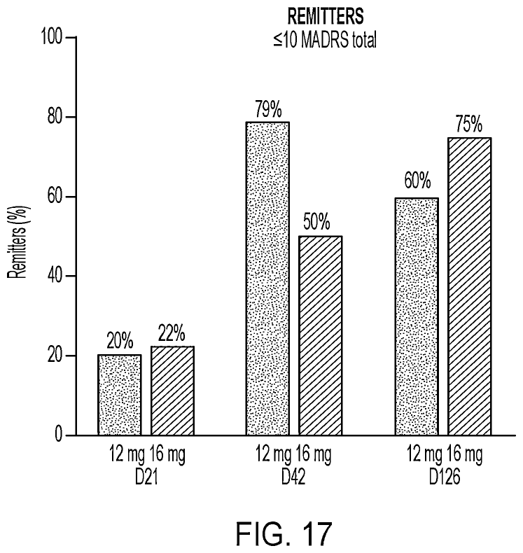

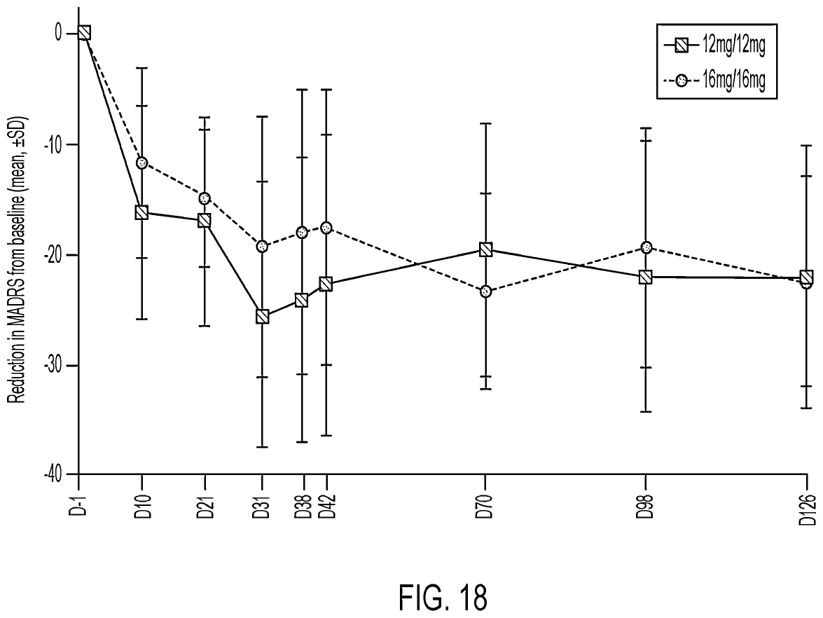

The percentages of participants responding to psilocin-d₁₀ or achieving remission were also consistent between the 12 mg and 16 mg psilocin-d₁₀ treatment groups (see FIG. 16 for response rates and FIG. 17 for remission rates). At Day 21, 53.3% (8 out of 15) of participants responded to 12 mg psilocin-d₁₀ treatment while 20% (3 out of 15) of participants achieved remission. This is comparable to the 44.4% (4 out of 9) of participants treated with 16 mg psilocin-d₁₀ responding to treatment and the 22.2% (2 out of 9) of participants in remission at Day 21 following 16 mg psilocin-d₁₀ treatment. Following a second dose of psilocin-d₁₀, 78.6% (11 out of 14) of participants treated with 12 mg psilocin-d₁₀ met criteria for both response and remission at Day 42. Following a second 16 mg dose of psilocin-d₁₀, 75% (6 out of 8) of participants treated with 16 mg psilocin-d₁₀ met response criteria at Day 42 while 50.0% (4 out of 8) of participants were in remission at Day 42. Notably, durable and sustained effects were seen through the 4-month timepoint (through Day 126) following two doses of either 12 mg or 16 mg psilocin-d₁₀ (dosing on Day 1 and Day 22). As shown in FIG. 16, 73% and 75% of participants treated with two doses of 12 mg and 16 mg psilocin-d₁₀, respectively, continued to meet criteria for response at Day 126. As shown in FIG. 17, 60% and 75% of participants treated with two doses of 12 mg and 16 mg psilocin-d₁₀, respectively, continued to meet criteria for remission at Day 126. These robust and durable effects can also be seen in the mean MADRS reduction from baseline versus time lapse graph of FIG. 18.

TABLE 13
Summary of MADRS Responder Analysis at Day 21

| | | | | | Cohorts 4, 5, and 6 | |
|---|---|---|---|---|---|---|
| | Cohort 4 | Cohort 5 | Cohorts 4 and 5 | Cohort 6 | Psilocin- | |
| Responder | Psilocin- | Psilocin- | Psilocin- | Psilocin- | d₁₀ - pooled | |
| Analysis | d₁₀ - 12 mg | d₁₀ - 12 mg | d₁₀ - 12 mg | d₁₀ - 16 mg | 12 mg and 16 mg | Placebo |
| at Day 21 | (N = 9) | (N = 9) | (N = 18) | (N = 9) | (N = 27) | (N = 9) |

Remission - n (%)

| n | 7 | | 8 | | 15 | | 9 | | 24 | | 9 | |
|---|---|---|---|---|---|---|---|---|---|---|---|---|
| Yes | 2 | (28.6) | 1 | (12.5) | 3 | (20.0) | 2 | (22.2) | 5 | (20.8) | 0 | (0.0) |
| No | 5 | (71.4) | 7 | (87.5) | 12 | (80.0) | 7 | (77.8) | 19 | (79.2) | 9 | (100.0) |

Response - n (%)

| n | 7 | | 8 | | 15 | | 9 | | 24 | | 9 | |
|---|---|---|---|---|---|---|---|---|---|---|---|---|
| Yes | 4 | (57.1) | 4 | (50.0) | 8 | (53.3) | 4 | (44.4) | 12 | (50.0) | 0 | (0.0) |
| No | 3 | (42.9) | 4 | (50.0) | 7 | (46.7) | 5 | (55.6) | 12 | (50.0) | 9 | (100.0) |

Relapse - n (%)

| n | 7 | | 8 | | 15 | | 9 | | 24 | | 9 | |
|---|---|---|---|---|---|---|---|---|---|---|---|---|
| Yes | 0 | (0.0) | 0 | (0.0) | 0 | (0.0) | 0 | (0.0) | 0 | (0.0) | 0 | (0.0) |
| No | 7 | (100.0) | 8 | (100.0) | 15 | (100.0) | 9 | (100.0) | 24 | (100.0) | 9 | (100.0) |

TABLE 14
Summary of MADRS Responder Analysis at Day 42

| | | | | | | |
|---|---|---|---|---|---|---|
| | | | | | Cohorts 4, 5, and 6 | |
| | Cohort 4 | Cohort 5 | Cohorts 4 and 5 | Cohort 6 | Psilocin- | Placebo/active |
| Responder | Psilocin- | Psilocin- | Psilocin- | Psilocin- | d₁₀ - pooled | (pooled 12 mg |
| Analysis | d₁₀ - 12 mg | d₁₀ - 12 mg | d₁₀ - 12 mg | d₁₀ - 16 mg | 12 mg and 16 mg | and 16 mg) |
| at Day 42 | (N = 9) | (N = 9) | (N = 18) | (N = 9) | (N = 27) | (N = 9) |

Remission - n (%)

| n | 7 | | 7 | | 14 | | 8 | | 22 | | 9 | |
|---|---|---|---|---|---|---|---|---|---|---|---|---|
| Yes | 5 | (71.4) | 6 | (85.7) | 11 | (78.6) | 4 | (50.0) | 15 | (68.2) | 4 | (44.4) |
| No | 2 | (28.6) | 1 | (14.3) | 3 | (21.4) | 4 | (50.0) | 7 | (31.8) | 5 | (55.6) |

Response - n (%)

| n | 7 | | 7 | | 14 | | 8 | | 22 | | 9 | |
|---|---|---|---|---|---|---|---|---|---|---|---|---|
| Yes | 5 | (71.4) | 6 | (85.7) | 11 | (78.6) | 6 | (75.0) | 17 | (77.3) | 4 | (44.4) |
| No | 2 | (28.6) | 1 | (14.3) | 3 | (21.4) | 2 | (25.0) | 5 | (22.7) | 5 | (55.6) |

Relapse - n (%)

| n | 7 | | 7 | | 14 | | 8 | | 22 | | 9 | |
|---|---|---|---|---|---|---|---|---|---|---|---|---|
| Yes | 0 | (0.0) | 0 | (0.0) | 0 | (0.0) | 0 | (0.0) | 0 | (0.0) | 0 | (0.0) |
| No | 7 | (100.0) | 8 | (100.0) | 15 | (100.0) | 8 | (100.0) | 22 | (100.0) | 9 | (100.0) |

### Results—Safety

Dosage levels of psilocin-d₁₀ up to 16 mg were well tolerated without significant adverse effects based upon the occurrence of adverse events observed in 8 healthy volunteers (Cohorts 2 and 3) and 38 participants with MDD (Cohorts 1, 4, 5, and 6). All adverse events (AEs) were mild or moderate in severity, with no serious adverse events (SAEs). The most common AEs considered at least possibly related to study treatment were nausea (2 out of 4 subjects treated with 8 mg, 6 out of 24 subjects treated with 12 mg, and 3 out of 12 subjects treated with 16 mg psilocin-d₁₀), vomiting (1 subject in Cohort 4, and 3 subjects in Cohort 6) and headache (1 subject each in Cohorts 3 and 4). Three participants experienced a slight elevation of diastolic blood pressure. However, the elevation was present before or near the time of dosing and resolved without intervention, and likely represents an anticipatory response to dosing rather than a drug related response. No participants discontinued due to AEs. The drug-related, treatment-emergent adverse events are shown in Table 15.

TABLE 15
Drug-related, treatment-emergent adverse eventsª

| | psilocin-d₁₀ | Placebo |
|---|---|---|
| | n (%) | n (%) |
| At least one AE | 21 (60.0) | 8 (61.5) |
| Nausea | 6 (17.1) | 3 (23.1) |
| Blood pressure increased | 5 (14.3) | 3 (23.1) |
| Headache | 3 (8.6) | 0 |
| Vomiting | 2 (5.7) | 1 (7.7) |
| Eructation | 1 (2.9) | 0 |
| Dizziness | 1 (2.9) | 0 |
| Affect lability | 1 (2.9) | 0 |
| Anxiety | 2 (5.7) | 1 (7.7) |
| Panic Attack | 1 (2.9) | 0 |
| Confusional state | 1 (2.9) | 0 |
| Nasal congestion | 1 (2.9) | 0 |
| Tachycardia | 1 (2.9) | 0 |

^{a}Includes participants from all cohorts, including healthy volunteers (psilocin-d₁₀ n = 35, Placebo n = 13)

### Results—Summary

A clinically meaningful within-group change from baseline on the MADRS has been reported to range between a 6- to 9-point reduction in total score (Leucht S, Fennema H, Engel R R, et al. What does the MADRS mean? Equipercentile linking with the CGI using a company database of mirtazapine studies. J Affect Disord. 2017; 210:287-293; Turkoz I, Chow W, Alphs L, et al. Interpretation of change in patient-reported outcomes in treatment-resistant depression. Poster presented at: International Society for Pharmacoeconomics and Outcomes; May 19-23; Baltimore, MD. 2018). As the severity of depression at baseline increases, the magnitude of change needed to be considered meaningful increases. When groups are compared to each other, a 2-point difference between groups has been found to be clinically meaningful (Montgomery S A, Möller H J. Is the significant superiority of escitalopram compared with other antidepressants clinically relevant? Int Clin Psychopharmacol. 2009; 24(3): 111-118; Montgomery S A, Nielsen R Z, Poulsen L H, et al. A randomised, double-blind study in adults with major depressive disorder with an inadequate response to a single course of selective serotonin reuptake inhibitor or serotonin-noradrenaline reuptake inhibitor treatment switched to vortioxetine or agomelatine. Hum Psychopharmacol. 2014; 29 (5):470-482).

The above preliminary clinical evidence suggests that a single dose of psilocin-d₁₀ reaches both within and between group clinical relevance, with a mean 16.2-point reduction within group (pooled 12 mg and 16 mg), a between group difference of −13.75, and a 50% absolute difference in response rates compared to placebo at Day 21. A mean 16.2 point reduction in MADRS score from baseline at Day 21 within the active psilocin-d₁₀ group compares favorably to the average reduction on the 17 item Hamilton Rating Scale for Depression (HAMD17) of −9.8 points (95% CI 9.5 to 10.0) (approximately-14 points on the MADRS) observed within active treatment groups of 232 randomized, double-blind, placebo controlled monotherapy trials in MDD conducted between 1979 to 2016 (Stone M B, Yaseen Z S, Miller B J, Richardville K, Kalaria S N, Kirsch I. Response to acute monotherapy for major depressive disorder in randomized, placebo controlled trials submitted to the US Food and Drug Administration: individual participant data analysis. BMJ. 2022 Aug. 2; 378:e067606). Additionally, treatment with a second dose of psilocin-d₁₀ further reduced the mean reduction in MADRS score by 20.9 points at Day 42.

The between group differences for a single dose of 12 mg psilocin-d₁₀ against placebo at Day 21 in the double-blind phase of the study could also be converted a Cohen's d of 2.15 (95% CI: 0.92, 3.37), which is a surprisingly large effect size. A single dose of 16 mg psilocin-d₁₀ against placebo at Day 21 likewise provided a surprisingly large Cohen's d of 2.54. By comparison, a meta-analysis of results from 48 published antidepressant trials indicated that the mean weighted (Hedges's g) effect-size value was 0.37 (95% CI, 0.33 to 0.41) for antidepressant studies with positive results (Turner E H, Matthews A M, Linardatos E, Tell R A, Rosenthal R. Selective publication of antidepressant trials and its influence on apparent efficacy. N Engl J Med. 2008 Jan. 17; 358(3):252-60).

The between group difference between a single dose of psilocin-d₁₀ (12 mg and 16 mg pooled) and placebo of −13.75 at Day 21 compares favorably to data from clinical trials of 4 representative antidepressants approved within the last 12 years for the treatment of MDD: vortioxetine, levomilnacipram, vilazodone hydrochloride, and combination dextromethorphan hydrobromide and bupropion hydrochloride (see FIG. 19 for an illustration of the placebo subtracted change from baseline in MADRS following 12 mg psilocin-d₁₀ treatment after Day 21 compared to pivotal studies of approved antidepressants). A meta-analysis of 11 placebo-controlled trials of vortioxetine reported a difference in mean change from baseline between placebo and 20 mg vortioxetine of MADRS scores of 4.57 (±1.02 standard error) (Thase M E, Mahableshwarkar A R, Dragheim M, Loft H, Vieta E. A meta-analysis of randomized, placebo-controlled trials of vortioxetine for the treatment of major depressive disorder in adults. Eur Neuropsychopharmacol. 2016 June; 26(6):979-93). Similarly, data from 4 pivotal clinical studies of vortioxetine which utilized the MADRS as the primary measure demonstrated placebo subtracted differences in least-squares mean (LSM) change from baseline ranging from −2.8 to −5.9 (TRINTELLIX [package insert]. Lexington, MA: Takeda Pharmaceuticals America, Inc.; 2021). Data from 3 pivotal clinical studies of levomilnacipram demonstrated placebo subtracted differences in least-squares mean (LSM) change from baseline on MADRS scores ranging from −3.1 to −4.9 (FETZIMA [package insert]. Irvine, CA: Allergan USA, Inc.; 2016), while 4 clinical studies of vilazodone demonstrated placebo subtracted differences in LSM change from baseline on MADRS scores ranging from −2.5 to −5.1 (VIIBRYD [package insert]. Madison, NJ: Allergan USA, Inc.; 2021). Most recently, dextromethorphan hydrobromide and bupropion hydrochloride demonstrated a placebo subtracted LSM difference of −3.9 (95% CI: −6.4, −1.4) in change from baseline on the MADRS in a pivotal clinical trial (AUVELITY [package insert]. New York, NY: AXSOME Therapeutics, Inc.; 2022).

The between group difference of −13.75 between psilocin-d₁₀ (12 mg and 16 mg pooled) and placebo also compares favorably to placebo subtracted differences observed in clinical trials of cariprazine for use as adjunctive therapy to antidepressants for the treatment of MDD (FIG. 19). In these two trials, placebo subtracted differences in LSM change from baseline on MADRS score was −2.5 (95% CI: −4.2, −0.9) for 1.5 mg/day fixed dose in one study and −2.2 (95% CI: −3.7, −0.6) for flexible doses of 2-4.5 mg/day in a second study (VRAYLAR [package insert]. Madison, NJ: Allergan USA, Inc.; 2022).

Post psilocin-d₁₀ treatment (12 mg and 16 mg pooled), 50% of patients (12 of 24) met criteria for response (≥50% reduction from baseline MADRS) at Day 21 and 77% of patients (17 of 22) met criteria for response at Day 42 after a second dose of psilocin-d₁₀ at Day 22. Additionally, 44.4% of patients (4 out of 9) randomized to placebo met criteria for response at Day 42 following a first dose of psilocin-d₁₀ at Day 22. These results compare favorably to results of the STAR*D trial. Response rates (as measured by ≥50% reduction from baseline QIDS-SR₁₆ score) ranged from 48.6% for the first treatment step, 28.5% for the second treatment step, and ˜16% for the third and fourth treatment steps (Rush A J, Trivedi M H, Wisniewski S R, Nierenberg A A, Stewart J W, Warden D, Niederehe G, Thase M E, Lavori P W, Lebowitz B D, McGrath P J, Rosenbaum J F, Sackeim H A, Kupfer D J, Luther J, Fava M. Acute and longer-term outcomes in depressed outpatients requiring one or several treatment steps: a STAR*D report. Am J Psychiatry. 2006 November; 163(11):1905-17). As eligibility criteria for the psilocin-d₁₀ study reported herein required an inadequate response to antidepressant treatment, the results of the psilocin-d₁₀ study may be most appropriately compared to the results of the second treatment step of the STAR*D trial.

Further, a single dose of psilocin-d₁₀ (12 mg and 16 mg pooled) produced a rapid response with a mean 55% reduction in baseline MADRS seen at 10 days post first dose (mean total score 32.2 at baseline vs 17.7 at Day 10; FIG. 13), which was maintained at Day 21 (mean 50% decrease from baseline). A 66% reduction in baseline MADRS was observed 10 days post second dose (mean 15.9 at Day 21 versus 6.7 at Day 31). By comparison, the mean times to response to daily antidepressant treatment in the STAR*D trial ranged from 5.5 weeks (38.5 days) for the first step, 6.5 weeks (45.4 days) for the second step, 6.4 weeks (44.8 days) for the third step, and 8.3 weeks (58.1 days) for the fourth step (Rush A J, Trivedi M H, Wisniewski S R, Nierenberg A A, Stewart J W, Warden D, Niederehe G, Thase M E, Lavori P W, Lebowitz B D, McGrath P J, Rosenbaum J F, Sackeim H A, Kupfer D J, Luther J, Fava M. Acute and longer-term outcomes in depressed outpatients requiring one or several treatment steps: a STAR*D report. Am J Psychiatry. 2006 November; 163(11):1905-17).

The safety profile of psilocin-d₁₀ while administered adjunctively with a stable dose of an antidepressant also is preliminarily indicative of an improvement in the incidence of intolerable side effects when compared to initiation of antidepressant treatment. While psilocin-d₁₀ was well tolerated with mainly mild to moderate nausea, vomiting, and headaches of a transient nature, 16-30% of MDD patients participating in the STAR*D trial reported intolerable side effects due to daily antidepressant treatment (Rush A J, Trivedi M H, Wisniewski S R, Nierenberg A A, Stewart J W, Warden D, Niederehe G, Thase M E, Lavori P W, Lebowitz B D, McGrath P J, Rosenbaum J F, Sackeim H A, Kupfer D J, Luther J, Fava M. Acute and longer-term outcomes in depressed outpatients requiring one or several treatment steps: a STAR*D report. Am J Psychiatry. 2006 November; 163 (11):1905-17). No participants receiving psilocin-d₁₀ withdrew from the study due to adverse events.

Given the magnitude of these clinical effects seen following psilocin-d₁₀ treatment, breakthrough therapy designation (BTD) was granted by the U.S. Food and Drug Administration (FDA) for the treatment of MDD.

The data above/method described above was a portion of the clinical trial protocol described in Example III.

### III. Clinical Trial Protocol

Phase I/IIa, Randomized, Double-Blind, Placebo-Controlled Study with an Open-Label Relative Bioavailability (BA) Cohort to Assess Safety, Tolerability, Pharmacokinetics and Pharmacodynamics of Multiple Ascending Oral Doses of Psilocin-d₁₀ in Healthy Participants with and without Major Depressive Disorder (MDD) (ClinicalTrials.gov Identifier: NCT05385783)

### LIST OF ABBREVIATIONS

ADT antidepressant therapy
AE adverse event
ALT alanine aminotransferase
API active pharmaceutical ingredient
aPTT activated partial thromboplastin time
AST aspartate aminotransferase
ANOVA analysis of variance
AUC area under the plasma concentration-time curve
AUC₀₋ₜ area under the plasma concentration-time curve from time 0 to the last quantifiable time point
AUC_{0-¥} area under the plasma concentration-time curve from time 0 to infinity
AUC₀₋₂₄ area under the plasma concentration-time curve from time 0 to 24 hours
BA Bioavailability
BLOQ Below limit of quantitation
BMI body mass index
BP blood pressure
BUN blood urea nitrogen
CBC complete blood count
CI confidence interval
Cₘₐₓ maximum plasma concentration
CFR Code of Federal Regulations
CK creatine kinase
CNS central nervous system
COVID-19 coronavirus disease caused by SARS-CoV-2 virus
C-SSRS Columbia-Suicide Severity Rating Scale
C-SSRS_{SLV} C-SSRS since last visit
Compound I-3 Investigational product being evaluated in this study, an oral, deuterated (d-10) psilocin, IUPAC name: 3-(2-(bis(methyl-d₃)amino)ethyl-1,1,2,2-d₄)-1H-indol-4-ol
CRF case report form
CYP cytochrome P450
DBP diastolic blood pressure
DRF dose range finding
DSM-V (5) Diagnostic and Statistical Manual of Mental Disorders, 5^{th} Edition
EC ethics committee
ECG electrocardiogram
EMBARK a six-domain framework for psychedelic-assisted psychotherapy; the six domains are: Existential-Spiritual, Mindfulness, Body Aware, Affective-Cognitive, Relational, and Keeping Momentum
FDA Food and Drug Administration
GCP Good Clinical Practice
GGT gamma glutamyl transferase
GI gastrointestinal
GLP Good Laboratory Practice
h or hr hour(s)
HRS Hallucinogen Rating Scale
HBsAg hepatitis B surface antigen
HCV hepatitis C virus
HIV human immunodeficiency virus
IC informed consent
ICH International Conference on Harmonisation
INR international normalized ratio
IRB Institutional Review Board
Kel Apparent terminal rate-constant
kg kilogram(s)
L liter
LC-MS/MS liquid chromatography tandem mass spectrometry
Ln natural logarithm
LSM Least-squares mean
MADRS Montgomery-Åsberg Depression Rating Scale
MCH mean corpuscular or cell hemoglobin
MCHC mean corpuscular or cell hemoglobin concentration
MCV mean corpuscular or cell volume
MDD major depressive disorder
MedDRA Medical Dictionary for Regulatory Activities
MEQ30 Revised Mystical Experience Questionnaire
mg milligram(s)
min minute or minutes
MINI Mini International Neuropsychiatric Interview, Version 7.0.2
mL milliliter(s)
NCE new chemical entity
ng nanogram(s)
NHV normal healthy volunteer
NIMH National Institute of Mental Health
OR Odds ratio
OTC over-the-counter
PAP psychedelic-assisted psychotherapy
PEQ Persisting Effects Questionnaire
PK pharmacokinetic(s)
PD pharmacodynamic(s)
PrEP preexposure prophylaxis
PT prothrombin time
QD once daily
QIDS SR-16 Quick Inventory of Depressive Symptomatology, Self Report 16 item scale
QTcF QT interval corrected for heart rate using Fridericia's formula
R reference formulation
RBC red blood cell
SAE serious adverse event
SARS-CoV-2 severe acute respiratory syndrome coronavirus 2
SBP systolic blood pressure
SD Standard deviation
SF-36 36-item Short Form Survey
SOC System Organ Class
SRC Safety Review Committee
STAR*D Sequenced Treatment Alternatives to Relieve Depression
T test formulation
TK toxicokinetic(s)
Tₘₐₓ time to maximum plasma concentration
TOX toxicology
T_{1/2} half-life
USA United States of America
VAS visual analogue scale
WBC white blood cell
5D-ASC 5-Dimensional Altered States of Consciousness Rating Scale

### Trial Objectives

### Primary Objective

The primary objective of this study is to determine the safety and tolerability of ascending oral doses of psilocin-d₁₀ in healthy participants with and without major depressive disorder (MDD).

### Secondary Objectives

The secondary objectives of this study are:
To determine the PK of ascending oral doses of psilocin-d₁₀.
To characterize the psychedelic effects of psilocin-d₁₀ administration.
To evaluate the efficacy of psilocin-d₁₀ in treating symptoms of MDD.

### Exploratory Objectives

To assess the relative bioavailability (BA) of psilocin-d₁₀ oral powder in solution compared to powder-filled capsules.
To evaluate the effect of psilocin-d₁₀ on ECG parameters, including concentration-QTc (C-QTc) analysis.

### Trial Design

### Study Endpoints

The primary endpoints of the study are:
Clinical safety data from AE reporting, 12-lead ECG, Holter monitoring, clinical safety laboratory evaluations, physical examinations and suicide risk as assessed by the C-SSRS The secondary endpoints of the study are:
Plasma psilocin-d₁₀ concentrations and PK parameters including but not limited to: AUC₀₋ₜ, AUC_{0-∞}, Cₘₐₓ, tₘₐₓ and T_{1/2}
Mystical Experience Questionnaire (MEQ30), the 5-Dimensional Altered States of Consciousness Rating Scale (5D-ASC), the Hallucinogen Rating Scale (HRS) and visual analogue scale (VAS) ratings of ‘Any drug effect’ and a 5-item Persisting Effects Questionnaire (PEQ) assessing the meaningfulness, spirituality and psychological insights and challenges of the experience and overall drug effect
Change from baseline in Montgomery-Åsberg Depression Rating Scale (MADRS) scores in healthy participants with MDD

The exploratory endpoints of the study are:
PK parameters, AUC₀₋ₜ, AUC_{0-¥}, Cₘₐₓ and tₘₐₓ for psilocin-d₁₀ powder in solution compared to powder-filled capsules in the fasted state.
Change-from-baseline heart rate, PR, QTcF and QRS intervals (ΔHR, ΔPR, ΔQTcF and ΔQRS).
Placebo-corrected ΔHR, ΔPR, ΔQTcF and ΔQRS.
Categorical outliers for HR, PR, QRS and QTc
Frequency of treatment emergent T- and U-wave abnormalities

### Study Design

This is a Phase I/IIa, randomized, double-blind, placebo-controlled study in healthy participants with and without MDD. The study also includes an open-label relative bioavailability (BA) cohort in healthy participants without MDD. For sake of clarity throughout the protocol, otherwise healthy participants with MDD will be referred to as MDD participants, and healthy participants without MDD will be referred to as normal healthy volunteer (NHV) participants, or simply, NHV participants.

This study initially evaluated the lowest dose of psilocin-d₁₀ or placebo (1 mg) in MDD participants. Cohort 1 MDD participants have been dosed at the starting dose of psilocin-d₁₀ 1 mg (4 subjects dosed with 1 early dropout). The protocol has been amended following completion of Cohort 1 to enroll the following Cohorts 2-6 and a relative BA cohort:
Cohort 2 will enroll 4 NHV participants (3 active and 1 placebo);
A decision whether to enroll NHVs or MDD patients in Cohort 3 will be dependent on ratings on the VAS-any drug effect score. If the psilocin-d₁₀ doses administered in this cohort are associated with ratings on the VAS any drug effect” of <7 in 75% of NHV participants, then Cohort 3 will enroll 4 NHV participants (3 active and 1 placebo).
If the psilocin-d₁₀ doses administered in this Cohort 2 are associated with ratings on the VAS—any drug effect of >7 in 75% of NHV participants, then Cohort 3 will enroll 8 MDD participants (6 active and 2 placebo).
Cohort 4 is planned for up to 12 MDD participants (9 active and 3 placebo). After 12 participants have been dosed and evaluated (first dose) in Cohort 4, and if dose escalation has reached 12 mg of psilocin-d₁₀, data will be submitted to FDA for review.
Up to 12 MDD participants (9 active and 3 placebo) will be enrolled in Cohort 5.
After Cohort 5 is completed, a soft lock of the data will be performed.
Cohort 6 will enroll up to 12 MDD participants (9 active and 3 placebo). Doses>12 mg will only be administered in Cohort 6, subject to FDA approval.
A single cohort of 8 NHV participants will be enrolled to assess the relative bioavailability (BA) of the oral powder in solution compared to powder-filled capsules in a cross-over design. This cohort can be initiated any time after Cohort 4 dosing commences.

The study will investigate the safety, tolerability, PK and PD of ascending oral doses of psilocin-d₁₀ in MDD participants and NHV participants, the relative bioavailability of psilocin-d₁₀ powder in solution compared to powder-filled capsules.

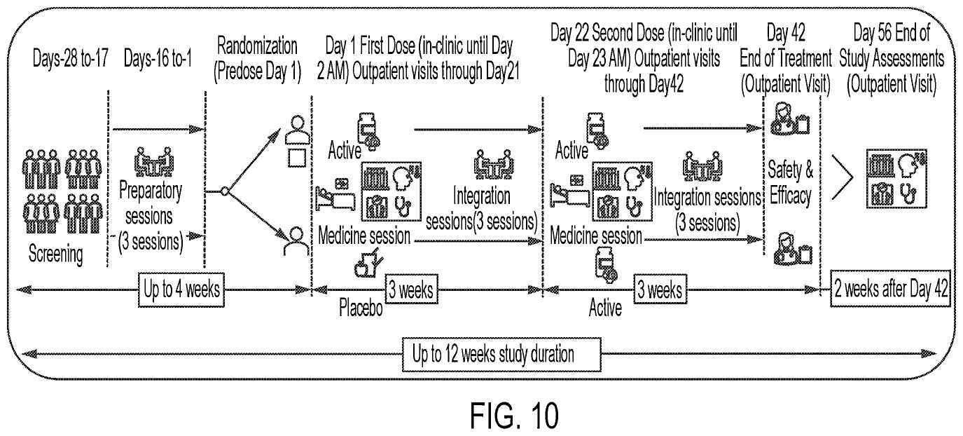

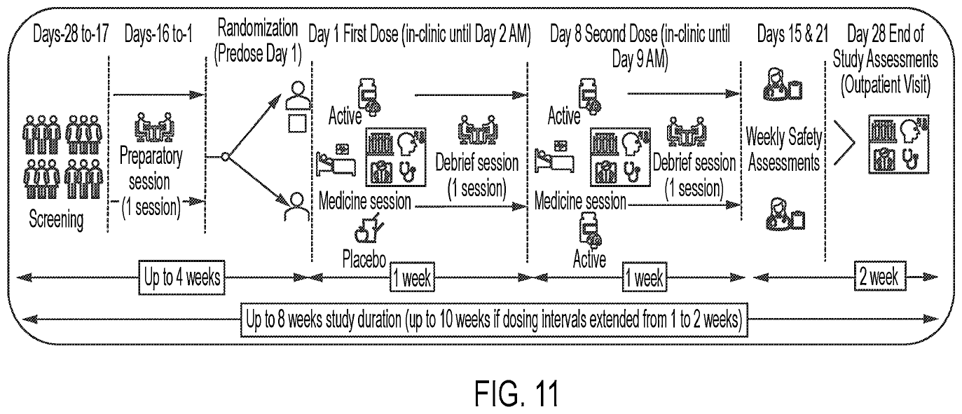

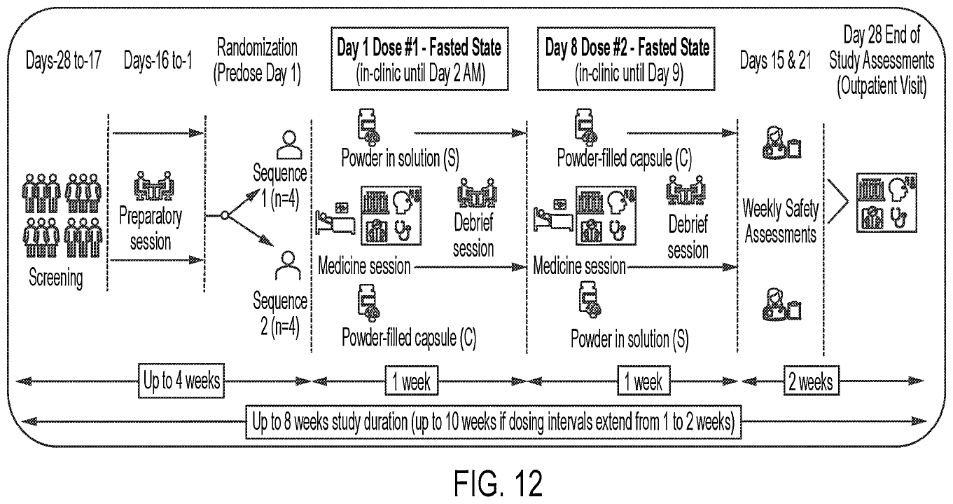

A study schematic is included in FIG. 10 for MDD Participants, FIG. 11 for NHV Participants (Cohorts 2-6), and FIG. 12 for NHV Participants (Relative BA Cohort). A schedule of assessments is included in Table 16 for MDD Participants, Table 17 for NHV Participants (Cohorts 2-6), and Table 18 for NHV Participants (Relative BA Cohort).

TABLE 16
MDD Participants - Schedule of Assessments

| | Screening Period¹ | | First Dosing Period | | | | |
|---|---|---|---|---|---|---|---|
| | Study Days (D) | | | | | | |
| | D −28 | | D 1 | | | | |
| Evaluations³ | to D −2 | D −1 | (First Dose) | D 2 | D 10 +/− 3 | D 17 +/− 3 | D 21 + 7 |
| Informed Consent | X | | | | | | |
| Inclusion/Exclusion Criteria | X | X | | | | | |
| Medical History/Demographics | X | | | | | | |
| Physical Examination | X⁴ | X | | X⁵ | | | X |
| Vital Signs⁷ | X | X | X⁸ | X | | | X |
| Clinical Safety Labs | X | X | | X | | | X |
| TSH | X | | | | | | |
| Serology | X | | | | | | |
| Urine Drugs of Abuse, | X | X | | | | | X |
| Alcohol Breath Screen⁹ | | | | | | | |
| COVID-19 Testing¹⁰ | X | X | | | | | X |
| Pregnancy Test¹¹ | X | X | | | | | X |
| Diagnostic Interview¹² | X | | | | | | |
| MINI¹³ | X | | | | | | |
| MADRS (in clinic) | X | X | X¹⁴ | | | | X |
| MADRS (remote) | | | | | X | X | |
| MEQ30 | | | X¹⁴ | | | | |
| 5D-ASC | | | X¹⁴ | | | | |
| HRS | | | X¹⁴ | | | | |
| PEQ | | | X¹⁴ | | | | |
| VAS Ratings of “Any Drug | | | X¹⁵ | | | | |
| Effect” | | | | | | | |
| QIDS SR-16¹⁶ | | X | | | | | X |
| SF-36 | X | X | | X | | | X |
| C-SSRS-Lifetime | X | | | | | | |
| C-SSRS-SLV | | X | | X | X¹⁷ | X¹⁷ | X |
| ECG¹⁸ | X | X | X | X | | | X |
| 12-lead Holter Monitoring¹⁹ | | X | X | | | | |
| Admission to Inpatient Unit | | X | | | | | X |
| Randomization²⁰ | | | X | | | | |
| Study Drug administration²¹ | | | X | | | | |
| Blood PK sampling²² | | | X²³ | X²³ | | | |
| Treatment-emergent AE | | | X ------------------------------------------------------------------------------------------------------------X | | | | |
| Monitoring | | | | | | | |
| Prior and Concomitant | X | X | X ------------------------------------------------------------------------------------------------------------X | | | | |
| medications | | | | | | | |
| Discharge from Inpatient Unit | | | | X | | | |
| Discharge from Study | | | | | | | |

| | | | | | | End of Study | Post-Study |
|---|---|---|---|---|---|---|---|
| | | | | | | Follow-Up | Remote |
| | Second Dosing Period + Weekly Safety Assessments | | | | | Assessments² | Assessments¹⁶ |
| | Study Days (D) | | | | | | |
| | D 22 | | | | | | |
| Evaluations³ | (Second Dose) | D 23 | D 31 +/− 3 | D 38 +/− 3 | D 42 | D 56² +/− 3 | |
| Informed Consent | | | | | | | |
| Inclusion/Exclusion Criteria | | | | | | | |
| Medical History/Demographics | | | | | | | |
| Physical Examination | | X | | | X | X⁶ | |
| Vital Signs⁷ | X⁸ | X | | | X | X | |
| Clinical Safety Labs | | X | | | X | | |
| TSH | | | | | X | | |
| Serology | | | | | | | |
| Urine Drugs of Abuse, | | | | | | | |
| Alcohol Breath Screen⁹ | | | | | | | |
| COVID-19 Testing¹⁰ | | | | | | | |
| Pregnancy Test¹¹ | | | | | | X | |
| Diagnostic Interview¹² | | | | | | | |
| MINI¹³ | | | | | | | |
| MADRS (in clinic) | X¹⁴ | | | | X | | |
| MADRS (remote) | | | X | X | | | X¹⁶ |
| MEQ30 | X¹⁴ | | | | | | |
| 5D-ASC | X¹⁴ | | | | | | |
| HRS | X¹⁴ | | | | | | |
| PEQ | X¹⁴ | | | | | | |
| VAS Ratings of “Any Drug | X¹⁵ | | | | | | |
| Effect” | | | | | | | |
| QIDS SR-16¹⁶ | | | | | X | | X¹⁶ |
| SF-36 | | X | | | X | | |
| C-SSRS-Lifetime | | | | | | | |
| C-SSRS-SLV | | X | X¹⁷ | X¹⁷ | X | X | |
| ECG¹⁸ | X | X | | | | | |
| 12-lead Holter Monitoring¹⁹ | X | | | | | | |
| Admission to Inpatient Unit | | | | | | | |
| Randomization²⁰ | | | | | | | |
| Study Drug administration²¹ | X | | | | | | |
| Blood PK sampling²² | X²³ | X²³ | | | | | |

| | |
|---|---|
| Treatment-emergent AE | X ------------------------------------------------------------------------------------------------------------X |

Monitoring

| | |
|---|---|
| Prior and Concomitant | X ------------------------------------------------------------------------------------------------------------X |

| | | |
|---|---|---|
| medications | | |
| Discharge from Inpatient Unit | X | |
| Discharge from Study | | X |

Abbreviations: 5D-ASC—5-Dimensional Altered States of Consciousness Rating Scale; AE—adverse event; COVID-19—Coronavirus disease caused by SARS-CoV-2 virus; C-SSRS—Columbia-Suicide Severity Rating Scale; C-SSRS-SLV—Columbia-Suicide Severity Rating Scale - Since Last Visit; ECG—electrocardiogram; HRS—Hallucinogen Rating Scale; MADRS—Montgomery-Åsberg Depression Rating Scale; MEQ30—Mystical Experience Questionnaire, 30-item version; MINI—Mini-International Neuropsychiatric Interview; PEQ—Persist Effects Questionnaire; PK—pharmacokinetics; SF-36—36-item Short Form Survey; VAS—Visual Analogue Scale.
Note that Day 21 is the end of the first dose session evaluations and is also re-admission to the inpatient unit for the start of the second dose session. The timing of Day 21 and the start of the second dose session may be delayed up to 7 days for some cohorts to review available safety, PK and PD data.
¹Screening Period will include Days −28 to −17 screening assessments and Preparatory Psychotherapy Sessions as described in Table 22.
²An End of Study final outpatient visit will take place 2 weeks (±3 days) after the Day 42 visit or at early discontinuation (if applicable).
³For timepoints with multiple assessments, the ECG will be collected first, then vital signs, then the PK blood draw (the PK sample will be collected at the scheduled time postdose).
⁴Physical examinations at Screening will include body weight, BMI and height.
⁵Body weight and BMI only.
⁶Physical examinations at the EOS visit will also include body weight and BMI.
⁷Vital signs measurements include BP, pulse, respiratory rate and body temperature. BP and pulse will be measured in the supine position. Measurements will be obtained after participants have rested in the supine position for at least 5 minutes.
⁸On Day 1 and Day 22, BP, pulse, respiratory rate, and body temperature will be evaluated predose and at 0.5, 1, 2, 4, 6, 8, and 12 hours postdose. On Day 2 and Day 23, BP, pulse, respiratory rate, and body temperature will be evaluated at 24 hours postdose. All collection timepoints are ±10 min
⁹Urine dipsticks may be used on Day −1 and Day 21 for drugs of abuse testing. A breathalyser may be used on Day −1 and Day 21 for alcohol testing.
¹⁰A COVID-19 test will be performed at Screening and on Day −1 and Day 21. Participants who test positive for COVID-19 infection at Screening will be excluded from participating in the study.
¹¹Women of childbearing potential only. A serum pregnancy test will be conducted at Screening; a serum or urine pregnancy test will be conducted on Day −1 and Day 21. A urine pregnancy test will be conducted at subsequent visits; if a urine pregnancy test is positive, a serum pregnancy test will be conducted.
¹²A diagnostic interview will be conducted by a clinician done in coordination with research site staff during the Screening assessments prior to the preparatory sessions, to confirm the MDD diagnosis and that the participant does not have treatment-resistant depression.
¹³The MINI may be performed remotely, if required.
¹⁴The MADRS, MEQ30, 5D-ASC, HRS and PEQ will be administered at the end of the study drug administration session (4 to 10 hours postdose).
¹⁵VAS ratings of ‘Any drug effect’ will be conducted predose and at 0.5, 1, 2, 4, 6, 8, and 12 hours postdose. All collection timepoints are ±10 min
¹⁶Participants will complete a remote MADRS follow-up assessment post-study at 4-, 8- and 12-weeks post-Day 42 timepoints (allowable time window for completion is ±3 days). Participants will be asked to complete an optional QIDS SR-16 follow-up assessment post-study done via ePRO at 4-, 8- and 12-weeks post-Day 42 timepoints.
¹⁷C-SSRS-SLV may be administered remotely on D 10, D 17, D 31 and D 38 visits.
¹⁸12-lead safety ECGs will be printed in triplicates from the Holter device with 5 minutes between each recording. ECGs will be obtained after participants have rested in the supine position for at least 10 minutes at the following time points on Day 1 and Day 22: 3 time points prior to dosing (−45, −30 and −15 minutes) and at 0.5, 1, 2, 4, 6, 8, and 12 hours postdose and on Day 2 and Day 23 at 24 hours postdose, immediately before the PK sample. All collection timepoints are ±10 min. When ECG recordings coincide with safety ECGs, vital signs assessment and blood draws, procedures will be carried out in said order.
¹⁹12-lead Holter data will be collected for 6 hours (±10 minutes) on Day −1 to establish a baseline; and from 1 hour predose until 12 hours (±10 minutes) postdose on Day 1 and Day 22.
²⁰Participants will be randomized on Day −1 or the morning of Day 1 after completion of all 3 preparatory sessions and re-confirmation by the Investigator that all inclusion criteria are met and none of the exclusion criteria are met.
²¹Study drug will be administered in the fasting state (following an overnight fast of at least 10 hours and except for the ingestion of the orange drink study drug solution).
²²Pharmacokinetic blood samples will be conducted within the following allowable time windows for timepoints: Predose (from 0 to 30 minutes predose), 1 h timepoint (±2 min), 6 h timepoint (±5 min), 8 and 12 h timepoints (±10 min), and 24 h timepoint (±15 min).
²³On Day 1 and Day 22, plasma samples for PK analysis will be collected predose and at 1, 6, 8, and 12 hours postdose. On Day 2 and Day 23, plasma samples for PK analysis will be collected at 24 hours postdose.

TABLE 17
NHV Participants- Schedule of Assessments

| | | | | | | | | | | End of Study |
|---|---|---|---|---|---|---|---|---|---|---|
| | Screening | | | | | Second Dosing Period + | | | | Follow-Up |
| | Period¹ | First Dosing Period | | | | Weekly Safety Assessments | | | | Assessments² |
| | Study Days | | | | | | | | | |
| | D −28 | | D 1 | | | D 8 | | | | |
| Evaluations⁴ | to D −2 | D −1 | (First Dose) | D 2 | D 7 | (Second Dose)² | D 9 | D 15 | D 21 | D 28³ +/− 3 |
| Informed Consent | X | | | | | | | | | |
| Inclusion/Exclusion Criteria | X | X | | | | | | | | |
| Medical History/Demographics | X | | | | | | | | | |
| Physical Examination | X⁵ | X | | X⁶ | X | | X | | X | X⁷ |
| Vital Signs⁸ | X | X | X⁹ | X | X | X⁹ | X | | X | X |
| Clinical Safety Labs | X | X | | X | X | | X | | X | |
| TSH | X | | | | | | | | X | |
| Serology | X | | | | | | | | | |
| Urine Drugs of Abuse, | X | X | | | X | | | | | |
| Alcohol Breath Screen¹⁰ | | | | | | | | | | |
| COVID-19 Testing¹¹ | X | X | | | X | | | | | |
| Pregnancy Test¹² | X | X | | | X | | | | | X |
| MEQ30 | | | X¹³ | | | X¹³ | | | | |
| 5D-ASC | | | X¹³ | | | X¹³ | | | | |
| HRS | | | X¹³ | | | X¹³ | | | | |
| PEQ | | | X¹³ | | | X¹³ | | | | |
| VAS Ratings of “Any Drug Effect” | | | X¹⁴ | | | X¹⁴ | | | | |
| C-SSRS-Lifetime | X | | | | | | | | | |
| C-SSRS-SLV | | X | | X | X | | X | X | X | X |
| ECG¹⁵ | X | X | X | X | X | X | X | | | |
| 12-lead Holter Monitoring¹⁶ | | X | X | | | X | | | | |
| Admission to Inpatient Unit | | X | | | X | | | | | |
| Predose Preparation Session | | X | | | | | | | | |
| Randomization¹⁷ | | | X | | | | | | | |
| Study Drug administration¹⁸ | | | X | | | X | | | | |
| Blood PK sampling¹⁹ | | | X²⁰ | X²⁰ | | X²⁰ | X²⁰ | | | |
| Medicine Session | | | X | | | X | | | | |
| Postdose Debrief Session | | | | X | | | X | | | |
| Treatment-emergent AE Monitoring | | X ----------------------------------------------------------------------------------------------------------------------X | | | | | | | | |
| Prior and Concomitant medications | X | X ----------------------------------------------------------------------------------------------------------------------X | | | | | | | | |
| Discharge from Inpatient Unit | | | | X | | | X | | | |
| Telephone Safety Assessments | | | | | | | | X | X | |
| Outpatient Safety Assessment Visit | | | | | | | | | | X |
| Discharge from Study | | | | | | | | | | X |

Abbreviations: 5D-ASC—5-Dimensional Altered States of Consciousness Rating Scale; AE—adverse event; COVID-19—Coronavirus disease caused by SARS-CoV-2 virus; C-SSRS—Columbia-Suicide Severity Rating Scale; C-SSRS-SLV—Columbia-Suicide Severity Rating Scale - Since Last Visit; ECG—electrocardiogram; HRS—Hallucinogen Rating Scale; MEQ30—Mystical Experience Questionnaire, 30-item version; PEQ—Persist Effects Questionnaire; PK—pharmacokinetics; VAS—Visual Analogue Scale.
¹Screening Period will include Days −28 to −2 screening assessments and predose Preparation Session on Day −1 prior to Day 1 dosing.
²The second dose may be administered 2 weeks instead of 1 week after the first dose on Day 15 at the Investigator's discretion, and all visit days for the second dosing period would shift by 1 week; e.g., D 14 admission, D 15 second dose, D 16, D 21 ± 3 days, D 28 visits during second dosing period, and follow-up assessments on D 35 ± 3 days. If the interval between doses is extended, participants should receive a telephonic safety assessment at approximately weekly intervals. Weekly telephonic safety assessments have an allowable visit window of ±3 days.
³An End of Study final outpatient visit will take place on Day 28 ± 3 days visit or at early discontinuation (if applicable).
⁴For timepoints with multiple assessments, the ECG will be collected first, then vital signs, then the PK blood draw (the PK sample will be collected at the scheduled time postdose).
⁵Physical examinations at Screening will include body weight, BMI and height.
⁶Body weight and BMI only.
⁷Physical examinations at the EOS visit will also include body weight and BMI.
⁸Vital signs measurements include BP, pulse, respiratory rate and body temperature. BP and pulse will be measured in the supine position. Measurements will be obtained after participants have rested in the supine position for at least 5 minutes.
⁹On Day 1 and Day 8, BP, pulse, respiratory rate, and body temperature will be evaluated predose and at 0.5, 1, 2, 4, 6, 8, and 12 hours postdose. On Day 2 and Day 9, BP, pulse, respiratory rate, and body temperature will be evaluated at 24 hours postdose. All collection timepoints are ±10 min
¹⁰Urine dipsticks may be used on Day −1 and Day 7 for drugs of abuse testing. A breathalyser may be used on Day −1 and Day 7 for alcohol testing.
¹¹A COVID-19 test will be performed at Screening, Day −1 and Day 7. Participants who test positive for COVID-19 infection at Screening will be excluded from participating in the study.
¹²Women of childbearing potential only. A serum pregnancy test will be conducted at Screening; a serum or urine pregnancy test will be conducted on Day −1 and Day 7. A urine pregnancy test will be conducted at subsequent visits; if a urine pregnancy test is positive, a serum pregnancy test will be conducted.
¹³The MEQ30, 5D-ASC, HRS and PEQ will be administered at the end of the study drug administration session (4 to 10 hours postdose).
¹⁴VAS ratings of ‘Any drug effect’ will be conducted predose and at 0.5, 1, 2, 4, 6, 8 and 12 hours postdose. All collection timepoints are ±10 min
¹⁵12-lead safety ECGs will be printed in triplicates from the Holter device with 5 minutes between each recording. ECGs will be obtained after participants have rested in the supine position for at least 10 minutes at the following time points on Day 1 and Day 8: 3 time points prior to dosing (−45, −30 and −15 minutes) and at 0.5, 1, 2, 4, 6, 8, and 12 hours postdose and on Day 2 and Day 9 at 24 hours postdose, immediately before the PK sample. All collection timepoints are ±10 min. When ECG recordings coincide with safety ECGs, vital signs assessment and blood draws, procedures will be carried out in said order.
¹⁶12-lead Holter data will be collected for 6 hours (±10 minutes) on Day −1 to establish a baseline; and from 1 hour predose until 12 hours (±10 minutes) postdose on Day 1 and Day 8.
¹⁷Participants will be randomized on Day −1 or the morning of Day 1 after completion of the predose preparation session and re-confirmation by the Investigator that all inclusion criteria are met and none of the exclusion criteria are met.
¹⁸Study drug will be administered in the fasting state (following an overnight fast of at least 10 hours and except for the ingestion of the orange drink study drug solution).
¹⁹Pharmacokinetic blood samples will be conducted within the following allowable time windows for timepoints: Predose (from 0 to 30 minutes predose), 0.5 and 1 h timepoints (±2 min), 2 to 6 h timepoints (±5 min), 8 and 12 h timepoints (±10 min), and 24 h timepoint (±15 min).
²⁰On Day 1 and Day 8, plasma samples for PK analysis will be collected predose and at 0.5, 1, 2, 4, 6, 8, and 12 hours postdose. On Day 2 and Day 9, plasma samples for PK analysis will be collected at 24 hours postdose.

TABLE 18
NHV Participants (Relative Bioavailability) - Schedule of Assessments

| | | | | | | | | | | End of Study |
|---|---|---|---|---|---|---|---|---|---|---|
| | Screening | | | | | Second Dosing Period + | | | | Follow-Up |
| | Period¹ | First Dosing Period | | | | Weekly Safety Assessments | | | | Assessments² |
| | Study Days | | | | | | | | | |
| | D −28 | | D 1 | | | D 8 | | | | |
| Evaluations⁴ | to D −2 | D −1 | (First Dose) | D 2 | D 7 | (Second Dose)² | D 9 | D 15 | D 21 | D 28³ +/− 3 |
| Informed Consent | X | | | | | | | | | |
| Inclusion/Exclusion Criteria | X | X | | | | | | | | |
| Medical History/Demographics | X | | | | | | | | | |
| Physical Examination | X⁵ | X | | X⁶ | X | | X | | X | X⁷ |
| Vital Signs⁸ | X | X | X⁹ | X | X | X⁹ | X | | X | X |
| Clinical Safety Labs | X | X | | X | X | | X | | X | |
| TSH | X | | | | | | | | X | |
| Serology | X | | | | | | | | | |
| Urine Drugs of Abuse, | X | X | | | X | | | | | |
| Alcohol Breath Screen¹⁰ | | | | | | | | | | |
| COVID-19 Testing¹¹ | X | X | | | X | | | | | |
| Pregnancy Test¹² | X | X | | | X | | | | | X |
| MEQ30 | | | X¹³ | | | X¹³ | | | | |
| 5D-ASC | | | X¹³ | | | X¹³ | | | | |
| HRS | | | X¹³ | | | X¹³ | | | | |
| PEQ | | | X¹³ | | | X¹³ | | | | |
| VAS Ratings of “Any Drug Effect” | | | X¹⁴ | | | X¹⁴ | | | | |
| C-SSRS-Lifetime | X | | | | | | | | | |
| C-SSRS-SLV | | X | | X | X | | X | X | X | X |
| ECG¹⁵ | X | X | X | X | X | X | X | | | |
| 12-lead Holter Monitoring¹⁶ | | X | X | | | X | | | | |
| Admission to Inpatient Unit | | X | | | X | | | | | |
| Predose Preparation Session | | X | | | | | | | | |
| Randomization¹⁷ | | | X | | | | | | | |
| Study Drug administration¹⁸ | | | X | | | X | | | | |
| Blood PK sampling¹⁹ | | | X²⁰ | X²⁰ | | X²⁰ | X²⁰ | | | |
| Medicine Session | | | X | | | X | | | | |
| Postdose Debrief Session | | | | X | | | X | | | |
| Treatment-emergent AE Monitoring | | X ----------------------------------------------------------------------------------------------------------------------X | | | | | | | | |
| Prior and Concomitant medications | X | X ----------------------------------------------------------------------------------------------------------------------X | | | | | | | | |
| Discharge from Inpatient Unit | | | | X | | | X | | | |
| Telephone Safety Assessments | | | | | | | | X | X | |
| Outpatient Safety Assessment Visit | | | | | | | | | | X |
| Discharge from Study | | | | | | | | | | X |

Abbreviations: 5D-ASC—5-Dimensional Altered States of Consciousness Rating Scale; AE—adverse event; COVID-19—Coronavirus disease caused by SARS-CoV-2 virus; C-SSRS—Columbia-Suicide Severity Rating Scale; C-SSRS-SLV—Columbia-Suicide Severity Rating Scale - Since Last Visit; ECG—electrocardiogram; HRS—Hallucinogen Rating Scale; MEQ30—Mystical Experience Questionnaire, 30-item version; PEQ—Persist Effects Questionnaire; PK—pharmacokinetics; VAS—Visual Analogue Scale.
¹Screening Period will include Days −28 to −2 screening assessments and predose Preparation Session on Day −1 prior to Day 1 dosing.
²The preferred dosing interval for each dose is 1 week between the first and second doses, however, this interval may be increased up to 2 weeks between doses at the Investigator's discretion. All visit days for the second dosing day would shift. If the interval between doses is extended, participants should receive a telephonic safety assessment at approximately weekly intervals. Weekly telephonic safety assessments have an allowable visit window of ±3 days.
³An End of Study final outpatient visit will take place on Day 28 ± 3 days visit or at early discontinuation (if applicable).
⁴For timepoints with multiple assessments, the ECG will be collected first, then vital signs, then the PK blood draw (the PK sample will be collected at the scheduled time postdose).
⁵Physical examinations at Screening will include body weight, BMI and height.
⁶Body weight and BMI only.
⁷Physical examinations at the EOS visit will also include body weight and BMI.
⁸Vital signs measurements include BP, pulse, respiratory rate and body temperature. BP and pulse will be measured in the supine position. Measurements will be obtained after participants have rested in the supine position for at least 5 minutes.
⁹On Day 1 and Day 8, BP, pulse, respiratory rate, and body temperature will be evaluated predose and at 0.5, 1, 2, 4, 6, 8, and 12 hours postdose. On Day 2 and Day 9, BP, pulse, respiratory rate, and body temperature will be evaluated at 24 hours postdose. All collection timepoints are ±10 min.
¹⁰Urine dipsticks may be used on Day −1 and Day 7 for drugs of abuse testing. A breathalyser may be used on Day −1 and Day 7 for alcohol testing.
¹¹A COVID-19 test will be performed at Screening, Day −1 and Day 7. Participants who test positive for COVID-19 infection at Screening will be excluded from participating in the study.
¹²Women of childbearing potential only. A serum pregnancy test will be conducted at Screening; a serum or urine pregnancy test will be conducted on Day −1 and Day 7. A urine pregnancy test will be conducted at subsequent visits; if a urine pregnancy test is positive, a serum pregnancy test will be conducted.
¹³The MEQ30, 5D-ASC, HRS and PEQ will be administered at the end of the study drug administration session (4 to 10 hours postdose).
¹⁴VAS ratings of ‘Any drug effect’ will be conducted predose and at 0.5, 1, 2, 4, 6, 8 and 12 hours postdose. All collection timepoints are ±10 min.
¹⁵12-lead safety ECGs will be printed in triplicates from the Holter device with 5 minutes between each recording. ECGs will be obtained after participants have rested in the supine position for at least 10 minutes at the following time points on Day 1 and Day 8: 3 time points prior to dosing (−45, −30 and −15 minutes) and at 0.5, 1, 2, 4, 6, 8, 12 hours postdose and on Day 2 and Day 9 at 24 hours postdose, immediately before the PK sample. All collection timepoints are ±10 min. When ECG recordings coincide with safety ECGs, vital signs assessment and blood draws, procedures will be carried out in said order.
¹⁶12-lead Holter data will be collected for 6 hours (±10 minutes) on Day −1 to establish a baseline; and from 1 hour predose until 12 hours (±10 minutes) postdose on Day 1 and Day 8.
¹⁷Participants will be randomized on Day −1 or the morning of Day 1 after completion of the predose preparation session and re-confirmation by the Investigator that all inclusion criteria are met and none of the exclusion criteria are met.
¹⁸Study Drug Dose #1 and Dose #2 will be administered in the fasting state (following an overnight fast of at least 10 hours and except for the ingestion of the orange drink study drug solution).
¹⁹Pharmacokinetic blood samples will be conducted within the following allowable time windows for timepoints: Predose (from 0 to 30 minutes predose), 0.5 and 1 h timepoints (±2 min), 2 to 6 h timepoints (±5 min), 8 and 12 h timepoints (±10 min), and 24 h timepoint (±15 min).
²⁰On Day 1 and Day 8, plasma samples for PK analysis will be collected predose and at 0.5, 1, 2, 4, 6, 8, and 12 hours postdose. On Day 2 and Day 9, plasma samples for PK analysis will be collected at 24 hours postdose.

Up to 64 participants are planned to be enrolled into 7 cohorts, including up to 48 MDD participants and 16 NHV participants.

All participants will have a Screening visit between 28 and 17 days prior to first dosing to ensure they meet the inclusion/exclusion criteria and are in good general health. Some of the Screening procedures may be performed remotely, while others will be required on-site. All screening assessments must be completed and participants confirmed eligible by the Investigator based on inclusion and exclusion criteria review before the psychological support sessions described below can commence.

Cohorts 1-6: For the first dose (Day 1), participants will receive either an active psilocin-d₁₀ dose or placebo (3:1 ratio). For the second dose (Day 22 for MDD participants or Day 8 for NHV participants), all participants will receive an active psilocin-d₁₀ dose. For NHV participants, the second dose may be given after a two-week interval (Day 15) instead of on Day 8, at the discretion of the Investigator.

The starting dose of psilocin-d₁₀ in Cohort 1 was 1 mg, based on initial human dose safety calculations per FDA guidance, modelling of predicted exposures, and taking into account safety data from non-clinical studies. The dose will be escalated in increments up to 3× from the previous dose following satisfactory review of available safety, tolerability, pharmacodynamic (PD) and PK data from the preceding cohort(s).

In Cohorts 1, 2, 3, 5 and 6, there may be a dose escalation within the cohort, such that participants will receive one dose level of psilocin-d₁₀ for the first dose. A review of available safety, PK and PD data by the SRC will take place on Day 7 or Day 21. For these cohorts, the scheduled Day 7 or Day 21 visit may be delayed up to 7 days to allow for this review. Contingent on this review, it is proposed to test the same or another dose level in the second period of these cohorts, as presented in the dose escalation Table 19. Proposed doses may be adjusted based on emerging PK, and safety data. The estimated number and type of participants (MDD or NHV) are summarized by cohort and proposed dose in Table 20.

The maximum dose of psilocin-d₁₀ in this study will be determined based on maximum Cₘₐₓ and AUC₀₋₂₄ exposures of 343 ng/ml and 3630 hr*ng/ml, respectively, obtained from the rat 14-day dose range finding (DRF) study at 40 mg/kg in female rats, as well as emerging pharmacodynamic, safety and tolerability data.

The proposed first dose (Day 1) and second dose (Day 8 or Day 22) dose escalations and fed or fasted dosing conditions for study drug are presented in Table 19.

TABLE 19
Proposed Dose Escalation Scheme for Double-Blind Cohorts 1-6

| | Day 1 | Day 1 | Day 8 or | Highest Day 8 | Day 8 or |
|---|---|---|---|---|---|
| | Dose | Fed/ | Day 22 | or Day 22 | Day 22 |
| Cohort | (mg) | Fasted | Dose (mg) | Dose (mg) | Fasted |
| 1 | 1 | Fasted | 1 | 1 | Fasted |
| 2 | 3 | Fasted | 6 | 8 | Fasted |
| 3 | 9 | Fasted | 12 | 12 | Fasted |
| 4 | 12 | Fasted | 12 | 12 | Fasted |
| 5 | 12 | Fasted | 12 | 12 | Fasted |
| 6 | 9 or up | Fasted | Up to 16 | Up to 16 | Fasted |
| | to 16 | | | | |

Dose levels >12 mg pending FDA review.
Note:
Study participants who have received Day 1 dose may receive the same dose level on Day 8 or Day 22 dose if agreed upon by the Investigator and Sponsor to allow for flexibility and more time with respect to recruitment of a sufficient number of participants at the Day 1 dose level.

TABLE 20
Estimated Number and Type of Participants
by Cohort and Proposed Dose

| | | Potential |
|---|---|---|
| | | psilocin-d₁₀ |
| Cohort | Cohort size | doses (mg) |
| 1 | 4 MDD | 1 |
| 2 | 4 NHVs | 3 and 6 |
| 3 | 4 NHVs or 8 MDD | 9 and 12 |
| 4 | Up to 12 MDD *** | 12 |
| Relative BA | 8 NHV | ≤12 |
| 5 | Up to 12 MDD *** | 12 ** |
| 6 | Up to 12 MDD *** | Up to 16* |

*Pending FDA review and SRC recommendation; ** Soft lock and data review will occur after Cohort 5 is completed. *** a minimum of 8 completers is intended.

Sentinel dosing will occur at each dose level. Each cohort will include 2 sentinel participants; 1 participant will receive psilocin-d₁₀ and 1 participant will receive matched placebo. The remaining participants in the cohort will commence treatment at least 24 hours after commencement of treatment in the sentinel participants after satisfactory review of the safety and tolerability data by the Investigator. A licensed physician will be on call and able to reach the clinical site within 15 minutes on study drug dosing Day 1, Day 8 and Day 22 in the event of a physiological or psychiatric emergency. The physician should be available at least until 4 hours after all vital signs and mental status/neurological effects return to baseline.

Relative Bioavailability Cohort: This open-label cohort can commence after at least 4 participants have received the first dose in Cohort 4, and the dose in this cohort will be determined based on data generated in previous cohorts. Eight NHV participants will receive two doses of active psilocin-d₁₀ study medication on separate study days (Table 21). For Dose 1 (Day 1), 4 participants (Sequence 1) will receive psilocin-d₁₀ powder in solution and 4 participants (Sequence 2) will receive psilocin-d₁₀ powder-filled capsules. For Dose 2 (Day 8), participants will cross over to the alternate formulation (i.e., psilocin-d₁₀ powder-filled capsules or powder in solution). Dose 1 and Dose 2 will be administered in the fasting state. For this cohort, the preferred interval between doses is 1 week, however, this interval between doses may be stretched from 1 to 2 weeks at the Investigator's discretion.

TABLE 21
Proposed Dosing Scheme for Relative Bioavailability Cohort

| Sequence | Dose 1 | Dose 2 |
|---|---|---|
| 1 | 4 NHV participants, | 4 NHV participants, |
| | psilocin-d₁₀ powder | psilocin-d₁₀ powder- |
| | in solution, fasted | filled capsules, fasted |
| 2 | 4 NHV participants, | 4 NHV participants, |
| | psilocin-d₁₀ powder- | psilocin-d₁₀ powder in |
| | filled capsules, fasted | solution, fasted |

### Psychological Support Sessions:

For MDD Participants: Supportive psychotherapy, EMBARK for MDD (“A Treatment Manual for EMBARK: A Six-Domain Framework of Psilocin-Assisted Therapy for Major Depressive Disorder”), will be provided during at least three sessions of preparatory psychotherapy (predose), medicine sessions, and at least three sessions of integration psychotherapy (postdose) following each dose administration. During these sessions, participants will be paired with a dyad of two facilitators who will have completed specialized training in EMBARK for MDD; one or both facilitators will be a licensed psychotherapist.

Participants will have a preparatory psychotherapy session A1, A2 and A3 occurring within a flexible time window, with the first two preparatory sessions occurring any time after eligibility screening and the third preparatory session occurring on or about Day-1 (see Table 22 for time windows). Each preparatory session will last for 90 minutes. Flexibility is allowed for the scheduling of these sessions at the facilitator's discretion and coordination with the participant and Investigator provided that all three preparatory sessions are completed prior to any study medication dosing.

TABLE 22
MDD Participants - Preparatory and Integration Psychotherapy Sessions

| | Baseline Preparatory | | | Post-First Dose Integration | | | Post-Second Dose Integration | | |
|---|---|---|---|---|---|---|---|---|---|
| | Psychotherapy Sessions | | | Psychotherapy Sessions | | | Psychotherapy Sessions | | |
| | Study Days | | | | | | | | |
| | After | After | | | | | | | |
| | Eligibility | Eligibility | | | | | | | |
| Evaluations | Confirmation | Confirmation | D −1 − 7 | D 2 + 7 | D 10 +/− 4 | D 21 − 7 | D 23 + 7 | D 31 +/− 4 | D 38 +/− 4 |
| Psychotherapy - Preparation | X | X³ | X | | | | | | |
| (predose)¹ | | | | | | | | | |
| Psychotherapy Integration - | | | | X | X³ | X | X | X³ | X³ |
| (postdose)² | | | | | | | | | |

¹Three baseline preparatory psychotherapy sessions lasting 90 minutes each will be performed, with the first two preparatory psychotherapy sessions occurring after eligibility confirmation and the third session occurring on Day −1 (−7 day window). The preparatory sessions have a flexible time window as indicated in the table, provided that all the screening assessments are performed and the participant is determined to be eligible by the Investigator. Additional preparatory psychotherapy sessions and integration sessions may be performed at the discretion of the facilitator in coordination with the Investigator and research staff.
²Three postdose integration psychotherapy sessions lasting 90 minutes will be performed following the first dose, and again following the second dose. The time windows for these postdose sessions are flexible as indicated in the table. D 1 and D 22 dose columns are listed in this table as reference anchors relative to the psychotherapy sessions.
³All psychotherapy visits (preparatory & integration) are preferred in person but psychotherapy session facilitators have the option to conduct any two of the three preparatory sessions and all of the integration sessions as remote/telemedicine visits.

For the last of three preparatory psychotherapy predose sessions, the preferred default schedule is the final preparatory session on Day-1 in the afternoon or evening before dosing, first dosing on Day 1, and the first postdose integration session on the morning of Day 2, so the participant completes these study activities during one extended visit. Similarly, for the last of three integration psychotherapy sessions before the second dose administration, the preferred default schedule is the final session on Day 21 in the afternoon or evening before dosing, then dosing on Day 22, and the first postdose integration session on the morning of Day 23, so the participant completes these study activities during one extended visit.

After each dose, postdose integration psychotherapy sessions, Sessions B1, B2 and B3, will occur on approximately Day 2, 10 and 21 following the first dose and Sessions C1, C2 and C3 will occur on approximately Day 23, 31 and 38 following the second dose. Each postdose integration session will last for 90 minutes. These integration sessions are meant to be flexible and may occur on different days rather than a fixed weekly schedule depending on an assessment of an individual study participant by the facilitator, provided there are at least three postdose integration sessions following each study medication dose. Additional preparatory psychotherapy sessions or integration psychotherapy sessions may be conducted by study facilitators at the Investigator's discretion.

For NHV participants: Healthy volunteers will receive at least one predose preparation session, a medicine session on Day 1 (first dose) and Day 8 (second dose), and at least one debrief session following each dose. The predose preparation sessions and debrief sessions can be done remotely. Additional preparation sessions or debrief sessions may be conducted by study facilitators at the Investigator's discretion.

### Double-Blind Treatment Period:

All participants will be admitted to the research site in the morning of Day-1 for the collection of baseline safety assessments and mood and psychological health questionnaires. Participants will wear a Holter monitor for approximately 6 hours initiated in the morning which will serve as a baseline Holter session. This baseline Holter recording must be reviewed prior to dosing on Day 1 to confirm that there are no abnormal findings that may preclude a participant's involvement in the clinical trial.

Dose 1: Randomization to double-blind study medication will occur on Day 1 after successful completion of the three preparatory psychotherapy sessions (MDD participants) or the predose preparation session (NHV participants) and eligibility re-confirmation by the Investigator prior to dosing on Day 1.

All participants who are confirmed eligible and who are randomized will receive an oral dose of psilocin-d₁₀ or matched placebo in the morning of Day 1 in the fasted state. Participants will wear a Holter monitor for approximately 12 hours following dosing (Holter monitor should be placed approximately 1 hour prior to dosing and continue until approximately 12 hours postdose). Serial PK blood sampling will be collected predose and at 1, 6, 8, 12 and 24 hours postdose for MDD participants and predose and at 0.5, 1, 2, 4, 6, 8, 12 and 24 hours postdose for NHV participants. Other safety and PD assessments will be performed as described in Table 16 for MDD Participants and Table 17 for NHV Participants.

For all participants, the psilocin-d₁₀ treatment session may last from 4 to 8 hours, and two trained facilitators will be present to support the participant throughout the dosing session. Participants will reside at the research site until the morning of Day 2 (until approximately 25 hours postdose) and will be discharged if the Investigator deems it safe to do so, following collection of safety assessments and PK blood samples, and completion of mood and psychological health questionnaires.

MDD participants will have at least three postdose integration psychotherapy sessions as described above (supportive psychotherapy). NHV participants will have at least one debrief session following the study medication dose. Adverse event and concomitant medication monitoring will also be performed during each of these visits.

Dose 2: Participants will be re-admitted to the research site on Day 7 (NHV participants) or Day 21 (MDD participants) for the collection of safety assessments and mood and psychological health questionnaires to assess their response to the first dosing session. The same study procedures will be performed on Day 7 through Day 9 (NHV participants) or Day 21 through Day 23 (MDD participants) as were done on Days-1 through Day 2. All participants will receive an oral dose of psilocin-d₁₀ in the morning of Day 8 (NHV participants) or Day 22 (MDD participants). Two trained facilitators will be present to support the participant throughout the dosing session. There will be a +7 day window allowed for the Day 7 or Day 21 visit to allow for review of safety data, if needed.

Participants will reside at the research site until the morning of Day 9 (NHV participants) or Day 23 (MDD participants) (approximately 25 hours postdose) and will be discharged if the Investigator deems it safe to do so, following collection of safety assessments and PK blood samples, and completion of mood and psychological health questionnaires.

MDD participants will have at least three postdose integration psychotherapy sessions as described above (supportive psychotherapy). NHV participants will have at least one debrief session following the study medication dose. Adverse event and concomitant medication monitoring will also be performed during each of these visits.

Study medication will be administered in the fasted state, following an overnight fast of approximately 10 hours in cohorts 1-6.

### Open-Label Treatment Period:

NHV participants will be admitted to the research site in the morning of Day-1 for the collection of baseline safety assessments and mood and psychological health questionnaires. Participants will wear a Holter monitor for approximately 6 hours initiated in the morning which will serve as a baseline Holter session. This baseline Holter recording must be reviewed prior to dosing on Day 1 to confirm that there are no abnormal findings that may preclude a participant's involvement in the clinical trial.

Dose 1: Randomization to open-label study medication sequence 1 or 2 will occur on Day 1 after successful completion of the predose preparation session and eligibility re-confirmation by the Investigator prior to dosing on Day 1.

Participants who are confirmed eligible and who are randomized will receive an oral dose of psilocin-d₁₀ powder in solution or powder-filled capsules in the morning of Day 1 in the fasted state. Participants will wear a Holter monitor for approximately 12 hours following dosing (Holter monitor should be placed approximately 1 hour prior to dosing and continue until approximately 12 hours postdose). Serial PK blood sampling will be collected predose and at 0.5, 1, 2, 4, 6, 8, 12 and 24 hours postdose. Other safety and PD assessments will be performed as described in Table 18.

The psilocin-d₁₀ medicine session may last from 4 to 8 hours, and two trained facilitators will be present to support the participant throughout the dosing session. Participants will reside at the research site until the morning of Day 2 (until approximately 25 hours postdose) and will be discharged if the Investigator deems it safe to do so, following collection of safety assessments and PK blood samples, and completion of mood and psychological health questionnaires.

Dose 2: Participants will be re-admitted to the research site on Day 7 for the collection of safety assessments and mood and psychological health questionnaires. The same study procedures will be performed on Day 7 through Day 9 as were done on Days-1 through Day 2. Participants will crossover to the alternate study medication (psilocin-d₁₀ powder in solution or powder-filled capsule) in the morning of Day 8 in the fasted state.

Two trained facilitators will be present to support the participant throughout each dosing session. Participants will have at least one debrief session following each study medication dose. Adverse event and concomitant medication monitoring will also be performed during each of these visits. The preferred schedule is for participants to receive the two doses 1 week apart (i.e., Days 1 and 8). However, the time interval may be extended up to 2 weeks between doses at the Investigator's discretion. If the time interval between doses is extended to 2 weeks, participants will have approximately weekly telephonic safety assessments.

### Cohorts 1-6 Follow-Up Assessments and Visits:

MDD participants will have approximately weekly telephonic assessments for the three weeks following study drug administration for MADRS assessments and for safety assessments. MDD participants will return to the research site for an outpatient visit on Day 42 for collection of safety assessments and completion of mood and psychological health questionnaires to assess their response to the second dosing session. MDD participants will return to the research site for a final in-person Follow-up visit on Day 56 (±3 days) for collection of end-of-study safety assessments and completion of mood and psychological health questionnaires. Remote MADRS assessments will be completed at 4-, 8- and 12-weeks post-Day 42 visit (allowable time window for completion is ±3 days). Optional follow up assessments using QIDS-SR16 will be delivered to and completed by participants at 4-, 8- and 12-weeks post-Day 42 visit using ePRO. The maximum expected duration of participation for an MDD participant is approximately 12 weeks, including a screening and preparatory period of up to 4 weeks and a treatment phase and follow-up of up to 8 weeks (but excluding the additional 12 weeks for remote MADRS assessments and optional ePRO follow up).

NHV participants will have approximately weekly telephonic assessments for two weeks following the second dose study drug administration for safety assessments. NHV participants will return to the research site for a final in-person Follow-up visit on Day 28 (±3 days) for collection of end-of-study safety assessments. The maximum expected duration of participation for a NHV participant is approximately 8 weeks, including a screening and preparatory period of up to 4 weeks and a treatment phase and follow-up of up to 4 weeks. If the Investigator extends the time between the two doses from 1 to 2 weeks, the treatment phase and follow-up will be up to 6 weeks, for a total expected duration of participation of approximately 10 weeks.

### Relative Bioavailability Cohort Follow-Up Assessments and Visits:

NHV participants will have approximately weekly telephonic assessments for two weeks following the third dose study drug administration for safety assessments. NHV participants will return to the research site one week later for a final in-person Follow-up visit on Day 28 (±3 days) for collection of end-of-study safety assessments. The maximum expected duration of participation for a NHV participant is approximately 8 weeks, including a screening and preparatory period of up to 4 weeks and a treatment phase and follow-up of up to 4 weeks. If the Investigator extends the time between the two doses by up to 2 weeks after each dose, the treatment phase and follow-up will be up to 16 weeks, for a total expected duration of participation of approximately 10 weeks.

### Study Duration

The duration of the double-blind portion of the study for individual MDD participants is up to 12 weeks including a screening visit and preparatory psychotherapy sessions of up to 4 weeks, a first dose with integration psychotherapy sessions of 3 weeks, a second dose with integration psychotherapy sessions of 3 weeks, and a follow-up period of 2 weeks.

The duration of the double-blind portion of the study for individual NHV participants is up to 8 weeks including a screening visit and a predose preparation session of up to 4 weeks, a first dose with medicine and debrief sessions of 1 week, a second dose with medicine and debrief sessions of 1 week, and a follow-up period of 2 weeks. If the Investigator decides to extend the time between doses to 2 weeks for each dose, this will extend the duration of the study for these NHV participants to 10 weeks.

The duration of the open-label relative BA portion of the study for individual NHV participants is up to 8 weeks including a screening visit and a predose preparation session of up to 4 weeks, two doses with medicine and debrief sessions of 1 week each, and a follow-up period of 2 weeks. If the Investigator decides to extend the time between doses to 2 weeks for each dose, this will extend the duration of the study for these NHV participants to 10 weeks.

### Study Drug Dosing

The starting dose of psilocin-d₁₀ will be 1 mg in Cohort 1 participants. Subsequent dose escalations in additional cohorts will be decided upon after review of all relevant safety, PK and clinical data and agreed upon by the Investigator, Sponsor and SRC. The doses for subsequent cohorts will be documented in writing and communicated to the investigational pharmacist for implementation. Participants in each cohort will receive a dose of psilocin-d₁₀ or matched placebo in the first dosing session (Day 1), and an active dose of psilocin-d₁₀ in the second dosing session (Day 8 or Day 22).

Psilocin-d₁₀ will be provided to the Investigator and research pharmacist (or other appropriately trained personnel as designated by the Investigator) as active pharmaceutical ingredient (psilocin-d₁₀ free base API) for the powder in solution dosage form and as powder-filled capsules (containing the psilocin d₁₀ benzenesulfonate salt). For the double-blind portion of the study, an unblinded pharmacist or other designee will prepare active psilocin-d₁₀ and placebo doses and will administer doses to the participants in a manner that preserves the integrity of the blind for the Investigator, the participants, and other research personnel. For the open-label portion of the study, a pharmacist or other designee will prepare and dispense the appropriate dose of powder in solution or powder-filled capsule(s) for participants.

API will be dissolved in 0.1M citric acid to give a 1 mg/mL stock solution. For the powder in solution dose, an appropriate volume of study drug (e.g., 1 mL for a 1 mg dose) will be mixed with Tang orange drink to mask taste and to make a 30 mL dose in a dose cup. Matching placebo will consist of similar volume of Tang orange drink without the psilocin-d₁₀ study drug. Participants will ingest the 30 mL dose from a dose cup and will then drink 210 mL Tang orange drink for a total volume of approximately 240 mL. For the psilocin-d₁₀ powder-filled capsule dose, participants will ingest the capsule(s) with approximately 240 mL room temperature water. Detailed instructions for preparing and administering individual study drug doses will be provided in a Pharmacy Manual. All study medication doses must be documented with date and time of administering and by all study personnel involved in dosing.

For Cohorts 1-6, study drug will be administered in the fasting state (following an overnight fast of at least 10 hours and except for the ingestion of the orange drink study drug solution).

For the relative BA cohort, 8 NHV participants will be enrolled and will receive two doses followed by a 7-day washout period between each dose. Participants will be randomly assigned to sequence 1 or 2 as presented in Table 21.
For Dose 1, 4 participants will receive psilocin-d₁₀ powder in solution and 4 participants will receive psilocin-d₁₀ powder-filled capsules, in the fasted state (following an overnight fast of at least 10 hours).
For Dose 2, after a 7-day washout period, participants will crossover and receive the alternate study medication, powder in solution or powder-filled capsules, again in the fasted state.

On days where participants are confined in the research clinic, standardized meals will be served to participants (e.g., lunch at approximately 4.5 hours and dinner at 10 hours postdose) will be served and an evening snack will be offered, if desired by the participant. The mealtimes are approximately and may be modified so the meals do not interfere with study procedures (but should be kept generally consistent for all participants to the extent feasible).

The study medication oral powder in solution or powder-filled capsule doses must be completely ingested as described above. Participants will remain sitting upright and ambulation will be limited during the immediate 4 hour postdose period, except for bathroom privileges if necessary. On the study drug dosing days, participants must not consume any liquids other than the study drug Tang orange drink or water ingested with the study medication within 1 hour before and 1 hour after dosing.

Water will be offered ad libitum after the 1 hour postdose PK blood sample is collected. Soft drinks (sodas) or non-citrus fruit juices will be offered with meals and ad libitum beginning 4.5 hours postdose. Caffeinated beverages, such as coffee or tea may be consumed in moderation. Participants must refrain from ingesting alcohol or excessive amounts of caffeine at screening and within 24 hours prior to each dose and until after the last PK blood sample is obtained.

### Randomization

A randomization schedule for the double-blind study part and another for the open-label study part will be generated by a statistical programmer not directly involved in the analysis or conduct of the study. The randomization schedules will be provided to an unblinded pharmacist at the research site who will implement the schedule as participants qualify for randomization.

Randomization to double-blind study medication in Cohorts 1-6 will occur on Day 1 after successful completion of the three preparatory psychotherapy sessions for MDD participants and the predose preparation session for NHV participants and eligibility re-confirmation of all participants by the Investigator prior to dosing on Day 1. Participants in each cohort will receive a dose of psilocin-d₁₀ or matched placebo in the first dosing session (Day 1), and a dose of psilocin-d₁₀ in the second dosing session (Day 8 or Day 22).

Randomization to open-label study medication in the relative BA cohort will occur on Day 1 after successful completion of the predose preparation session for NHV participants and eligibility re-confirmation of all participants by the Investigator prior to dosing on Day 1. Participants in this cohort will receive the first dose of psilocin-d₁₀ in the fasted state on Day 1, and the second dose in the fasted state on Day 8, unless the time between dosing intervals is extended by the Investigator.

### Selection and Withdrawal of Participants

Participants for the study will be recruited from a research participant data base and/or general advertising and selected during screening based on the inclusion and exclusion criteria and clinical assessment listed below.

### Inclusion Criteria

### MDD and NHV Participants:

Participants meeting the following criteria will be included in the study:
1. Participant is assigned female or male at birth.
2. Participant is aged between 21 to 65 years, inclusive, at Screening.
3. Participant has a BMI of 18 to 30 kg/m², inclusive, at Screening.
4. Participant is ≥60 kg.
5. Negative severe acute respiratory syndrome coronavirus 2 (SARS-CoV-2) test at Screening and Day-1.
6. Participants capable of producing sperm must use a condom during the trial and for 3 months after their final dose of trial medication, if their partner is a person of childbearing potential. In addition, their partner of childbearing potential must use a highly effective method of contraception (i.e., failure rate less than 1% when used consistently and correctly) from first dosing until 3 months following final dosing.
7. Participants of childbearing potential must agree to use a highly effective method of contraception (i.e., failure rate less than 1% when used consistently and correctly) in combination with a partner who is capable of producing sperm must use a condom during the trial and for 3 months after their final dose of trial medication. Such participants must have a negative pregnancy test at Screening and Day-1.
8. Participants of non-childbearing potential who are or were capable of producing eggs (ova) must be postmenopausal or permanently sterile following hysterectomy, bilateral salpingectomy or bilateral oophorectomy. Postmenopausal is defined as spontaneous amenorrhea for at least 12 months, and a serum follicle stimulating hormone (FSH) level in the menopausal range, unless the participant is taking hormone replacement therapy or is using hormonal contraception.
9. Provision of giving written informed consent, which includes compliance with the requirements and restrictions listed in the consent form.

### MDD Participants:

Participants meeting the following criteria will be included in the study:
1. Participant has a diagnosis of MDD (as defined in the Diagnostic and Statistical Manual of Mental Disorders, 5^{th} edition [DSM-V] of moderate to severe degree (scoring≥21 on the Montgomery-Åsberg Depression Scale [MADRS], established through a full psychiatric work up that includes the Mini-International Neuropsychiatric Interview [MINI]), who are otherwise healthy.
2. Confirmation of MDD diagnosis, a depression severity of moderate to severe based on MADRS score, inadequate response to current antidepressant medication, and absence of treatment-resistant depression, based on a diagnostic interview conducted by a clinician.
3. Participant has been on a stable dose of antidepressant medication (no more than 50% change) in the last month prior to Screening and has had an inadequate response, as judged by the Investigator.

### Exclusion Criteria

### MDD Participants:

Participants with the following will be excluded from study participation:
1. A reduction in the MADRS score of 25% or more between Screening and randomization.
2. Current or previous diagnosis of treatment-resistant MDD, defined as failure to respond to 2 or more antidepressant treatments given at an adequate dose for an adequate duration.
3. Current or previously diagnosed schizophrenia spectrum or other psychotic disorders, including schizophrenia, schizoaffective disorder, schizotypal disorder, schizophreniform disorder or brief psychotic disorder; current or previous history of bipolar disorder, or current personality disorder (as determined by MINI at Screening).
4. Currently receiving a monoamine oxidase inhibitor, tricyclic antidepressant, mirtazapine, an antipsychotic or a mood stabilizer.
5. Exposure to psilocin, or any other psychedelics, such as ayahuasca, mescaline, LSD or peyote more than 10 times in the last 10 years, or any psychedelic use within 6 months prior to Screening.
6. Use of a prescription medicine (except participants may take a stable chronic dose of antidepressant medication(s) and/or sedatives/hypnotics, and may take hormonal contraceptives, if applicable), certain herbal supplements (to be reviewed by the Investigator), or over-the-counter (OTC) medicine, during the 28 days before dosing. Exceptions include the following medications:
   a. Antianxiety medication, other than buspirone, is allowed if taken chronically (as needed or daily), provided that the participant must refrain from taking a dose of the antianxiety medication for at least 12 hours prior to a dose of study medication.
   b. Preexposure prophylaxis (PrEP) medication for prevention of HIV infection, such as TRUVADA® (emtricitabine and tenofovir disoproxil fumarate) or DESCOVY® (emtricitabine and tenofovir alafenamide), is allowed.
   c. Acetaminophen/paracetamol (less or equal 4 g/day) may be taken for up to 48 hours prior to dosing.
   d. Stable chronic therapy with hormone replacement medication is also allowed.

The Investigator and study team may review medication on a case-by-case basis to determine if its use would compromise participant safety or interfere with study procedures or data interpretation.

### NHV Participants:

Participants with the following will be excluded from study participation:
1. Current or previously diagnosed mental health disorder as defined by DSM-V criteria.
2. Exposure to psilocin, or any other psychedelics, such as ayahuasca, mescaline, LSD or peyote or any psychedelic use within 6 months prior to Screening.
3. Use of any prescription medicine (except participants may take hormonal contraceptives, if applicable), certain herbal supplements (to be reviewed by the Investigator), or over-the-counter (OTC) medicine, during the 28 days before dosing or need to take any prescription or OTC medicine or herbal supplement during the study. Acetaminophen/paracetamol (less or equal 4 g/day) may be taken for up to 48 hours prior to dosing.

The Investigator and study team may review medication on a case-by-case basis to determine if its use would compromise participant safety or interfere with study procedures or data interpretation.

### MDD and NHV Participants:

Participants with the following will be excluded from study participation:
1. Clinically significant risk of suicidality, as determined through a comprehensive psychiatric interview that incorporates the Columbia Suicide Severity Rating Scale (CSSRS); a score of 4 or higher on the suicidal ideation subscale of C-SSRS (lifetime) or any suicidal behaviour (lifetime), would be exclusionary.
2. Clinically relevant history of abnormal physical health interfering with the study as determined by medical history and physical examinations obtained during Screening as judged by the Investigator (including [but not limited to], neurological, endocrine, cardiovascular, respiratory, gastrointestinal (including dyspepsia or gastroesophageal reflux disease), hepatic, or renal disorder).
3. Diagnosis of hypertension or an arrhythmia.
4. Clinically relevant abnormal results for heart rate (resting supine heart rate>100 bpm) or blood pressure (resting supine systolic blood pressure (SBP) above 140 mmHg or diastolic blood pressure (DBP) above 90 mmHg) at screening. Screening supine SBP, DBP and heart rate for evaluation will be the average of 3 readings obtained after at least 5 minutes rest.
5. History of hypothyroidism and/or current abnormal thyroid function tests. In case of uncertain or questionable screening thyroid function test results, the TSH test may be repeated once during screening. The TSH test must be reviewed to ensure that it is within normal limits before randomizing a participant into the study.
6. Clinically relevant abnormal laboratory results (including hepatic and renal panels, complete blood count, chemistry panel and urinalysis), 12-lead ECG and vital signs, or physical findings at Screening. In case of uncertain or questionable results, tests performed during Screening may be repeated once to confirm eligibility or judged to be clinically irrelevant for healthy participants.
7. A family history of schizophrenia or schizoaffective disorder (first degree relatives), or bipolar disorder type 1 (first degree relatives).
8. Other eligibility considerations (i.e., participant personal circumstances, behavior, and/or any current problem that might interfere with participation or that is incompatible with establishment of rapport or safe exposure to psilocin), as judged by the Investigator.
9. Participants with abnormal vital signs which are out of range and deemed clinically significant by the Investigator at Screening or Day 1, following triplicate readings.
10. QT interval corrected for heart rate using Fridericia's formula (QTcF)>450 msec at Screening, following triplicate ECG readings.
11. Presence of clinically significant ECG abnormalities at the Screening visit, as defined by medical judgement.
12. History or clinical evidence of any disease and/or existence of any surgical or medical condition which might interfere with the absorption, distribution, metabolism or excretion (ADME) of the study drug.
13. Any other concomitant disease or condition that could interfere with, or for which the treatment might interfere with, the conduct of the study as outlined in this Protocol, or that would, in the opinion of the Investigator, pose an unacceptable risk to the participant in this study.
14. Participant is not fluent in English.
15. Participant has a presence or relevant history of any of the following medical conditions: organic brain disorders (e.g., epilepsy, seizure, intracranial hypertension, intracranial bleed and aneurysmal disease, brain tumor or other medical conditions associated with seizures or convulsions).
16. Aspartate aminotransferase (AST), alanine transaminase (ALT), gamma-glutamyl transferase (GGT) or total bilirubin levels≥1.5×the upper limit of normal (ULN) at Screening. These laboratory evaluations may be repeated once at the discretion of the Investigator. If the repeat test is within the reference range, the participant may be included only if the Investigator considers that the previous finding will not introduce additional risk factors and will not interfere with interpretation of safety data.
17. Positive test for hepatitis B surface antigen (HbsAg), anti-hepatitis C antibody (anti-HCV) or human immunodeficiency virus I and II (anti-HIV I/II) at Screening.
18. Positive urine test for drugs of abuse or alcohol breath test at Screening or Day-1. A positive test for cannabinoids (e.g., marijuana) at Screening may not exclude a participant if after discussion with and evaluation by the Investigator, the participant agrees not to use any marijuana or other cannabinoid products during the study, and if allowed to participate, the participant must test negative for cannabinoids on Day-1 (all participants), Day7 (NHV participants, double-blind study part), Day 14 (NHV participants, open-label study part), and Day 21 (MDD participants).
19. History of substance use disorder within the last year, as assessed by a structured clinical interview (Mini International Neuropsychiatric Interview [MINI], Version 7.0.2) or determined by self-report, or intake of >21 units of alcohol weekly, and the inability to refrain from alcohol use from 48 hours before Screening and each scheduled visit until discharge from the study site. One unit is equivalent to a 285 mL glass of full-strength beer or 1 (30 mL) measure of spirits or 1 glass (100 mL) of wine.
20. Participant who consumes excessive amounts of caffeine (e.g., coffee, tea, caffeinated sodas) or (methyl) xanthines (e.g., chocolate) based on the Investigator's determination and discretion.
21. The participant has participated in a clinical study and has received a medication or a new chemical entity within 3 months prior to dosing of current study medication.
22. Known sensitivity to psilocin and/or any excipients present in the formulation. Known fructose malabsorption or intolerance, since the orange drink vehicle for study drug contains fructose.
23. Participant is taking or has taken any drugs known to inhibit monoamine oxidase within 28 days prior to study drug administration.
24. Participant is taking or has taken OTC doses of 5-hydroxytryptophan or St John's Wort within 28 days prior to study drug administration.
25. Strenuous exercise within 48 hours prior to each visit, and while at the study site.
26. Donation of blood or plasma of >400 mL within 1 month prior to first dosing until 4 weeks after final dosing.
27. Participants capable of producing sperm who will not abstain from sperm donation between first dosing and 3 months after final dosing.
28. Participants of childbearing potential who are pregnant, breastfeeding or planning to conceive.
29. Known difficulty with obtaining intravenous access.

### Dose Stopping and Adjustment Criteria

### Cohort Stopping Criteria

If any of the following safety concerns are observed in a cohort, dosing of all participants at the given dose level or higher may be suspended/halted and all available data will be evaluated by the Safety Review Committee (SRC). Dose continuation (remaining participants within a cohort) or escalation will not proceed, without SRC review, for any of, but not limited to, the following reasons:
1. One or more Hy's Law cases in participants who have been administered psilocin-d₁₀; Hy's Law is defined as AST or ALT≥3×ULN and bilirubin≥2×ULN, in the absence of a significant increase in alkaline phosphatase (ALP) and in the absence of an alternative diagnosis that could explain the increase in bilirubin.
2. The occurrence of one serious adverse event (SAE) considered related to psilocin-d₁₀.
3. Two severe drug-related Aes in the same cohort, independent of within, or not within, the same system-organ class (SOC), considered related to psilocin-d₁₀.
4. Other findings that indicate dose escalation should be halted, e.g., if there is an unacceptable tolerability profile based on the nature, frequency and intensity of observed Aes and/or clinical safety monitoring in the opinion of the Investigator.
5. Two or more participants in a cohort find anxiety, fear, or distress caused by psilocin-d₁₀ to be intolerable during the dosing session, requiring rescue medication.
6. Two or more participants in a cohort develop clinically significant anxiety, fear, or distress following the dosing session that persists beyond the day of dosing, as assessed by the Investigator.
7. Psychedelic effects scales indicate either a complete mystical experience has been achieved in at least 75% of participants in two consecutive cohorts (MEQ30 scores>60), or that scores on the oceanic boundlessness subscale of the 5D-ASC>60 (percent of total maximum score) have been achieved in at least 75% of participants in two consecutive cohorts, or scores on the Intensity subscale of the HRS>2.79 have been achieved in at least 75% of participants in two consecutive cohorts or scores on the VAS “any drug effect” of 7 or higher have been achieved in at least 75% of participants in two consecutive cohorts.
8. If the psilocin-d₁₀ doses administered in two consecutive cohorts are associated with ratings on the VAS-any drug effect of >9 (>90 mm) in 75% of the participants, dose escalation will be stopped.
9. Clinically significant abnormalities in the laboratory parameters, vital signs or ECGs in 2 or more participants within the same cohort indicating dose-related intolerance, considered related to psilocin-d₁₀.
10. Two or more participants that receive psilocin-d₁₀ have QTc prolongation defined as an absolute QTcF value of >500 msec, or a change from baseline of >60 msec, that persists for at least 5 minutes following triplicate ECGs.
11. Two or more participants that receive psilocin-d₁₀ have tachycardia defined as resting supine heart rate>125 bpm, persisting for at least 10 minutes.
12. Two or more participants who receive psilocin-d₁₀ have symptomatic bradycardia defined as resting supine heart rate<45 bpm, or asymptomatic bradycardia defined as resting supine heart rate<30 bpm while awake persisting for at least 10 minutes.
13. Two or more participants who receive psilocin-d₁₀ develop hypertension defined as an increase in resting supine systolic blood pressure (BP)>40 mmHg to above 180 mmHg and persisting for at least 10 minutes.
14. Two or more participants who receive psilocin-d₁₀ develop more than one episode of symptomatic orthostatic hypotension, defined as a decrease of >20 mmHg for systolic BP, or asymptomatic systolic BP decrease of >30 mmHg which causes the systolic BP to fall below 100 mmHg, or >20 mmHg which causes the systolic BP to fall below 90 mmHg; or for single episodes associated with prolonged hypotension or a clinically significant event such as syncope, or other serious or severe cardiovascular signs or symptoms.
15. Two or more participants who are receiving psilocin-d₁₀ report treatment-emergent suicidality (an increase in Suicidal Ideation score to 4 or 5, or any Suicidal Behavior on the Since Last Visit of C-SSRS) that is considered related to the study drug.

### Individual Stopping Criteria

Individual study participant stopping criteria will include:
1. Any participant who experiences severe nausea or vomiting that can't be controlled by medical and/or nursing support.
2. Any participant who experiences a severe headache or migraine that can't be controlled by medical and/or nursing support.
3. Any participant who experiences intolerable anxiety, panic, or suicidal thoughts that can't be controlled by medical and/or nursing and/or psychological support.
4. Any participant who experiences enduring perceptual distortions beyond the day of dosing.
5. Any participant who experiences tachycardia defined as resting supine heart rate>125 bpm, persisting for at least 10 minutes.
6. Any participant who develops hypertension defined as an increase in resting supine systolic blood pressure (BP)>40 mmHg to above 180 mmHg and persisting for at least 10 minutes.
7. Investigator discretion based on clinical safety grounds.

For clarification, psilocin-d₁₀ is expected to cause commonly seen transient psychedelic drug effects, including auditory, visual, somatic, gustatory and olfactory hallucinations, synaesthesia, ideas of reference, mood elevation, time distortion, anxiety, agitation/restlessness, confusion, connectedness, loosening of associations, mystical experiences and ego dissolution. psilocin-d₁₀ is also expected to produce some physical effects including nausea, vomiting, headache and transient HR and BP increases. The Investigator and SRC may take these expected class effects into consideration in their safety monitoring and decision-making with respect to whether to continue or discontinue a participant from the study.

### Trial Stopping Rules

Dose escalation will be stopped if PK data indicates the predefined maximum exposure levels (i.e., Cₘₐₓ of 63.0 ng/ml or AUC₀₋₂₄ of 123 hr*ng/ml, combined male and female data) based on toxicokinetic data from the 28-day toxicology study in dogs have been achieved or is predicted to be achieved or exceeded should the dose be increased as planned. The exposure limits will be applied on mean values within a cohort.

The Sponsor will terminate the study if the occurrence of AEs or other findings suggests an unacceptable risk to the health of the participants. The study may also be terminated by regulatory authorities for safety or administrative reasons.

### Withdrawal Criteria

In addition to any participant meeting the individual stopping criteria specified in Individual Stopping Criteria, participants will be discontinued from the study prematurely if:
1. The participant requests to be withdrawn from the study;
2. Unacceptable toxicity develops;
3. A need for a concomitant medication prohibited by the protocol arises;
4. The Principal Investigator decides that it is in the participant's best interest;
5. The participant is noncompliant with the protocol, including any participant found to be positive for alcohol and/or urine drug testing;
6. Participant becomes pregnant during the study;
7. Sponsor decides to discontinue study.

### Handling of Withdrawals

If a participant withdraws from the study at any time, either at his or her request or at the Principal Investigator's discretion, the reason(s) for withdrawal will be recorded on the relevant page of the CRF. All final visit tasks (discharge procedures) will be completed for all participants who withdraw from the study. Participants withdrawn due to AEs will be monitored until resolution or stabilization of the AE. Withdrawn participants who withdraw for reasons other than for safety reasons and/or who meet stopping criteria may be replaced at the discretion and agreement between the Investigator and Sponsor.

### Study Medication Dosing and Clinical Assessments

### Participant Screening

After providing written informed consent, participants will have a Screening visit between 28 and 17 days prior to first dosing to ensure they meet the inclusion/exclusion criteria and are in good general health. Some of the Screening procedures may be performed remotely, while others will be required on-site. All screening assessments must be completed, and participants confirmed eligible by the Investigator based on inclusion and exclusion criteria review before the preparatory psychotherapy sessions described below can commence.

### Psychological Support Sessions

For MDD participants: Participants will have at least three preparatory psychotherapy session A1, A2 and A3 employing the EMBARK method, with the first two preparatory sessions occurring any time after eligibility screening and the third preparatory session occurring on or about Day −1 (see Table 22 for time windows). Each preparatory session will last for 90 minutes. Flexibility is allowed for the scheduling of these sessions at the facilitator's discretion and coordination with the participant and Investigator provided that all three preparatory sessions are completed prior to any study medication dosing. All preparatory psychotherapy visits are preferred in person, but psychotherapy session facilitators have the option to conduct any two of the three preparatory sessions and all of the integration sessions as remote/telemedicine visits.

For the last of three preparatory psychotherapy predose sessions, the preferred default schedule is the final preparatory session on Day-1 in the afternoon or evening before dosing, first dosing on Day 1, and the first postdose integration session on the morning of Day 2, so the participant completes these study activities during one extended visit.

For NHV participants: Participants will have at least one preparation session (prior to the first dose), a medicine session on Day 1 (first dose) and Day 8 (second dose), and at least one debrief session following each dose. The predose preparation sessions and debrief sessions can be done remotely. Additional preparation sessions or debrief sessions may be conducted by study facilitators at the Investigator's discretion.

Participants who are deemed eligible will be scheduled to return to the CRU for admission to the treatment period.

### Double-Blind Treatment Period (Cohorts 1-6)

### First Dosing Session

All participants will be admitted to the research site in the morning of Day −1 for the collection of baseline safety assessments and mood and psychological health questionnaires. Participants will wear a Holter monitor for approximately 6 hours initiated in the morning which will serve as a baseline Holter monitoring session.

Randomization to double-blind study medication will occur on Day 1 after successful completion of the three preparatory psychotherapy sessions (MDD participants) or one preparation session (NHV participants) and eligibility re-confirmation by the Investigator prior to dosing on Day 1.

Participants who are confirmed eligible and who are randomized will receive an oral dose of psilocin-d₁₀ or matched placebo in the morning of Day 1 in the fasted state. Participants will wear a Holter monitor for approximately 12 hours following dosing (Holter monitor should be placed approximately 1 hour prior to dosing and continue until approximately 12 hours postdose). Serial PK blood sampling will be collected predose and at 1, 6, 8, 12 and 24 hours postdose for MDD participants and predose and at 0.5, 1, 2, 4, 6, 8, 12 and 24 hours postdose for NHV participants. Other safety, efficacy and PD assessments will be performed as described in Table 16 for MDD participants and Table 17 for NHV participants.

The psilocin-d₁₀ medicine session may last up to 4 to 8 hours, and two trained facilitators will be present to support the participant throughout the dosing session for all participants. Participants will reside at the research site until the morning of Day 2 (until approximately 25 hours postdose) and will be discharged if the Investigator deems it safe to do so, following collection of safety assessments and PK blood samples, and completion of mood and psychological health questionnaires.

For NHV participants, after the Day 1 dose, participants will have at least one debrief session done either in person or remotely.

For MDD participants, after the Day 1 dose, postdose integration psychotherapy sessions, Sessions B1, B2 and B3, will occur on approximately Day 2, 10 and 21 following the dose (see Table 22 for time windows). Each postdose integration session will last for 90 minutes. These integration sessions are meant to be flexible and may occur on different days rather than a fixed weekly schedule depending on an assessment of an individual study participant by the facilitator, provided there are at least three postdose integration sessions following each study medication dose. Additional integration psychotherapy sessions may be conducted by study facilitators at the Investigator's discretion. All integration psychotherapy visits are preferred in person, but psychotherapy session facilitators have the option to conduct all of the integration sessions as remote/telemedicine visits.

Adverse event and concomitant medication monitoring will be performed while participants are in-clinic, during remote visits and outpatient visits. Participants will have approximately weekly telephonic assessments for the three weeks following study drug administration for MADRS assessments and for safety assessments (MDD participants) or for 1 to 2 weeks following study drug administration for safety assessments (NHV participants).

### Second Dosing Session

All participants will be re-admitted to the research site on Day 7 (NHV participants) or Day 21 (MDD participants) for the collection of safety assessments and mood and psychological health questionnaires to assess their response to the first dosing session. The same study procedures will be performed on Day 7 through Day 9 (NHV participants) or Day 21 through Day 23 (MDD participants) as were done on Days-1 through Day 2. All participants will receive an oral dose of psilocin-d₁₀ in the morning of Day 8 (NHV participants) or Day 22 (MDD participants). Two trained facilitators will be present to support the participant throughout the dosing session. There will be a +7 day window allowed for the Day 7 or Day 21 visit to allow for review of safety data, if needed.

Participants will reside at the research site until the morning of Day 9 (NHV participants) or Day 23 (MDD participants) and will be discharged if the Investigator deems it safe to do so, following collection of safety assessments and PK blood samples, and completion of mood and psychological health questionnaires.

For MDD participants, after the Day 22 dose, postdose integration psychotherapy sessions, Sessions C1, C2 and C3, will occur on approximately Day 23, 31 and 38 following the dose (see Table 22 for time windows). Each postdose integration session will last for 90 minutes. These integration sessions are meant to be flexible and may occur on different days rather than a fixed weekly schedule depending on an assessment of an individual study participant by the facilitator, provided there are at least three postdose integration sessions following each study medication dose. Additional integration psychotherapy sessions may be conducted by study facilitators at the Investigator's discretion. All integration psychotherapy visits are preferred in person, but psychotherapy session facilitators have the option to conduct all of the integration sessions as remote/telemedicine visits.

For NHV participants, after the Day 8 dose, participants will have a debrief session done either in person or remotely.

Adverse event and concomitant medication monitoring will be performed while participants are in-clinic, during remote visits and outpatient visits. Participants will have approximately weekly telephonic assessments for the three weeks following study drug administration for MADRS assessments and for safety assessments (MDD participants) or for 1 to 2 weeks following study drug administration for safety assessments (NHV participants).

### End of Treatment Visit (Day 42)

MDD participants will return to the research site for an outpatient visit on Day 42 for collection of safety assessments and completion of mood and psychological health questionnaires to assess their response to the second dosing session. Adverse event and concomitant medication monitoring will also be performed.

For participants who discontinue prematurely, this visit should be performed as an early termination visit and all assessments should be performed to the extent feasible.

### End of Study Visit (Day 28 or Day 56)

Participants will return to the research site for an outpatient end-of-study visit on Day 28 (±3 days) (NHV participants) or Day 56 (±3 days) (MDD participants) for collection of end-of-study safety. Adverse event and concomitant medication monitoring will also be performed.

### Follow-Up Remote Assessments

Remote MADRS assessments will be completed at 4-, 8- and 12-weeks post-Day 42 visit. The allowable time window for these assessments is ±3 days.

Optional follow up assessments using QIDS-SR16 will be delivered to and completed by participants at 4-, 8- and 12-weeks post-Day 42 visit using ePRO.

### Open-Label Treatment Period (Relative BA Cohort)

### First Dosing Session

NHV participants will be admitted to the research site in the morning of Day-1 for the collection of baseline safety assessments and mood and psychological health questionnaires. Participants will wear a Holter monitor for approximately 6 hours initiated in the morning which will serve as a baseline Holter session. This baseline Holter recording must be reviewed prior to dosing on Day 1 to confirm that there are no abnormal findings that may preclude a participant's involvement in the clinical trial.

Randomization to open-label study medication sequence 1 or 2 (with study medication assignment as described in Table 21) will occur on Day 1 after successful completion of the predose preparation session and eligibility re-confirmation by the Investigator prior to dosing on Day 1.

Participants who are confirmed eligible and who are randomized will receive an oral dose of psilocin-d₁₀ powder in solution or powder-filled capsules in the morning of Day 1 in the fasted state. Participants will wear a Holter monitor for approximately 12 hours following dosing (Holter monitor should be placed approximately 1 hour prior to dosing and continue until approximately 12 hours postdose). Serial PK blood sampling will be collected predose and at 0.5, 1, 2, 4, 6, 8, 12 and 24 hours postdose. Other safety and PD assessments will be performed as described in Table 18.

The psilocin-d₁₀ medicine session may last from 4 to 8 hours, and two trained facilitators will be present to support the participant throughout the dosing session. Participants will reside at the research site until the morning of Day 2 (until approximately 25 hours postdose) and will be discharged if the Investigator deems it safe to do so, following collection of safety assessments and PK blood samples, and completion of mood and psychological health questionnaires.

### Second Dosing Session

Participants will be re-admitted to the research site on Day 7 for the collection of safety assessments and mood and psychological health questionnaires. The same study procedures will be performed on Day 7 through Day 9 as were done on Days-1 through Day 2. Participants will crossover to the alternate study medication (psilocin-d₁₀ powder in solution or powder-filled capsule) in the morning of Day 8 in the fasted state.

Two trained facilitators will be present to support the participant throughout each dosing session. Participants will have at least one debrief session following each study medication dose. Adverse event and concomitant medication monitoring will also be performed during each of these visits. The preferred schedule is for participants to receive the two doses 1 week apart (i.e., Days 1 and 8). However, the time interval may be extended up to 2 weeks between doses at the Investigator's discretion. If the time interval between doses is extended to 2 weeks, participants will have approximately weekly telephonic safety assessments.

### End of Study Visit (Day 28)

Participants will return to the research site one week later for a final in-person Follow-up visit on Day 28 (±3 days) for collection of end-of-study safety assessments. Adverse event and concomitant medication monitoring will also be performed.

### Assessment of Efficacy and Pharmacokinetics

### Efficacy (Pharmacodynamics) Assessments

In addition to standard screening assessments, including medical and medication history, participants will undergo a number of screening tests to confirm the diagnosis of Major Depressive Disorder and establish disease severity as it relates to eligibility criteria. These tests will include the MADRS, MINI, and remote diagnostic interview by the central rater clinician.

### Montgomery-Åsberg Depression Rating Scale (MADRS)

The MADRS is a 10-item scale with ratings based on a clinical interview which moves from broadly phrased questions about symptoms to more detailed ones allowing a precise rating of severity. A rating scale from 0 to 6 is employed and the rater must decide whether the rating lies on defined scale steps (0, 2, 4, 6) or between them (1, 3, 5). The MADRS can be used for any time interval between ratings, be it weekly or otherwise, and the time interval should be recorded. The symptoms that are rated include: 1) apparent sadness, 2) reported sadness, 3) inner tension, 4) reduced sleep, 5) reduced appetite, 6) concentration difficulties, 7) lassitude, 8) inability to feel, 9) pessimistic thoughts, and 10) suicidal thoughts. Scores for each symptom are recorded and tallied, and the total score can range from 0 to 60 (Montgomery S A, Åsberg M (1979). A new depression scale designed to be sensitive to change. Br J Psychiatry, 134:382-389).

For MDD participants, the MADRS will be completed as part of the eligibility assessment at the Screening visit and must be completed prior to the start of the preparatory psychotherapy sessions. MADRS is also the primary efficacy endpoint assessed at various scheduled timepoints in-clinic and remotely as described in Table 16.

### Mini International Neuropsychiatric Interview (MINI 7.0.2)

The Mini International Neuropsychiatric Interview (MINI) for DSM-5, MINI 7.0.2 is a short, structured psychiatric interview that takes approximately 15 minutes to complete (Sheehan D V, Lecrubier Y, Harnett-Sheehan K, Amorim P, Janavs J, Weiller E, Hergueta T, Baker R, Dunbar G (1998): The Mini International Neuropsychiatric Interview (M.I.N.I.): The Development and Validation of a Structured Diagnostic Psychiatric Interview. J. Clin Psychiatry, 59 (suppl 20): 22-33). The major depressive episode component of the MINI asks over the past week, how much did you: 1) feel depressed, sad, empty or hopeless, 2) feel less interested in things or less able to enjoy the things you used to enjoy, 3) have a change in appetite or weight, 4) have trouble sleeping (difficulty falling asleep, waking up in the middle of the night, early morning awakening or sleeping excessively), 5) talk or move more slowly than normal or feel fidgety, or restless, 6) feel tired or without energy, 7) feel worthless or guilty, 8) have difficulty concentrating, thinking or making decisions, 9) have thoughts of hurting yourself, feel suicidal or wish that you were dead or any intent, or plan or attempt to kill yourself. Responses are scored on a scale of 0 to 4 (0=not at all, 1=a little, 2-moderately, 3=very, 4-extremely). Scores are summed to give a total score and divided by 9 to give a standardized score.

For MDD participants, the MINI will be completed as part of the eligibility assessment at the Screening visit and must be completed prior to the start of the preparatory psychotherapy sessions.

### Remote Diagnostic Interview

A remote diagnostic interview will be conducted by a central rater clinician for determination and confirmation of participant eligibility in conjunction with the Investigator's assessment. This interview will confirm confirmation of eligibility (i.e., diagnosis, severity, treatment history) and will include confirmation of MDD using the MADRS, and assessment of current antidepressant therapy (ADT), adequacy of duration and dose of prior and current antidepressant therapy, as well as degree of improvement.

For MDD participants, site staff will schedule the interview for the central rater, who will contact the participant as scheduled and conduct the interview. The remote diagnostic interview must be completed before the preparatory psychotherapy sessions are started.

### Psychedelic-Assisted Psychotherapy (EMBARK)

EMBARK for MDD is a six-domain framework for psychedelic-assisted psychotherapy; the six domains are: Existential-Spiritual, Mindfulness, Body Aware, Affective-Cognitive, Relational, and Keeping Momentum. A Treatment Manual for EMBARK: A Six-Domain Framework of Psilocin-Assisted Therapy for Major Depressive Disorder”, Version 1.0, 8 Jan. 2022 describes this approach to psychotherapy in great detail and will be used as a reference manual for this clinical trial.

For MDD participants, supportive psychotherapy, EMBARK for MDD will be provided during three or more sessions of preparatory psychotherapy (predose) sessions, and three or more sessions of integration psychotherapy (postdose) following each dose administration. During these sessions, participants will be paired with a dyad of two facilitators who will have completed specialized training in EMBARK for MDD; one or both facilitators will be a licensed psychotherapist. These sessions will be audio and videotape recorded which will be explained to participants and must be agreed to by participants as part of the informed consent process.

Participants will have a preparatory psychotherapy session A1, A2 and A3, with the first two preparatory sessions occurring after eligibility confirmation and the third session occurring and Day −1. Each preparatory session will last for 90 minutes. Flexibility is allowed for the scheduling of these sessions at the facilitator's discretion and coordination with the participant and Investigator provided that all three preparatory sessions are completed prior to any study medication dosing.

For the last of three preparatory psychotherapy predose sessions, the preferred default schedule is the final preparatory session on Day-1 in the afternoon or evening before dosing, first dosing on Day 1, and the first postdose integration session on the morning of Day 2, so the participant completes these study activities during one extended visit.

After each dose, a postdose integration psychotherapy session will occur on the morning after dosing and two additional postdose integration psychotherapy sessions will occur approximately 1 and 2 weeks after each study medication dose. Sessions B1, B2 and B3 will occur on approximately Day 2, 10 and 21 following the first dose and Sessions C1, C2 and C3 will occur on approximately Day 23, 31 and 38 following the second dose. Each postdose integration session will last for 90 minutes. These integration sessions are meant to be flexible and may occur on different days rather than a fixed weekly schedule depending on an assessment of an individual study participant by the facilitator, provided there are three postdose integration sessions following each study medication dose.

NHV participants will have at least one preparation session (prior to the first dose), a medicine session during each dose, and at least one debrief session following each dose. The predose preparation sessions and debrief sessions can be done remotely. Additional preparation sessions or debrief sessions may be conducted by study facilitators at the Investigator's discretion.

### Mystical Experience Questionnaire (MEQ30)

The revised Mystical Experience Questionnaire (MEQ30) consists of 30 questions looking back on the entirety of a dosing session, participants are asked to answer each question according to one's feelings, thoughts, and experiences at the time of the session. A rating scale from 0 to 5 is used with the categories of: 0=none, not at all; 1-so slight cannot decide; 2=slight; 3-moderate; 4=strong (equivalent in degree to any other strong experience); and 5=extreme (more than any other time in my life and stronger than 4).

The 30 questions are divided into grouped into factors or domains of mystical, positive mood, transcendence of time and space, and ineffability. The factor scores are computed by calculating the average response to individual questions within the factor. The MEQ30-total score is computed by taking the average response to all items.

The MEQ30 will be administered to all participants at the end of each study drug administration session, 4 to 10 hours postdose.

### 5-Dimensional Altered States of Consciousness Rating Scale (5D-ASC)

The 5-Dimensional Altered States of Consciousness Rating Scale (5D-ASC) consists of a set of 94 items that participants are asked to rate to what extent the statements apply to one's particular experience, compared to normal waking consciousness, by making a vertical mark on a line below each statement, whereby the mark to the far left of the line represents a response of “No, not more than usually” and the mark on the far right of the line represents a response of “Yes, much more than usually”. The questions are organized into 11 subscales consisting of experience of unity, spiritual experience, blissful state, insightfulness, disembodiment, impaired control and cognition, anxiety, complex imagery, elementary imagery, audio-visual synesthesia, and changed meaning of percepts. See Dittrich A (1998). The standardized psychometric assessment of altered states of consciousness (ASCs) in humans. Pharmacopsychiatry, 31(Suppl. 2):80-4; Dittrich A, Lamparter D, Maurer M (2010). 5D-ASC: Questionnaire for the assessment of altered states of consciousness. A short introduction. Zurich, Switzerland: PSIN PLUS (Translation from the original German by Felix Hasler and Rael Cahn); Curic S, Andreou C, Nolte G, Steinmann S, Thiebes S, Polomac N, Haaf M, Rauh J, Leicht G, Mulert C (2021). Ketamine Alters Functional Gamma and Theta Resting-State Connectivity in Healthy Humans: Implications for Schizophrenia Treatment Targeting the Glutamate System. Front. Psychiatry; Studerus E, Gamma A, Vollenweider F X (2010). Psychometric evaluation of the altered states of consciousness rating scale (OAV). PLOS One 5 (8), e12412.

The 5D-ASC will be administered to all participants at the end of each study drug administration session, 4 to 10 hours postdose.

### Hallucinogen Rating Scale (HRS)

The Hallucinogen Rating Scale (HRS) is a questionnaire with up to 100 items, and the questionnaire is designed to assess the subjective effects of hallucinogenic substances. Participants rate responses to the majority of questions on a 5-point intensity scale: 0=not at all; 1=slightly; 2-moderately; 3=quite a bit; and 4-extremely. Some questions have a slightly modified scale, and one question asks to rate the amount of time between when the drug was administered and feeling an effect from: no effect, 0-5 minutes, 5-15 minutes, 15-30 minutes, 30-60 minutes, or more than one hour. Questions are grouped into factors or domains including: 1) somaesthesia, 2) affect, 3) perception, 4) cognition, 5) volition, and 6) intensity (Strassman, 1994).

The HRS will be administered to all participants at the end of the study drug administration session, 4 to 10 hours postdose.

### Persisting Effects Questionnaire (PEQ)

The Persisting Effects Questionnaire (PEQ) is a 5-item questionnaire that assesses the meaningfulness, spiritual significance, psychological insightfulness, and how psychologically challenging was the experience during the study drug dosing session. Participants provide a response on an 8-point rating scale from ‘no more than routine’ experience to ‘single most meaningful experience’ for the first 3 items and from ‘no more than routine’ to ‘single most difficult or challenging’ experience to the fourth item above. In addition, participants rate the overall drug effect at peak intensity on a 0-4 point scale from 0=none, 1=possibly slight, 2=moderate, 3-strong, and 4=extreme (Griffiths R R, Richards W A, McCann U, Jesse R (2006). Psilocybin can occasion mystical-type experiences having substantial and sustained personal meaning and spiritual significance. Psychopharmacology (Berl). 187 (3):268-83; discussion 284-92).

The PEQ will be administered to all participants at the end of each study drug administration session, 4 to 10 hours postdose.

### 36-Item Short Form Survey (SF-36)

The 36-Item Short Form Health Survey (SF-36, Rand Corporation) is a set of generic quality-of-life measures, covering a range of items, including items such as general health and attitude, activity level, physical health or emotional problems with work, social activities, or regular daily activities, pain, energy level and mood. The SF-36 will be answered by MDD participants at various scheduled days in-clinic as described in Table 16.

### Any Drug Effect Visual Analogue Scale

A visual analogue scale will be completed by all participants on each day of dosing (Day 1 and Day 8 or Day 22) and will consist of a 100 mm bipolar line with the far left side of the line marked not at all, and the far right side of the line marked extremely. Participants will be asked “Right now, do you feel any drug effects at all?” and participants in response will make a vertical mark on the line on study dosing days predose and at 0.5, 1, 2, 4, 6, 8 and 12 hours postdose.

### Quick Inventory of Depressive Symptomatology (QIDS SR-16)

The self-report Quick Inventory of Depressive Symptomatology (QIDS SR-16) is a 16-item depression scale, yielding scores from 0-27, which covers the symptom domains of major depressive disorder, for the time frame of the past week: sleep disturbance (Questions 1-4), sad mood (Question 5), decrease or increase in appetite or weight (Questions 6-9), concentration (Question 10), self-criticism (Question 11), suicidal ideation (Question 12), interest (Question 13), energy/fatigue (Question 14), and psychomotor agitation/retardation (Questions 15-16) (Rush A J, Trivedi M H, Ibrahim H M, et al. (2003). The 16-item quick inventory of depressive symptomatology (QIDS), clinician rating (QIDS-C), and self-report (QIDS-SR): a psychometric evaluation in patients with chronic major depression. Biol Psychiatry 54 (5): 573-83). For MDD participants, QIDS SR-16 assessments will be completed prior to each study medication dose (Day −1, Day 21 and Day 42) and optional follow up assessments using QIDS-SR16 will be delivered to and completed by participants at 4-, 8-, and 12-weeks post-study using ePRO.

### Pharmacokinetics

### Blood Sample Collection and Processing

Serial blood samples (approximately 6 mL per sample will be collected into blood collection tubes containing K₂EDTA) for determination of plasma psilocin-d₁₀ concentrations.

For the double-blind part of the study, PK blood samples will be collected on Day 1 (first dose) and Day 8 or Day 22 (second dose) at predose (within 30 minutes prior to dosing, all participants) and at 1, 6, 8, 12 and 24 hours postdose (total: 12 PK samples per MDD participant) and at 0.5, 1, 2, 4, 6, 8, 12 and 24 hours postdose (total: 18 PK samples per NHV participant).

For the open-label part of the study, PK blood samples will be collected on Day 1 (first dose) and Day 8 (second dose) at predose (within 30 minutes prior to dosing) and at 0.5, 1, 2, 4, 6, 8, 12 and 24 hours postdose (total: 18 PK samples per NHV participant).

The plasma samples harvested will be stored at −70° C. until shipment to the bioanalytical laboratory for analysis. The study site will obtain the blood collection tubes, crushed ice and all required shipping supplies.

For the double-blind part of the study, a total of 72 mL (6 mL×6 samples×2 dosing sessions) of blood for MDD participants and 108 mL (6 mL×9 samples×2 dosing sessions) of blood for NHV participants will be collected over the 2 dosing sessions for plasma determination of psilocin-d₁₀. Additional blood samples (approximately 120 mL) will be obtained for clinical laboratory, pregnancy and serology evaluations, for a total of approximately 192 mL (for MDD participants) and 228 mL (for NHV participants) of blood collected during the study.

For the open-label part of the study, a total of 108 mL (6 mL×9 samples×2 dosing sessions) of blood for NHV participants will be collected over the 2 dosing sessions for plasma determination of psilocin-d₁₀. Additional blood samples (approximately 120 mL) will be obtained for clinical laboratory, pregnancy and serology evaluations, for a total of approximately 228 mL of blood collected during the study. Detailed instructions for blood collection, processing, and sample shipment will be provided in a separate PK Manual.

### Bioanalysis

Plasma samples will be extracted and analyzed for plasma psilocin-d₁₀ concentration determination using a validated LC/MS/MS analysis method at Pyxant Labs. Plasma samples and bioanalytical data will be archived at Pyxant Labs as per applicable regulations and as agreed upon with Cybin and will be provided to Clinilabs Drug Development Corporation through an agreed upon data transfer agreement.

### Assessment of Safety

The safety and tolerability of psilocin-d₁₀ will be assessed by the incidence of treatment-emergent adverse events, study discontinuation information, clinical laboratory test results, ECGs, Holter data, vital signs and physical examination findings.

### Adverse Events

### Reporting Adverse Events

The occurrence of treatment-emergent adverse events (TEAEs) and serious AEs (SAEs), reported through the completion of study will be collected and evaluated. AEs will be categorized by severity (intensity), and relationship to study medication. Clinical laboratory parameters (hematology, chemistry, urinalysis), ECG parameters, Holter monitoring, vital signs (seated SBP, DBP and pulse rate), and physical examination data will be collected and evaluated relative to baseline values.

### Concomitant Medications

Over-the-counter or prescription medications should not be taken during the study, including during the washout interval, unless approved by the Sponsor and Principal Investigator on a case-by-case basis. Participants will continue stable doses of antidepressants during the study and should not modify the doses unless medically necessary with physician's guidance and approval. Acetaminophen may be used sparingly (occasional prn use at the Investigator's discretion) if needed for mild analgesia. Participants may take sedative/hypnotic drugs (e.g. zopiclone, zaleplon or zolpidem) for sleep on an as needed basis, but not more than 3 times a week and not 12 hours before any scheduled assessment. MDD participants may take antianxiety drugs (e.g., benzodiazepines, hydroxyzine) on a chronic or as needed basis, but not 12 hours before study drug administration. Buspirone is an excluded/prohibited medication during the study.

Preexposure prophylaxis (PrEP) medication for prevention of HIV infection, such as TRUVADA® (emtricitabine and tenofovir disoproxil fumarate) or DESCOVY® (emtricitabine and tenofovir alafenamide), is allowed for MDD participants.

Participants are allowed to continue taking stable chronic doses of hormone replacement medications during the study. Participants using oral hormonal contraceptives should continue these medications during the study. Any prescription or over-the-counter medication taken during the study, including during the washout interval, will be recorded in the appropriate section of the CRF.

No other medication is allowed, including herbal remedies or vitamin supplements, without the consent of the Investigator.

Medication, which is considered necessary for the participant's safety and well-being, for example to manage intolerable and persistent anxiety, persistent elevations/changes in blood pressure, etc., may be given at the discretion of the Investigator during the residential period. When any such medication is required, it should be prescribed by the Investigator. Following consultation with the Sponsor Lead Physician or the Medical Monitor, and the Investigator should determine whether or not the participant should continue in the study.

### Statistical Methods

Details of the statistical and pharmacokinetic analyses presented below will be provided in the study's statistical analysis plan (SAP). A change to the data analysis methods described in the protocol will require a protocol amendment only if it alters a principal feature of the protocol. The SAP will be finalized prior to database lock. Any changes to the methods described in the final SAP will be described and justified in the clinical study report.

### General Considerations

Data collected in this study will be presented using summary tables and participant data listings. Continuous variables will be summarized using descriptive statistics, specifically the mean, median, standard deviation (SD), minimum and maximum. Categorical variables will be summarized by frequencies and percentages.

Statistical calculations will be done using SAS software (Version 9.4 or higher, SAS Institute, Cary, North Carolina, USA).

### Sample Size Determination

Up to 64 participants are planned to be enrolled into up to 7 cohorts, with up to 48 MDD participants and 16 NHV participants planned. Given the exploratory nature of this study, the sample size is not based on power calculations. The number of participants to be included is typical for this kind of study, and the results give sufficient safety and tolerability data, in addition to adequate description of the PK and PD parameters.

### Analysis Populations

Safety Population: All participants who receive at least one dose of study drug, psilocin-d₁₀ or placebo, will be included in the safety population and safety analyses.

Pharmacokinetic Population: All participants who receive at least one dose of psilocin-d₁₀ without any major protocol violations, who have sufficient plasma psilocin-d₁₀ concentration data for reliable estimates of the key pharmacokinetic variables, and who do not vomit within 4 hours of psilocin-d₁₀ study medication will be included in PK population and PK analyses.

Relative Bioavailability Population: All participants in the open-label part of the study who receive both powder in solution and powder-filled capsule formulations in the fasted state, who have sufficient plasma psilocin-d₁₀ concentration data for reliable estimates of the key pharmacokinetic variables, and who do not vomit within 4 hours of psilocin-d₁₀ study medication.

Pharmacodynamic (Efficacy) Population: All MDD participants who receive at least one dose of psilocin-d₁₀ who have at least one post-baseline MADRS assessment will be included in the PD population and PD (efficacy) analyses.

### Efficacy (Pharmacodynamics)

These questionnaires and rating scales will evaluate the psychedelic effects of psilocin-d₁₀ administration:
Mystical Experience Questionnaire (MEQ30)
5-Dimensional Altered States of Consciousness Rating Scale (5D-ASC)
Hallucinogen Rating Scale (HRS)
Visual Analogue Scale (VAS) ratings of ‘Any drug effect’
Persisting Effects Questionnaire (PEQ)

This standard efficacy assessment tool will be used to evaluate the clinical efficacy of psilocin-d₁₀ in treating the symptoms of MDD:
Montgomery-Åsberg Depression Rating Scale (MADRS) score

Between-group effects will use the MADRS baseline score after the 3 preparatory psychotherapy sessions (post-randomization) for change from baseline scores, and within-group effects for the entire treatment will use MADRS screening scores to reflect the entire course of treatment, including all the preparatory psychotherapy sessions, as well as post-randomization study drug dosing and integration psychotherapy sessions.

Descriptive statistics will be calculated for quantitative efficacy data and frequency counts will be compiled for classification of categorical efficacy data. Summary statistics will be calculated for each endpoint by treatment, time, and group.

Summary statistics will be calculated for each PD endpoint by treatment, time and group. Total values and changes from baseline will be calculated.

Pooled analyses across cohorts (pools consisting of cohorts 1-6, 2-6, 3-6, 4-6, 5-6) of within-group and between group effects will be calculated.

### Pharmacokinetics

Plasma psilocin-d₁₀ concentrations will be listed at each time point by participant and summarized by treatment at each time point using descriptive statistics (mean, standard deviation, coefficient of variation, median, minimum and maximum values). PK calculations will be performed based on actual time of blood sample collection, using noncompartmental methods, with the linear up-log down trapezoidal method, using Phoenix WinNonlin Version 8.3 or higher (Certara Inc, Princeton, New Jersey, USA).

PK parameters (AUC_{0-∞}, AUC0-t, and Cₘₐₓ, Tₘₐₓ, V_{z}/F, CL/F, Kₑₗ, and T_{1/2}) will be listed by participant and summarized by treatment. In addition to the descriptive statistics listed above, geometric means will be reported for the PK endpoints (AUC0-∞, AUC0-t, and Cₘₐₓ).

All plasma concentrations below the lower limit of quantitation (BLOQ) for the assay will be treated as “missing” in the PK analyses and summary statistics, except those that occur prior to the first quantifiable concentration on the day of dosing, or after the last quantifiable concentration, which will be treated as “0”.

No value of Kₑₗ (lz), AUC0-∞ or T_{1/2} will be reported for cases that do not exhibit a terminal log-linear phase in the concentration versus time profile. If the adjusted R-squared value (Rsq_adjusted) is <0.8, the analyst may evaluate if other time points are more appropriate and use those for the calculation of Kₑₗ, or no value of Kₑₗ, AUC0-∞ or T_{1/2} will be reported.

For determination of AUC0-∞, if calculated AUC from last to infinity is greater than 20% of the calculated AUC from 0 to infinity, then AUC_{0-¥} will not be reported.

### Planned Pharmacokinetic/Statistical Analyses:

The primary PK endpoints will be AUC0-∞, AUC0-t and Cmax, and the secondary PK endpoints will be Tₘₐₓ, V_{z}/F, CL/F, Kₑₗ, and T_{1/2}.

Relative Bioavailability Comparison: The geometric mean ratios and associated 90% confidence intervals between the powder in solution (Reference) and powder-filled capsule (Test) formulations administered in the fasted state will be provided for AUC0-∞, AUC0-t and Cₘₐₓ and will be used to assess the relative bioavailability of the two dosage forms.

### Demographic and Baseline Characteristics

Demographic and baseline characteristics will be summarized for all participants and for MDD and NHV participants using descriptive statistics. The presence/absence of any current medical condition and/or other significant medical history will be coded using Medical Dictionary for Regulatory Activities (MedDRA) Version 24.0, or higher. Medical and surgical history conditions will be listed, including reported term, System Organ Class (SOC) and Preferred Term, and year of diagnosis.

### Exposure to Study Drug

Each participant's exposure to study drug (psilocin-d₁₀ and placebo) will be presented in data listings.

### Prior/Concomitant Medications

Prior and concomitant medications will be coded using the World Health Organization (WHO) Drug Dictionary (March 2021 version or later). Prior medications will be those that start and end prior to first dose of study drug. Concomitant medications will be those that have a known end date after the first dose of study drug, or have a missing end date. Medications will be listed by participant including Anatomical Therapeutic Class (ATC) classification, preferred term and reported term; the start and end dates (or ongoing status); and dose, unit and indication. Medications taken by participants from 30 days prior to Screening until the end of treatment will be included in the listing.

Any non-drug therapies that are incurred during the course of the study will also be coded using MedDRA or WHO Drug Enhanced Dictionary, as appropriate, and will be listed by participant including start date, end date and indication.

### Safety Analyses

The safety and tolerability of a psilocin-d₁₀ will be assessed by the evaluation of treatment-emergent adverse events, study discontinuation information, laboratory test results, vital signs and physical examination findings.

Safety variables will be tabulated and presented for all participants and for MDD and NHV participants in the Safety Population. Data will be summarized by treatment. Adverse events will be coded using the Medical Dictionary for Regulatory Activities (MedDRA) for preferred term and system organ class and will be listed by participant. Treatment-emergent AEs (TEAEs) will be summarized by treatment, where treatment-emergent is defined as any event with a start date on or after the date and time of the first dose date and time of study drug within a treatment period. All TEAEs will be summarized by relationship to study drug and by intensity.

Potential abuse-related TEAEs will be summarized by category (i.e., euphoria-related terms; terms indicative of impaired attention, cognition, and mood; dissociative/psychotic terms; and related terms not captured elsewhere) and individual MedDRA Preferred Term.

12-lead ECGs will be centrally evaluated and measured by the central ECG laboratory (Clario), using a standard semi-automated method with measurements in lead II on 3 beats in each of 3 recordings. For the purpose of decisions on dose escalation, descriptive statistics on the interval data will be reported.

An evaluation of ECG effects of psilocin-d₁₀ may also be undertaken using 12-lead ECGs extracted from the Holter recordings with the EPQT method. The primary analysis will then be based concentration-QTc (C-QTc) modeling of the relationship between the plasma concentrations of psilocin-d₁₀ and change-from-baseline QTcF (ΔQTcF) with the intent to exclude an effect of placebo-corrected ΔQTcF (ΔΔQTcF)>10 msec at clinically relevant plasma concentrations. The effect of psilocin-d₁₀ on the placebo-corrected ΔQTcF, ΔHR (heart rate), ΔPR, and ΔQRS (ΔΔQTcF, ΔΔHR, ΔΔPR, and ΔΔQRS) will also be evaluated at each post-dosing time point (‘by-time point’ analysis) using the Intersection Union Test. In addition, an analysis of categorical outliers will be performed for changes in HR, PR, QRS, QTcF, T-wave morphology and U-wave presence.

Deaths, SAEs, and AEs resulting in study discontinuation will be tabulated and detailed in narratives.

All patents, patent applications, and other scientific or technical writings referred to anywhere herein are incorporated by reference herein in their entirety. The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the claims. Thus, it should be understood that although the present methods and compositions have been specifically disclosed by embodiments and optional features, modifications and variations of the concepts herein disclosed can be resorted to by those skilled in the art, and that such modifications and variations are considered to be within the scope of the compositions and methods as defined by the description and the appended claims.

Any single term, single element, single phrase, group of terms, group of phrases, or group of elements described herein can each be specifically excluded from the claims.

Whenever a range is given in the specification, for example, a temperature range, a time range, a composition, or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods.

In addition, where features or aspects of the compositions and methods are described in terms of Markush groups or other grouping of alternatives, those skilled in the art will recognize that the compositions and methods are also thereby described in terms of any individual member or subgroup of members of the Markush group or other group.

Accordingly, the preceding merely illustrates the principles of the methods and compositions. It will be appreciated that those skilled in the art will be able to devise various arrangements which, although not explicitly described or shown herein, embody the principles of the disclosure and are included within its spirit and scope. Furthermore, all examples and conditional language recited herein are principally intended to aid the reader in understanding the principles of the disclosure and the concepts contributed by the inventors to furthering the art, and are to be construed as being without limitation to such specifically recited examples and conditions. Moreover, all statements herein reciting principles, aspects, and embodiments of the disclosure as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present disclosure, therefore, is not intended to be limited to the exemplary embodiments shown and described herein. Rather, the scope and spirit of present disclosure is embodied by the following.

1. A method of treating a depressive disorder in a subject in need thereof, comprising administering orally to the subject about 8 mg to about 16 mg (free base equivalence) of a compound of Formula (I-3) 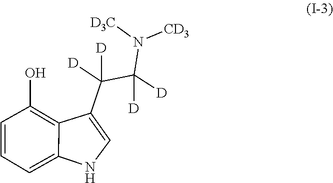 [SMILES: [2H]C([2H])([2H])N(C([2H])([2H])[2H])C([2H])([2H])C([2H])([2H])c1c[nH]c2cccc(O)c12]
or a pharmaceutically acceptable salt, polymorph, or solvate thereof.

2. The method of claim 1, comprising administering about 12 mg to about 16 mg (free base equivalence) of the compound of Formula (I-3), or a pharmaceutically acceptable salt, polymorph, or solvate thereof, to the subject.

3. The method of claim 1, comprising administering about 12 mg (free base equivalence) of the compound of Formula (I-3), or a pharmaceutically acceptable salt, polymorph, or solvate thereof, to the subject.

4. The method of claim 1, comprising administering about 14 mg (free base equivalence) of the compound of Formula (I-3), or a pharmaceutically acceptable salt, polymorph, or solvate thereof, to the subject.

5. The method of claim 1, comprising administering about 16 mg (free base equivalence) of the compound of Formula (I-3), or a pharmaceutically acceptable salt, polymorph, or solvate thereof, to the subject.

6. The method of claim 1, wherein a pharmaceutically acceptable salt of the compound of Formula (I-3) is administered.

7. The method of claim 6, wherein the pharmaceutically acceptable salt of the compound of Formula (I-3) is selected from the group consisting of a benzenesulfonate, a tartrate, a hemi-fumarate, an acetate, a citrate, a malonate, a fumarate, a succinate, an oxalate, a benzoate, a salicylate, an ascorbate, a hydrochloride, a maleate, a malate, a methanesulfonate, a toluenesulfonate, a glucuronate, and a glutarate salt, of the compound of Formula (I-3).

8. The method of claim 6, wherein the pharmaceutically acceptable salt of the compound of Formula (I-3) is a benzenesulfonate salt of the compound of Formula (I-3).

9. The method of claim 8, wherein the benzenesulfonate salt of the compound of Formula (I-3) is a crystalline benzenesulfonate salt (I-3a), characterized by an X-ray powder diffraction pattern containing at least three characteristic peaks selected from 7.023, 7.767, 11.822, 12.550, 12.860, 13.994, 15.521, 18.436, 19.503, 20.760, 21.070, 22.007, 22.745, 23.340, 24.187, 25.532, 26.880, 27.856, 28.163, 31.267, 33.024, 35.030, 36.835, 39.312, 40.545, and 40.988 °2θ (±0.2° 2θ).

10. The method of claim 6, wherein the pharmaceutically acceptable salt of the compound of Formula (I-3) is a tartrate salt of the compound of Formula (I-3).

11. The method of claim 10, wherein the tartrate salt of the compound of Formula (I-3) is a crystalline tartrate salt (I-3b), characterized by an X-ray powder diffraction pattern containing at least three characteristic peaks selected from 6.732, 12.708, 13.470, 14.774, 15.921, 16.268, 17.295, 18.869, 20.079, 20.208, 20.877, 21.894, 22.657, 23.491, 23.702, 24.636, 24.882, 25.569, 26.685, 27.060, 27.502, 28.179, 28.597, 29.035, 29.257, 29.527, 31.017, 31.527, 32.059, 32.307, 33.012, 34.024, 34.388, 34.905, 35.361, 36.183, 37.372, 37.764, 38.657, and 41.049 °2θ (±0.2° 2θ).

12. The method of claim 6, wherein the pharmaceutically acceptable salt of the compound of Formula (I-3) is a hemi-fumarate salt of the compound of Formula (I-3).

13. The method of claim 12, wherein the hemi-fumarate salt of the compound of Formula (I-3) is a crystalline hemi-fumarate salt (I-3c), characterized by an X-ray powder diffraction pattern containing at least three characteristic peaks selected from 9.713, 11.209, 11.605, 12.338, 12.852, 13.718, 15.117, 16.066, 16.627, 19.026, 19.427, 20.108, 21.068, 21.335, 21.837, 22.429, 23.262, 23.478, 23.900, 24.720, 25.318, 27.912, 28.532, 29.565, 30.457°, 32.698, 34.155, 37.910, 39.566, and 40.999 °2θ (±0.2° 2θ).

14. The method of claim 6, wherein the pharmaceutically acceptable salt of the compound of Formula (I-3) is a citrate salt of the compound of Formula (I-3).

15. The method of claim 14, wherein the citrate salt (I-3e) is amorphous by X-ray powder diffraction.

16. The method of claim 6, wherein the pharmaceutically acceptable salt of the compound of Formula (I-3) is a benzoate salt of the compound of Formula (I-3).

17. The method of claim 16, wherein the benzoate salt of the compound of Formula (I-3) is a crystalline benzoate salt (I-3j), characterized by an X-ray powder diffraction pattern containing at least three characteristic peaks selected from 9.486, 11.006, 12.379, 13.428, 14.608, 15.446, 16.389, 18.247, 18.977, 19.346, 19.831, 20.868, 21.447, 22.860, 23.878, 24.944, 25.737, 26.144, 26.341, 26.990°, 27.708, 28.595, 30.048, 30.763, 31.127, 31.839, 32.800, 34.460, 35.444, 37.725, and 38.597 °2θ (±0.2° 2θ).

18. The method of claim 1, wherein the depressive disorder is major depressive disorder (MDD).

19. The method of claim 18, wherein, prior to treatment, the subject has been diagnosed with moderate to severe major depressive disorder as defined by the Diagnostic and Statistical Manual of Mental Disorders, 5th edition (DSM-5).

20. The method of claim 1, wherein, prior to treatment, the subject has scored greater than or equal to 21 on the Montgomery-Åsberg Depression Scale (MADRS).

21. The method of claim 1, wherein a first dose and a second dose, each being about 8 mg to about 16 mg (free base equivalence) of the compound of Formula (I-3), or a pharmaceutically acceptable salt, polymorph, or solvate thereof, are administered to the subject one to four weeks apart (±3 days).

22. The method of claim 1, wherein a first dose and a second dose, each being about 8 mg to about 16 mg (free base equivalence) of the compound of Formula (I-3), or a pharmaceutically acceptable salt, polymorph, or solvate thereof, are administered to the subject three weeks apart (±3 days).

23. The method of claim 1, wherein the subject is taking antidepressant medication as part of ongoing treatment and the method is used as adjunctive therapy.

24. A pharmaceutical composition, comprising:
about 8 mg to about 16 mg (free base equivalence) of a compound of Formula (I-3) 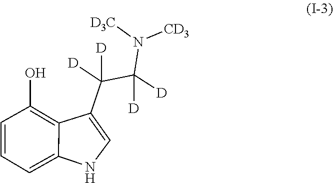 [SMILES: [2H]C([2H])([2H])N(C([2H])([2H])[2H])C([2H])([2H])C([2H])([2H])c1c[nH]c2cccc(O)c12]
or a pharmaceutically acceptable salt, polymorph, or solvate thereof; and
a pharmaceutically acceptable vehicle.

25. The pharmaceutical composition of claim 24, wherein the pharmaceutical composition comprises about 12 mg to about 16 mg (free base equivalence) of the compound of Formula (I-3), or a pharmaceutically acceptable salt, polymorph, or solvate thereof.

26. The pharmaceutical composition of claim 24, wherein the pharmaceutical composition comprises about 12 mg (free base equivalence) of the compound of Formula (I-3), or a pharmaceutically acceptable salt, polymorph, or solvate thereof.

27. The pharmaceutical composition of claim 24, wherein the pharmaceutical composition comprises about 14 mg (free base equivalence) of the compound of Formula (I-3), or a pharmaceutically acceptable salt, polymorph, or solvate thereof.

28. The pharmaceutical composition of claim 24, wherein the pharmaceutical composition comprises about 16 mg (free base equivalence) of the compound of Formula (I-3), or a pharmaceutically acceptable salt, polymorph, or solvate thereof.

29. The pharmaceutical composition of claim 24, wherein the pharmaceutical composition comprises a pharmaceutically acceptable salt of the compound of Formula (I-3).

30. The pharmaceutical composition of claim 29, wherein the pharmaceutically acceptable salt of the compound of Formula (I-3) is selected from the group consisting of a benzenesulfonate, a tartrate, a hemi-fumarate, an acetate, a citrate, a malonate, a fumarate, a succinate, an oxalate, a benzoate, a salicylate, an ascorbate, a hydrochloride, a maleate, a malate, a methanesulfonate, a toluenesulfonate, a glucuronate, and a glutarate salt, of the compound of Formula (I-3).

31. The pharmaceutical composition of claim 29, wherein the pharmaceutically acceptable salt of the compound of Formula (I-3) is a benzenesulfonate salt of the compound of Formula (I-3).

32. The pharmaceutical composition of claim 31, wherein the benzenesulfonate salt of the compound of Formula (I-3) is a crystalline benzenesulfonate salt (I-3a), characterized by an X-ray powder diffraction pattern containing at least three characteristic peaks selected from 7.023, 7.767, 11.822, 12.550, 12.860, 13.994, 15.521, 18.436, 19.503, 20.760, 21.070, 22.007, 22.745, 23.340, 24.187, 25.532, 26.880, 27.856, 28.163, 31.267, 33.024, 35.030, 36.835, 39.312, 40.545, and 40.988 °2θ (±0.2° 2θ).

33. The pharmaceutical composition of claim 29, wherein the pharmaceutically acceptable salt of the compound of Formula (I-3) is a tartrate salt of the compound of Formula (I-3).

34. The pharmaceutical composition of claim 33, wherein the tartrate salt of the compound of Formula (I-3) is a crystalline tartrate salt (I-3b), characterized by an X-ray powder diffraction pattern containing at least three characteristic peaks selected from 6.732, 12.708, 13.470, 14.774, 15.921, 16.268, 17.295, 18.869, 20.079, 20.208, 20.877, 21.894, 22.657, 23.491, 23.702, 24.636, 24.882, 25.569, 26.685, 27.060, 27.502, 28.179, 28.597, 29.035, 29.257, 29.527, 31.017, 31.527, 32.059, 32.307, 33.012, 34.024, 34.388, 34.905, 35.361, 36.183, 37.372, 37.764, 38.657, and 41.049 °2θ (±0.2° 2θ).

35. The pharmaceutical composition of claim 29, wherein the pharmaceutically acceptable salt of the compound of Formula (I-3) is a hemi-fumarate salt of the compound of Formula (I-3).

36. The pharmaceutical composition of claim 35, wherein the hemi-fumarate salt of the compound of Formula (I-3) is a crystalline hemi-fumarate salt (I-3c), characterized by an X-ray powder diffraction pattern containing at least three characteristic peaks selected from 9.713, 11.209, 11.605, 12.338, 12.852, 13.718, 15.117, 16.066, 16.627, 19.026, 19.427, 20.108, 21.068, 21.335, 21.837, 22.429, 23.262, 23.478, 23.900, 24.720, 25.318, 27.912, 28.532, 29.565, 30.457°, 32.698, 34.155, 37.910, 39.566, and 40.999°2θ (±0.2° 2θ).

37. The pharmaceutical composition of claim 29, wherein the pharmaceutically acceptable salt of the compound of Formula (I-3) is a citrate salt of the compound of Formula (I-3).

38. The pharmaceutical composition of claim 37, wherein the citrate salt (I-3e) is amorphous by X-ray powder diffraction.

39. The pharmaceutical composition of claim 29, wherein the pharmaceutically acceptable salt of the compound of Formula (I-3) is a benzoate salt of the compound of Formula (I-3).

40. The pharmaceutical composition of claim 39, wherein the benzoate salt of the compound of Formula (I-3) is a crystalline benzoate salt (I-3j), characterized by an X-ray powder diffraction pattern containing at least three characteristic peaks selected from 9.486, 11.006, 12.379, 13.428, 14.608, 15.446, 16.389, 18.247, 18.977, 19.346, 19.831, 20.868, 21.447, 22.860, 23.878, 24.944, 25.737, 26.144, 26.341, 26.990°, 27.708, 28.595, 30.048, 30.763, 31.127, 31.839, 32.800, 34.460, 35.444, 37.725, and 38.597°2θ (±0.2° 2θ).

41. The pharmaceutical composition of claim 24, wherein the pharmaceutically acceptable vehicle comprises an organic acid agent.

42. The pharmaceutical composition of claim 41, wherein the organic acid agent is citric acid.

43. The pharmaceutical composition of claim 41, wherein the organic acid agent is present in the pharmaceutical composition in an amount of at least 2% by weight and up to 10% by weight, based on a total weight of the pharmaceutical composition (on a dry basis).

44. The pharmaceutical composition of claim 24, wherein the pharmaceutical composition is in a solid dosage form.

45. The pharmaceutical composition of claim 44, wherein the solid dosage form is a solid dosage form adapted for oral administration.

46. The pharmaceutical composition of claim 44, wherein the solid dosage form is a powder in capsule dosage form.

47. The pharmaceutical composition of claim 24, wherein the pharmaceutical composition is an oral liquid dosage form.

48. A method of treating a depressive disorder in a subject in need thereof, comprising administering to the subject the pharmaceutical composition of claim 24.

49. The method of claim 48, wherein the pharmaceutical composition is administered orally to the subject.

50. The method of claim 48, wherein the pharmaceutical composition is administered by reconstituting the pharmaceutical composition in solid dosage form in a pharmaceutically acceptable aqueous medium to form an oral liquid dosage form, followed by administering orally to the subject the oral liquid dosage form.

51. The method of claim 48, wherein the depressive disorder is major depressive disorder (MDD).

52. The method of claim 51, wherein, prior to treatment, the subject has been diagnosed with moderate to severe major depressive disorder as defined by the Diagnostic and Statistical Manual of Mental Disorders, 5th edition (DSM-5).

53. The method of claim 48, wherein, prior to treatment, the subject has scored greater than or equal to 21 on the Montgomery-Åsberg Depression Scale (MADRS).

54. The method of claim 48, wherein a first dose and a second dose of the pharmaceutical composition, each comprising about 8 mg to about 16 mg (free base equivalence) of the compound of Formula (I-3), or a pharmaceutically acceptable salt, polymorph, or solvate thereof, are administered to the subject one to four weeks apart (±3 days).

55. The method of claim 48, wherein a first dose and a second dose of the pharmaceutical composition, each comprising about 8 mg to about 16 mg (free base equivalence) of the compound of Formula (I-3), or a pharmaceutically acceptable salt, polymorph, or solvate thereof, are administered to the subject three weeks apart (±3 days).

56. The method of claim 48, wherein the subject is taking antidepressant medication as part of ongoing treatment and the method is used as adjunctive therapy.

57. An adjunctive therapy method for treating a depressive disorder in a subject taking an antidepressant medication as part of ongoing treatment, the adjunctive therapy method comprising administering to the subject about 8 mg to about 16 mg (free base equivalence) of a compound of Formula (I-3) 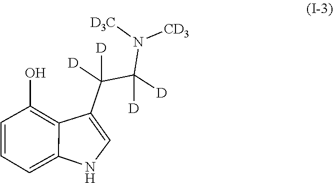 [SMILES: [2H]C([2H])([2H])N(C([2H])([2H])[2H])C([2H])([2H])C([2H])([2H])c1c[nH]c2cccc(O)c12]
or a pharmaceutically acceptable salt, polymorph, or solvate thereof.

58. The adjunctive therapy method of claim 57, wherein the depressive disorder is major depressive disorder (MDD).

59. The adjunctive therapy method of claim 58, wherein, prior to the adjunctive therapy, the subject has been diagnosed with moderate to severe major depressive disorder as defined by the Diagnostic and Statistical Manual of Mental Disorders, 5th edition (DSM-5).

60. The adjunctive therapy method of claim 57, wherein, prior to the adjunctive therapy, the subject has scored greater than or equal to 21 on the Montgomery-Åsberg Depression Scale (MADRS).

61. The adjunctive therapy method of claim 57, wherein a first dose and a second dose, each being about 8 mg to about 16 mg (free base equivalence) of the compound of Formula (I-3), or a pharmaceutically acceptable salt, polymorph, or solvate thereof, are administered to the subject one to four weeks apart (±3 days).

62. The adjunctive therapy method of claim 57, wherein a first dose and a second dose, each being about 8 mg to about 16 mg (free base equivalence) of the compound of Formula (I-3), or a pharmaceutically acceptable salt, polymorph, or solvate thereof, are administered to the subject three weeks apart (±3 days).

63. The adjunctive therapy method of claim 57, wherein the antidepressant medication is a selective serotonin reuptake inhibitor (SSRI), a serotonin and noradrenaline reuptake inhibitor (SNRI), or a combination thereof.

64. The adjunctive therapy method of claim 57, wherein the compound of Formula (I-3), or a pharmaceutically acceptable salt, polymorph, or solvate thereof, is administered orally to the subject.

65. A method of treating major depressive disorder (MDD) in a subject in need thereof, comprising administering orally to the subject a capsule comprising about 8 mg to about 16 mg (free base equivalence) of a benzenesulfonate salt of a compound of Formula (I-3)

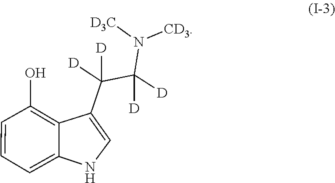 [SMILES: [2H]C([2H])([2H])N(C([2H])([2H])[2H])C([2H])([2H])C([2H])([2H])c1c[nH]c2cccc(O)c12]

66. A method of treating major depressive disorder (MDD) in a subject in need thereof, comprising administering orally to the subject an oral liquid dosage form comprising:
(i) about 8 mg to about 16 mg (free base equivalence) of a compound of Formula (I-3) 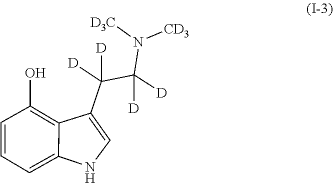 [SMILES: [2H]C([2H])([2H])N(C([2H])([2H])[2H])C([2H])([2H])C([2H])([2H])c1c[nH]c2cccc(O)c12]
or a pharmaceutically acceptable salt, polymorph, or solvate thereof; and
(ii) a pharmaceutically acceptable aqueous medium.
